SEQUENCE LISTING (1760 sequences)

SEQ ID NO: 1 - DNA, 25 bp, Artificial Sequence
  synthetic nucleotide probe
atggagctgactctcatcgactatg

SEQ ID NO: 2 - DNA, 25 bp, Artificial Sequence
  synthetic nucleotide probe
atatggtgcattatcatccttctaa

SEQ ID NO: 3 - DNA, 25 bp, Artificial Sequence
  synthetic nucleotide probe
agtcctctggtctatctcatgaaac

SEQ ID NO: 4 - DNA, 25 bp, Artificial Sequence
  synthetic nucleotide probe
cttgcctccccactgataggaaggt

SEQ ID NO: 5 - DNA, 25 bp, Artificial Sequence
  synthetic nucleotide probe
caaaagccgtcaaagaccttgcctc

SEQ ID NO: 6 - DNA, 25 bp, Artificial Sequence
  synthetic nucleotide probe
gattttgtacatagtcctctggtct

SEQ ID NO: 7 - DNA, 25 bp, Artificial Sequence
  synthetic nucleotide probe
gccactacacttcttaaggcgagca

SEQ ID NO: 8 - DNA, 25 bp, Artificial Sequence
  synthetic nucleotide probe
gataggaaggtcctaggctgccgtg

SEQ ID NO: 9 - DNA, 25 bp, Artificial Sequence
  synthetic nucleotide probe
atccttctaaggtagcagcagctgc

SEQ ID NO: 10 - DNA, 25 bp, Artificial Sequence
  synthetic nucleotide probe
actccgtaacagattctggaccaac

SEQ ID NO: 11 - DNA, 25 bp, Artificial Sequence
  synthetic nucleotide probe
gaccaaccttcaactatcttcttga

SEQ ID NO: 12 - DNA, 25 bp, Artificial Sequence
  synthetic nucleotide probe
gaaagatgaactctgcaggctaaga

SEQ ID NO: 13 - DNA, 25 bp, Artificial Sequence
  synthetic nucleotide probe
acaagatgaacaagtcggacttcct

SEQ ID NO: 14 - DNA, 25 bp, Artificial Sequence
  synthetic nucleotide probe
tggctcctaggaatgcttggtgctg

SEQ ID NO: 15 - DNA, 25 bp, Artificial Sequence
  synthetic nucleotide probe
gatatgattcggatcctgtgaaggc

SEQ ID NO: 16 - DNA, 25 bp, Artificial Sequence
  synthetic nucleotide probe
aaagaaagagtccatcagatttgtg

SEQ ID NO: 17 - DNA, 25 bp, Artificial Sequence
  synthetic nucleotide probe
gaataatcaggctcgctttatctta

SEQ ID NO: 18 - DNA, 25 bp, Artificial Sequence
  synthetic nucleotide probe
cttggtgctgaatctgctaaactga

SEQ ID NO: 19 - DNA, 25 bp, Artificial Sequence
  synthetic nucleotide probe
aagaacaagagctggacacattaaa

SEQ ID NO: 20 - DNA, 25 bp, Artificial Sequence
  synthetic nucleotide probe
gagacttttttgaactcagacttaa

SEQ ID NO: 21 - DNA, 25 bp, Artificial Sequence
  synthetic nucleotide probe
gtacaactcgtatttatctctgatg

SEQ ID NO: 22 - DNA, 25 bp, Artificial Sequence
  synthetic nucleotide probe
gaatgtttgacttcgtattgaccct

SEQ ID NO: 23 - DNA, 25 bp, Artificial Sequence
  synthetic nucleotide probe
ggatgctgaaatttttcccatggaa

SEQ ID NO: 24 - DNA, 25 bp, Artificial Sequence
  synthetic nucleotide probe
acttcgtattgacccttatctgtaa

SEQ ID NO: 25 - DNA, 25 bp, Artificial Sequence
  synthetic nucleotide probe
caatatatcatcctttggcatccca

SEQ ID NO: 26 - DNA, 25 bp, Artificial Sequence
  synthetic nucleotide probe
cgctctcgtttcattttctgcagcg

SEQ ID NO: 27 - DNA, 25 bp, Artificial Sequence
  synthetic nucleotide probe
tattcttctctttagacgacctctt

SEQ ID NO: 28 - DNA, 25 bp, Artificial Sequence
  synthetic nucleotide probe
tctctttagacgacctcttccaaaa

SEQ ID NO: 29 - DNA, 25 bp, Artificial Sequence
  synthetic nucleotide probe
ctacctccatttagggatttgcttg

SEQ ID NO: 30 - DNA, 25 bp, Artificial Sequence
  synthetic nucleotide probe
gtgtctcagagctgttaagggctta

SEQ ID NO: 31 - DNA, 25 bp, Artificial Sequence
  synthetic nucleotide probe
ccctcaatctagaacgctacacaag

SEQ ID NO: 32 - DNA, 25 bp, Artificial Sequence
  synthetic nucleotide probe
gaggccatgtgtctcagagctgtta

SEQ ID NO: 33 - DNA, 25 bp, Artificial Sequence
  synthetic nucleotide probe
ttagggatttgcttgggatacagaa

SEQ ID NO: 34 - DNA, 25 bp, Artificial Sequence
  synthetic nucleotide probe
gtgctctacctccatttagggattt

SEQ ID NO: 35 - DNA, 25 bp, Artificial Sequence
  synthetic nucleotide probe
aaataggaacacgtgctctacctcc

SEQ ID NO: 36 - DNA, 25 bp, Artificial Sequence
  synthetic nucleotide probe
gggatacagaagaggccatgtgtct

SEQ ID NO: 37 - DNA, 25 bp, Artificial Sequence
  synthetic nucleotide probe
gaagaggccatgtgtctcagagctg

SEQ ID NO: 38 - DNA, 25 bp, Artificial Sequence
  synthetic nucleotide probe
cagagctgttaagggcttatttttt

SEQ ID NO: 39 - DNA, 25 bp, Artificial Sequence
  synthetic nucleotide probe
cattggagtcatagcatgtgtgtaa

SEQ ID NO: 40 - DNA, 25 bp, Artificial Sequence
  synthetic nucleotide probe
gaacttgctatgtaatttgtgtcta

SEQ ID NO: 41 - DNA, 25 bp, Artificial Sequence
  synthetic nucleotide probe
gatggtgatgttcacctggcaatca

SEQ ID NO: 42 - DNA, 25 bp, Artificial Sequence
  synthetic nucleotide probe
gagccaccaggaaggcgcatcttag

SEQ ID NO: 43 - DNA, 25 bp, Artificial Sequence
  synthetic nucleotide probe
ttgacccaccttgagagagagccac

SEQ ID NO: 44 - DNA, 25 bp, Artificial Sequence
  synthetic nucleotide probe
ggacactaagtccattgttacatga

SEQ ID NO: 45 - DNA, 25 bp, Artificial Sequence
  synthetic nucleotide probe
gaaatgatttcctggctggccaact

SEQ ID NO: 46 - DNA, 25 bp, Artificial Sequence
  synthetic nucleotide probe
acactggttttcatgcgctgttttt

SEQ ID NO: 47 - DNA, 25 bp, Artificial Sequence
  synthetic nucleotide probe
actgattactggctgtgtcttgggt

SEQ ID NO: 48 - DNA, 25 bp, Artificial Sequence
  synthetic nucleotide probe
tggacctagactgtgcttttctgtc

SEQ ID NO: 49 - DNA, 25 bp, Artificial Sequence
  synthetic nucleotide probe
ttgggtgggcagaaactcgaacttg

SEQ ID NO: 50 - DNA, 25 bp, Artificial Sequence
  synthetic nucleotide probe
acctggcaatcagctgagttgagac

SEQ ID NO: 51 - DNA, 25 bp, Artificial Sequence
  synthetic nucleotide probe
gaaggcactgctgggagccattaga

SEQ ID NO: 52 - DNA, 25 bp, Artificial Sequence
  synthetic nucleotide probe
caggctcatgggcaggcacaagaag

SEQ ID NO: 53 - DNA, 25 bp, Artificial Sequence
  synthetic nucleotide probe
gtcttagtcgtgaccacatggctgt

SEQ ID NO: 54 - DNA, 25 bp, Artificial Sequence
  synthetic nucleotide probe
cacagatacaagagaagccaggagg

SEQ ID NO: 55 - DNA, 25 bp, Artificial Sequence
  synthetic nucleotide probe
aaggggctgtgtgaaggcactgctg

SEQ ID NO: 56 - DNA, 25 bp, Artificial Sequence
  synthetic nucleotide probe
agaagaactgaggagtggtggacca

SEQ ID NO: 57 - DNA, 25 bp, Artificial Sequence
  synthetic nucleotide probe
gccaggaggtctatgatggtgacga

SEQ ID NO: 58 - DNA, 25 bp, Artificial Sequence
  synthetic nucleotide probe
cccacctggcatatactggctggcc

SEQ ID NO: 59 - DNA, 25 bp, Artificial Sequence
  synthetic nucleotide probe
acatggctgttgtcaggtcgtgctg

SEQ ID NO: 60 - DNA, 25 bp, Artificial Sequence
  synthetic nucleotide probe
tctatggggatgtgctctacttcct

SEQ ID NO: 61 - DNA, 25 bp, Artificial Sequence
  synthetic nucleotide probe
gcatggaaaccatcacagcttgcct

SEQ ID NO: 62 - DNA, 25 bp, Artificial Sequence
  synthetic nucleotide probe
gagtggtggaccaggctcgaacact

SEQ ID NO: 63 - DNA, 25 bp, Artificial Sequence
  synthetic nucleotide probe
ttggagggacaaagctaattgatct

SEQ ID NO: 64 - DNA, 25 bp, Artificial Sequence
  synthetic nucleotide probe
gatgccaaggccacaaaagccaaga

SEQ ID NO: 65 - DNA, 25 bp, Artificial Sequence
  synthetic nucleotide probe
ccaggctcgaacactggccgaggag

SEQ ID NO: 66 - DNA, 25 bp, Artificial Sequence
  synthetic nucleotide probe
tgacagagcaccgcgacggattcca

SEQ ID NO: 67 - DNA, 25 bp, Artificial Sequence
  synthetic nucleotide probe
gggagccattagaacacagatacaa

SEQ ID NO: 68 - DNA, 25 bp, Artificial Sequence
  synthetic nucleotide probe
tttgtcttcatgaatgccctgtggc

SEQ ID NO: 69 - DNA, 25 bp, Artificial Sequence
  synthetic nucleotide probe
ggagaccaagccttcttatctcaac

SEQ ID NO: 70 - DNA, 25 bp, Artificial Sequence
  synthetic nucleotide probe
tgcagtgtgtgggttcattcacctg

SEQ ID NO: 71 - DNA, 25 bp, Artificial Sequence
  synthetic nucleotide probe
ctccgcttcattctacagcttgtgg

SEQ ID NO: 72 - DNA, 25 bp, Artificial Sequence
  synthetic nucleotide probe
tgtgtcagagcactgagctccaccc

SEQ ID NO: 73 - DNA, 25 bp, Artificial Sequence
  synthetic nucleotide probe
tacaagttcggctttgagcttcctc

SEQ ID NO: 74 - DNA, 25 bp, Artificial Sequence
  synthetic nucleotide probe
aaaggatgcggactcatcctcagcc

SEQ ID NO: 75 - DNA, 25 bp, Artificial Sequence
  synthetic nucleotide probe
actttgttcactgtcctgtgtcaga

SEQ ID NO: 76 - DNA, 25 bp, Artificial Sequence
  synthetic nucleotide probe
gaaagggttgctgctgtcagccttg

SEQ ID NO: 77 - DNA, 25 bp, Artificial Sequence
  synthetic nucleotide probe
agatagggatattggcccctcactg

SEQ ID NO: 78 - DNA, 25 bp, Artificial Sequence
  synthetic nucleotide probe
ggcaatctcctgggccttaaaggat

SEQ ID NO: 79 - DNA, 25 bp, Artificial Sequence
  synthetic nucleotide probe
cttagcctctgacttcctaatgtag

SEQ ID NO: 80 - DNA, 25 bp, Artificial Sequence
  synthetic nucleotide probe
gcaaaggggtttcctcgatttggag

SEQ ID NO: 81 - DNA, 25 bp, Artificial Sequence
  synthetic nucleotide probe
aaatggcctcctggcgtaagctttt

SEQ ID NO: 82 - DNA, 25 bp, Artificial Sequence
  synthetic nucleotide probe
aaaccaactcagttccatcatggtg

SEQ ID NO: 83 - DNA, 25 bp, Artificial Sequence
  synthetic nucleotide probe
ttccaccgtcatttctaactcttaa

SEQ ID NO: 84 - DNA, 25 bp, Artificial Sequence
  synthetic nucleotide probe
ggttttctcttcatgtaagtccttg

SEQ ID NO: 85 - DNA, 25 bp, Artificial Sequence
  synthetic nucleotide probe
cggagtacctgcgctatgaagacac

SEQ ID NO: 86 - DNA, 25 bp, Artificial Sequence
  synthetic nucleotide probe
ccctgcatcctcaacaacaatgtgc

SEQ ID NO: 87 - DNA, 25 bp, Artificial Sequence
  synthetic nucleotide probe
gtgttctcctactgcaaatattttc

SEQ ID NO: 88 - DNA, 25 bp, Artificial Sequence
  synthetic nucleotide probe
aattgaggccttttcgatagtttcg

SEQ ID NO: 89 - DNA, 25 bp, Artificial Sequence
  synthetic nucleotide probe
ggaggtggtcagcaggcaatctcct

SEQ ID NO: 90 - DNA, 25 bp, Artificial Sequence
  synthetic nucleotide probe
ccagcgcccatgttgtgatacaggg

SEQ ID NO: 91 - DNA, 25 bp, Artificial Sequence
  synthetic nucleotide probe
gaaaaactcaggcccatccattttc

SEQ ID NO: 92 - DNA, 25 bp, Artificial Sequence
  synthetic nucleotide probe
tgaggtggtctttaacgaccctgaa

SEQ ID NO: 93 - DNA, 25 bp, Artificial Sequence
  synthetic nucleotide probe
tgttcttagcactttaattcctgtc

SEQ ID NO: 94 - DNA, 25 bp, Artificial Sequence
  synthetic nucleotide probe
agctccacccttttctgagagttat

SEQ ID NO: 95 - DNA, 25 bp, Artificial Sequence
  synthetic nucleotide probe
cactctcagccatagcactttgttc

SEQ ID NO: 96 - DNA, 25 bp, Artificial Sequence
  synthetic nucleotide probe
gaagcagctttacctacttgtttct

SEQ ID NO: 97 - DNA, 25 bp, Artificial Sequence
  synthetic nucleotide probe
gaagttactcgtgtcaaagccgtta

SEQ ID NO: 98 - DNA, 25 bp, Artificial Sequence
  synthetic nucleotide probe
ggtggatgtcaatacccctgattta

SEQ ID NO: 99 - DNA, 25 bp, Artificial Sequence
  synthetic nucleotide probe
ccgagccagccaactcaagagacaa

SEQ ID NO: 100 - DNA, 25 bp, Artificial Sequence
  synthetic nucleotide probe
tggatgcagggatcccagcagtgca

SEQ ID NO: 101 - DNA, 25 bp, Artificial Sequence
  synthetic nucleotide probe
gatcctggcttgcggagtggctaaa

SEQ ID NO: 102 - DNA, 25 bp, Artificial Sequence
  synthetic nucleotide probe
gctgaaactggtctactgtgtctct

SEQ ID NO: 103 - DNA, 25 bp, Artificial Sequence
  synthetic nucleotide probe
tttctaagactttttcacatccaaa

SEQ ID NO: 104 - DNA, 25 bp, Artificial Sequence
  synthetic nucleotide probe
gttttactccaatcagctggcaatt

SEQ ID NO: 105 - DNA, 25 bp, Artificial Sequence
  synthetic nucleotide probe
ggcacccttagctgatggaaacaat

SEQ ID NO: 106 - DNA, 25 bp, Artificial Sequence
  synthetic nucleotide probe
attttgagctgccggttatacacca

SEQ ID NO: 107 - DNA, 25 bp, Artificial Sequence
  synthetic nucleotide probe
tgttctgttcagtacctagctctgc

SEQ ID NO: 108 - DNA, 25 bp, Artificial Sequence
  synthetic nucleotide probe
gtaaatgccaaactaccgacttgat

SEQ ID NO: 109 - DNA, 25 bp, Artificial Sequence
  synthetic nucleotide probe
tacgcttagaatcagttttactcca

SEQ ID NO: 110 - DNA, 25 bp, Artificial Sequence
  synthetic nucleotide probe
gttcagaaactcataggcaccctta

SEQ ID NO: 111 - DNA, 25 bp, Artificial Sequence
  synthetic nucleotide probe
tgagcagtggtgtccatcacatata

SEQ ID NO: 112 - DNA, 25 bp, Artificial Sequence
  synthetic nucleotide probe
atgtacaactcagatgtttctcatt

SEQ ID NO: 113 - DNA, 25 bp, Artificial Sequence
  synthetic nucleotide probe
gctctgctcttttatattgctttaa

SEQ ID NO: 114 - DNA, 25 bp, Artificial Sequence
  synthetic nucleotide probe
aatttcttactatacttttcataat

SEQ ID NO: 115 - DNA, 25 bp, Artificial Sequence
  synthetic nucleotide probe
atttcacctaccaaagctgtgctgt

SEQ ID NO: 116 - DNA, 25 bp, Artificial Sequence
  synthetic nucleotide probe
actagctcattatttccatctttgg

SEQ ID NO: 117 - DNA, 25 bp, Artificial Sequence
  synthetic nucleotide probe
aaatgatttttcacaactaacttgt

SEQ ID NO: 118 - DNA, 25 bp, Artificial Sequence
  synthetic nucleotide probe
ttgcagtctgcaccttatggatcac

SEQ ID NO: 119 - DNA, 25 bp, Artificial Sequence
  synthetic nucleotide probe
tgatacatctgtgatttaggtcatt

SEQ ID NO: 120 - DNA, 25 bp, Artificial Sequence
  synthetic nucleotide probe
ggagacgccaatagcaatatctagg

SEQ ID NO: 121 - DNA, 25 bp, Artificial Sequence
  synthetic nucleotide probe
ctgatgccacatagtcttgcataaa

SEQ ID NO: 122 - DNA, 25 bp, Artificial Sequence
  synthetic nucleotide probe
agctgtgctgttaatgccgtgaaag

SEQ ID NO: 123 - DNA, 25 bp, Artificial Sequence
  synthetic nucleotide probe
gaagtgcgccaatgttgtcttttct

SEQ ID NO: 124 - DNA, 25 bp, Artificial Sequence
  synthetic nucleotide probe
gtgaaaccttcatggatagtcttta

SEQ ID NO: 125 - DNA, 25 bp, Artificial Sequence
  synthetic nucleotide probe
tttactaaagtcctcctgccaggta

SEQ ID NO: 126 - DNA, 25 bp, Artificial Sequence
  synthetic nucleotide probe
ggacatcaaccacagacaatttaaa

SEQ ID NO: 127 - DNA, 25 bp, Artificial Sequence
  synthetic nucleotide probe
tgttgcatgcatatttcacctacca

SEQ ID NO: 128 - DNA, 25 bp, Artificial Sequence
  synthetic nucleotide probe
ataaaccttagacatatcacaccta

SEQ ID NO: 129 - DNA, 25 bp, Artificial Sequence
  synthetic nucleotide probe
tagtctttaatctctgatctttttg

SEQ ID NO: 130 - DNA, 25 bp, Artificial Sequence
  synthetic nucleotide probe
gagacagcgtgatacttacaactca

SEQ ID NO: 131 - DNA, 25 bp, Artificial Sequence
  synthetic nucleotide probe
gaccattgtattttccactagcagt

SEQ ID NO: 132 - DNA, 25 bp, Artificial Sequence
  synthetic nucleotide probe
ctgcagcagcaggttacatagcaaa

SEQ ID NO: 133 - DNA, 25 bp, Artificial Sequence
  synthetic nucleotide probe
gccgtgaaagtttaacgtttgcgat

SEQ ID NO: 134 - DNA, 25 bp, Artificial Sequence
  synthetic nucleotide probe
aactgccgtaattttgatacatctg

SEQ ID NO: 135 - DNA, 25 bp, Artificial Sequence
  synthetic nucleotide probe
tatttaccatcacatgctgcagctg

SEQ ID NO: 136 - DNA, 25 bp, Artificial Sequence
  synthetic nucleotide probe
aacgtttgcgataaactgccgtaat

SEQ ID NO: 137 - DNA, 25 bp, Artificial Sequence
  synthetic nucleotide probe
ggaaatttgcattgagaccattgta

SEQ ID NO: 138 - DNA, 25 bp, Artificial Sequence
  synthetic nucleotide probe
gcaccttatggatcacaattacctt

SEQ ID NO: 139 - DNA, 25 bp, Artificial Sequence
  synthetic nucleotide probe
agaagcaaaacacagcacctttacc

SEQ ID NO: 140 - DNA, 25 bp, Artificial Sequence
  synthetic nucleotide probe
cccttagtctcctcacataaatttc

SEQ ID NO: 141 - DNA, 25 bp, Artificial Sequence
  synthetic nucleotide probe
tgtgtaaggtgatgttcccggtcgc

SEQ ID NO: 142 - DNA, 25 bp, Artificial Sequence
  synthetic nucleotide probe
ctgccaggtagttcccactgatgga

SEQ ID NO: 143 - DNA, 25 bp, Artificial Sequence
  synthetic nucleotide probe
gccttcctttaccttgtagtacaag

SEQ ID NO: 144 - DNA, 25 bp, Artificial Sequence
  synthetic nucleotide probe
tttttcctcttgcaacaatgacggt

SEQ ID NO: 145 - DNA, 25 bp, Artificial Sequence
  synthetic nucleotide probe
gtcaatttacaaggccagggataga

SEQ ID NO: 146 - DNA, 25 bp, Artificial Sequence
  synthetic nucleotide probe
ttccacttcattttacatgccacta

SEQ ID NO: 147 - DNA, 25 bp, Artificial Sequence
  synthetic nucleotide probe
gtgctagacaattactgttcttttc

SEQ ID NO: 148 - DNA, 25 bp, Artificial Sequence
  synthetic nucleotide probe
aatatctataactgcattttgtgct

SEQ ID NO: 149 - DNA, 25 bp, Artificial Sequence
  synthetic nucleotide probe
gatagaaaacactccataattgctt

SEQ ID NO: 150 - DNA, 25 bp, Artificial Sequence
  synthetic nucleotide probe
cattgattttattaagccttccttt

SEQ ID NO: 151 - DNA, 25 bp, Artificial Sequence
  synthetic nucleotide probe
tacatgccactatattgactttaat

SEQ ID NO: 152 - DNA, 25 bp, Artificial Sequence
  synthetic nucleotide probe
tctggtatgaaaggctccattgatt

SEQ ID NO: 153 - DNA, 25 bp, Artificial Sequence
  synthetic nucleotide probe
gctttccttgattttgctgaggatt

SEQ ID NO: 154 - DNA, 25 bp, Artificial Sequence
  synthetic nucleotide probe
ggatatcagcgtttctctgtgtgct

SEQ ID NO: 155 - DNA, 25 bp, Artificial Sequence
  synthetic nucleotide probe
gaaagctttgtctgccaatgttgtg

SEQ ID NO: 156 - DNA, 25 bp, Artificial Sequence
  synthetic nucleotide probe
cagagagtcctctctaatgctccta

SEQ ID NO: 157 - DNA, 25 bp, Artificial Sequence
  synthetic nucleotide probe
gatattgcacagtactggtcagtat

SEQ ID NO: 158 - DNA, 25 bp, Artificial Sequence
  synthetic nucleotide probe
gaccagggacttgacccattagggt

SEQ ID NO: 159 - DNA, 25 bp, Artificial Sequence
  synthetic nucleotide probe
agatatggtatcctctgagcgccac

SEQ ID NO: 160 - DNA, 25 bp, Artificial Sequence
  synthetic nucleotide probe
aatgctcctagaccagggacttgac

SEQ ID NO: 161 - DNA, 25 bp, Artificial Sequence
  synthetic nucleotide probe
atcgtgacactttcaacatgtaggg

SEQ ID NO: 162 - DNA, 25 bp, Artificial Sequence
  synthetic nucleotide probe
ttggatgccctaactgctgatgtga

SEQ ID NO: 163 - DNA, 25 bp, Artificial Sequence
  synthetic nucleotide probe
gtgttttctgcttcatacgatattg

SEQ ID NO: 164 - DNA, 25 bp, Artificial Sequence
  synthetic nucleotide probe
aagggtgacatccaggaggggcctc

SEQ ID NO: 165 - DNA, 25 bp, Artificial Sequence
  synthetic nucleotide probe
caggaggggcctctgaaatttccca

SEQ ID NO: 166 - DNA, 25 bp, Artificial Sequence
  synthetic nucleotide probe
gatgcgggagtggttttcagagaca

SEQ ID NO: 167 - DNA, 25 bp, Artificial Sequence
  synthetic nucleotide probe
cagcaaggattcaggagtgcccctc

SEQ ID NO: 168 - DNA, 25 bp, Artificial Sequence
  synthetic nucleotide probe
gtgaactttctgccaagatgcggga

SEQ ID NO: 169 - DNA, 25 bp, Artificial Sequence
  synthetic nucleotide probe
caaggctcgggaactcatcagccgc

SEQ ID NO: 170 - DNA, 25 bp, Artificial Sequence
  synthetic nucleotide probe
aacacactggaggacaaggctcggg

SEQ ID NO: 171 - DNA, 25 bp, Artificial Sequence
  synthetic nucleotide probe
gacgtctccagtgccttggataagc

SEQ ID NO: 172 - DNA, 25 bp, Artificial Sequence
  synthetic nucleotide probe
ccaagccctccagcaaggattcagg

SEQ ID NO: 173 - DNA, 25 bp, Artificial Sequence
  synthetic nucleotide probe
tcatcagccgcatcaaacagagtga

SEQ ID NO: 174 - DNA, 25 bp, Artificial Sequence
  synthetic nucleotide probe
gttctgtcgatcgtcttggaaggcc

SEQ ID NO: 175 - DNA, 25 bp, Artificial Sequence
  synthetic nucleotide probe
atcgccacctggtgctcatgaggtg

SEQ ID NO: 176 - DNA, 25 bp, Artificial Sequence
  synthetic nucleotide probe
actcgtcttggtattgcactgttgt

SEQ ID NO: 177 - DNA, 25 bp, Artificial Sequence
  synthetic nucleotide probe
attctcttcccatttttgtacattt

SEQ ID NO: 178 - DNA, 25 bp, Artificial Sequence
  synthetic nucleotide probe
tgctccgtgaaaggacatcgccacc

SEQ ID NO: 179 - DNA, 25 bp, Artificial Sequence
  synthetic nucleotide probe
ggagacaaacctcgtcagatgctca

SEQ ID NO: 180 - DNA, 25 bp, Artificial Sequence
  synthetic nucleotide probe
tgaagtctttggtgttgctccgtga

SEQ ID NO: 181 - DNA, 25 bp, Artificial Sequence
  synthetic nucleotide probe
tgattgttgccatgtgagagtttta

SEQ ID NO: 182 - DNA, 25 bp, Artificial Sequence
  synthetic nucleotide probe
actcctaatgtttggtgctatgttt

SEQ ID NO: 183 - DNA, 25 bp, Artificial Sequence
  synthetic nucleotide probe
gtatgggtcatttcaaagagggctt

SEQ ID NO: 184 - DNA, 25 bp, Artificial Sequence
  synthetic nucleotide probe
tggtgctatgttttcctgaggagat

SEQ ID NO: 185 - DNA, 25 bp, Artificial Sequence
  synthetic nucleotide probe
aagtttctctagtgttttctgtgga

SEQ ID NO: 186 - DNA, 25 bp, Artificial Sequence
  synthetic nucleotide probe
gtatttttggctcgaagtttctcta

SEQ ID NO: 187 - DNA, 25 bp, Artificial Sequence
  synthetic nucleotide probe
gaagccatagcactcctaatgtttg

SEQ ID NO: 188 - DNA, 25 bp, Artificial Sequence
  synthetic nucleotide probe
aagagggcttatgaggctgtgaaac

SEQ ID NO: 189 - DNA, 25 bp, Artificial Sequence
  synthetic nucleotide probe
cccagagctcttaacgctgtgacca

SEQ ID NO: 190 - DNA, 25 bp, Artificial Sequence
  synthetic nucleotide probe
gaggctgtgaaacccagagctctta

SEQ ID NO: 191 - DNA, 25 bp, Artificial Sequence
  synthetic nucleotide probe
atttctcttcttcagggcaaacttg

SEQ ID NO: 192 - DNA, 25 bp, Artificial Sequence
  synthetic nucleotide probe
gctggggagcacttaattcttccca

SEQ ID NO: 193 - DNA, 25 bp, Artificial Sequence
  synthetic nucleotide probe
ggagctccgatgtgaggggcaaggc

SEQ ID NO: 194 - DNA, 25 bp, Artificial Sequence
  synthetic nucleotide probe
tctggctggggtccgtgaagcccag

SEQ ID NO: 195 - DNA, 25 bp, Artificial Sequence
  synthetic nucleotide probe
gccaaaccagctcatttcaacaaag

SEQ ID NO: 196 - DNA, 25 bp, Artificial Sequence
  synthetic nucleotide probe
atgtgaacaccatcatgcctttata

SEQ ID NO: 197 - DNA, 25 bp, Artificial Sequence
  synthetic nucleotide probe
tgccatcacatccctgatactgtgt

SEQ ID NO: 198 - DNA, 25 bp, Artificial Sequence
  synthetic nucleotide probe
tttggactacgttcttggctccaga

SEQ ID NO: 199 - DNA, 25 bp, Artificial Sequence
  synthetic nucleotide probe
gcagccaaatcttgcctgtgctggg

SEQ ID NO: 200 - DNA, 25 bp, Artificial Sequence
  synthetic nucleotide probe
gctttgaagcaccttccctgagaag

SEQ ID NO: 201 - DNA, 25 bp, Artificial Sequence
  synthetic nucleotide probe
tctgctttcacatctctgagctata

SEQ ID NO: 202 - DNA, 25 bp, Artificial Sequence
  synthetic nucleotide probe
taatgttgcctggggcttaacccac

SEQ ID NO: 203 - DNA, 25 bp, Artificial Sequence
  synthetic nucleotide probe
gctgaagaggcggaagaaagctcgg

SEQ ID NO: 204 - DNA, 25 bp, Artificial Sequence
  synthetic nucleotide probe
ctcctgcccacgggaggacaagcaa

SEQ ID NO: 205 - DNA, 25 bp, Artificial Sequence
  synthetic nucleotide probe
cctgcccacgggaggacaagcaata

SEQ ID NO: 206 - DNA, 25 bp, Artificial Sequence
  synthetic nucleotide probe
ggacaagcaataactctctacagga

SEQ ID NO: 207 - DNA, 25 bp, Artificial Sequence
  synthetic nucleotide probe
aactctctacaggaatatatttttt

SEQ ID NO: 208 - DNA, 25 bp, Artificial Sequence
  synthetic nucleotide probe
gttgactacgagcagatcaagataa

SEQ ID NO: 209 - DNA, 25 bp, Artificial Sequence
  synthetic nucleotide probe
aatgcgcgttgactacgagcagatc

SEQ ID NO: 210 - DNA, 25 bp, Artificial Sequence
  synthetic nucleotide probe
cacaatgcgcgttgactacgagcag

SEQ ID NO: 211 - DNA, 25 bp, Artificial Sequence
  synthetic nucleotide probe
gcgcgttgactacgagcagatcaag

SEQ ID NO: 212 - DNA, 25 bp, Artificial Sequence
  synthetic nucleotide probe
aagcaataactctctacaggaatat

SEQ ID NO: 213 - DNA, 25 bp, Artificial Sequence
  synthetic nucleotide probe
agacagaactgtccacatctgcctc

SEQ ID NO: 214 - DNA, 25 bp, Artificial Sequence
  synthetic nucleotide probe
aatctttgagacaagcatatgctac

SEQ ID NO: 215 - DNA, 25 bp, Artificial Sequence
  synthetic nucleotide probe
cggccgtgcgtacttagacatgcat

SEQ ID NO: 216 - DNA, 25 bp, Artificial Sequence
  synthetic nucleotide probe
ccattcgcagtttcactgtaccggc

SEQ ID NO: 217 - DNA, 25 bp, Artificial Sequence
  synthetic nucleotide probe
gtgcgtacttagacatgcatggctt

SEQ ID NO: 218 - DNA, 25 bp, Artificial Sequence
  synthetic nucleotide probe
ggagcgagcgaccaaaggaaccata

SEQ ID NO: 219 - DNA, 25 bp, Artificial Sequence
  synthetic nucleotide probe
gcatatgctactggcaggatcaacc

SEQ ID NO: 220 - DNA, 25 bp, Artificial Sequence
  synthetic nucleotide probe
aaccataactgatttaatgagccat

SEQ ID NO: 221 - DNA, 25 bp, Artificial Sequence
  synthetic nucleotide probe
gacatgcatggcttaatctttgaga

SEQ ID NO: 222 - DNA, 25 bp, Artificial Sequence
  synthetic nucleotide probe
gagcgaccaaaggaaccataactga

SEQ ID NO: 223 - DNA, 25 bp, Artificial Sequence
  synthetic nucleotide probe
caagtaggagaggagcgagcgacca

SEQ ID NO: 224 - DNA, 25 bp, Artificial Sequence
  synthetic nucleotide probe
aatgagccattcgcagtttcactgt

SEQ ID NO: 225 - DNA, 25 bp, Artificial Sequence
  synthetic nucleotide probe
tctctaatcccatcctgaggttgcc

SEQ ID NO: 226 - DNA, 25 bp, Artificial Sequence
  synthetic nucleotide probe
ggaagcttcatctgaccaatgtggg

SEQ ID NO: 227 - DNA, 25 bp, Artificial Sequence
  synthetic nucleotide probe
aaatgcaagggtcttaccctcctct

SEQ ID NO: 228 - DNA, 25 bp, Artificial Sequence
  synthetic nucleotide probe
ccacccacccaggtgtctaagatag

SEQ ID NO: 229 - DNA, 25 bp, Artificial Sequence
  synthetic nucleotide probe
gcaaagccaatatgaccactactga

SEQ ID NO: 230 - DNA, 25 bp, Artificial Sequence
  synthetic nucleotide probe
atcccctgaatgtgaattgctatcc

SEQ ID NO: 231 - DNA, 25 bp, Artificial Sequence
  synthetic nucleotide probe
agataggacatgctcctttctttct

SEQ ID NO: 232 - DNA, 25 bp, Artificial Sequence
  synthetic nucleotide probe
ttgctatccttattgccctattaaa

SEQ ID NO: 233 - DNA, 25 bp, Artificial Sequence
  synthetic nucleotide probe
tggtatggtgaaactaatcccctga

SEQ ID NO: 234 - DNA, 25 bp, Artificial Sequence
  synthetic nucleotide probe
ttgccatcccccaaatgtgtggtat

SEQ ID NO: 235 - DNA, 25 bp, Artificial Sequence
  synthetic nucleotide probe
cttgtgaaatgtgtccctaagcctc

SEQ ID NO: 236 - DNA, 25 bp, Artificial Sequence
  synthetic nucleotide probe
taccctccaattcagactcagctga

SEQ ID NO: 237 - DNA, 25 bp, Artificial Sequence
  synthetic nucleotide probe
cagaacttcaaatacttcctaccct

SEQ ID NO: 238 - DNA, 25 bp, Artificial Sequence
  synthetic nucleotide probe
cctgccccaaggaatcgtgcgggag

SEQ ID NO: 239 - DNA, 25 bp, Artificial Sequence
  synthetic nucleotide probe
gacagctgggtctccaagtggcagc

SEQ ID NO: 240 - DNA, 25 bp, Artificial Sequence
  synthetic nucleotide probe
atcttctttggactacaggtggggt

SEQ ID NO: 241 - DNA, 25 bp, Artificial Sequence
  synthetic nucleotide probe
taggatgctgattttcctacccgtg

SEQ ID NO: 242 - DNA, 25 bp, Artificial Sequence
  synthetic nucleotide probe
gtatatgactgcactagctcttcct

SEQ ID NO: 243 - DNA, 25 bp, Artificial Sequence
  synthetic nucleotide probe
gagagcagcacatcattttatcatt

SEQ ID NO: 244 - DNA, 25 bp, Artificial Sequence
  synthetic nucleotide probe
gtcgaggagtggcctacaaatccag

SEQ ID NO: 245 - DNA, 25 bp, Artificial Sequence
  synthetic nucleotide probe
tgcaaggctgccagcatctttgctc

SEQ ID NO: 246 - DNA, 25 bp, Artificial Sequence
  synthetic nucleotide probe
atatgcggtgtcagtcactggtcgc

SEQ ID NO: 247 - DNA, 25 bp, Artificial Sequence
  synthetic nucleotide probe
aagactgttatgatcgctttgattt

SEQ ID NO: 248 - DNA, 25 bp, Artificial Sequence
  synthetic nucleotide probe
gcccatctgtcattatgttactgtc

SEQ ID NO: 249 - DNA, 25 bp, Artificial Sequence
  synthetic nucleotide probe
agggcgatgcctgggtttacaaaag

SEQ ID NO: 250 - DNA, 25 bp, Artificial Sequence
  synthetic nucleotide probe
agctcttaactatgtctctttgtaa

SEQ ID NO: 251 - DNA, 25 bp, Artificial Sequence
  synthetic nucleotide probe
gattcttccatcctgccggatgagt

SEQ ID NO: 252 - DNA, 25 bp, Artificial Sequence
  synthetic nucleotide probe
gaatctaaatcaagcccatctgtca

SEQ ID NO: 253 - DNA, 25 bp, Artificial Sequence
  synthetic nucleotide probe
gagctatcttaagaccaatatctct

SEQ ID NO: 254 - DNA, 25 bp, Artificial Sequence
  synthetic nucleotide probe
ggaagacatcctatctagctgcaag

SEQ ID NO: 255 - DNA, 25 bp, Artificial Sequence
  synthetic nucleotide probe
gtgtgggtgtgatacagcctacata

SEQ ID NO: 256 - DNA, 25 bp, Artificial Sequence
  synthetic nucleotide probe
atgtggtgggtatcaggaggcagcg

SEQ ID NO: 257 - DNA, 25 bp, Artificial Sequence
  synthetic nucleotide probe
ggaggcagcggcttaagggcgatgc

SEQ ID NO: 258 - DNA, 25 bp, Artificial Sequence
  synthetic nucleotide probe
ttgtgcttctcacctttgggtggga

SEQ ID NO: 259 - DNA, 25 bp, Artificial Sequence
  synthetic nucleotide probe
ggatgcctgcgtgtggtttaggtgt

SEQ ID NO: 260 - DNA, 25 bp, Artificial Sequence
  synthetic nucleotide probe
tagcaggatgtctggctgcacctgg

SEQ ID NO: 261 - DNA, 25 bp, Artificial Sequence
  synthetic nucleotide probe
tctccactcagtaccttggatcaga

SEQ ID NO: 262 - DNA, 25 bp, Artificial Sequence
  synthetic nucleotide probe
gagtcatgcggattatccactcgcc

SEQ ID NO: 263 - DNA, 25 bp, Artificial Sequence
  synthetic nucleotide probe
ctggcatgactgtttgtttctccaa

SEQ ID NO: 264 - DNA, 25 bp, Artificial Sequence
  synthetic nucleotide probe
ccccactctcttatttgtgcattcg

SEQ ID NO: 265 - DNA, 25 bp, Artificial Sequence
  synthetic nucleotide probe
agcacttgatgaactcggggtacta

SEQ ID NO: 266 - DNA, 25 bp, Artificial Sequence
  synthetic nucleotide probe
gccagtgtgtcttacttggttgctg

SEQ ID NO: 267 - DNA, 25 bp, Artificial Sequence
  synthetic nucleotide probe
ccctcttggcgtgagttgcgtattc

SEQ ID NO: 268 - DNA, 25 bp, Artificial Sequence
  synthetic nucleotide probe
ttggttgctgaacatcttgccacct

SEQ ID NO: 269 - DNA, 25 bp, Artificial Sequence
  synthetic nucleotide probe
gaaggaccgaggtctttccattcct

SEQ ID NO: 270 - DNA, 25 bp, Artificial Sequence
  synthetic nucleotide probe
gagtactaatcttttgcccagaggc

SEQ ID NO: 271 - DNA, 25 bp, Artificial Sequence
  synthetic nucleotide probe
agtgaaagtgacatcctggccacct

SEQ ID NO: 272 - DNA, 25 bp, Artificial Sequence
  synthetic nucleotide probe
acagtggcatagactccttcacaca

SEQ ID NO: 273 - DNA, 25 bp, Artificial Sequence
  synthetic nucleotide probe
acagggacagtcttcatttacttgt

SEQ ID NO: 274 - DNA, 25 bp, Artificial Sequence
  synthetic nucleotide probe
tccattcctcgagcagatactcttg

SEQ ID NO: 275 - DNA, 25 bp, Artificial Sequence
  synthetic nucleotide probe
gcaccagcaactacttctttatatt

SEQ ID NO: 276 - DNA, 25 bp, Artificial Sequence
  synthetic nucleotide probe
aaaaggagagtgacacacccctcca

SEQ ID NO: 277 - DNA, 25 bp, Artificial Sequence
  synthetic nucleotide probe
cacctcacgcatttgatcacagact

SEQ ID NO: 278 - DNA, 25 bp, Artificial Sequence
  synthetic nucleotide probe
ccttcacacatcactgtggcaccag

SEQ ID NO: 279 - DNA, 25 bp, Artificial Sequence
  synthetic nucleotide probe
ccttcacctcagtggagcttctgag

SEQ ID NO: 280 - DNA, 25 bp, Artificial Sequence
  synthetic nucleotide probe
ggctttgcttagtatcatgtccatg

SEQ ID NO: 281 - DNA, 25 bp, Artificial Sequence
  synthetic nucleotide probe
tgtaccttcgttcaaatatcctcat

SEQ ID NO: 282 - DNA, 25 bp, Artificial Sequence
  synthetic nucleotide probe
catctgtcactcactatattcacaa

SEQ ID NO: 283 - DNA, 25 bp, Artificial Sequence
  synthetic nucleotide probe
gttttatactgctcaagatcgtcat

SEQ ID NO: 284 - DNA, 25 bp, Artificial Sequence
  synthetic nucleotide probe
gggatagaaaggccacctcttcact

SEQ ID NO: 285 - DNA, 25 bp, Artificial Sequence
  synthetic nucleotide probe
aactgcagtcttctgctagccaata

SEQ ID NO: 286 - DNA, 25 bp, Artificial Sequence
  synthetic nucleotide probe
actcattctaacattgcttacttaa

SEQ ID NO: 287 - DNA, 25 bp, Artificial Sequence
  synthetic nucleotide probe
cacctcttcactctctatagaatat

SEQ ID NO: 288 - DNA, 25 bp, Artificial Sequence
  synthetic nucleotide probe
gctacatagccctatcgaaatgcga

SEQ ID NO: 289 - DNA, 25 bp, Artificial Sequence
  synthetic nucleotide probe
tcctcatgtaattgccatctgtcac

SEQ ID NO: 290 - DNA, 25 bp, Artificial Sequence
  synthetic nucleotide probe
ttgcacatgtcactactggggaggt

SEQ ID NO: 291 - DNA, 25 bp, Artificial Sequence
  synthetic nucleotide probe
cctctcaatcactactcttcttgaa

SEQ ID NO: 292 - DNA, 25 bp, Artificial Sequence
  synthetic nucleotide probe
gttctcaaaagcttaccagtgtgga

SEQ ID NO: 293 - DNA, 25 bp, Artificial Sequence
  synthetic nucleotide probe
gtgttcagttctgttacacagtgca

SEQ ID NO: 294 - DNA, 25 bp, Artificial Sequence
  synthetic nucleotide probe
gagctgtctttgtcgtggagatctg

SEQ ID NO: 295 - DNA, 25 bp, Artificial Sequence
  synthetic nucleotide probe
acacagtgcattgccctttgttggg

SEQ ID NO: 296 - DNA, 25 bp, Artificial Sequence
  synthetic nucleotide probe
acacatgcttgtcggaacgctttct

SEQ ID NO: 297 - DNA, 25 bp, Artificial Sequence
  synthetic nucleotide probe
acttggtgttagccacgctgtttac

SEQ ID NO: 298 - DNA, 25 bp, Artificial Sequence
  synthetic nucleotide probe
gtgtccgaagttgagatggcctgcc

SEQ ID NO: 299 - DNA, 25 bp, Artificial Sequence
  synthetic nucleotide probe
gggagtctgtttgttccaatgggtt

SEQ ID NO: 300 - DNA, 25 bp, Artificial Sequence
  synthetic nucleotide probe
ggagatctggaactttgcacatgtc

SEQ ID NO: 301 - DNA, 25 bp, Artificial Sequence
  synthetic nucleotide probe
gatgagcagtcacactgttactcca

SEQ ID NO: 302 - DNA, 25 bp, Artificial Sequence
  synthetic nucleotide probe
gcactctctaaatcttccttgtgag

SEQ ID NO: 303 - DNA, 25 bp, Artificial Sequence
  synthetic nucleotide probe
ggattatgggtcctgcaattctaca

SEQ ID NO: 304 - DNA, 25 bp, Artificial Sequence
  synthetic nucleotide probe
gaaaggatgttttgtcccatttcct

SEQ ID NO: 305 - DNA, 25 bp, Artificial Sequence
  synthetic nucleotide probe
aattgccaaggcagcgggatctcgt

SEQ ID NO: 306 - DNA, 25 bp, Artificial Sequence
  synthetic nucleotide probe
tcctctctgagactgctaagtaggg

SEQ ID NO: 307 - DNA, 25 bp, Artificial Sequence
  synthetic nucleotide probe
tgctcaaccactgtggcgttctgct

SEQ ID NO: 308 - DNA, 25 bp, Artificial Sequence
  synthetic nucleotide probe
tgattgacacacagcactctctaaa

SEQ ID NO: 309 - DNA, 25 bp, Artificial Sequence
  synthetic nucleotide probe
ctggacactagggtcaggcggggtg

SEQ ID NO: 310 - DNA, 25 bp, Artificial Sequence
  synthetic nucleotide probe
agaggcccaggaatcggagcgaagc

SEQ ID NO: 311 - DNA, 25 bp, Artificial Sequence
  synthetic nucleotide probe
ggggcagtgatggttgccaggacga

SEQ ID NO: 312 - DNA, 25 bp, Artificial Sequence
  synthetic nucleotide probe
tcatgcagcaactcagctcgtcaat

SEQ ID NO: 313 - DNA, 25 bp, Artificial Sequence
  synthetic nucleotide probe
atgttcaaaattccgcttcaagcct

SEQ ID NO: 314 - DNA, 25 bp, Artificial Sequence
  synthetic nucleotide probe
aaagcgctactcgattaacaggtgg

SEQ ID NO: 315 - DNA, 25 bp, Artificial Sequence
  synthetic nucleotide probe
actcatcagcctttgtgcaactgtg

SEQ ID NO: 316 - DNA, 25 bp, Artificial Sequence
  synthetic nucleotide probe
gaacatttgcaactcatcagccttt

SEQ ID NO: 317 - DNA, 25 bp, Artificial Sequence
  synthetic nucleotide probe
tcaacagcagcgattactagacatt

SEQ ID NO: 318 - DNA, 25 bp, Artificial Sequence
  synthetic nucleotide probe
acccctgacctctagatgttcaaaa

SEQ ID NO: 319 - DNA, 25 bp, Artificial Sequence
  synthetic nucleotide probe
gtgatgcagcagttatctcagaatt

SEQ ID NO: 320 - DNA, 25 bp, Artificial Sequence
  synthetic nucleotide probe
gatggagtatttgctgcctgtcaga

SEQ ID NO: 321 - DNA, 25 bp, Artificial Sequence
  synthetic nucleotide probe
aagccattacatttcttcaaagcgc

SEQ ID NO: 322 - DNA, 25 bp, Artificial Sequence
  synthetic nucleotide probe
tcagctcgtcaatcaactccatgat

SEQ ID NO: 323 - DNA, 25 bp, Artificial Sequence
  synthetic nucleotide probe
gtgacaatcattttgctgacagaat

SEQ ID NO: 324 - DNA, 25 bp, Artificial Sequence
  synthetic nucleotide probe
aagggcattttctttgattctcaaa

SEQ ID NO: 325 - DNA, 25 bp, Artificial Sequence
  synthetic nucleotide probe
ggagccatcatatgtcacagtgttc

SEQ ID NO: 326 - DNA, 25 bp, Artificial Sequence
  synthetic nucleotide probe
agagaaacgtgtgccctatacttcc

SEQ ID NO: 327 - DNA, 25 bp, Artificial Sequence
  synthetic nucleotide probe
gaaatccatctatctacagcctaag

SEQ ID NO: 328 - DNA, 25 bp, Artificial Sequence
  synthetic nucleotide probe
tagctcactgtcactcactgaatag

SEQ ID NO: 329 - DNA, 25 bp, Artificial Sequence
  synthetic nucleotide probe
gagacaccacttttcaaaggacttc

SEQ ID NO: 330 - DNA, 25 bp, Artificial Sequence
  synthetic nucleotide probe
agttctttccagtgttttgtagctc

SEQ ID NO: 331 - DNA, 25 bp, Artificial Sequence
  synthetic nucleotide probe
ggacttcttggtttcagcataacct

SEQ ID NO: 332 - DNA, 25 bp, Artificial Sequence
  synthetic nucleotide probe
gagaagcctatacatttagctgaca

SEQ ID NO: 333 - DNA, 25 bp, Artificial Sequence
  synthetic nucleotide probe
tgccctatacttcctgtgacaatca

SEQ ID NO: 334 - DNA, 25 bp, Artificial Sequence
  synthetic nucleotide probe
cttcccccactaaggtttgagacag

SEQ ID NO: 335 - DNA, 25 bp, Artificial Sequence
  synthetic nucleotide probe
gacgcaggacgagctcagttgtaga

SEQ ID NO: 336 - DNA, 25 bp, Artificial Sequence
  synthetic nucleotide probe
gacgtatccagcaagcggaacctct

SEQ ID NO: 337 - DNA, 25 bp, Artificial Sequence
  synthetic nucleotide probe
ttcaatatcccagtaaacccatgta

SEQ ID NO: 338 - DNA, 25 bp, Artificial Sequence
  synthetic nucleotide probe
agcagtgataccaaccacatctgaa

SEQ ID NO: 339 - DNA, 25 bp, Artificial Sequence
  synthetic nucleotide probe
cttgagaccaggagacgtatccagc

SEQ ID NO: 340 - DNA, 25 bp, Artificial Sequence
  synthetic nucleotide probe
actgatcatcataactctgtatctg

SEQ ID NO: 341 - DNA, 25 bp, Artificial Sequence
  synthetic nucleotide probe
gaacccaagctcaagacgcaggacg

SEQ ID NO: 342 - DNA, 25 bp, Artificial Sequence
  synthetic nucleotide probe
gcaagcggaacctctgggaaaagca

SEQ ID NO: 343 - DNA, 25 bp, Artificial Sequence
  synthetic nucleotide probe
gcggaatgtgtgcagtatctagaaa

SEQ ID NO: 344 - DNA, 25 bp, Artificial Sequence
  synthetic nucleotide probe
tctgtggataaggtcacttccccca

SEQ ID NO: 345 - DNA, 25 bp, Artificial Sequence
  synthetic nucleotide probe
ggttggtgcagcagtcattaaaagt

SEQ ID NO: 346 - DNA, 25 bp, Artificial Sequence
  synthetic nucleotide probe
gagtcaaggccagactagatcagcc

SEQ ID NO: 347 - DNA, 25 bp, Artificial Sequence
  synthetic nucleotide probe
ttctcatggagcttcctttctagag

SEQ ID NO: 348 - DNA, 25 bp, Artificial Sequence
  synthetic nucleotide probe
caaaggggcgtgtcatgtgcctcat

SEQ ID NO: 349 - DNA, 25 bp, Artificial Sequence
  synthetic nucleotide probe
caaggccagactagatcagcctaag

SEQ ID NO: 350 - DNA, 25 bp, Artificial Sequence
  synthetic nucleotide probe
catggagcttcctttctagagggga

SEQ ID NO: 351 - DNA, 25 bp, Artificial Sequence
  synthetic nucleotide probe
ctctattctcatggagcttcctttc

SEQ ID NO: 352 - DNA, 25 bp, Artificial Sequence
  synthetic nucleotide probe
atttgagtagatttggcctctattc

SEQ ID NO: 353 - DNA, 25 bp, Artificial Sequence
  synthetic nucleotide probe
gactttcaaaggggcgtgtcatgtg

SEQ ID NO: 354 - DNA, 25 bp, Artificial Sequence
  synthetic nucleotide probe
ttggcctctattctcatggagcttc

SEQ ID NO: 355 - DNA, 25 bp, Artificial Sequence
  synthetic nucleotide probe
gattctaataggttggtgcagcagt

SEQ ID NO: 356 - DNA, 25 bp, Artificial Sequence
  synthetic nucleotide probe
gctacggcatcatggggctgtacgt

SEQ ID NO: 357 - DNA, 25 bp, Artificial Sequence
  synthetic nucleotide probe
atcatggggctgtacgtgtccatcg

SEQ ID NO: 358 - DNA, 25 bp, Artificial Sequence
  synthetic nucleotide probe
cattatgttcgaggagctgccgtgc

SEQ ID NO: 359 - DNA, 25 bp, Artificial Sequence
  synthetic nucleotide probe
gctggcgcccgagagggaaggagcc

SEQ ID NO: 360 - DNA, 25 bp, Artificial Sequence
  synthetic nucleotide probe
gctggtcatcggcaagttcgtgcgc

SEQ ID NO: 361 - DNA, 25 bp, Artificial Sequence
  synthetic nucleotide probe
gaggagttgtacgccaagctcatct

SEQ ID NO: 362 - DNA, 25 bp, Artificial Sequence
  synthetic nucleotide probe
cgctcaccggagaccatgatcaagt

SEQ ID NO: 363 - DNA, 25 bp, Artificial Sequence
  synthetic nucleotide probe
gcggattcttcagcgagatctcgca

SEQ ID NO: 364 - DNA, 25 bp, Artificial Sequence
  synthetic nucleotide probe
ttcgtgcgcggattcttcagcgaga

SEQ ID NO: 365 - DNA, 25 bp, Artificial Sequence
  synthetic nucleotide probe
tcccccacgtgtactgtagagtttt

SEQ ID NO: 366 - DNA, 25 bp, Artificial Sequence
  synthetic nucleotide probe
agatctcgcactccattatgttcga

SEQ ID NO: 367 - DNA, 25 bp, Artificial Sequence
  synthetic nucleotide probe
ggcccctggtggaacagggccgcgt

SEQ ID NO: 368 - DNA, 25 bp, Artificial Sequence
  synthetic nucleotide probe
tggtggaagacatgcagcgccagtg

SEQ ID NO: 369 - DNA, 25 bp, Artificial Sequence
  synthetic nucleotide probe
gaagcgcctggcagtgtaccaggcc

SEQ ID NO: 370 - DNA, 25 bp, Artificial Sequence
  synthetic nucleotide probe
agcaggcccagcagatacgcctgca

SEQ ID NO: 371 - DNA, 25 bp, Artificial Sequence
  synthetic nucleotide probe
gtgcccagcgacaatcactgaacgc

SEQ ID NO: 372 - DNA, 25 bp, Artificial Sequence
  synthetic nucleotide probe
tggggcccctggtggaacagggccg

SEQ ID NO: 373 - DNA, 25 bp, Artificial Sequence
  synthetic nucleotide probe
aagcgcctggcagtgtaccaggccg

SEQ ID NO: 374 - DNA, 25 bp, Artificial Sequence
  synthetic nucleotide probe
gcccagcgacaatcactgaacgccg

SEQ ID NO: 375 - DNA, 25 bp, Artificial Sequence
  synthetic nucleotide probe
gcgcgcgcggatggaggagatgggc

SEQ ID NO: 376 - DNA, 25 bp, Artificial Sequence
  synthetic nucleotide probe
gcgacaatcactgaacgccgaagcc

SEQ ID NO: 377 - DNA, 25 bp, Artificial Sequence
  synthetic nucleotide probe
ccctggtggaacagggccgcgtgcg

SEQ ID NO: 378 - DNA, 25 bp, Artificial Sequence
  synthetic nucleotide probe
cataagtcctttcaattccaccagg

SEQ ID NO: 379 - DNA, 25 bp, Artificial Sequence
  synthetic nucleotide probe
gctagacaatgatttggccaggcct

SEQ ID NO: 380 - DNA, 25 bp, Artificial Sequence
  synthetic nucleotide probe
cagtgcatcacatctgcacactctc

SEQ ID NO: 381 - DNA, 25 bp, Artificial Sequence
  synthetic nucleotide probe
ctgaccttggaatcgtccctatatc

SEQ ID NO: 382 - DNA, 25 bp, Artificial Sequence
  synthetic nucleotide probe
tggaatcgtccctatatcagggcct

SEQ ID NO: 383 - DNA, 25 bp, Artificial Sequence
  synthetic nucleotide probe
gcagcccctaagtgcaaacacagca

SEQ ID NO: 384 - DNA, 25 bp, Artificial Sequence
  synthetic nucleotide probe
tctgcatatatgtctctttggagtt

SEQ ID NO: 385 - DNA, 25 bp, Artificial Sequence
  synthetic nucleotide probe
gaaggctggattctatctacataag

SEQ ID NO: 386 - DNA, 25 bp, Artificial Sequence
  synthetic nucleotide probe
gcccttaactatcaccagtgcatca

SEQ ID NO: 387 - DNA, 25 bp, Artificial Sequence
  synthetic nucleotide probe
caccactgtgcacttagccatgatg

SEQ ID NO: 388 - DNA, 25 bp, Artificial Sequence
  synthetic nucleotide probe
accacgaggctgattcctctcattt

SEQ ID NO: 389 - DNA, 25 bp, Artificial Sequence
  synthetic nucleotide probe
tgcaaagtggcagggaccggcagag

SEQ ID NO: 390 - DNA, 25 bp, Artificial Sequence
  synthetic nucleotide probe
gcaaacacagcatgggtccagaaga

SEQ ID NO: 391 - DNA, 25 bp, Artificial Sequence
  synthetic nucleotide probe
gcatatatgtctctttggagttgga

SEQ ID NO: 392 - DNA, 25 bp, Artificial Sequence
  synthetic nucleotide probe
agacgtggtctagaccagggctgct

SEQ ID NO: 393 - DNA, 25 bp, Artificial Sequence
  synthetic nucleotide probe
acttactgcctgaccttggaatcgt

SEQ ID NO: 394 - DNA, 25 bp, Artificial Sequence
  synthetic nucleotide probe
ggcctagtaaccaaggccctgtctc

SEQ ID NO: 395 - DNA, 25 bp, Artificial Sequence
  synthetic nucleotide probe
gcatgggtccagaagacgtggtcta

SEQ ID NO: 396 - DNA, 25 bp, Artificial Sequence
  synthetic nucleotide probe
gcatctgtctgctctgcatatatgt

SEQ ID NO: 397 - DNA, 25 bp, Artificial Sequence
  synthetic nucleotide probe
tctcagtttctgcctgggcaatggc

SEQ ID NO: 398 - DNA, 25 bp, Artificial Sequence
  synthetic nucleotide probe
ttactgcctgaccttggaatcgtcc

SEQ ID NO: 399 - DNA, 25 bp, Artificial Sequence
  synthetic nucleotide probe
gcaggaacttctagctcatttaaca

SEQ ID NO: 400 - DNA, 25 bp, Artificial Sequence
  synthetic nucleotide probe
actcctaatgtttggtgctatgttt

SEQ ID NO: 401 - DNA, 25 bp, Artificial Sequence
  synthetic nucleotide probe
tggtgctatgttttcctgaggagat

SEQ ID NO: 402 - DNA, 25 bp, Artificial Sequence
  synthetic nucleotide probe
gaagccatagcactcctaatgtttg

SEQ ID NO: 403 - DNA, 25 bp, Artificial Sequence
  synthetic nucleotide probe
aagagggcttatgaggctgtgaaac

SEQ ID NO: 404 - DNA, 25 bp, Artificial Sequence
  synthetic nucleotide probe
cccagagctcttaacgctgtgacca

SEQ ID NO: 405 - DNA, 25 bp, Artificial Sequence
  synthetic nucleotide probe
gaggctgtgaaacccagagctctta

SEQ ID NO: 406 - DNA, 25 bp, Artificial Sequence
  synthetic nucleotide probe
tgctccattggagtagtctcccacc

SEQ ID NO: 407 - DNA, 25 bp, Artificial Sequence
  synthetic nucleotide probe
ggtagaggccttctaggtgagacac

SEQ ID NO: 408 - DNA, 25 bp, Artificial Sequence
  synthetic nucleotide probe
tacttatctactgtccgaaggcctt

SEQ ID NO: 409 - DNA, 25 bp, Artificial Sequence
  synthetic nucleotide probe
cctgcatttcccattaatctacata

SEQ ID NO: 410 - DNA, 25 bp, Artificial Sequence
  synthetic nucleotide probe
aatgctgagaaatttgccactggag

SEQ ID NO: 411 - DNA, 25 bp, Artificial Sequence
  synthetic nucleotide probe
tatacagtttaataagcctcttgca

SEQ ID NO: 412 - DNA, 25 bp, Artificial Sequence
  synthetic nucleotide probe
atttaaaatattgatccttcccttg

SEQ ID NO: 413 - DNA, 25 bp, Artificial Sequence
  synthetic nucleotide probe
atctcacttgttttagttctgatcc

SEQ ID NO: 414 - DNA, 25 bp, Artificial Sequence
  synthetic nucleotide probe
atttgggtccaacttcaataatgta

SEQ ID NO: 415 - DNA, 25 bp, Artificial Sequence
  synthetic nucleotide probe
gactgtgggtcaaatgtttccattt

SEQ ID NO: 416 - DNA, 25 bp, Artificial Sequence
  synthetic nucleotide probe
aaatgaaactgttgctccattggag

SEQ ID NO: 417 - DNA, 25 bp, Artificial Sequence
  synthetic nucleotide probe
gtatctgtaaccacaatcacacata

SEQ ID NO: 418 - DNA, 25 bp, Artificial Sequence
  synthetic nucleotide probe
ggaccaccttcatgttagttgggta

SEQ ID NO: 419 - DNA, 25 bp, Artificial Sequence
  synthetic nucleotide probe
ttgcaagttacttgttctctcacct

SEQ ID NO: 420 - DNA, 25 bp, Artificial Sequence
  synthetic nucleotide probe
ctttttgcccacactgctttggata

SEQ ID NO: 421 - DNA, 25 bp, Artificial Sequence
  synthetic nucleotide probe
agatcacccctctaattatttctga

SEQ ID NO: 422 - DNA, 25 bp, Artificial Sequence
  synthetic nucleotide probe
tatttcctccataataaccctgcat

SEQ ID NO: 423 - DNA, 25 bp, Artificial Sequence
  synthetic nucleotide probe
gggatgttgcttactctttttgccc

SEQ ID NO: 424 - DNA, 25 bp, Artificial Sequence
  synthetic nucleotide probe
gtactatgtgactcatgcttctgga

SEQ ID NO: 425 - DNA, 25 bp, Artificial Sequence
  synthetic nucleotide probe
gttctctcacctgaggtattttttt

SEQ ID NO: 426 - DNA, 25 bp, Artificial Sequence
  synthetic nucleotide probe
gccactggagacaagcaatctgaat

SEQ ID NO: 427 - DNA, 25 bp, Artificial Sequence
  synthetic nucleotide probe
tcatcctgtgagttatttcctccat

SEQ ID NO: 428 - DNA, 25 bp, Artificial Sequence
  synthetic nucleotide probe
tgcaactagcaactcatcttcggaa

SEQ ID NO: 429 - DNA, 25 bp, Artificial Sequence
  synthetic nucleotide probe
ctgcccatagtcaccaaattctgtt

SEQ ID NO: 430 - DNA, 25 bp, Artificial Sequence
  synthetic nucleotide probe
tggaaaaggattctctgcctcgctt

SEQ ID NO: 431 - DNA, 25 bp, Artificial Sequence
  synthetic nucleotide probe
gctaattgtcctatgatgctattat

SEQ ID NO: 432 - DNA, 25 bp, Artificial Sequence
  synthetic nucleotide probe
ttcctcttctccctttgcaagagga

SEQ ID NO: 433 - DNA, 25 bp, Artificial Sequence
  synthetic nucleotide probe
atgacatttatcttcaaaacaccaa

SEQ ID NO: 434 - DNA, 25 bp, Artificial Sequence
  synthetic nucleotide probe
gagtagtctcccacctaaatatcaa

SEQ ID NO: 435 - DNA, 25 bp, Artificial Sequence
  synthetic nucleotide probe
ttcccacagcagctttgctcagtga

SEQ ID NO: 436 - DNA, 25 bp, Artificial Sequence
  synthetic nucleotide probe
ctcgctttgtgcgctctgagtttta

SEQ ID NO: 437 - DNA, 25 bp, Artificial Sequence
  synthetic nucleotide probe
atccatttgtaagcatttatcccat

SEQ ID NO: 438 - DNA, 25 bp, Artificial Sequence
  synthetic nucleotide probe
atgtatttatgctgctagactgtgg

SEQ ID NO: 439 - DNA, 25 bp, Artificial Sequence
  synthetic nucleotide probe
tcttcaccacagacaccttcttgtg

SEQ ID NO: 440 - DNA, 25 bp, Artificial Sequence
  synthetic nucleotide probe
gagcctaacactatcctgtaattca

SEQ ID NO: 441 - DNA, 25 bp, Artificial Sequence
  synthetic nucleotide probe
gtccctgtctatacattctctgtat

SEQ ID NO: 442 - DNA, 25 bp, Artificial Sequence
  synthetic nucleotide probe
taacctttgtaatgttcttcaccac

SEQ ID NO: 443 - DNA, 25 bp, Artificial Sequence
  synthetic nucleotide probe
actctgctcagccctgtaacagggt

SEQ ID NO: 444 - DNA, 25 bp, Artificial Sequence
  synthetic nucleotide probe
ttttacttacccatgtgagcctaac

SEQ ID NO: 445 - DNA, 25 bp, Artificial Sequence
  synthetic nucleotide probe
ttcattgcctttttcacctaagcat

SEQ ID NO: 446 - DNA, 25 bp, Artificial Sequence
  synthetic nucleotide probe
ttctctgtatcttttgggggtaact

SEQ ID NO: 447 - DNA, 25 bp, Artificial Sequence
  synthetic nucleotide probe
aggaagagctttgacttgtccctgt

SEQ ID NO: 448 - DNA, 25 bp, Artificial Sequence
  synthetic nucleotide probe
gtttttcagtgttcagccatgtcag

SEQ ID NO: 449 - DNA, 25 bp, Artificial Sequence
  synthetic nucleotide probe
attatgatcatctaccaccaactct

SEQ ID NO: 450 - DNA, 25 bp, Artificial Sequence
  synthetic nucleotide probe
gcaggggatggcctgatcgagctgc

SEQ ID NO: 451 - DNA, 25 bp, Artificial Sequence
  synthetic nucleotide probe
tgagcgacgaccttacagagcgagc

SEQ ID NO: 452 - DNA, 25 bp, Artificial Sequence
  synthetic nucleotide probe
gaccttcgggaaggagttcacagaa

SEQ ID NO: 453 - DNA, 25 bp, Artificial Sequence
  synthetic nucleotide probe
gagttcacagaagcggtggaagcca

SEQ ID NO: 454 - DNA, 25 bp, Artificial Sequence
  synthetic nucleotide probe
cagccccgatgattcttaacacagc

SEQ ID NO: 455 - DNA, 25 bp, Artificial Sequence
  synthetic nucleotide probe
gcaggtgagcgacgaccttacagag

SEQ ID NO: 456 - DNA, 25 bp, Artificial Sequence
  synthetic nucleotide probe
caggggatggcctgatcgagctgcg

SEQ ID NO: 457 - DNA, 25 bp, Artificial Sequence
  synthetic nucleotide probe
gagcaacagaaaaaggcggccatca

SEQ ID NO: 458 - DNA, 25 bp, Artificial Sequence
  synthetic nucleotide probe
tcctggatgacgtgtccttgacaca

SEQ ID NO: 459 - DNA, 25 bp, Artificial Sequence
  synthetic nucleotide probe
tcgggaaggagttcacagaagcggt

SEQ ID NO: 460 - DNA, 25 bp, Artificial Sequence
  synthetic nucleotide probe
tggatgacgtgtccttgacacatct

SEQ ID NO: 461 - DNA, 25 bp, Artificial Sequence
  synthetic nucleotide probe
tgttgaacttgtatccttcagcctt

SEQ ID NO: 462 - DNA, 25 bp, Artificial Sequence
  synthetic nucleotide probe
taatattgagtcttctggcctataa

SEQ ID NO: 463 - DNA, 25 bp, Artificial Sequence
  synthetic nucleotide probe
ggtctgtcttcctaggtattaatgt

SEQ ID NO: 464 - DNA, 25 bp, Artificial Sequence
  synthetic nucleotide probe
agttttcagtgtacaggtctaccat

SEQ ID NO: 465 - DNA, 25 bp, Artificial Sequence
  synthetic nucleotide probe
gccttgctaaactgtgagttctcat

SEQ ID NO: 466 - DNA, 25 bp, Artificial Sequence
  synthetic nucleotide probe
ggcctataaacaaggtctgtcttcc

SEQ ID NO: 467 - DNA, 25 bp, Artificial Sequence
  synthetic nucleotide probe
gtagtcacagttacacggcaggctg

SEQ ID NO: 468 - DNA, 25 bp, Artificial Sequence
  synthetic nucleotide probe
gttgggcaccttgattgagattgca

SEQ ID NO: 469 - DNA, 25 bp, Artificial Sequence
  synthetic nucleotide probe
aattctaaccacttgttgctagtaa

SEQ ID NO: 470 - DNA, 25 bp, Artificial Sequence
  synthetic nucleotide probe
aggtctaccatgtcagcatttcata

SEQ ID NO: 471 - DNA, 25 bp, Artificial Sequence
  synthetic nucleotide probe
aatggtgccatattgtcactccttc

SEQ ID NO: 472 - DNA, 25 bp, Artificial Sequence
  synthetic nucleotide probe
accagcccaggcaacatagcgtaaa

SEQ ID NO: 473 - DNA, 25 bp, Artificial Sequence
  synthetic nucleotide probe
gtgtttgttccaattagctttgttg

SEQ ID NO: 474 - DNA, 25 bp, Artificial Sequence
  synthetic nucleotide probe
taggttgtcccctaaagattctgaa

SEQ ID NO: 475 - DNA, 25 bp, Artificial Sequence
  synthetic nucleotide probe
tgcttagattgttgtactgctgcca

SEQ ID NO: 476 - DNA, 25 bp, Artificial Sequence
  synthetic nucleotide probe
ttaaacggttgataatgcctctaca

SEQ ID NO: 477 - DNA, 25 bp, Artificial Sequence
  synthetic nucleotide probe
tattctaccctcttttttggcaagg

SEQ ID NO: 478 - DNA, 25 bp, Artificial Sequence
  synthetic nucleotide probe
caagtcattgcattgtgttctaatt

SEQ ID NO: 479 - DNA, 25 bp, Artificial Sequence
  synthetic nucleotide probe
catagcgtaaaccctatctctaaaa

SEQ ID NO: 480 - DNA, 25 bp, Artificial Sequence
  synthetic nucleotide probe
aaccttggatggatatcttctcttt

SEQ ID NO: 481 - DNA, 25 bp, Artificial Sequence
  synthetic nucleotide probe
attgttgtactgctgccatttttat

SEQ ID NO: 482 - DNA, 25 bp, Artificial Sequence
  synthetic nucleotide probe
cacagtggcttctcaggaggctgag

SEQ ID NO: 483 - DNA, 25 bp, Artificial Sequence
  synthetic nucleotide probe
ggatagaatcatggtgggcacagtg

SEQ ID NO: 484 - DNA, 25 bp, Artificial Sequence
  synthetic nucleotide probe
tctccttgtttaccctggtattcta

SEQ ID NO: 485 - DNA, 25 bp, Artificial Sequence
  synthetic nucleotide probe
aagtgtctagttcttgctaaaatca

SEQ ID NO: 486 - DNA, 25 bp, Artificial Sequence
  synthetic nucleotide probe
tggagaattctttaggttgtcccct

SEQ ID NO: 487 - DNA, 25 bp, Artificial Sequence
  synthetic nucleotide probe
ggagggaggtttgcttgagtccagg

SEQ ID NO: 488 - DNA, 25 bp, Artificial Sequence
  synthetic nucleotide probe
tggcaaggaggacaaatacgcaatg

SEQ ID NO: 489 - DNA, 25 bp, Artificial Sequence
  synthetic nucleotide probe
tcatctttgaataacgtctccttgt

SEQ ID NO: 490 - DNA, 25 bp, Artificial Sequence
  synthetic nucleotide probe
gataatgcctctacaacaacaagaa

SEQ ID NO: 491 - DNA, 25 bp, Artificial Sequence
  synthetic nucleotide probe
tgaacttgatacgtccgtgtgtccc

SEQ ID NO: 492 - DNA, 25 bp, Artificial Sequence
  synthetic nucleotide probe
gggcagttttgaggcatgactaatg

SEQ ID NO: 493 - DNA, 25 bp, Artificial Sequence
  synthetic nucleotide probe
aaaagcgaggtggccatgttatgct

SEQ ID NO: 494 - DNA, 25 bp, Artificial Sequence
  synthetic nucleotide probe
taactataaggtgcctcagttttcc

SEQ ID NO: 495 - DNA, 25 bp, Artificial Sequence
  synthetic nucleotide probe
agatgctgtcattagtctatatggt

SEQ ID NO: 496 - DNA, 25 bp, Artificial Sequence
  synthetic nucleotide probe
ggaattctcaagacctgagtatttt

SEQ ID NO: 497 - DNA, 25 bp, Artificial Sequence
  synthetic nucleotide probe
ctgacctacctcaaagggcagtttt

SEQ ID NO: 498 - DNA, 25 bp, Artificial Sequence
  synthetic nucleotide probe
acaacgcattgccacggaaacatac

SEQ ID NO: 499 - DNA, 25 bp, Artificial Sequence
  synthetic nucleotide probe
agcattttgggatccttcagcacag

SEQ ID NO: 500 - DNA, 25 bp, Artificial Sequence
  synthetic nucleotide probe
gaagctaattgtactaatctgagat

SEQ ID NO: 501 - DNA, 25 bp, Artificial Sequence
  synthetic nucleotide probe
atgtccaccatgaacttgatacgtc

SEQ ID NO: 502 - DNA, 25 bp, Artificial Sequence
  synthetic nucleotide probe
cattctgtcgaccctggatgttgaa

SEQ ID NO: 503 - DNA, 25 bp, Artificial Sequence
  synthetic nucleotide probe
ttgagagttttgacctgcctgaaga

SEQ ID NO: 504 - DNA, 25 bp, Artificial Sequence
  synthetic nucleotide probe
aattgccacctgtttgctgtgacat

SEQ ID NO: 505 - DNA, 25 bp, Artificial Sequence
  synthetic nucleotide probe
gtgcctctcatgatccttgacgagg

SEQ ID NO: 506 - DNA, 25 bp, Artificial Sequence
  synthetic nucleotide probe
tgcagtctccttcaagcattctgtc

SEQ ID NO: 507 - DNA, 25 bp, Artificial Sequence
  synthetic nucleotide probe
cctgcctcagatgatgcctatccag

SEQ ID NO: 508 - DNA, 25 bp, Artificial Sequence
  synthetic nucleotide probe
aaaacagccaagcttttctgccaaa

SEQ ID NO: 509 - DNA, 25 bp, Artificial Sequence
  synthetic nucleotide probe
gggaatccaatctgttgcagtctcc

SEQ ID NO: 510 - DNA, 25 bp, Artificial Sequence
  synthetic nucleotide probe
tgaagagcaccagattgcgcacctc

SEQ ID NO: 511 - DNA, 25 bp, Artificial Sequence
  synthetic nucleotide probe
aagcaaaaagctctgttcctgcctc

SEQ ID NO: 512 - DNA, 25 bp, Artificial Sequence
  synthetic nucleotide probe
ttcccttcaatcctctagactttga

SEQ ID NO: 513 - DNA, 25 bp, Artificial Sequence
  synthetic nucleotide probe
ggtcaaagttgctcagcggagccca

SEQ ID NO: 514 - DNA, 25 bp, Artificial Sequence
  synthetic nucleotide probe
tgcacagaagttagcgctatcccca

SEQ ID NO: 515 - DNA, 25 bp, Artificial Sequence
  synthetic nucleotide probe
tacccctgggaggtgccagtcattg

SEQ ID NO: 516 - DNA, 25 bp, Artificial Sequence
  synthetic nucleotide probe
gtttggaagcactgatcacctgtta

SEQ ID NO: 517 - DNA, 25 bp, Artificial Sequence
  synthetic nucleotide probe
gaaggcactttgtgtgtcagtaccc

SEQ ID NO: 518 - DNA, 25 bp, Artificial Sequence
  synthetic nucleotide probe
gatcacctgttagcattgccattcc

SEQ ID NO: 519 - DNA, 25 bp, Artificial Sequence
  synthetic nucleotide probe
gagcccagtagattcaggcaccatc

SEQ ID NO: 520 - DNA, 25 bp, Artificial Sequence
  synthetic nucleotide probe
gtactctttagatctcccatgtgta

SEQ ID NO: 521 - DNA, 25 bp, Artificial Sequence
  synthetic nucleotide probe
tgagggtcaagcgaggccgacttgt

SEQ ID NO: 522 - DNA, 25 bp, Artificial Sequence
  synthetic nucleotide probe
ttgccattcctctactgcaatgtaa

SEQ ID NO: 523 - DNA, 25 bp, Artificial Sequence
  synthetic nucleotide probe
cgaaatccgtcccagtcaataatct

SEQ ID NO: 524 - DNA, 25 bp, Artificial Sequence
  synthetic nucleotide probe
ggacagtgtttcaacaagcctaggc

SEQ ID NO: 525 - DNA, 25 bp, Artificial Sequence
  synthetic nucleotide probe
gcatctaagggactttgttgaactt

SEQ ID NO: 526 - DNA, 25 bp, Artificial Sequence
  synthetic nucleotide probe
gatggcctctgatttacactggttc

SEQ ID NO: 527 - DNA, 25 bp, Artificial Sequence
  synthetic nucleotide probe
agaagtctgccctagctgttaaatt

SEQ ID NO: 528 - DNA, 25 bp, Artificial Sequence
  synthetic nucleotide probe
gagttaattgttcctttcttcagtg

SEQ ID NO: 529 - DNA, 25 bp, Artificial Sequence
  synthetic nucleotide probe
tagacagcttggatcctttctctga

SEQ ID NO: 530 - DNA, 25 bp, Artificial Sequence
  synthetic nucleotide probe
ggtttaccaggatgtagtcccactg

SEQ ID NO: 531 - DNA, 25 bp, Artificial Sequence
  synthetic nucleotide probe
gaaaacacttagttcattggcttta

SEQ ID NO: 532 - DNA, 25 bp, Artificial Sequence
  synthetic nucleotide probe
gcgtatttttacactattggctcaa

SEQ ID NO: 533 - DNA, 25 bp, Artificial Sequence
  synthetic nucleotide probe
atttacacagctagatttggaagat

SEQ ID NO: 534 - DNA, 25 bp, Artificial Sequence
  synthetic nucleotide probe
ggatgagattgatgcagcccttgat

SEQ ID NO: 535 - DNA, 25 bp, Artificial Sequence
  synthetic nucleotide probe
attgatgcagcccttgattttaaaa

SEQ ID NO: 536 - DNA, 25 bp, Artificial Sequence
  synthetic nucleotide probe
agttcataataatttctcttcgaaa

SEQ ID NO: 537 - DNA, 25 bp, Artificial Sequence
  synthetic nucleotide probe
ggaaggactttcggtattgtattag

SEQ ID NO: 538 - DNA, 25 bp, Artificial Sequence
  synthetic nucleotide probe
cctttcttcagtgggccattgtttt

SEQ ID NO: 539 - DNA, 25 bp, Artificial Sequence
  synthetic nucleotide probe
ttagtatttgctcttcaccactaca

SEQ ID NO: 540 - DNA, 25 bp, Artificial Sequence
  synthetic nucleotide probe
gataccttgagtaatgtttgcctat

SEQ ID NO: 541 - DNA, 25 bp, Artificial Sequence
  synthetic nucleotide probe
cactcccctttacttcatggatgag

SEQ ID NO: 542 - DNA, 25 bp, Artificial Sequence
  synthetic nucleotide probe
aagcctaggctatctcgtaagttga

SEQ ID NO: 543 - DNA, 25 bp, Artificial Sequence
  synthetic nucleotide probe
ggacgccgaactcgagcttgtagac

SEQ ID NO: 544 - DNA, 25 bp, Artificial Sequence
  synthetic nucleotide probe
aatatcccactatagttgcttcatg

SEQ ID NO: 545 - DNA, 25 bp, Artificial Sequence
  synthetic nucleotide probe
cccaatttctcaatgaagatctaag

SEQ ID NO: 546 - DNA, 25 bp, Artificial Sequence
  synthetic nucleotide probe
gattatgtccagttatttgctttaa

SEQ ID NO: 547 - DNA, 25 bp, Artificial Sequence
  synthetic nucleotide probe
ggtggaatcctttaagattatgtcc

SEQ ID NO: 548 - DNA, 25 bp, Artificial Sequence
  synthetic nucleotide probe
aagacgatggaggtggaatccttta

SEQ ID NO: 549 - DNA, 25 bp, Artificial Sequence
  synthetic nucleotide probe
gagccaaaaccgcagcactagaaaa

SEQ ID NO: 550 - DNA, 25 bp, Artificial Sequence
  synthetic nucleotide probe
gagactaccaagacgagccaaaacc

SEQ ID NO: 551 - DNA, 25 bp, Artificial Sequence
  synthetic nucleotide probe
ttccagaaccagaatcagaaatgaa

SEQ ID NO: 552 - DNA, 25 bp, Artificial Sequence
  synthetic nucleotide probe
ccaagacgagccaaaaccgcagcac

SEQ ID NO: 553 - DNA, 25 bp, Artificial Sequence
  synthetic nucleotide probe
ggacgaacaggaggtgtcagactgc

SEQ ID NO: 554 - DNA, 25 bp, Artificial Sequence
  synthetic nucleotide probe
gaagatgagactaccaagacgagcc

SEQ ID NO: 555 - DNA, 25 bp, Artificial Sequence
  synthetic nucleotide probe
gtgtcagactgctgaagccgactct

SEQ ID NO: 556 - DNA, 25 bp, Artificial Sequence
  synthetic nucleotide probe
cttgctttgtatgctcagtgtgttg

SEQ ID NO: 557 - DNA, 25 bp, Artificial Sequence
  synthetic nucleotide probe
gccctctttggtactatatgccatg

SEQ ID NO: 558 - DNA, 25 bp, Artificial Sequence
  synthetic nucleotide probe
acacctggcatgacacttgctttgt

SEQ ID NO: 559 - DNA, 25 bp, Artificial Sequence
  synthetic nucleotide probe
gaatttcccatagaagctggtgaca

SEQ ID NO: 560 - DNA, 25 bp, Artificial Sequence
  synthetic nucleotide probe
gaagctggtgacatgttcctggaag

SEQ ID NO: 561 - DNA, 25 bp, Artificial Sequence
  synthetic nucleotide probe
caagaaggacaagcccctgtgtaag

SEQ ID NO: 562 - DNA, 25 bp, Artificial Sequence
  synthetic nucleotide probe
tgacatgttcctggaagctctgggc

SEQ ID NO: 563 - DNA, 25 bp, Artificial Sequence
  synthetic nucleotide probe
atatgccatggatgtgaatttccca

SEQ ID NO: 564 - DNA, 25 bp, Artificial Sequence
  synthetic nucleotide probe
gcccctgtgtaagaaacatgctcat

SEQ ID NO: 565 - DNA, 25 bp, Artificial Sequence
  synthetic nucleotide probe
ggaaggtcagacctttttctccaag

SEQ ID NO: 566 - DNA, 25 bp, Artificial Sequence
  synthetic nucleotide probe
ttattatgccctctttggtactata

SEQ ID NO: 567 - DNA, 25 bp, Artificial Sequence
  synthetic nucleotide probe
gagaggaccaaccagaattcccttt

SEQ ID NO: 568 - DNA, 25 bp, Artificial Sequence
  synthetic nucleotide probe
aagcatgtgtcatccatatttctct

SEQ ID NO: 569 - DNA, 25 bp, Artificial Sequence
  synthetic nucleotide probe
ctacctggaggggatcagcccactg

SEQ ID NO: 570 - DNA, 25 bp, Artificial Sequence
  synthetic nucleotide probe
agttgaacagtgtgccctagctttt

SEQ ID NO: 571 - DNA, 25 bp, Artificial Sequence
  synthetic nucleotide probe
ggagaatcgtgtgatcagtgtgcta

SEQ ID NO: 572 - DNA, 25 bp, Artificial Sequence
  synthetic nucleotide probe
gtagtgtatcactgagtcatttgca

SEQ ID NO: 573 - DNA, 25 bp, Artificial Sequence
  synthetic nucleotide probe
tgggctgccttatattgtgtgtgtg

SEQ ID NO: 574 - DNA, 25 bp, Artificial Sequence
  synthetic nucleotide probe
tgttttctgccacagacctttgggc

SEQ ID NO: 575 - DNA, 25 bp, Artificial Sequence
  synthetic nucleotide probe
tgttctctccgtgaaacttaccttt

SEQ ID NO: 576 - DNA, 25 bp, Artificial Sequence
  synthetic nucleotide probe
aaatgctcttatttttccaacagct

SEQ ID NO: 577 - DNA, 25 bp, Artificial Sequence
  synthetic nucleotide probe
cctagcttttcttgcaaccagagta

SEQ ID NO: 578 - DNA, 25 bp, Artificial Sequence
  synthetic nucleotide probe
gaattccctttggacatttgtgttt

SEQ ID NO: 579 - DNA, 25 bp, Artificial Sequence
  synthetic nucleotide probe
gccaaccttggctaaatggagcagc

SEQ ID NO: 580 - DNA, 25 bp, Artificial Sequence
  synthetic nucleotide probe
attactgctgtggctagagagtttg

SEQ ID NO: 581 - DNA, 25 bp, Artificial Sequence
  synthetic nucleotide probe
ttggagtgagggaggctacctggag

SEQ ID NO: 582 - DNA, 25 bp, Artificial Sequence
  synthetic nucleotide probe
atatggcatccttcaatttctgtat

SEQ ID NO: 583 - DNA, 25 bp, Artificial Sequence
  synthetic nucleotide probe
cagcccactgacagaccttaatctt

SEQ ID NO: 584 - DNA, 25 bp, Artificial Sequence
  synthetic nucleotide probe
atcagtggccaggccaaccttggct

SEQ ID NO: 585 - DNA, 25 bp, Artificial Sequence
  synthetic nucleotide probe
ttttccaacagctaaactactctta

SEQ ID NO: 586 - DNA, 25 bp, Artificial Sequence
  synthetic nucleotide probe
gcaactcgtacccaaatttccaaga

SEQ ID NO: 587 - DNA, 25 bp, Artificial Sequence
  synthetic nucleotide probe
acatgacctatccaagcgcattacc

SEQ ID NO: 588 - DNA, 25 bp, Artificial Sequence
  synthetic nucleotide probe
gtaaaagctttggtttgtgttcgtg

SEQ ID NO: 589 - DNA, 25 bp, Artificial Sequence
  synthetic nucleotide probe
tagttctctcctcacttgtaaactt

SEQ ID NO: 590 - DNA, 25 bp, Artificial Sequence
  synthetic nucleotide probe
gtatatgtatcttcctcaatttccc

SEQ ID NO: 591 - DNA, 25 bp, Artificial Sequence
  synthetic nucleotide probe
ggaggcagtgaagggcttgccctgc

SEQ ID NO: 592 - DNA, 25 bp, Artificial Sequence
  synthetic nucleotide probe
catcttcccctgtgagtgacatgtc

SEQ ID NO: 593 - DNA, 25 bp, Artificial Sequence
  synthetic nucleotide probe
aggttggaagtgtgatgggtggggg

SEQ ID NO: 594 - DNA, 25 bp, Artificial Sequence
  synthetic nucleotide probe
ggtatctttttgtcacaccaaaatc

SEQ ID NO: 595 - DNA, 25 bp, Artificial Sequence
  synthetic nucleotide probe
cccctcccattaaagatccgggcag

SEQ ID NO: 596 - DNA, 25 bp, Artificial Sequence
  synthetic nucleotide probe
aaagtaacatcaacactgtcccatc

SEQ ID NO: 597 - DNA, 25 bp, Artificial Sequence
  synthetic nucleotide probe
gatcccctcagacattctcaggatt

SEQ ID NO: 598 - DNA, 25 bp, Artificial Sequence
  synthetic nucleotide probe
gactgtcagagtggggaacccctcc

SEQ ID NO: 599 - DNA, 25 bp, Artificial Sequence
  synthetic nucleotide probe
gggttggggtgcttgtatatgtatc

SEQ ID NO: 600 - DNA, 25 bp, Artificial Sequence
  synthetic nucleotide probe
tatgttctcattctatggccattgt

SEQ ID NO: 601 - DNA, 25 bp, Artificial Sequence
  synthetic nucleotide probe
gagtctcagaatgctcaggaccaag

SEQ ID NO: 602 - DNA, 25 bp, Artificial Sequence
  synthetic nucleotide probe
tgtggccagacagatgacacctttt

SEQ ID NO: 603 - DNA, 25 bp, Artificial Sequence
  synthetic nucleotide probe
gtctgctctggtgtgatctgaaaag

SEQ ID NO: 604 - DNA, 25 bp, Artificial Sequence
  synthetic nucleotide probe
gctttatctcatgatgcttgcttgt

SEQ ID NO: 605 - DNA, 25 bp, Artificial Sequence
  synthetic nucleotide probe
ggggtagaaactggtgtctgctctg

SEQ ID NO: 606 - DNA, 25 bp, Artificial Sequence
  synthetic nucleotide probe
caggagacccagcgatctgagcata

SEQ ID NO: 607 - DNA, 25 bp, Artificial Sequence
  synthetic nucleotide probe
gaaaaggcgtcttcactgctttatc

SEQ ID NO: 608 - DNA, 25 bp, Artificial Sequence
  synthetic nucleotide probe
tatggccattgtgttgcctctgtta

SEQ ID NO: 609 - DNA, 25 bp, Artificial Sequence
  synthetic nucleotide probe
atcactagtgcatgctgtggccaga

SEQ ID NO: 610 - DNA, 25 bp, Artificial Sequence
  synthetic nucleotide probe
aacatcttcatgtgggctggggtag

SEQ ID NO: 611 - DNA, 25 bp, Artificial Sequence
  synthetic nucleotide probe
cccttcccgcatttattggtgtatt

SEQ ID NO: 612 - DNA, 25 bp, Artificial Sequence
  synthetic nucleotide probe
acctttgtagctagcaccagtgcca

SEQ ID NO: 613 - DNA, 25 bp, Artificial Sequence
  synthetic nucleotide probe
ttcatctcagatttgttcatcacag

SEQ ID NO: 614 - DNA, 25 bp, Artificial Sequence
  synthetic nucleotide probe
gtcttcagtagacaagtcacctttg

SEQ ID NO: 615 - DNA, 25 bp, Artificial Sequence
  synthetic nucleotide probe
ttaaggactccatgaacctgggcta

SEQ ID NO: 616 - DNA, 25 bp, Artificial Sequence
  synthetic nucleotide probe
taatgttgctactcccatggcaaag

SEQ ID NO: 617 - DNA, 25 bp, Artificial Sequence
  synthetic nucleotide probe
gtttttgtcctaatgttgctactcc

SEQ ID NO: 618 - DNA, 25 bp, Artificial Sequence
  synthetic nucleotide probe
cagaggacatcttggggagggggag

SEQ ID NO: 619 - DNA, 25 bp, Artificial Sequence
  synthetic nucleotide probe
caccttctttagtcttgattgccct

SEQ ID NO: 620 - DNA, 25 bp, Artificial Sequence
  synthetic nucleotide probe
ccattgcaccttctttagtcttgat

SEQ ID NO: 621 - DNA, 25 bp, Artificial Sequence
  synthetic nucleotide probe
gatgtggcttttgtgatattctatc

SEQ ID NO: 622 - DNA, 25 bp, Artificial Sequence
  synthetic nucleotide probe
cacggtcagttgtaactttgccttc

SEQ ID NO: 623 - DNA, 25 bp, Artificial Sequence
  synthetic nucleotide probe
gactatccaacttaacatgaaactt

SEQ ID NO: 624 - DNA, 25 bp, Artificial Sequence
  synthetic nucleotide probe
gagatagcattagctgcccaggatg

SEQ ID NO: 625 - DNA, 25 bp, Artificial Sequence
  synthetic nucleotide probe
aatggagctatgtcttgttttaagt

SEQ ID NO: 626 - DNA, 25 bp, Artificial Sequence
  synthetic nucleotide probe
gagagggaggatgtcacggtcagtt

SEQ ID NO: 627 - DNA, 25 bp, Artificial Sequence
  synthetic nucleotide probe
agttggtcttttgacgagagggagg

SEQ ID NO: 628 - DNA, 25 bp, Artificial Sequence
  synthetic nucleotide probe
gtgcctccttgacatttcgttcaag

SEQ ID NO: 629 - DNA, 25 bp, Artificial Sequence
  synthetic nucleotide probe
gaaacttgtcaccatgagatagcat

SEQ ID NO: 630 - DNA, 25 bp, Artificial Sequence
  synthetic nucleotide probe
aaagctacaatctgttcaatgtttt

SEQ ID NO: 631 - DNA, 25 bp, Artificial Sequence
  synthetic nucleotide probe
ctgcccaggatgctgctatatatat

SEQ ID NO: 632 - DNA, 25 bp, Artificial Sequence
  synthetic nucleotide probe
aaaaactcatttatacctgtgtatt

SEQ ID NO: 633 - DNA, 25 bp, Artificial Sequence
  synthetic nucleotide probe
tgaccgagcagcagagtgtaacatc

SEQ ID NO: 634 - DNA, 25 bp, Artificial Sequence
  synthetic nucleotide probe
gtaacatcgtagtaactcagcccag

SEQ ID NO: 635 - DNA, 25 bp, Artificial Sequence
  synthetic nucleotide probe
atcagtgcggtttctgtggcagagc

SEQ ID NO: 636 - DNA, 25 bp, Artificial Sequence
  synthetic nucleotide probe
gactttatccagaatgaccgagcag

SEQ ID NO: 637 - DNA, 25 bp, Artificial Sequence
  synthetic nucleotide probe
tagaggggctactggatgtgggaaa

SEQ ID NO: 638 - DNA, 25 bp, Artificial Sequence
  synthetic nucleotide probe
ctcagcccagaagaatcagtgcggt

SEQ ID NO: 639 - DNA, 25 bp, Artificial Sequence
  synthetic nucleotide probe
ggcttatcctgaagttcgcattgtt

SEQ ID NO: 640 - DNA, 25 bp, Artificial Sequence
  synthetic nucleotide probe
tgggaaaaccacacaggttccccag

SEQ ID NO: 641 - DNA, 25 bp, Artificial Sequence
  synthetic nucleotide probe
tgtactgtaggtgtgctcctgagaa

SEQ ID NO: 642 - DNA, 25 bp, Artificial Sequence
  synthetic nucleotide probe
gcgtgatgttgttcaggcttatcct

SEQ ID NO: 643 - DNA, 25 bp, Artificial Sequence
  synthetic nucleotide probe
ggaggacttacccagttcaagaata

SEQ ID NO: 644 - DNA, 25 bp, Artificial Sequence
  synthetic nucleotide probe
ggatggcttctaacaaaaactacac

SEQ ID NO: 645 - DNA, 25 bp, Artificial Sequence
  synthetic nucleotide probe
gtgtgctatggatggcttctaacaa

SEQ ID NO: 646 - DNA, 25 bp, Artificial Sequence
  synthetic nucleotide probe
tagttacacatcttcaacagacccc

SEQ ID NO: 647 - DNA, 25 bp, Artificial Sequence
  synthetic nucleotide probe
agggtgaagctatttatctgtagta

SEQ ID NO: 648 - DNA, 25 bp, Artificial Sequence
  synthetic nucleotide probe
ttagggcccaattaataatcagcaa

SEQ ID NO: 649 - DNA, 25 bp, Artificial Sequence
  synthetic nucleotide probe
cttccatagcctaatccctgggcat

SEQ ID NO: 650 - DNA, 25 bp, Artificial Sequence
  synthetic nucleotide probe
cacatatgtattcctgatcgccaac

SEQ ID NO: 651 - DNA, 25 bp, Artificial Sequence
  synthetic nucleotide probe
cagaccccctctagaaatttttcag

SEQ ID NO: 652 - DNA, 25 bp, Artificial Sequence
  synthetic nucleotide probe
ttgaatgggtccattttgcccttcc

SEQ ID NO: 653 - DNA, 25 bp, Artificial Sequence
  synthetic nucleotide probe
cagggttaatagggcctggtccctg

SEQ ID NO: 654 - DNA, 25 bp, Artificial Sequence
  synthetic nucleotide probe
ttaaaccctggatcagtcctttgat

SEQ ID NO: 655 - DNA, 25 bp, Artificial Sequence
  synthetic nucleotide probe
gtattcagtatttgaacgtcgtcct

SEQ ID NO: 656 - DNA, 25 bp, Artificial Sequence
  synthetic nucleotide probe
gtcttgcattgtgaggtacaggcgg

SEQ ID NO: 657 - DNA, 25 bp, Artificial Sequence
  synthetic nucleotide probe
ttttaccttggatgctgacttctaa

SEQ ID NO: 658 - DNA, 25 bp, Artificial Sequence
  synthetic nucleotide probe
gtacaggcggaagttggaatcaggt

SEQ ID NO: 659 - DNA, 25 bp, Artificial Sequence
  synthetic nucleotide probe
gaccctttagtgagcttagcacagc

SEQ ID NO: 660 - DNA, 25 bp, Artificial Sequence
  synthetic nucleotide probe
cctggctggctgtgacttaccatag

SEQ ID NO: 661 - DNA, 25 bp, Artificial Sequence
  synthetic nucleotide probe
gaacgtcgtcctgtttattgttagt

SEQ ID NO: 662 - DNA, 25 bp, Artificial Sequence
  synthetic nucleotide probe
ctcacaaccagtcctgtctgtttat

SEQ ID NO: 663 - DNA, 25 bp, Artificial Sequence
  synthetic nucleotide probe
gaagtgttaccaactagccacacca

SEQ ID NO: 664 - DNA, 25 bp, Artificial Sequence
  synthetic nucleotide probe
atgtgaggattaacttctgccagct

SEQ ID NO: 665 - DNA, 25 bp, Artificial Sequence
  synthetic nucleotide probe
ctagccacaccatgaattgtccgta

SEQ ID NO: 666 - DNA, 25 bp, Artificial Sequence
  synthetic nucleotide probe
cagcctcactgcttcaacgcagatt

SEQ ID NO: 667 - DNA, 25 bp, Artificial Sequence
  synthetic nucleotide probe
ttaggattctgtctctcattagctg

SEQ ID NO: 668 - DNA, 25 bp, Artificial Sequence
  synthetic nucleotide probe
gtgctggtagtatcaccttttgcca

SEQ ID NO: 669 - DNA, 25 bp, Artificial Sequence
  synthetic nucleotide probe
tatggtccttatatgtgtacaacat

SEQ ID NO: 670 - DNA, 25 bp, Artificial Sequence
  synthetic nucleotide probe
gaagatgtgcccttacttggctgat

SEQ ID NO: 671 - DNA, 25 bp, Artificial Sequence
  synthetic nucleotide probe
taaacagtcctttaaccagcacagc

SEQ ID NO: 672 - DNA, 25 bp, Artificial Sequence
  synthetic nucleotide probe
tgaagtatttttatgctctgaatgt

SEQ ID NO: 673 - DNA, 25 bp, Artificial Sequence
  synthetic nucleotide probe
caaagatcaagggctgtccgcaaca

SEQ ID NO: 674 - DNA, 25 bp, Artificial Sequence
  synthetic nucleotide probe
gatgaatatttttctactggtattt

SEQ ID NO: 675 - DNA, 25 bp, Artificial Sequence
  synthetic nucleotide probe
gacatagtgtttattagcagccatc

SEQ ID NO: 676 - DNA, 25 bp, Artificial Sequence
  synthetic nucleotide probe
gaaagctttttgtctaagtgaattc

SEQ ID NO: 677 - DNA, 25 bp, Artificial Sequence
  synthetic nucleotide probe
gtgaattcttatgccttggtcagag

SEQ ID NO: 678 - DNA, 25 bp, Artificial Sequence
  synthetic nucleotide probe
tgttaactatacaacctggctaaag

SEQ ID NO: 679 - DNA, 25 bp, Artificial Sequence
  synthetic nucleotide probe
aaatgttctcatcagtttcttgcca

SEQ ID NO: 680 - DNA, 25 bp, Artificial Sequence
  synthetic nucleotide probe
tgctaagttcaagtttcgtaatgct

SEQ ID NO: 681 - DNA, 25 bp, Artificial Sequence
  synthetic nucleotide probe
aagggctgtccgcaacagggaagaa

SEQ ID NO: 682 - DNA, 25 bp, Artificial Sequence
  synthetic nucleotide probe
cttatcttggctttcgagtctgagt

SEQ ID NO: 683 - DNA, 25 bp, Artificial Sequence
  synthetic nucleotide probe
ggcctctaatatctttgggacacct

SEQ ID NO: 684 - DNA, 25 bp, Artificial Sequence
  synthetic nucleotide probe
gaaggaactcctctgaagcaagctc

SEQ ID NO: 685 - DNA, 25 bp, Artificial Sequence
  synthetic nucleotide probe
gacttggagtcatctggactgcaga

SEQ ID NO: 686 - DNA, 25 bp, Artificial Sequence
  synthetic nucleotide probe
agagtgaagagaagcccgtgcctgc

SEQ ID NO: 687 - DNA, 25 bp, Artificial Sequence
  synthetic nucleotide probe
gaccccaacagcaggaatagctccc

SEQ ID NO: 688 - DNA, 25 bp, Artificial Sequence
  synthetic nucleotide probe
gtggtggatccaatttttcattagg

SEQ ID NO: 689 - DNA, 25 bp, Artificial Sequence
  synthetic nucleotide probe
catccagaagaaatccccctggcgg

SEQ ID NO: 690 - DNA, 25 bp, Artificial Sequence
  synthetic nucleotide probe
acaaccaccaccttcaagggagtcg

SEQ ID NO: 691 - DNA, 25 bp, Artificial Sequence
  synthetic nucleotide probe
gcaagctccggagacttcttagatc

SEQ ID NO: 692 - DNA, 25 bp, Artificial Sequence
  synthetic nucleotide probe
gatgtaccttttttgtcaactctta

SEQ ID NO: 693 - DNA, 25 bp, Artificial Sequence
  synthetic nucleotide probe
tagtgataccttgatctttcccact

SEQ ID NO: 694 - DNA, 25 bp, Artificial Sequence
  synthetic nucleotide probe
gtttcattgacctctagtgatacct

SEQ ID NO: 695 - DNA, 25 bp, Artificial Sequence
  synthetic nucleotide probe
gtacagcctagtgttaacattcttg

SEQ ID NO: 696 - DNA, 25 bp, Artificial Sequence
  synthetic nucleotide probe
gattggcttttgtcatccactattg

SEQ ID NO: 697 - DNA, 25 bp, Artificial Sequence
  synthetic nucleotide probe
aacatttctcgatcactggtttcag

SEQ ID NO: 698 - DNA, 25 bp, Artificial Sequence
  synthetic nucleotide probe
tttcccactttctgttttcggattg

SEQ ID NO: 699 - DNA, 25 bp, Artificial Sequence
  synthetic nucleotide probe
ggcctcctatgatgcagacatggtg

SEQ ID NO: 700 - DNA, 25 bp, Artificial Sequence
  synthetic nucleotide probe
tcttggtatctttttgtgccttatc

SEQ ID NO: 701 - DNA, 25 bp, Artificial Sequence
  synthetic nucleotide probe
aggagctgggactggtttgaacaca

SEQ ID NO: 702 - DNA, 25 bp, Artificial Sequence
  synthetic nucleotide probe
cagatggggagggggtactggcctt

SEQ ID NO: 703 - DNA, 25 bp, Artificial Sequence
  synthetic nucleotide probe
atagagcctctgatgtacgaagtag

SEQ ID NO: 704 - DNA, 25 bp, Artificial Sequence
  synthetic nucleotide probe
cagtctctacaaacttacagctcat

SEQ ID NO: 705 - DNA, 25 bp, Artificial Sequence
  synthetic nucleotide probe
aatcccctgcaagctattcaaatgg

SEQ ID NO: 706 - DNA, 25 bp, Artificial Sequence
  synthetic nucleotide probe
gaagaaatcacaaatcccctgcaag

SEQ ID NO: 707 - DNA, 25 bp, Artificial Sequence
  synthetic nucleotide probe
gtgatggatacgcttggcattcctt

SEQ ID NO: 708 - DNA, 25 bp, Artificial Sequence
  synthetic nucleotide probe
gcattccttttatcccagaaacacc

SEQ ID NO: 709 - DNA, 25 bp, Artificial Sequence
  synthetic nucleotide probe
gttgtttgttggctattttactgaa

SEQ ID NO: 710 - DNA, 25 bp, Artificial Sequence
  synthetic nucleotide probe
ggagcttttgcagatataccgagaa

SEQ ID NO: 711 - DNA, 25 bp, Artificial Sequence
  synthetic nucleotide probe
tcagatatgctgtgcaagtcttgct

SEQ ID NO: 712 - DNA, 25 bp, Artificial Sequence
  synthetic nucleotide probe
gttgttgaccgtatttacagtctct

SEQ ID NO: 713 - DNA, 25 bp, Artificial Sequence
  synthetic nucleotide probe
gttgtaatcgcagtattccttgtat

SEQ ID NO: 714 - DNA, 25 bp, Artificial Sequence
  synthetic nucleotide probe
agtagctctctgcttgctgatagat

SEQ ID NO: 715 - DNA, 25 bp, Artificial Sequence
  synthetic nucleotide probe
attttagtttagcttcctgatttat

SEQ ID NO: 716 - DNA, 25 bp, Artificial Sequence
  synthetic nucleotide probe
gatggtttcccagtgtgagatttgt

SEQ ID NO: 717 - DNA, 25 bp, Artificial Sequence
  synthetic nucleotide probe
atggtttggttggtcccagcaaagt

SEQ ID NO: 718 - DNA, 25 bp, Artificial Sequence
  synthetic nucleotide probe
gttctcggtgttcaaactcttctaa

SEQ ID NO: 719 - DNA, 25 bp, Artificial Sequence
  synthetic nucleotide probe
tgcaaatatgctgtgggttctcggt

SEQ ID NO: 720 - DNA, 25 bp, Artificial Sequence
  synthetic nucleotide probe
aaggaattgctgttactgtactgca

SEQ ID NO: 721 - DNA, 25 bp, Artificial Sequence
  synthetic nucleotide probe
taaatctagtgtttctattttagtt

SEQ ID NO: 722 - DNA, 25 bp, Artificial Sequence
  synthetic nucleotide probe
atataccatcccatatatatgtggg

SEQ ID NO: 723 - DNA, 25 bp, Artificial Sequence
  synthetic nucleotide probe
ctgcttgctgatagatggtttccca

SEQ ID NO: 724 - DNA, 25 bp, Artificial Sequence
  synthetic nucleotide probe
atgccagagacataccaggcgcagc

SEQ ID NO: 725 - DNA, 25 bp, Artificial Sequence
  synthetic nucleotide probe
ggaagatcacatcctacttcctcaa

SEQ ID NO: 726 - DNA, 25 bp, Artificial Sequence
  synthetic nucleotide probe
actgattatggctcaggctacccca

SEQ ID NO: 727 - DNA, 25 bp, Artificial Sequence
  synthetic nucleotide probe
tgcagcaaagttttcccgggattga

SEQ ID NO: 728 - DNA, 25 bp, Artificial Sequence
  synthetic nucleotide probe
ctcaatgaagggtcccaagcccgtc

SEQ ID NO: 729 - DNA, 25 bp, Artificial Sequence
  synthetic nucleotide probe
tcgtggacaccgtagggatgccaga

SEQ ID NO: 730 - DNA, 25 bp, Artificial Sequence
  synthetic nucleotide probe
gaggactcagcatccgagaatcagg

SEQ ID NO: 731 - DNA, 25 bp, Artificial Sequence
  synthetic nucleotide probe
tcttcccaccgatatttcctggaac

SEQ ID NO: 732 - DNA, 25 bp, Artificial Sequence
  synthetic nucleotide probe
tggcctcacaggtgcatcatagccg

SEQ ID NO: 733 - DNA, 25 bp, Artificial Sequence
  synthetic nucleotide probe
gggcaacgactttgtgtccattgtc

SEQ ID NO: 734 - DNA, 25 bp, Artificial Sequence
  synthetic nucleotide probe
ccctgatgcccaccaaggacgtgga

SEQ ID NO: 735 - DNA, 25 bp, Artificial Sequence
  synthetic nucleotide probe
agcaccgctgcgacaagaagcgcca

SEQ ID NO: 736 - DNA, 25 bp, Artificial Sequence
  synthetic nucleotide probe
tcaactttgtccacgtgatcgtcac

SEQ ID NO: 737 - DNA, 25 bp, Artificial Sequence
  synthetic nucleotide probe
gtgaggacttcaagcttggcaccat

SEQ ID NO: 738 - DNA, 25 bp, Artificial Sequence
  synthetic nucleotide probe
tggactacgagtgcaacctggtgtc

SEQ ID NO: 739 - DNA, 25 bp, Artificial Sequence
  synthetic nucleotide probe
gtattaataaggcattgccccctgt

SEQ ID NO: 740 - DNA, 25 bp, Artificial Sequence
  synthetic nucleotide probe
tgtaaggctgcattgtgggtttggg

SEQ ID NO: 741 - DNA, 25 bp, Artificial Sequence
  synthetic nucleotide probe
gttttgtaacactgtcctactttat

SEQ ID NO: 742 - DNA, 25 bp, Artificial Sequence
  synthetic nucleotide probe
agtcgttgcagggtttggatcagct

SEQ ID NO: 743 - DNA, 25 bp, Artificial Sequence
  synthetic nucleotide probe
ggatcagctgtaagttaggtatgcc

SEQ ID NO: 744 - DNA, 25 bp, Artificial Sequence
  synthetic nucleotide probe
aaggctgttacaatcaagtcgttgc

SEQ ID NO: 745 - DNA, 25 bp, Artificial Sequence
  synthetic nucleotide probe
agatgagtcctatacgtggcaattt

SEQ ID NO: 746 - DNA, 25 bp, Artificial Sequence
  synthetic nucleotide probe
tctgaagccagcattatcttccaaa

SEQ ID NO: 747 - DNA, 25 bp, Artificial Sequence
  synthetic nucleotide probe
gccccctgtttgcactcagggttaa

SEQ ID NO: 748 - DNA, 25 bp, Artificial Sequence
  synthetic nucleotide probe
cgtggcaatttttcaatgtcatctg

SEQ ID NO: 749 - DNA, 25 bp, Artificial Sequence
  synthetic nucleotide probe
tcagaactcatggccatttcctgcc

SEQ ID NO: 750 - DNA, 25 bp, Artificial Sequence
  synthetic nucleotide probe
gagatacttgtcaagttgctcttaa

SEQ ID NO: 751 - DNA, 25 bp, Artificial Sequence
  synthetic nucleotide probe
tgggccgtcgacaaaggaaatctga

SEQ ID NO: 752 - DNA, 25 bp, Artificial Sequence
  synthetic nucleotide probe
aatttcgaaaagcagttacagacct

SEQ ID NO: 753 - DNA, 25 bp, Artificial Sequence
  synthetic nucleotide probe
aatctacccatggctacagttgata

SEQ ID NO: 754 - DNA, 25 bp, Artificial Sequence
  synthetic nucleotide probe
gtgggaacaagagctatacaataac

SEQ ID NO: 755 - DNA, 25 bp, Artificial Sequence
  synthetic nucleotide probe
tagtcctagaggatattttcatacc

SEQ ID NO: 756 - DNA, 25 bp, Artificial Sequence
  synthetic nucleotide probe
gatcccccaaatggtcctaatctac

SEQ ID NO: 757 - DNA, 25 bp, Artificial Sequence
  synthetic nucleotide probe
agaaaagacgagatcccccaaatgg

SEQ ID NO: 758 - DNA, 25 bp, Artificial Sequence
  synthetic nucleotide probe
tacccatggctacagttgatataaa

SEQ ID NO: 759 - DNA, 25 bp, Artificial Sequence
  synthetic nucleotide probe
ttcatacctttgctggagatacttg

SEQ ID NO: 760 - DNA, 25 bp, Artificial Sequence
  synthetic nucleotide probe
atacctttgctggagatacttgtca

SEQ ID NO: 761 - DNA, 25 bp, Artificial Sequence
  synthetic nucleotide probe
gcctgtgctgaactgatctcttaaa

SEQ ID NO: 762 - DNA, 25 bp, Artificial Sequence
  synthetic nucleotide probe
aatactggtgctcttgtcacaggta

SEQ ID NO: 763 - DNA, 25 bp, Artificial Sequence
  synthetic nucleotide probe
aaaatgctgatcttctctggagtct

SEQ ID NO: 764 - DNA, 25 bp, Artificial Sequence
  synthetic nucleotide probe
tgctcttccaacagtgggttctagc

SEQ ID NO: 765 - DNA, 25 bp, Artificial Sequence
  synthetic nucleotide probe
acaactgacaagacaccagcccata

SEQ ID NO: 766 - DNA, 25 bp, Artificial Sequence
  synthetic nucleotide probe
ttaggccttttgtgcataccattac

SEQ ID NO: 767 - DNA, 25 bp, Artificial Sequence
  synthetic nucleotide probe
ggtgcatgtacaacagcatccaaca

SEQ ID NO: 768 - DNA, 25 bp, Artificial Sequence
  synthetic nucleotide probe
tctttttgtcctcatcactcaatac

SEQ ID NO: 769 - DNA, 25 bp, Artificial Sequence
  synthetic nucleotide probe
gcaatcttggaatcctcaactgcag

SEQ ID NO: 770 - DNA, 25 bp, Artificial Sequence
  synthetic nucleotide probe
ggtagaacagcttgtttcttttcca

SEQ ID NO: 771 - DNA, 25 bp, Artificial Sequence
  synthetic nucleotide probe
gcatccaacatatctgtcttgttcc

SEQ ID NO: 772 - DNA, 25 bp, Artificial Sequence
  synthetic nucleotide probe
ttcagccctttaataatggagcatc

SEQ ID NO: 773 - DNA, 25 bp, Artificial Sequence
  synthetic nucleotide probe
tttactatgatatccattttccaga

SEQ ID NO: 774 - DNA, 25 bp, Artificial Sequence
  synthetic nucleotide probe
gagactaactctccacttgtatggg

SEQ ID NO: 775 - DNA, 25 bp, Artificial Sequence
  synthetic nucleotide probe
gggaactacatttcactcttggttt

SEQ ID NO: 776 - DNA, 25 bp, Artificial Sequence
  synthetic nucleotide probe
acctgtaaccccaagcaaatataga

SEQ ID NO: 777 - DNA, 25 bp, Artificial Sequence
  synthetic nucleotide probe
aactctccacttgtatgggaactac

SEQ ID NO: 778 - DNA, 25 bp, Artificial Sequence
  synthetic nucleotide probe
tcaggatataacagcacttcaccga

SEQ ID NO: 779 - DNA, 25 bp, Artificial Sequence
  synthetic nucleotide probe
atttcactcttggttttcaggatat

SEQ ID NO: 780 - DNA, 25 bp, Artificial Sequence
  synthetic nucleotide probe
taacagcacttcaccgaaatattct

SEQ ID NO: 781 - DNA, 25 bp, Artificial Sequence
  synthetic nucleotide probe
tattagcacacaactattttcagcc

SEQ ID NO: 782 - DNA, 25 bp, Artificial Sequence
  synthetic nucleotide probe
aagtttgtttatattcagaagtctg

SEQ ID NO: 783 - DNA, 25 bp, Artificial Sequence
  synthetic nucleotide probe
gctgaaggcagatgtcgtcccaaag

SEQ ID NO: 784 - DNA, 25 bp, Artificial Sequence
  synthetic nucleotide probe
tggcaagcatgttgtgttcggtcac

SEQ ID NO: 785 - DNA, 25 bp, Artificial Sequence
  synthetic nucleotide probe
gcctgaaacgatacgtgtgcccact

SEQ ID NO: 786 - DNA, 25 bp, Artificial Sequence
  synthetic nucleotide probe
taatgctggtcctaacaccaacggc

SEQ ID NO: 787 - DNA, 25 bp, Artificial Sequence
  synthetic nucleotide probe
ccgctttcctgacgagaactttaca

SEQ ID NO: 788 - DNA, 25 bp, Artificial Sequence
  synthetic nucleotide probe
gtggccagggtgctggcatggtggc

SEQ ID NO: 789 - DNA, 25 bp, Artificial Sequence
  synthetic nucleotide probe
agaagggcttcggctacaaaggctc

SEQ ID NO: 790 - DNA, 25 bp, Artificial Sequence
  synthetic nucleotide probe
aagtccatctacggaagccgctttc

SEQ ID NO: 791 - DNA, 25 bp, Artificial Sequence
  synthetic nucleotide probe
tgtcctgtccatggctaatgctggt

SEQ ID NO: 792 - DNA, 25 bp, Artificial Sequence
  synthetic nucleotide probe
gttcggtcacgtcaaagagggcatg

SEQ ID NO: 793 - DNA, 25 bp, Artificial Sequence
  synthetic nucleotide probe
gactgtggccagttgagctaatctg

SEQ ID NO: 794 - DNA, 25 bp, Artificial Sequence
  synthetic nucleotide probe
tacagcaggagtggccatgtggtcc

SEQ ID NO: 795 - DNA, 25 bp, Artificial Sequence
  synthetic nucleotide probe
gatgaggtactcgtggttctggagc

SEQ ID NO: 796 - DNA, 25 bp, Artificial Sequence
  synthetic nucleotide probe
ggcagatggtgcagccaacaatgct

SEQ ID NO: 797 - DNA, 25 bp, Artificial Sequence
  synthetic nucleotide probe
aacaatgctgaccggtgcttatcct

SEQ ID NO: 798 - DNA, 25 bp, Artificial Sequence
  synthetic nucleotide probe
ggacattcttcaattccacatctgt

SEQ ID NO: 799 - DNA, 25 bp, Artificial Sequence
  synthetic nucleotide probe
gggctgaatttagactctctcacag

SEQ ID NO: 800 - DNA, 25 bp, Artificial Sequence
  synthetic nucleotide probe
cagcctctggagagtactcagtctc

SEQ ID NO: 801 - DNA, 25 bp, Artificial Sequence
  synthetic nucleotide probe
aaataagtcattctccctagcagag

SEQ ID NO: 802 - DNA, 25 bp, Artificial Sequence
  synthetic nucleotide probe
ctctaagccctgatccacaataaaa

SEQ ID NO: 803 - DNA, 25 bp, Artificial Sequence
  synthetic nucleotide probe
ggagctctagaaacacttctgatgc

SEQ ID NO: 804 - DNA, 25 bp, Artificial Sequence
  synthetic nucleotide probe
tataagctctcgctagagttcccca

SEQ ID NO: 805 - DNA, 25 bp, Artificial Sequence
  synthetic nucleotide probe
gagctctggaaaaaccccacagctt

SEQ ID NO: 806 - DNA, 25 bp, Artificial Sequence
  synthetic nucleotide probe
ggaaaagtggtctgccctgtatgat

SEQ ID NO: 807 - DNA, 25 bp, Artificial Sequence
  synthetic nucleotide probe
gggtaacatatgcctggacatcctg

SEQ ID NO: 808 - DNA, 25 bp, Artificial Sequence
  synthetic nucleotide probe
caatgcgcccacagtgaagttcctc

SEQ ID NO: 809 - DNA, 25 bp, Artificial Sequence
  synthetic nucleotide probe
gggtagggaccatccatggagcagc

SEQ ID NO: 810 - DNA, 25 bp, Artificial Sequence
  synthetic nucleotide probe
gccttccctgaatcagacaaccttt

SEQ ID NO: 811 - DNA, 25 bp, Artificial Sequence
  synthetic nucleotide probe
tccatccagagccttctaggagaac

SEQ ID NO: 812 - DNA, 25 bp, Artificial Sequence
  synthetic nucleotide probe
atgatgtcaggaccattctgctctc

SEQ ID NO: 813 - DNA, 25 bp, Artificial Sequence
  synthetic nucleotide probe
agatggtctgtcctttttgtgattt

SEQ ID NO: 814 - DNA, 25 bp, Artificial Sequence
  synthetic nucleotide probe
tgatagtcccttgaacacacatgct

SEQ ID NO: 815 - DNA, 25 bp, Artificial Sequence
  synthetic nucleotide probe
gagggtatctgacaatgctgaggtt

SEQ ID NO: 816 - DNA, 25 bp, Artificial Sequence
  synthetic nucleotide probe
gcatttcaatcccacgtacttataa

SEQ ID NO: 817 - DNA, 25 bp, Artificial Sequence
  synthetic nucleotide probe
atctgtccgtgacctatcagttatt

SEQ ID NO: 818 - DNA, 25 bp, Artificial Sequence
  synthetic nucleotide probe
gtacaatccgcatccaactattaaa

SEQ ID NO: 819 - DNA, 25 bp, Artificial Sequence
  synthetic nucleotide probe
aaatggctgtttagggtgctttcaa

SEQ ID NO: 820 - DNA, 25 bp, Artificial Sequence
  synthetic nucleotide probe
tcacaagctattccctcaaatctga

SEQ ID NO: 821 - DNA, 25 bp, Artificial Sequence
  synthetic nucleotide probe
aactgtgccagttctttccataata

SEQ ID NO: 822 - DNA, 25 bp, Artificial Sequence
  synthetic nucleotide probe
gagggagctgagtaacaccatcgat

SEQ ID NO: 823 - DNA, 25 bp, Artificial Sequence
  synthetic nucleotide probe
ggggttgggctggatgccgaggtaa

SEQ ID NO: 824 - DNA, 25 bp, Artificial Sequence
  synthetic nucleotide probe
aaagctcaggattcttcgaaaagtt

SEQ ID NO: 825 - DNA, 25 bp, Artificial Sequence
  synthetic nucleotide probe
gaactaggtcaaaaatctgtccgtg

SEQ ID NO: 826 - DNA, 25 bp, Artificial Sequence
  synthetic nucleotide probe
attaataccatgacatcttgcttat

SEQ ID NO: 827 - DNA, 25 bp, Artificial Sequence
  synthetic nucleotide probe
gctgtagtgttgaatacttggcccc

SEQ ID NO: 828 - DNA, 25 bp, Artificial Sequence
  synthetic nucleotide probe
gccatgcctttctgtatagtacaca

SEQ ID NO: 829 - DNA, 25 bp, Artificial Sequence
  synthetic nucleotide probe
tgagctgtctgtcatttacctactt

SEQ ID NO: 830 - DNA, 25 bp, Artificial Sequence
  synthetic nucleotide probe
gtagcactcactgaatagttttaaa

SEQ ID NO: 831 - DNA, 25 bp, Artificial Sequence
  synthetic nucleotide probe
ttggttgggcttttaatcctgtgtg

SEQ ID NO: 832 - DNA, 25 bp, Artificial Sequence
  synthetic nucleotide probe
ctctgtagttcaaatctgttagctt

SEQ ID NO: 833 - DNA, 25 bp, Artificial Sequence
  synthetic nucleotide probe
gatatttcggaattggttttactgt

SEQ ID NO: 834 - DNA, 25 bp, Artificial Sequence
  synthetic nucleotide probe
aaatattccattgctctgtagttca

SEQ ID NO: 835 - DNA, 25 bp, Artificial Sequence
  synthetic nucleotide probe
tgaatacttggccccatgagccatg

SEQ ID NO: 836 - DNA, 25 bp, Artificial Sequence
  synthetic nucleotide probe
ggtatgcttacaattgtcatgtcta

SEQ ID NO: 837 - DNA, 25 bp, Artificial Sequence
  synthetic nucleotide probe
acctgtatttctcagttgcagcact

SEQ ID NO: 838 - DNA, 25 bp, Artificial Sequence
  synthetic nucleotide probe
cccatgcatctgcctggcatttagg

SEQ ID NO: 839 - DNA, 25 bp, Artificial Sequence
  synthetic nucleotide probe
gcaattgaggcagttgaccatattc

SEQ ID NO: 840 - DNA, 25 bp, Artificial Sequence
  synthetic nucleotide probe
tcctcatctcatttggctgtgtaaa

SEQ ID NO: 841 - DNA, 25 bp, Artificial Sequence
  synthetic nucleotide probe
cctgccagcagttcttgaagcttct

SEQ ID NO: 842 - DNA, 25 bp, Artificial Sequence
  synthetic nucleotide probe
tccaagtccaccagtctctgaaatt

SEQ ID NO: 843 - DNA, 25 bp, Artificial Sequence
  synthetic nucleotide probe
tggcatttaggcagcagagcccctg

SEQ ID NO: 844 - DNA, 25 bp, Artificial Sequence
  synthetic nucleotide probe
ggagtggaatgttctatccccacaa

SEQ ID NO: 845 - DNA, 25 bp, Artificial Sequence
  synthetic nucleotide probe
cagcccaggagtagtcttacctctg

SEQ ID NO: 846 - DNA, 25 bp, Artificial Sequence
  synthetic nucleotide probe
cttacctctgaggaactttctagat

SEQ ID NO: 847 - DNA, 25 bp, Artificial Sequence
  synthetic nucleotide probe
ggctgctaaagccattgctgcactc

SEQ ID NO: 848 - DNA, 25 bp, Artificial Sequence
  synthetic nucleotide probe
gatgaggcatcgtgcctcacatccg

SEQ ID NO: 849 - DNA, 25 bp, Artificial Sequence
  synthetic nucleotide probe
taaagccattgctgcactctgaggg

SEQ ID NO: 850 - DNA, 25 bp, Artificial Sequence
  synthetic nucleotide probe
gtgagggaaatctaccttcgttcat

SEQ ID NO: 851 - DNA, 25 bp, Artificial Sequence
  synthetic nucleotide probe
tggtgcaagagcctctagcggcttc

SEQ ID NO: 852 - DNA, 25 bp, Artificial Sequence
  synthetic nucleotide probe
ttcttctttcaaaatttcctctcca

SEQ ID NO: 853 - DNA, 25 bp, Artificial Sequence
  synthetic nucleotide probe
aactggtgaaggtgtcagccatgtc

SEQ ID NO: 854 - DNA, 25 bp, Artificial Sequence
  synthetic nucleotide probe
catccgctccacatggtgcaagagc

SEQ ID NO: 855 - DNA, 25 bp, Artificial Sequence
  synthetic nucleotide probe
agatgacatggttgttgccccagtc

SEQ ID NO: 856 - DNA, 25 bp, Artificial Sequence
  synthetic nucleotide probe
aaatatgcagactcaccgggagcct

SEQ ID NO: 857 - DNA, 25 bp, Artificial Sequence
  synthetic nucleotide probe
catgttcctggtgctgatattctca

SEQ ID NO: 858 - DNA, 25 bp, Artificial Sequence
  synthetic nucleotide probe
ttatgccggtctagcctgtgtggaa

SEQ ID NO: 859 - DNA, 25 bp, Artificial Sequence
  synthetic nucleotide probe
tcacccatgtgtccgtcacattaga

SEQ ID NO: 860 - DNA, 25 bp, Artificial Sequence
  synthetic nucleotide probe
gatattctcaatagttatgccggtc

SEQ ID NO: 861 - DNA, 25 bp, Artificial Sequence
  synthetic nucleotide probe
gatgaacgacagcttggtgatccag

SEQ ID NO: 862 - DNA, 25 bp, Artificial Sequence
  synthetic nucleotide probe
ttggtgatccagctatttttcctgc

SEQ ID NO: 863 - DNA, 25 bp, Artificial Sequence
  synthetic nucleotide probe
ggcactcctcaatatggattcccct

SEQ ID NO: 864 - DNA, 25 bp, Artificial Sequence
  synthetic nucleotide probe
gctgcctgatacgtgaatcttcttg

SEQ ID NO: 865 - DNA, 25 bp, Artificial Sequence
  synthetic nucleotide probe
gacatcacccttacagttgaagctt

SEQ ID NO: 866 - DNA, 25 bp, Artificial Sequence
  synthetic nucleotide probe
gtgcatgtttgggaactggcattac

SEQ ID NO: 867 - DNA, 25 bp, Artificial Sequence
  synthetic nucleotide probe
ttctcagtactccctcaagactgga

SEQ ID NO: 868 - DNA, 25 bp, Artificial Sequence
  synthetic nucleotide probe
actccagagctgcagtgccactgga

SEQ ID NO: 869 - DNA, 25 bp, Artificial Sequence
  synthetic nucleotide probe
gtgccactggaggagtcagactacc

SEQ ID NO: 870 - DNA, 25 bp, Artificial Sequence
  synthetic nucleotide probe
atagatgagctctgagtttctcagt

SEQ ID NO: 871 - DNA, 25 bp, Artificial Sequence
  synthetic nucleotide probe
gtcagactaccatgacatcgtaggg

SEQ ID NO: 872 - DNA, 25 bp, Artificial Sequence
  synthetic nucleotide probe
tcttctaacctccttgggctatatt

SEQ ID NO: 873 - DNA, 25 bp, Artificial Sequence
  synthetic nucleotide probe
caggcctgagggggaaccatttttg

SEQ ID NO: 874 - DNA, 25 bp, Artificial Sequence
  synthetic nucleotide probe
ggacatcttggtctttttctcaggc

SEQ ID NO: 875 - DNA, 25 bp, Artificial Sequence
  synthetic nucleotide probe
tgctgagggtggagtgtcccatcct

SEQ ID NO: 876 - DNA, 25 bp, Artificial Sequence
  synthetic nucleotide probe
gaggtatattggaactcttctaacc

SEQ ID NO: 877 - DNA, 25 bp, Artificial Sequence
  synthetic nucleotide probe
actgaccatacatttttcttagccc

SEQ ID NO: 878 - DNA, 25 bp, Artificial Sequence
  synthetic nucleotide probe
gacatttttacataccgagcctgag

SEQ ID NO: 879 - DNA, 25 bp, Artificial Sequence
  synthetic nucleotide probe
ttcatctgagcccccaaaagcatta

SEQ ID NO: 880 - DNA, 25 bp, Artificial Sequence
  synthetic nucleotide probe
ttcttagcccctcaagtaatatagc

SEQ ID NO: 881 - DNA, 25 bp, Artificial Sequence
  synthetic nucleotide probe
aaactggtcataaaggcactctgtg

SEQ ID NO: 882 - DNA, 25 bp, Artificial Sequence
  synthetic nucleotide probe
ccttccagagtcctggctgattggt

SEQ ID NO: 883 - DNA, 25 bp, Artificial Sequence
  synthetic nucleotide probe
gggactgacatcttaagctctcacc

SEQ ID NO: 884 - DNA, 25 bp, Artificial Sequence
  synthetic nucleotide probe
tacactactgtgtttcactgaccat

SEQ ID NO: 885 - DNA, 25 bp, Artificial Sequence
  synthetic nucleotide probe
tgattggtgttcgctgttcatctga

SEQ ID NO: 886 - DNA, 25 bp, Artificial Sequence
  synthetic nucleotide probe
ggacttctcatttttggagctttcc

SEQ ID NO: 887 - DNA, 25 bp, Artificial Sequence
  synthetic nucleotide probe
gaatgacaattcccctaaccattcc

SEQ ID NO: 888 - DNA, 25 bp, Artificial Sequence
  synthetic nucleotide probe
tttgcacttccttcggagagcatct

SEQ ID NO: 889 - DNA, 25 bp, Artificial Sequence
  synthetic nucleotide probe
cttccagttatgcagcacctggcta

SEQ ID NO: 890 - DNA, 25 bp, Artificial Sequence
  synthetic nucleotide probe
caacattacctgtcatatactgaag

SEQ ID NO: 891 - DNA, 25 bp, Artificial Sequence
  synthetic nucleotide probe
tggacaccaactctacaacattacc

SEQ ID NO: 892 - DNA, 25 bp, Artificial Sequence
  synthetic nucleotide probe
tggcaccatgtgccatctgtacata

SEQ ID NO: 893 - DNA, 25 bp, Artificial Sequence
  synthetic nucleotide probe
actatgacatggtgcactttcctcc

SEQ ID NO: 894 - DNA, 25 bp, Artificial Sequence
  synthetic nucleotide probe
ggaactaactatgttggactatgac

SEQ ID NO: 895 - DNA, 25 bp, Artificial Sequence
  synthetic nucleotide probe
gaatctcttcttccagttatgcagc

SEQ ID NO: 896 - DNA, 25 bp, Artificial Sequence
  synthetic nucleotide probe
gcacctggctaagaatgtagtcatg

SEQ ID NO: 897 - DNA, 25 bp, Artificial Sequence
  synthetic nucleotide probe
tcctccttctcaaattgcagcagga

SEQ ID NO: 898 - DNA, 25 bp, Artificial Sequence
  synthetic nucleotide probe
agattggagaggttgatgtcgagca

SEQ ID NO: 899 - DNA, 25 bp, Artificial Sequence
  synthetic nucleotide probe
attccatcagctttctctaagtctt

SEQ ID NO: 900 - DNA, 25 bp, Artificial Sequence
  synthetic nucleotide probe
atagagggtctcttcacgttgatgc

SEQ ID NO: 901 - DNA, 25 bp, Artificial Sequence
  synthetic nucleotide probe
atccactgtgtgcatagagggtctc

SEQ ID NO: 902 - DNA, 25 bp, Artificial Sequence
  synthetic nucleotide probe
tcagctttctctaagtctttgctca

SEQ ID NO: 903 - DNA, 25 bp, Artificial Sequence
  synthetic nucleotide probe
aagttcaaccttaaaatgatgttag

SEQ ID NO: 904 - DNA, 25 bp, Artificial Sequence
  synthetic nucleotide probe
tctcttcacgttgatgcttggcatt

SEQ ID NO: 905 - DNA, 25 bp, Artificial Sequence
  synthetic nucleotide probe
gccaggcataacatatccactgtgt

SEQ ID NO: 906 - DNA, 25 bp, Artificial Sequence
  synthetic nucleotide probe
tcacgttgatgcttggcattccatc

SEQ ID NO: 907 - DNA, 25 bp, Artificial Sequence
  synthetic nucleotide probe
gtgtgcatagagggtctcttcacgt

SEQ ID NO: 908 - DNA, 25 bp, Artificial Sequence
  synthetic nucleotide probe
tacagccaggcataacatatccact

SEQ ID NO: 909 - DNA, 25 bp, Artificial Sequence
  synthetic nucleotide probe
ccatcagctttctctaagtctttgc

SEQ ID NO: 910 - DNA, 25 bp, Artificial Sequence
  synthetic nucleotide probe
aggccataatcatcttttctggtta

SEQ ID NO: 911 - DNA, 25 bp, Artificial Sequence
  synthetic nucleotide probe
aaatagcatcagtttgtccaatagt

SEQ ID NO: 912 - DNA, 25 bp, Artificial Sequence
  synthetic nucleotide probe
atgttgacaccttaatcggtcccag

SEQ ID NO: 913 - DNA, 25 bp, Artificial Sequence
  synthetic nucleotide probe
gaagctccttgaccagggatgagaa

SEQ ID NO: 914 - DNA, 25 bp, Artificial Sequence
  synthetic nucleotide probe
caaccatcagttagagaagctcctt

SEQ ID NO: 915 - DNA, 25 bp, Artificial Sequence
  synthetic nucleotide probe
aagaacacttctgggagccgaaagc

SEQ ID NO: 916 - DNA, 25 bp, Artificial Sequence
  synthetic nucleotide probe
gtgcctataggaagactagtctcat

SEQ ID NO: 917 - DNA, 25 bp, Artificial Sequence
  synthetic nucleotide probe
gccatctgcgaaatatcaaccatca

SEQ ID NO: 918 - DNA, 25 bp, Artificial Sequence
  synthetic nucleotide probe
aaacgtatgattcatccagccttcc

SEQ ID NO: 919 - DNA, 25 bp, Artificial Sequence
  synthetic nucleotide probe
atcggtcccaggtatgagctataat

SEQ ID NO: 920 - DNA, 25 bp, Artificial Sequence
  synthetic nucleotide probe
ggagccgaaagccatctgcgaaata

SEQ ID NO: 921 - DNA, 25 bp, Artificial Sequence
  synthetic nucleotide probe
agactgtttgtacccttcatgaaat

SEQ ID NO: 922 - DNA, 25 bp, Artificial Sequence
  synthetic nucleotide probe
tcacaatagcctttttatagtcagt

SEQ ID NO: 923 - DNA, 25 bp, Artificial Sequence
  synthetic nucleotide probe
gtaaatgactctttgctacatttta

SEQ ID NO: 924 - DNA, 25 bp, Artificial Sequence
  synthetic nucleotide probe
gcatgttcaactttttattgtggtc

SEQ ID NO: 925 - DNA, 25 bp, Artificial Sequence
  synthetic nucleotide probe
ggtaattttatccactagcaaatct

SEQ ID NO: 926 - DNA, 25 bp, Artificial Sequence
  synthetic nucleotide probe
gcatagatatgcgcatgttcaactt

SEQ ID NO: 927 - DNA, 25 bp, Artificial Sequence
  synthetic nucleotide probe
gaagttgccatcagttttactaatc

SEQ ID NO: 928 - DNA, 25 bp, Artificial Sequence
  synthetic nucleotide probe
cagttttactaatcttctgtgaaat

SEQ ID NO: 929 - DNA, 25 bp, Artificial Sequence
  synthetic nucleotide probe
tagctgtttcagagagagtacggta

SEQ ID NO: 930 - DNA, 25 bp, Artificial Sequence
  synthetic nucleotide probe
ttcctatttctttagggagtgctac

SEQ ID NO: 931 - DNA, 25 bp, Artificial Sequence
  synthetic nucleotide probe
gtatgtgccacttctgagagtagta

SEQ ID NO: 932 - DNA, 25 bp, Artificial Sequence
  synthetic nucleotide probe
agccttgttgggttgtcagccatgg

SEQ ID NO: 933 - DNA, 25 bp, Artificial Sequence
  synthetic nucleotide probe
aacagttagatcagcccatctcagc

SEQ ID NO: 934 - DNA, 25 bp, Artificial Sequence
  synthetic nucleotide probe
atggcacccttgcaactgtgggatc

SEQ ID NO: 935 - DNA, 25 bp, Artificial Sequence
  synthetic nucleotide probe
agaagtagtggctggagactctggc

SEQ ID NO: 936 - DNA, 25 bp, Artificial Sequence
  synthetic nucleotide probe
ctgcctcatctctgtacgaatttgg

SEQ ID NO: 937 - DNA, 25 bp, Artificial Sequence
  synthetic nucleotide probe
gatggtagattcagcttctgggaca

SEQ ID NO: 938 - DNA, 25 bp, Artificial Sequence
  synthetic nucleotide probe
ctcagcttgctgtttcaatcacaat

SEQ ID NO: 939 - DNA, 25 bp, Artificial Sequence
  synthetic nucleotide probe
aatggaatcgcgttccatcctgttc

SEQ ID NO: 940 - DNA, 25 bp, Artificial Sequence
  synthetic nucleotide probe
tggatttcaacccctggagaaaacg

SEQ ID NO: 941 - DNA, 25 bp, Artificial Sequence
  synthetic nucleotide probe
acgaatttgggtcccagccttgttg

SEQ ID NO: 942 - DNA, 25 bp, Artificial Sequence
  synthetic nucleotide probe
catatttgcatacgcttccagctac

SEQ ID NO: 943 - DNA, 25 bp, Artificial Sequence
  synthetic nucleotide probe
ggaaatcacatagcagttaccccat

SEQ ID NO: 944 - DNA, 25 bp, Artificial Sequence
  synthetic nucleotide probe
gtggtgctgagagactacatttata

SEQ ID NO: 945 - DNA, 25 bp, Artificial Sequence
  synthetic nucleotide probe
ataccgttacttgggaaatcatctt

SEQ ID NO: 946 - DNA, 25 bp, Artificial Sequence
  synthetic nucleotide probe
caactgctgtatttaacatctgcct

SEQ ID NO: 947 - DNA, 25 bp, Artificial Sequence
  synthetic nucleotide probe
gatatctatatccctacaacctaat

SEQ ID NO: 948 - DNA, 25 bp, Artificial Sequence
  synthetic nucleotide probe
taactgtggtttgcaccctaacact

SEQ ID NO: 949 - DNA, 25 bp, Artificial Sequence
  synthetic nucleotide probe
gttaccccatggcattgtgactaat

SEQ ID NO: 950 - DNA, 25 bp, Artificial Sequence
  synthetic nucleotide probe
ccacctcatacacacacaattcagt

SEQ ID NO: 951 - DNA, 25 bp, Artificial Sequence
  synthetic nucleotide probe
gtcatatagtaagcattttccccca

SEQ ID NO: 952 - DNA, 25 bp, Artificial Sequence
  synthetic nucleotide probe
catatttcaggtttgttctctttcc

SEQ ID NO: 953 - DNA, 25 bp, Artificial Sequence
  synthetic nucleotide probe
aaagtagtcactatacaactcccct

SEQ ID NO: 954 - DNA, 25 bp, Artificial Sequence
  synthetic nucleotide probe
aagaccttgttctcaaatctaggaa

SEQ ID NO: 955 - DNA, 25 bp, Artificial Sequence
  synthetic nucleotide probe
gaaggattcatttgttgcgagtgcc

SEQ ID NO: 956 - DNA, 25 bp, Artificial Sequence
  synthetic nucleotide probe
gttgcgagtgccagtatacttaaat

SEQ ID NO: 957 - DNA, 25 bp, Artificial Sequence
  synthetic nucleotide probe
ttttccaatccatttatccctgggg

SEQ ID NO: 958 - DNA, 25 bp, Artificial Sequence
  synthetic nucleotide probe
cctgattttcacacaaatactatat

SEQ ID NO: 959 - DNA, 25 bp, Artificial Sequence
  synthetic nucleotide probe
gtaaattgggttcttcatggaagtt

SEQ ID NO: 960 - DNA, 25 bp, Artificial Sequence
  synthetic nucleotide probe
ggaaatcatctgtgacggaagagta

SEQ ID NO: 961 - DNA, 25 bp, Artificial Sequence
  synthetic nucleotide probe
atgattttgtttgtatccctaccca

SEQ ID NO: 962 - DNA, 25 bp, Artificial Sequence
  synthetic nucleotide probe
ggaagccatagaattgctctggtca

SEQ ID NO: 963 - DNA, 25 bp, Artificial Sequence
  synthetic nucleotide probe
agcacaccatagccttaactgaata

SEQ ID NO: 964 - DNA, 25 bp, Artificial Sequence
  synthetic nucleotide probe
tgggtgccaaaggtcaactgtaatg

SEQ ID NO: 965 - DNA, 25 bp, Artificial Sequence
  synthetic nucleotide probe
gaccaatatctgccagtaacgctgt

SEQ ID NO: 966 - DNA, 25 bp, Artificial Sequence
  synthetic nucleotide probe
aattgggatacatttggctgtcaga

SEQ ID NO: 967 - DNA, 25 bp, Artificial Sequence
  synthetic nucleotide probe
taacgctgtttatctcacttgcttt

SEQ ID NO: 968 - DNA, 25 bp, Artificial Sequence
  synthetic nucleotide probe
gacaactttttaattcctttgatct

SEQ ID NO: 969 - DNA, 25 bp, Artificial Sequence
  synthetic nucleotide probe
gtctactgggtataacatgtctcac

SEQ ID NO: 970 - DNA, 25 bp, Artificial Sequence
  synthetic nucleotide probe
ggaaatctgcctaatctgcttatat

SEQ ID NO: 971 - DNA, 25 bp, Artificial Sequence
  synthetic nucleotide probe
gctctggtcaaaaccaagcacacca

SEQ ID NO: 972 - DNA, 25 bp, Artificial Sequence
  synthetic nucleotide probe
gattccctgcatcaactaagaaaag

SEQ ID NO: 973 - DNA, 25 bp, Artificial Sequence
  synthetic nucleotide probe
ttggtggcgaatgtgttgcgggtcc

SEQ ID NO: 974 - DNA, 25 bp, Artificial Sequence
  synthetic nucleotide probe
gcgtgttctgtaatatttcctctaa

SEQ ID NO: 975 - DNA, 25 bp, Artificial Sequence
  synthetic nucleotide probe
tctcctaaccctctagttttaattg

SEQ ID NO: 976 - DNA, 25 bp, Artificial Sequence
  synthetic nucleotide probe
tctcagtagtggtgctgtattgtac

SEQ ID NO: 977 - DNA, 25 bp, Artificial Sequence
  synthetic nucleotide probe
aattggacacttctttgctgttgca

SEQ ID NO: 978 - DNA, 25 bp, Artificial Sequence
  synthetic nucleotide probe
cttggattacaaaacctcgagccct

SEQ ID NO: 979 - DNA, 25 bp, Artificial Sequence
  synthetic nucleotide probe
ctgttgggctgctttctgtattgtc

SEQ ID NO: 980 - DNA, 25 bp, Artificial Sequence
  synthetic nucleotide probe
atctatgccgtgtatttttgcttta

SEQ ID NO: 981 - DNA, 25 bp, Artificial Sequence
  synthetic nucleotide probe
ttgctgttgcaatctatgccgtgta

SEQ ID NO: 982 - DNA, 25 bp, Artificial Sequence
  synthetic nucleotide probe
atagcatagtaccataccacttata

SEQ ID NO: 983 - DNA, 25 bp, Artificial Sequence
  synthetic nucleotide probe
gaacgcagagcgactcgaggtgtcc

SEQ ID NO: 984 - DNA, 25 bp, Artificial Sequence
  synthetic nucleotide probe
ggcaaagcatttcatccaacttatg

SEQ ID NO: 985 - DNA, 25 bp, Artificial Sequence
  synthetic nucleotide probe
gcctggaggagttgtacggcctggc

SEQ ID NO: 986 - DNA, 25 bp, Artificial Sequence
  synthetic nucleotide probe
gcagcagccggagatggcggcggtg

SEQ ID NO: 987 - DNA, 25 bp, Artificial Sequence
  synthetic nucleotide probe
ttaaataggtgttgccatctctttt

SEQ ID NO: 988 - DNA, 25 bp, Artificial Sequence
  synthetic nucleotide probe
gacacttgtcctgaggttggattct

SEQ ID NO: 989 - DNA, 25 bp, Artificial Sequence
  synthetic nucleotide probe
ggcagccctgggaaacatgtctaaa

SEQ ID NO: 990 - DNA, 25 bp, Artificial Sequence
  synthetic nucleotide probe
tcaacatatgttgcgtcccacaaaa

SEQ ID NO: 991 - DNA, 25 bp, Artificial Sequence
  synthetic nucleotide probe
tggcactgcgcttcttcaaagaaaa

SEQ ID NO: 992 - DNA, 25 bp, Artificial Sequence
  synthetic nucleotide probe
taagcaagttcttatgggcccatat

SEQ ID NO: 993 - DNA, 25 bp, Artificial Sequence
  synthetic nucleotide probe
ggcccatataatccagacacttgtc

SEQ ID NO: 994 - DNA, 25 bp, Artificial Sequence
  synthetic nucleotide probe
ccctgaacatgcctgagcttgtcat

SEQ ID NO: 995 - DNA, 25 bp, Artificial Sequence
  synthetic nucleotide probe
tccctttctgaattagctgtacata

SEQ ID NO: 996 - DNA, 25 bp, Artificial Sequence
  synthetic nucleotide probe
gcatttatctatgtctttaggtgtc

SEQ ID NO: 997 - DNA, 25 bp, Artificial Sequence
  synthetic nucleotide probe
ggtaatttccttctaatatgttggt

SEQ ID NO: 998 - DNA, 25 bp, Artificial Sequence
  synthetic nucleotide probe
taccattcttccactgtgctgttat

SEQ ID NO: 999 - DNA, 25 bp, Artificial Sequence
  synthetic nucleotide probe
gtagatcagaacatcaggctttcag

SEQ ID NO: 1000 - DNA, 25 bp, Artificial Sequence
  synthetic nucleotide probe
atgcctgagcttgtcataatatgtt

SEQ ID NO: 1001 - DNA, 25 bp, Artificial Sequence
  synthetic nucleotide probe
atgttggtactgtctatggccatac

SEQ ID NO: 1002 - DNA, 25 bp, Artificial Sequence
  synthetic nucleotide probe
ttaggtgtcattgttccctttctga

SEQ ID NO: 1003 - DNA, 25 bp, Artificial Sequence
  synthetic nucleotide probe
agagggactgtttaccattcttcca

SEQ ID NO: 1004 - DNA, 25 bp, Artificial Sequence
  synthetic nucleotide probe
gctgtacatataagccttcctttgg

SEQ ID NO: 1005 - DNA, 25 bp, Artificial Sequence
  synthetic nucleotide probe
cagaaagcaccgaatgacccacagc

SEQ ID NO: 1006 - DNA, 25 bp, Artificial Sequence
  synthetic nucleotide probe
ttccgtctgtactctcagaaagcac

SEQ ID NO: 1007 - DNA, 25 bp, Artificial Sequence
  synthetic nucleotide probe
gaagtgctgttatcggaaaccatca

SEQ ID NO: 1008 - DNA, 25 bp, Artificial Sequence
  synthetic nucleotide probe
gacttccagtctttcaccagatgac

SEQ ID NO: 1009 - DNA, 25 bp, Artificial Sequence
  synthetic nucleotide probe
gtatcatggtccagcagtactgttt

SEQ ID NO: 1010 - DNA, 25 bp, Artificial Sequence
  synthetic nucleotide probe
tatcccgtgttaccaaattaccatt

SEQ ID NO: 1011 - DNA, 25 bp, Artificial Sequence
  synthetic nucleotide probe
gaaaccatcagacatttccgtctgt

SEQ ID NO: 1012 - DNA, 25 bp, Artificial Sequence
  synthetic nucleotide probe
atgaaaaacctgctcatcgttcagc

SEQ ID NO: 1013 - DNA, 25 bp, Artificial Sequence
  synthetic nucleotide probe
ttaaaactaagtcatctcccagata

SEQ ID NO: 1014 - DNA, 25 bp, Artificial Sequence
  synthetic nucleotide probe
gcaaaattctgtttatcccgtgtta

SEQ ID NO: 1015 - DNA, 25 bp, Artificial Sequence
  synthetic nucleotide probe
ctcatcgttcagcttccaaaattct

SEQ ID NO: 1016 - DNA, 25 bp, Artificial Sequence
  synthetic nucleotide probe
ccttttcagcttgaccctgcaatat

SEQ ID NO: 1017 - DNA, 25 bp, Artificial Sequence
  synthetic nucleotide probe
aatctgcactgatattacatccaca

SEQ ID NO: 1018 - DNA, 25 bp, Artificial Sequence
  synthetic nucleotide probe
acctcatagctctcaagccagttga

SEQ ID NO: 1019 - DNA, 25 bp, Artificial Sequence
  synthetic nucleotide probe
aggagactgcccagcacataatgaa

SEQ ID NO: 1020 - DNA, 25 bp, Artificial Sequence
  synthetic nucleotide probe
gatattgtttgttcactcatttagt

SEQ ID NO: 1021 - DNA, 25 bp, Artificial Sequence
  synthetic nucleotide probe
acatgcacaggcctgcttgtgaatc

SEQ ID NO: 1022 - DNA, 25 bp, Artificial Sequence
  synthetic nucleotide probe
gccagttgaagaaaccttgcctttt

SEQ ID NO: 1023 - DNA, 25 bp, Artificial Sequence
  synthetic nucleotide probe
aagatttcccattcacttgatattg

SEQ ID NO: 1024 - DNA, 25 bp, Artificial Sequence
  synthetic nucleotide probe
tacatccacagtaccacagtattta

SEQ ID NO: 1025 - DNA, 25 bp, Artificial Sequence
  synthetic nucleotide probe
gaagaaacagcctacctcatagctc

SEQ ID NO: 1026 - DNA, 25 bp, Artificial Sequence
  synthetic nucleotide probe
gagcgcccatatgcatgcaacaaat

SEQ ID NO: 1027 - DNA, 25 bp, Artificial Sequence
  synthetic nucleotide probe
tggatgtctttgctgcagtcttctc

SEQ ID NO: 1028 - DNA, 25 bp, Artificial Sequence
  synthetic nucleotide probe
ggtcttgggttcctgtatggtggaa

SEQ ID NO: 1029 - DNA, 25 bp, Artificial Sequence
  synthetic nucleotide probe
caaaggaactttctggtcccttcag

SEQ ID NO: 1030 - DNA, 25 bp, Artificial Sequence
  synthetic nucleotide probe
ttggccaccagaccatgggccaaga

SEQ ID NO: 1031 - DNA, 25 bp, Artificial Sequence
  synthetic nucleotide probe
caaagggcagtgggtggaggaccgg

SEQ ID NO: 1032 - DNA, 25 bp, Artificial Sequence
  synthetic nucleotide probe
tctcctagacccgtgacctgagatg

SEQ ID NO: 1033 - DNA, 25 bp, Artificial Sequence
  synthetic nucleotide probe
cgtggctatacctggggacttgatg

SEQ ID NO: 1034 - DNA, 25 bp, Artificial Sequence
  synthetic nucleotide probe
cagccactcaaaggaactttctggt

SEQ ID NO: 1035 - DNA, 25 bp, Artificial Sequence
  synthetic nucleotide probe
ggtttggaaactgcaaatgtcccct

SEQ ID NO: 1036 - DNA, 25 bp, Artificial Sequence
  synthetic nucleotide probe
aggtttgagtccctccaaagggcag

SEQ ID NO: 1037 - DNA, 25 bp, Artificial Sequence
  synthetic nucleotide probe
ggcccaccggaacgacatggagacc

SEQ ID NO: 1038 - DNA, 25 bp, Artificial Sequence
  synthetic nucleotide probe
tctctgggcacagtgggcctgtgtg

SEQ ID NO: 1039 - DNA, 25 bp, Artificial Sequence
  synthetic nucleotide probe
ctctgggcacagtgggcctgtgtgt

SEQ ID NO: 1040 - DNA, 25 bp, Artificial Sequence
  synthetic nucleotide probe
tttggatgtctttgctgcagtcttc

SEQ ID NO: 1041 - DNA, 25 bp, Artificial Sequence
  synthetic nucleotide probe
acctggggacttgatgttccttcca

SEQ ID NO: 1042 - DNA, 25 bp, Artificial Sequence
  synthetic nucleotide probe
ggctatacctggggacttgatgttc

SEQ ID NO: 1043 - DNA, 25 bp, Artificial Sequence
  synthetic nucleotide probe
ctcctagacccgtgacctgagatgt

SEQ ID NO: 1044 - DNA, 25 bp, Artificial Sequence
  synthetic nucleotide probe
tggaggaccgggagctttgggtgac

SEQ ID NO: 1045 - DNA, 25 bp, Artificial Sequence
  synthetic nucleotide probe
caccagaccatgggccaagagccgc

SEQ ID NO: 1046 - DNA, 25 bp, Artificial Sequence
  synthetic nucleotide probe
gcagtgggtggaggaccgggagctt

SEQ ID NO: 1047 - DNA, 25 bp, Artificial Sequence
  synthetic nucleotide probe
tttctggtcccttcagtatcttcaa

SEQ ID NO: 1048 - DNA, 25 bp, Artificial Sequence
  synthetic nucleotide probe
caccggaacgacatggagaccatct

SEQ ID NO: 1049 - DNA, 25 bp, Artificial Sequence
  synthetic nucleotide probe
ggctcggaaagatggatttttgcat

SEQ ID NO: 1050 - DNA, 25 bp, Artificial Sequence
  synthetic nucleotide probe
gcagttttcggctgtcaaattatct

SEQ ID NO: 1051 - DNA, 25 bp, Artificial Sequence
  synthetic nucleotide probe
gggcgttcagatgcaattggaccaa

SEQ ID NO: 1052 - DNA, 25 bp, Artificial Sequence
  synthetic nucleotide probe
tagtcctccagagcagttttcggct

SEQ ID NO: 1053 - DNA, 25 bp, Artificial Sequence
  synthetic nucleotide probe
attgccctgaagtctggctatggaa

SEQ ID NO: 1054 - DNA, 25 bp, Artificial Sequence
  synthetic nucleotide probe
gcacggtggccctgctgcagttgat

SEQ ID NO: 1055 - DNA, 25 bp, Artificial Sequence
  synthetic nucleotide probe
ggcgatcagcgaggttctccaggac

SEQ ID NO: 1056 - DNA, 25 bp, Artificial Sequence
  synthetic nucleotide probe
ggaccttaggtttgatgcggaatct

SEQ ID NO: 1057 - DNA, 25 bp, Artificial Sequence
  synthetic nucleotide probe
ggttctccaggaccttaggtttgat

SEQ ID NO: 1058 - DNA, 25 bp, Artificial Sequence
  synthetic nucleotide probe
cagcgaggttctccaggaccttagg

SEQ ID NO: 1059 - DNA, 25 bp, Artificial Sequence
  synthetic nucleotide probe
aattaaaaccatggaggcgatcagc

SEQ ID NO: 1060 - DNA, 25 bp, Artificial Sequence
  synthetic nucleotide probe
ccttaggtttgatgcggaatctgcc

SEQ ID NO: 1061 - DNA, 25 bp, Artificial Sequence
  synthetic nucleotide probe
cgctgtacggatgcagcagctgaaa

SEQ ID NO: 1062 - DNA, 25 bp, Artificial Sequence
  synthetic nucleotide probe
tctgccgagtgatggcggctcccca

SEQ ID NO: 1063 - DNA, 25 bp, Artificial Sequence
  synthetic nucleotide probe
catggaggcgatcagcgaggttctc

SEQ ID NO: 1064 - DNA, 25 bp, Artificial Sequence
  synthetic nucleotide probe
gtttgatgcggaatctgccgagtga

SEQ ID NO: 1065 - DNA, 25 bp, Artificial Sequence
  synthetic nucleotide probe
actgcgctgctgaccttcctgcagt

SEQ ID NO: 1066 - DNA, 25 bp, Artificial Sequence
  synthetic nucleotide probe
attaaaaccatggaggcgatcagcg

SEQ ID NO: 1067 - DNA, 25 bp, Artificial Sequence
  synthetic nucleotide probe
ccaggaccttaggtttgatgcggaa

SEQ ID NO: 1068 - DNA, 25 bp, Artificial Sequence
  synthetic nucleotide probe
agtgcacggggtgacccaggccttc

SEQ ID NO: 1069 - DNA, 25 bp, Artificial Sequence
  synthetic nucleotide probe
atctgccgagtgatggcggctcccc

SEQ ID NO: 1070 - DNA, 25 bp, Artificial Sequence
  synthetic nucleotide probe
tgtgtgtgagacaggacgcagcggg

SEQ ID NO: 1071 - DNA, 25 bp, Artificial Sequence
  synthetic nucleotide probe
cagacccgtgggcaggtggggcatg

SEQ ID NO: 1072 - DNA, 25 bp, Artificial Sequence
  synthetic nucleotide probe
gttgagttaaaccccttgtgtgtga

SEQ ID NO: 1073 - DNA, 25 bp, Artificial Sequence
  synthetic nucleotide probe
tcccttccgcaggtctgcagatgaa

SEQ ID NO: 1074 - DNA, 25 bp, Artificial Sequence
  synthetic nucleotide probe
aatttcagggctcttggcgtgttgg

SEQ ID NO: 1075 - DNA, 25 bp, Artificial Sequence
  synthetic nucleotide probe
tgaaatgccactgcgcattttcaga

SEQ ID NO: 1076 - DNA, 25 bp, Artificial Sequence
  synthetic nucleotide probe
tcttttctcttcgtggcgacttaga

SEQ ID NO: 1077 - DNA, 25 bp, Artificial Sequence
  synthetic nucleotide probe
gcctttggtagctaacagtcactga

SEQ ID NO: 1078 - DNA, 25 bp, Artificial Sequence
  synthetic nucleotide probe
agaaatcctagtgcagcctttggta

SEQ ID NO: 1079 - DNA, 25 bp, Artificial Sequence
  synthetic nucleotide probe
tgaatggatgtttgttcctcctgat

SEQ ID NO: 1080 - DNA, 25 bp, Artificial Sequence
  synthetic nucleotide probe
gagttggcggatattcggaactgtg

SEQ ID NO: 1081 - DNA, 25 bp, Artificial Sequence
  synthetic nucleotide probe
tacctcggagctgatgctgggcgga

SEQ ID NO: 1082 - DNA, 25 bp, Artificial Sequence
  synthetic nucleotide probe
accaatggctgcaccggtgatgcca

SEQ ID NO: 1083 - DNA, 25 bp, Artificial Sequence
  synthetic nucleotide probe
caacacgtgtgaggactcgctgctg

SEQ ID NO: 1084 - DNA, 25 bp, Artificial Sequence
  synthetic nucleotide probe
gcctcaagcgagttggacccgtggc

SEQ ID NO: 1085 - DNA, 25 bp, Artificial Sequence
  synthetic nucleotide probe
gccacctaccctatgttgaacaaga

SEQ ID NO: 1086 - DNA, 25 bp, Artificial Sequence
  synthetic nucleotide probe
ggaaccaacacactggtgctgcaca

SEQ ID NO: 1087 - DNA, 25 bp, Artificial Sequence
  synthetic nucleotide probe
gtgagcttctgcactgacatggacc

SEQ ID NO: 1088 - DNA, 25 bp, Artificial Sequence
  synthetic nucleotide probe
aaccacatgctcctggaacacaaaa

SEQ ID NO: 1089 - DNA, 25 bp, Artificial Sequence
  synthetic nucleotide probe
gggcatctgcaagaggagcccccaa

SEQ ID NO: 1090 - DNA, 25 bp, Artificial Sequence
  synthetic nucleotide probe
caaaatggagcgcccagggcccagc

SEQ ID NO: 1091 - DNA, 25 bp, Artificial Sequence
  synthetic nucleotide probe
ccagcgcagctgcatcgagaacatc

SEQ ID NO: 1092 - DNA, 25 bp, Artificial Sequence
  synthetic nucleotide probe
aaattgatgacttgttcgtatgttc

SEQ ID NO: 1093 - DNA, 25 bp, Artificial Sequence
  synthetic nucleotide probe
gaagtgcctttatctgcttagacct

SEQ ID NO: 1094 - DNA, 25 bp, Artificial Sequence
  synthetic nucleotide probe
tgccctcaaacatacagaacttcca

SEQ ID NO: 1095 - DNA, 25 bp, Artificial Sequence
  synthetic nucleotide probe
ctctgacatcggatgccctcaaaca

SEQ ID NO: 1096 - DNA, 25 bp, Artificial Sequence
  synthetic nucleotide probe
agctattttgccaacatgtcagagt

SEQ ID NO: 1097 - DNA, 25 bp, Artificial Sequence
  synthetic nucleotide probe
agaacttccaaactcaagtccagcc

SEQ ID NO: 1098 - DNA, 25 bp, Artificial Sequence
  synthetic nucleotide probe
aagtccagccataagctattttgcc

SEQ ID NO: 1099 - DNA, 25 bp, Artificial Sequence
  synthetic nucleotide probe
agagctggggttaggatccggttgg

SEQ ID NO: 1100 - DNA, 25 bp, Artificial Sequence
  synthetic nucleotide probe
taggatccggttggactctgacatc

SEQ ID NO: 1101 - DNA, 25 bp, Artificial Sequence
  synthetic nucleotide probe
aaagctaacaccagtcatttatatt

SEQ ID NO: 1102 - DNA, 25 bp, Artificial Sequence
  synthetic nucleotide probe
gatgatgcttcaatttcttaatagt

SEQ ID NO: 1103 - DNA, 25 bp, Artificial Sequence
  synthetic nucleotide probe
aaacgttctcaccaaatccaatgct

SEQ ID NO: 1104 - DNA, 25 bp, Artificial Sequence
  synthetic nucleotide probe
atacgaggcgtcagctgctggcctc

SEQ ID NO: 1105 - DNA, 25 bp, Artificial Sequence
  synthetic nucleotide probe
aggagcttggaccttggtactacct

SEQ ID NO: 1106 - DNA, 25 bp, Artificial Sequence
  synthetic nucleotide probe
gatgcccgattctggaagggcccca

SEQ ID NO: 1107 - DNA, 25 bp, Artificial Sequence
  synthetic nucleotide probe
cagaccaaggctgcaagtggccctc

SEQ ID NO: 1108 - DNA, 25 bp, Artificial Sequence
  synthetic nucleotide probe
gcttaccatcctgtctaccagatga

SEQ ID NO: 1109 - DNA, 25 bp, Artificial Sequence
  synthetic nucleotide probe
ctctgtgatctcatttcatctgcac

SEQ ID NO: 1110 - DNA, 25 bp, Artificial Sequence
  synthetic nucleotide probe
gtggcacgtgacagctagggttcaa

SEQ ID NO: 1111 - DNA, 25 bp, Artificial Sequence
  synthetic nucleotide probe
tgagcgtttcccagaggatggaccc

SEQ ID NO: 1112 - DNA, 25 bp, Artificial Sequence
  synthetic nucleotide probe
tgagggtggtgacacaaccccttcc

SEQ ID NO: 1113 - DNA, 25 bp, Artificial Sequence
  synthetic nucleotide probe
tcatctgcactgccatacgtggagt

SEQ ID NO: 1114 - DNA, 25 bp, Artificial Sequence
  synthetic nucleotide probe
cctgtgctcgggaagtcccgcagaa

SEQ ID NO: 1115 - DNA, 25 bp, Artificial Sequence
  synthetic nucleotide probe
ccttggccgcttcgagcggatgctg

SEQ ID NO: 1116 - DNA, 25 bp, Artificial Sequence
  synthetic nucleotide probe
ccgcttcgagcggatgctggcggcg

SEQ ID NO: 1117 - DNA, 25 bp, Artificial Sequence
  synthetic nucleotide probe
tggcccgagccctgtgctcgggaag

SEQ ID NO: 1118 - DNA, 25 bp, Artificial Sequence
  synthetic nucleotide probe
ccgagccctgtgctcgggaagtccc

SEQ ID NO: 1119 - DNA, 25 bp, Artificial Sequence
  synthetic nucleotide probe
tgctcgggaagtcccgcagaacgcc

SEQ ID NO: 1120 - DNA, 25 bp, Artificial Sequence
  synthetic nucleotide probe
ctgctggtcgacgtgggtcagaggc

SEQ ID NO: 1121 - DNA, 25 bp, Artificial Sequence
  synthetic nucleotide probe
ctggtcgacgtgggtcagaggctgc

SEQ ID NO: 1122 - DNA, 25 bp, Artificial Sequence
  synthetic nucleotide probe
gcccgccagacttaagggacctggt

SEQ ID NO: 1123 - DNA, 25 bp, Artificial Sequence
  synthetic nucleotide probe
caggcccgccagacttaagggacct

SEQ ID NO: 1124 - DNA, 25 bp, Artificial Sequence
  synthetic nucleotide probe
acttaagggacctggtcaccacgct

SEQ ID NO: 1125 - DNA, 25 bp, Artificial Sequence
  synthetic nucleotide probe
ggttgctgcagtctggtcagatccc

SEQ ID NO: 1126 - DNA, 25 bp, Artificial Sequence
  synthetic nucleotide probe
agctgacggcggagctgatcgagca

SEQ ID NO: 1127 - DNA, 25 bp, Artificial Sequence
  synthetic nucleotide probe
gtgggccatctccaggggatgtaga

SEQ ID NO: 1128 - DNA, 25 bp, Artificial Sequence
  synthetic nucleotide probe
gtcagatccctggcagagaacgcag

SEQ ID NO: 1129 - DNA, 25 bp, Artificial Sequence
  synthetic nucleotide probe
tagcaaatgcttcaactctggctga

SEQ ID NO: 1130 - DNA, 25 bp, Artificial Sequence
  synthetic nucleotide probe
tgatcgagcaggcggcgcagtacac

SEQ ID NO: 1131 - DNA, 25 bp, Artificial Sequence
  synthetic nucleotide probe
aaggttccgcaagtcagagtactgg

SEQ ID NO: 1132 - DNA, 25 bp, Artificial Sequence
  synthetic nucleotide probe
tggcatctctcaaatcgctgactta

SEQ ID NO: 1133 - DNA, 25 bp, Artificial Sequence
  synthetic nucleotide probe
tccaggtgctggtttgccaactgac

SEQ ID NO: 1134 - DNA, 25 bp, Artificial Sequence
  synthetic nucleotide probe
tccgggggtgatctgaaccctctgg

SEQ ID NO: 1135 - DNA, 25 bp, Artificial Sequence
  synthetic nucleotide probe
aacgcagatcagggcccactgatga

SEQ ID NO: 1136 - DNA, 25 bp, Artificial Sequence
  synthetic nucleotide probe
tctccagcgaggaggtctcagtccc

SEQ ID NO: 1137 - DNA, 25 bp, Artificial Sequence
  synthetic nucleotide probe
gcgtgcacggtgttgaactgggaca

SEQ ID NO: 1138 - DNA, 25 bp, Artificial Sequence
  synthetic nucleotide probe
ctatgctccgagtagagttcatctt

SEQ ID NO: 1139 - DNA, 25 bp, Artificial Sequence
  synthetic nucleotide probe
ctccttggtagcgtgcacggtgttg

SEQ ID NO: 1140 - DNA, 25 bp, Artificial Sequence
  synthetic nucleotide probe
tgactgtgcagactctggctcgagc

SEQ ID NO: 1141 - DNA, 25 bp, Artificial Sequence
  synthetic nucleotide probe
aggtgtaggcaggtgggctctgctt

SEQ ID NO: 1142 - DNA, 25 bp, Artificial Sequence
  synthetic nucleotide probe
gaaaggggctttcatgtcgtttcct

SEQ ID NO: 1143 - DNA, 25 bp, Artificial Sequence
  synthetic nucleotide probe
tcttcctcatccctagggtgttgtg

SEQ ID NO: 1144 - DNA, 25 bp, Artificial Sequence
  synthetic nucleotide probe
gaactctttttaaactctatgctcc

SEQ ID NO: 1145 - DNA, 25 bp, Artificial Sequence
  synthetic nucleotide probe
gtctgcagacctcagagaggtccca

SEQ ID NO: 1146 - DNA, 25 bp, Artificial Sequence
  synthetic nucleotide probe
gaactgggacactggggagaaaggg

SEQ ID NO: 1147 - DNA, 25 bp, Artificial Sequence
  synthetic nucleotide probe
aaagtaccaggcactaggtcggcgc

SEQ ID NO: 1148 - DNA, 25 bp, Artificial Sequence
  synthetic nucleotide probe
gcgctgccgggagatcgagcaggtg

SEQ ID NO: 1149 - DNA, 25 bp, Artificial Sequence
  synthetic nucleotide probe
tggccccggtgcagattaaggttga

SEQ ID NO: 1150 - DNA, 25 bp, Artificial Sequence
  synthetic nucleotide probe
cagccagttcaccattgtgctggag

SEQ ID NO: 1151 - DNA, 25 bp, Artificial Sequence
  synthetic nucleotide probe
gccgagcaggaaatgcgctgactcc

SEQ ID NO: 1152 - DNA, 25 bp, Artificial Sequence
  synthetic nucleotide probe
cctggattccagttgggtttctcgg

SEQ ID NO: 1153 - DNA, 25 bp, Artificial Sequence
  synthetic nucleotide probe
gctggactcctacggggatgactac

SEQ ID NO: 1154 - DNA, 25 bp, Artificial Sequence
  synthetic nucleotide probe
aacgagcgggtcctgaacaggctcc

SEQ ID NO: 1155 - DNA, 25 bp, Artificial Sequence
  synthetic nucleotide probe
tcggggtccagacaaactgctgccc

SEQ ID NO: 1156 - DNA, 25 bp, Artificial Sequence
  synthetic nucleotide probe
ggttcctcatgagagtgctggactc

SEQ ID NO: 1157 - DNA, 25 bp, Artificial Sequence
  synthetic nucleotide probe
ggcggcgccagcgggaattaaatcg

SEQ ID NO: 1158 - DNA, 25 bp, Artificial Sequence
  synthetic nucleotide probe
tgtgtctttccagaaggtcacgtgg

SEQ ID NO: 1159 - DNA, 25 bp, Artificial Sequence
  synthetic nucleotide probe
caaagatggggtgccaagacggtgc

SEQ ID NO: 1160 - DNA, 25 bp, Artificial Sequence
  synthetic nucleotide probe
tcgcccaggatgacgcgggggccga

SEQ ID NO: 1161 - DNA, 25 bp, Artificial Sequence
  synthetic nucleotide probe
gtggaaatgtctcgggacttgggtc

SEQ ID NO: 1162 - DNA, 25 bp, Artificial Sequence
  synthetic nucleotide probe
cgtgaacagccttttatctccaagc

SEQ ID NO: 1163 - DNA, 25 bp, Artificial Sequence
  synthetic nucleotide probe
gaaactcatctccttcctgaggagc

SEQ ID NO: 1164 - DNA, 25 bp, Artificial Sequence
  synthetic nucleotide probe
gaatttcgtgaacagccttttatct

SEQ ID NO: 1165 - DNA, 25 bp, Artificial Sequence
  synthetic nucleotide probe
ccaggacaccgctgtcctggcattt

SEQ ID NO: 1166 - DNA, 25 bp, Artificial Sequence
  synthetic nucleotide probe
cagccttttatctccaagcggaaag

SEQ ID NO: 1167 - DNA, 25 bp, Artificial Sequence
  synthetic nucleotide probe
ttcctcattggattactgtgtttta

SEQ ID NO: 1168 - DNA, 25 bp, Artificial Sequence
  synthetic nucleotide probe
gaaggtcacgtggaaatgtctcggg

SEQ ID NO: 1169 - DNA, 25 bp, Artificial Sequence
  synthetic nucleotide probe
ctgggagaccgaccctgattttgtg

SEQ ID NO: 1170 - DNA, 25 bp, Artificial Sequence
  synthetic nucleotide probe
aatcagtccccaatgcctggaaatt

SEQ ID NO: 1171 - DNA, 25 bp, Artificial Sequence
  synthetic nucleotide probe
gcctggaaattcctcattggattac

SEQ ID NO: 1172 - DNA, 25 bp, Artificial Sequence
  synthetic nucleotide probe
cctgaggtgcattttctcatcatcc

SEQ ID NO: 1173 - DNA, 25 bp, Artificial Sequence
  synthetic nucleotide probe
catccttgctttaccacaatgagca

SEQ ID NO: 1174 - DNA, 25 bp, Artificial Sequence
  synthetic nucleotide probe
atttgtggccactcactttgtagga

SEQ ID NO: 1175 - DNA, 25 bp, Artificial Sequence
  synthetic nucleotide probe
gcatcgcatgcagcgcaaggttctc

SEQ ID NO: 1176 - DNA, 25 bp, Artificial Sequence
  synthetic nucleotide probe
gaggaaggagctgtagtgtcctgct

SEQ ID NO: 1177 - DNA, 25 bp, Artificial Sequence
  synthetic nucleotide probe
ctgggaagtgtgcttttggcatccg

SEQ ID NO: 1178 - DNA, 25 bp, Artificial Sequence
  synthetic nucleotide probe
cggcgagatccagcatcgcatgcag

SEQ ID NO: 1179 - DNA, 25 bp, Artificial Sequence
  synthetic nucleotide probe
ccagcatcgcatgcagcgcaaggtt

SEQ ID NO: 1180 - DNA, 25 bp, Artificial Sequence
  synthetic nucleotide probe
ctgcctgccattgacaatgttgctg

SEQ ID NO: 1181 - DNA, 25 bp, Artificial Sequence
  synthetic nucleotide probe
gcgagatccagcatcgcatgcagcg

SEQ ID NO: 1182 - DNA, 25 bp, Artificial Sequence
  synthetic nucleotide probe
gttctgggaagtgtgcttttggcat

SEQ ID NO: 1183 - DNA, 25 bp, Artificial Sequence
  synthetic nucleotide probe
gaaggagctgtagtgtcctgctgcc

SEQ ID NO: 1184 - DNA, 25 bp, Artificial Sequence
  synthetic nucleotide probe
tcgcatgcagcgcaaggttctctac

SEQ ID NO: 1185 - DNA, 25 bp, Artificial Sequence
  synthetic nucleotide probe
ttgacaatgttgctgggacctctgc

SEQ ID NO: 1186 - DNA, 25 bp, Artificial Sequence
  synthetic nucleotide probe
gttacattgtcttactctcttttac

SEQ ID NO: 1187 - DNA, 25 bp, Artificial Sequence
  synthetic nucleotide probe
gttacattgtcttactctcttttac

SEQ ID NO: 1188 - DNA, 25 bp, Artificial Sequence
  synthetic nucleotide probe
gaggccccagggccaatcaatttca

SEQ ID NO: 1189 - DNA, 25 bp, Artificial Sequence
  synthetic nucleotide probe
gtaccattcaggaagattacctaag

SEQ ID NO: 1190 - DNA, 25 bp, Artificial Sequence
  synthetic nucleotide probe
gaaacacaggccatcagggaaaacg

SEQ ID NO: 1191 - DNA, 25 bp, Artificial Sequence
  synthetic nucleotide probe
gaaaaatccaactctcatcctgggc

SEQ ID NO: 1192 - DNA, 25 bp, Artificial Sequence
  synthetic nucleotide probe
catcctgggcagaggttgcctagtt

SEQ ID NO: 1193 - DNA, 25 bp, Artificial Sequence
  synthetic nucleotide probe
gatacatggctgttcgtgacattct

SEQ ID NO: 1194 - DNA, 25 bp, Artificial Sequence
  synthetic nucleotide probe
aatgtcctgccagtttaagggtaca

SEQ ID NO: 1195 - DNA, 25 bp, Artificial Sequence
  synthetic nucleotide probe
gggtacattgtagagccgaactttg

SEQ ID NO: 1196 - DNA, 25 bp, Artificial Sequence
  synthetic nucleotide probe
gagccgaactttgagttactgtgca

SEQ ID NO: 1197 - DNA, 25 bp, Artificial Sequence
  synthetic nucleotide probe
tactttacaatgttcccttaagcaa

SEQ ID NO: 1198 - DNA, 25 bp, Artificial Sequence
  synthetic nucleotide probe
gataataaacctctaaacctgccca

SEQ ID NO: 1199 - DNA, 25 bp, Artificial Sequence
  synthetic nucleotide probe
aacctgcccagcggaagtgtgtttt

SEQ ID NO: 1200 - DNA, 25 bp, Artificial Sequence
  synthetic nucleotide probe
tacttttttttccatagctgggata

SEQ ID NO: 1201 - DNA, 25 bp, Artificial Sequence
  synthetic nucleotide probe
caagagtagcctcaacctgtgcttc

SEQ ID NO: 1202 - DNA, 25 bp, Artificial Sequence
  synthetic nucleotide probe
cagggtggcagatgaagctttctac

SEQ ID NO: 1203 - DNA, 25 bp, Artificial Sequence
  synthetic nucleotide probe
acaacccttcgctgacgtgattggt

SEQ ID NO: 1204 - DNA, 25 bp, Artificial Sequence
  synthetic nucleotide probe
accatccttgctacagcctagaaca

SEQ ID NO: 1205 - DNA, 25 bp, Artificial Sequence
  synthetic nucleotide probe
tgacattgacgagtcgtccccaggc

SEQ ID NO: 1206 - DNA, 25 bp, Artificial Sequence
  synthetic nucleotide probe
taaccccaagtctagcaagcaggcc

SEQ ID NO: 1207 - DNA, 25 bp, Artificial Sequence
  synthetic nucleotide probe
gcaagcaggccaaagatgtctcccg

SEQ ID NO: 1208 - DNA, 25 bp, Artificial Sequence
  synthetic nucleotide probe
gccaccctgtggaaacactacatct

SEQ ID NO: 1209 - DNA, 25 bp, Artificial Sequence
  synthetic nucleotide probe
atctgcaatatcttaatcctactca

SEQ ID NO: 1210 - DNA, 25 bp, Artificial Sequence
  synthetic nucleotide probe
gaagctcttcacagtcattggatta

SEQ ID NO: 1211 - DNA, 25 bp, Artificial Sequence
  synthetic nucleotide probe
tgtttgtgattgcatgtttccttcc

SEQ ID NO: 1212 - DNA, 25 bp, Artificial Sequence
  synthetic nucleotide probe
tacttttctttctaacatatcaatg

SEQ ID NO: 1213 - DNA, 25 bp, Artificial Sequence
  synthetic nucleotide probe
ataccatacttatatacctgcaact

SEQ ID NO: 1214 - DNA, 25 bp, Artificial Sequence
  synthetic nucleotide probe
atgctctgtaactctgtactgctag

SEQ ID NO: 1215 - DNA, 25 bp, Artificial Sequence
  synthetic nucleotide probe
aatacagccagtgcttaatgcttat

SEQ ID NO: 1216 - DNA, 25 bp, Artificial Sequence
  synthetic nucleotide probe
aatgtggatttgtcggcttttatgt

SEQ ID NO: 1217 - DNA, 25 bp, Artificial Sequence
  synthetic nucleotide probe
gcaagtgacaatacattccaccaca

SEQ ID NO: 1218 - DNA, 25 bp, Artificial Sequence
  synthetic nucleotide probe
aatacactcttgttcttctagcttt

SEQ ID NO: 1219 - DNA, 25 bp, Artificial Sequence
  synthetic nucleotide probe
aaaccgggtgcttcaaagtacatga

SEQ ID NO: 1220 - DNA, 25 bp, Artificial Sequence
  synthetic nucleotide probe
ggaacactatacctgtcatggatga

SEQ ID NO: 1221 - DNA, 25 bp, Artificial Sequence
  synthetic nucleotide probe
ggatgaactgaagactttgcctgtt

SEQ ID NO: 1222 - DNA, 25 bp, Artificial Sequence
  synthetic nucleotide probe
ggaggcccaatttcactcaaatgtt

SEQ ID NO: 1223 - DNA, 25 bp, Artificial Sequence
  synthetic nucleotide probe
gtttttctcaacactacttttctga

SEQ ID NO: 1224 - DNA, 25 bp, Artificial Sequence
  synthetic nucleotide probe
gctcagctgggagcatcattctcct

SEQ ID NO: 1225 - DNA, 25 bp, Artificial Sequence
  synthetic nucleotide probe
gctcagctgggagcatcattctcct

SEQ ID NO: 1226 - DNA, 25 bp, Artificial Sequence
  synthetic nucleotide probe
gagaatggcttatgggggcccaggt

SEQ ID NO: 1227 - DNA, 25 bp, Artificial Sequence
  synthetic nucleotide probe
gtttaatggtgatgcctcgcgtaca

SEQ ID NO: 1228 - DNA, 25 bp, Artificial Sequence
  synthetic nucleotide probe
tctctagtcctacccagttttaaag

SEQ ID NO: 1229 - DNA, 25 bp, Artificial Sequence
  synthetic nucleotide probe
gcctcgcgtacaggatctggttacc

SEQ ID NO: 1230 - DNA, 25 bp, Artificial Sequence
  synthetic nucleotide probe
gttgggcagatcagtgtctctagtc

SEQ ID NO: 1231 - DNA, 25 bp, Artificial Sequence
  synthetic nucleotide probe
caccatcatgtctaggcctatgcta

SEQ ID NO: 1232 - DNA, 25 bp, Artificial Sequence
  synthetic nucleotide probe
gacctcatctcccgcaaataaatgt

SEQ ID NO: 1233 - DNA, 25 bp, Artificial Sequence
  synthetic nucleotide probe
aacgcccgcagcacaacgaagaggc

SEQ ID NO: 1234 - DNA, 25 bp, Artificial Sequence
  synthetic nucleotide probe
ccgcagcacaacgaagaggccaatg

SEQ ID NO: 1235 - DNA, 25 bp, Artificial Sequence
  synthetic nucleotide probe
aagaggccaatgggcactcttccag

SEQ ID NO: 1236 - DNA, 25 bp, Artificial Sequence
  synthetic nucleotide probe
cactcttccagaggctttgtggtgc

SEQ ID NO: 1237 - DNA, 25 bp, Artificial Sequence
  synthetic nucleotide probe
tccagaggctttgtggtgcgggacc

SEQ ID NO: 1238 - DNA, 25 bp, Artificial Sequence
  synthetic nucleotide probe
acctgctccactgtttaacactaaa

SEQ ID NO: 1239 - DNA, 25 bp, Artificial Sequence
  synthetic nucleotide probe
aaccaaggtcatgagcattcgtgct

SEQ ID NO: 1240 - DNA, 25 bp, Artificial Sequence
  synthetic nucleotide probe
taagataacagactccagctcctgg

SEQ ID NO: 1241 - DNA, 25 bp, Artificial Sequence
  synthetic nucleotide probe
taggattccacttcctgtgtcatga

SEQ ID NO: 1242 - DNA, 25 bp, Artificial Sequence
  synthetic nucleotide probe
ccacttcctgtgtcatgacctcagg

SEQ ID NO: 1243 - DNA, 25 bp, Artificial Sequence
  synthetic nucleotide probe
gacctcaggaaataaacgtccttga

SEQ ID NO: 1244 - DNA, 25 bp, Artificial Sequence
  synthetic nucleotide probe
tgggatgttctggcagctgtgtcat

SEQ ID NO: 1245 - DNA, 25 bp, Artificial Sequence
  synthetic nucleotide probe
ttgttgccatcacgttcctacaaaa

SEQ ID NO: 1246 - DNA, 25 bp, Artificial Sequence
  synthetic nucleotide probe
gcctttctctccgtaaactatttag

SEQ ID NO: 1247 - DNA, 25 bp, Artificial Sequence
  synthetic nucleotide probe
aatagtgaacttgattcccctgctt

SEQ ID NO: 1248 - DNA, 25 bp, Artificial Sequence
  synthetic nucleotide probe
atgctgctgggttcattcattcatt

SEQ ID NO: 1249 - DNA, 25 bp, Artificial Sequence
  synthetic nucleotide probe
ctgcttccactaaatccagttgtga

SEQ ID NO: 1250 - DNA, 25 bp, Artificial Sequence
  synthetic nucleotide probe
gcactccgtaactcaacatggcatg

SEQ ID NO: 1251 - DNA, 25 bp, Artificial Sequence
  synthetic nucleotide probe
gagaatattggctgctgattgttgc

SEQ ID NO: 1252 - DNA, 25 bp, Artificial Sequence
  synthetic nucleotide probe
gtttagggatctttctgatggtctt

SEQ ID NO: 1253 - DNA, 25 bp, Artificial Sequence
  synthetic nucleotide probe
ttttcagtatctctgtacctgttaa

SEQ ID NO: 1254 - DNA, 25 bp, Artificial Sequence
  synthetic nucleotide probe
cttagttctaagtcattgttcccat

SEQ ID NO: 1255 - DNA, 25 bp, Artificial Sequence
  synthetic nucleotide probe
aaatcttgtcagaagatcccagaaa

SEQ ID NO: 1256 - DNA, 25 bp, Artificial Sequence
  synthetic nucleotide probe
aaagttctaattttcattagcaatt

SEQ ID NO: 1257 - DNA, 25 bp, Artificial Sequence
  synthetic nucleotide probe
atttgtactttttggcctgggatat

SEQ ID NO: 1258 - DNA, 25 bp, Artificial Sequence
  synthetic nucleotide probe
gcctgggatatgggttttaaatgga

SEQ ID NO: 1259 - DNA, 25 bp, Artificial Sequence
  synthetic nucleotide probe
aatggacattgtctgtaccagcttc

SEQ ID NO: 1260 - DNA, 25 bp, Artificial Sequence
  synthetic nucleotide probe
cattgtctgtaccagcttcattaaa

SEQ ID NO: 1261 - DNA, 25 bp, Artificial Sequence
  synthetic nucleotide probe
aatgacctttccttaacttgaagct

SEQ ID NO: 1262 - DNA, 25 bp, Artificial Sequence
  synthetic nucleotide probe
gacctttccttaacttgaagctact

SEQ ID NO: 1263 - DNA, 25 bp, Artificial Sequence
  synthetic nucleotide probe
gagggtctggaccaaaagaagagga

SEQ ID NO: 1264 - DNA, 25 bp, Artificial Sequence
  synthetic nucleotide probe
aggtgagagtaatgactaactccaa

SEQ ID NO: 1265 - DNA, 25 bp, Artificial Sequence
  synthetic nucleotide probe
ctaactccaaagatggcttcactga

SEQ ID NO: 1266 - DNA, 25 bp, Artificial Sequence
  synthetic nucleotide probe
aagttcgagttgtgctcttcacgtt

SEQ ID NO: 1267 - DNA, 25 bp, Artificial Sequence
  synthetic nucleotide probe
tgtgctcttcacgttggttcgataa

SEQ ID NO: 1268 - DNA, 25 bp, Artificial Sequence
  synthetic nucleotide probe
cacgttggttcgataatggccttta

SEQ ID NO: 1269 - DNA, 25 bp, Artificial Sequence
  synthetic nucleotide probe
gtaaatattccattctgatttcata

SEQ ID NO: 1270 - DNA, 25 bp, Artificial Sequence
  synthetic nucleotide probe
attaaacatttatgcctcccttttg

SEQ ID NO: 1271 - DNA, 25 bp, Artificial Sequence
  synthetic nucleotide probe
cctcccttttgtgttgacactgtag

SEQ ID NO: 1272 - DNA, 25 bp, Artificial Sequence
  synthetic nucleotide probe
gttgacactgtagctcatactggaa

SEQ ID NO: 1273 - DNA, 25 bp, Artificial Sequence
  synthetic nucleotide probe
gtgattatcgaccatggtatgcatg

SEQ ID NO: 1274 - DNA, 25 bp, Artificial Sequence
  synthetic nucleotide probe
ggtatgcatgatcgttgtaattgtt

SEQ ID NO: 1275 - DNA, 25 bp, Artificial Sequence
  synthetic nucleotide probe
ttttttgtttcagtaccagaggcac

SEQ ID NO: 1276 - DNA, 25 bp, Artificial Sequence
  synthetic nucleotide probe
gtaccagaggcactgacttcaataa

SEQ ID NO: 1277 - DNA, 25 bp, Artificial Sequence
  synthetic nucleotide probe
aataatctcttgttatgcagggagt

SEQ ID NO: 1278 - DNA, 25 bp, Artificial Sequence
  synthetic nucleotide probe
tatgcagggagtacagttcttttca

SEQ ID NO: 1279 - DNA, 25 bp, Artificial Sequence
  synthetic nucleotide probe
tcttttcattcatacataagttcag

SEQ ID NO: 1280 - DNA, 25 bp, Artificial Sequence
  synthetic nucleotide probe
taagttcagtagttgcttccctaac

SEQ ID NO: 1281 - DNA, 25 bp, Artificial Sequence
  synthetic nucleotide probe
gttgcttccctaactgcaaaggcaa

SEQ ID NO: 1282 - DNA, 25 bp, Artificial Sequence
  synthetic nucleotide probe
actgcaaaggcaatctcatttagtt

SEQ ID NO: 1283 - DNA, 25 bp, Artificial Sequence
  synthetic nucleotide probe
gagtagctcttgaaagcagctttga

SEQ ID NO: 1284 - DNA, 25 bp, Artificial Sequence
  synthetic nucleotide probe
agaagtatgtgtgttacaccctcac

SEQ ID NO: 1285 - DNA, 25 bp, Artificial Sequence
  synthetic nucleotide probe
tgctgtgtggggcagttcaacacaa

SEQ ID NO: 1286 - DNA, 25 bp, Artificial Sequence
  synthetic nucleotide probe
gttggcatgtcaaatgcatcctcta

SEQ ID NO: 1287 - DNA, 25 bp, Artificial Sequence
  synthetic nucleotide probe
acagcctgatgtttgggaccttttt

SEQ ID NO: 1288 - DNA, 25 bp, Artificial Sequence
  synthetic nucleotide probe
gttgggatttgcttcattgtttgac

SEQ ID NO: 1289 - DNA, 25 bp, Artificial Sequence
  synthetic nucleotide probe
tgcacagtctgttacaggttgacac

SEQ ID NO: 1290 - DNA, 25 bp, Artificial Sequence
  synthetic nucleotide probe
ttgacacattgcttgacctgattta

SEQ ID NO: 1291 - DNA, 25 bp, Artificial Sequence
  synthetic nucleotide probe
tagtgtagctttaatgtgctgcaca

SEQ ID NO: 1292 - DNA, 25 bp, Artificial Sequence
  synthetic nucleotide probe
gtgctgcacatgatactggcagccc

SEQ ID NO: 1293 - DNA, 25 bp, Artificial Sequence
  synthetic nucleotide probe
actggcagccctagagttcatagat

SEQ ID NO: 1294 - DNA, 25 bp, Artificial Sequence
  synthetic nucleotide probe
gttcatagatggacttttgggaccc

SEQ ID NO: 1295 - DNA, 25 bp, Artificial Sequence
  synthetic nucleotide probe
gggacccagcagttttgaaatgtgt

SEQ ID NO: 1296 - DNA, 25 bp, Artificial Sequence
  synthetic nucleotide probe
gcagcccctgtctaactgaaatttc

SEQ ID NO: 1297 - DNA, 25 bp, Artificial Sequence
  synthetic nucleotide probe
ctaactgaaatttctcttcaccttg

SEQ ID NO: 1298 - DNA, 25 bp, Artificial Sequence
  synthetic nucleotide probe
cttcaccttgtacacttgacagctg

SEQ ID NO: 1299 - DNA, 25 bp, Artificial Sequence
  synthetic nucleotide probe
agagtgtctgatgcggccactcatt

SEQ ID NO: 1300 - DNA, 25 bp, Artificial Sequence
  synthetic nucleotide probe
tgaagccgccctaaggattttcctt

SEQ ID NO: 1301 - DNA, 25 bp, Artificial Sequence
  synthetic nucleotide probe
ggggaacttcttcatgggtggttca

SEQ ID NO: 1302 - DNA, 25 bp, Artificial Sequence
  synthetic nucleotide probe
ttgtgtgttctgtacatgtgatgtt

SEQ ID NO: 1303 - DNA, 25 bp, Artificial Sequence
  synthetic nucleotide probe
ctcccaatgctgctcagcttgcagt

SEQ ID NO: 1304 - DNA, 25 bp, Artificial Sequence
  synthetic nucleotide probe
gaggaggatgcatttcaaaagcttg

SEQ ID NO: 1305 - DNA, 25 bp, Artificial Sequence
  synthetic nucleotide probe
gatgtcgtgcaaactgtactgtgaa

SEQ ID NO: 1306 - DNA, 25 bp, Artificial Sequence
  synthetic nucleotide probe
ataggttgtctctgcatacacgaac

SEQ ID NO: 1307 - DNA, 25 bp, Artificial Sequence
  synthetic nucleotide probe
gattctttacttagcttgtttttag

SEQ ID NO: 1308 - DNA, 25 bp, Artificial Sequence
  synthetic nucleotide probe
atttatatcccatctagaattcagc

SEQ ID NO: 1309 - DNA, 25 bp, Artificial Sequence
  synthetic nucleotide probe
tgcagtcatgcagggagccaacgtc

SEQ ID NO: 1310 - DNA, 25 bp, Artificial Sequence
  synthetic nucleotide probe
gtggtgcacttaacttgtggaattt

SEQ ID NO: 1311 - DNA, 25 bp, Artificial Sequence
  synthetic nucleotide probe
gtttgactgtaccattgactgttat

SEQ ID NO: 1312 - DNA, 25 bp, Artificial Sequence
  synthetic nucleotide probe
gatgaagttgcattacacctcactg

SEQ ID NO: 1313 - DNA, 25 bp, Artificial Sequence
  synthetic nucleotide probe
aagttcagcgttgtatgtctctctc

SEQ ID NO: 1314 - DNA, 25 bp, Artificial Sequence
  synthetic nucleotide probe
aatactgtaccatactggtctttgc

SEQ ID NO: 1315 - DNA, 25 bp, Artificial Sequence
  synthetic nucleotide probe
tctttctggtgcccaaactttcagg

SEQ ID NO: 1316 - DNA, 25 bp, Artificial Sequence
  synthetic nucleotide probe
tacacgaacctaacccaaatttgct

SEQ ID NO: 1317 - DNA, 25 bp, Artificial Sequence
  synthetic nucleotide probe
gaattcagctaggtgctgctgctgc

SEQ ID NO: 1318 - DNA, 25 bp, Artificial Sequence
  synthetic nucleotide probe
acgtcctcgtaactcagcggaaggg

SEQ ID NO: 1319 - DNA, 25 bp, Artificial Sequence
  synthetic nucleotide probe
ctctctctacactgtggtgcactta

SEQ ID NO: 1320 - DNA, 25 bp, Artificial Sequence
  synthetic nucleotide probe
aatgacttgagtccagtgaaatctc

SEQ ID NO: 1321 - DNA, 25 bp, Artificial Sequence
  synthetic nucleotide probe
tagcagtacctccctaaagcatttt

SEQ ID NO: 1322 - DNA, 25 bp, Artificial Sequence
  synthetic nucleotide probe
acacctcactgcaaggattctttac

SEQ ID NO: 1323 - DNA, 25 bp, Artificial Sequence
  synthetic nucleotide probe
ggctccccagaattcctagactggg

SEQ ID NO: 1324 - DNA, 25 bp, Artificial Sequence
  synthetic nucleotide probe
ctctgtttcctttgatgacgctttg

SEQ ID NO: 1325 - DNA, 25 bp, Artificial Sequence
  synthetic nucleotide probe
gaagtcacaggccctgtcattgcgc

SEQ ID NO: 1326 - DNA, 25 bp, Artificial Sequence
  synthetic nucleotide probe
gatgccaagtccaatagcctcatct

SEQ ID NO: 1327 - DNA, 25 bp, Artificial Sequence
  synthetic nucleotide probe
cccacaactacactctgtacttcat

SEQ ID NO: 1328 - DNA, 25 bp, Artificial Sequence
  synthetic nucleotide probe
ggagtacaaattcagcgtggatgtg

SEQ ID NO: 1329 - DNA, 25 bp, Artificial Sequence
  synthetic nucleotide probe
gattcagattgagtcctgaggcatt

SEQ ID NO: 1330 - DNA, 25 bp, Artificial Sequence
  synthetic nucleotide probe
gtaggaatcctggttgtggggacca

SEQ ID NO: 1331 - DNA, 25 bp, Artificial Sequence
  synthetic nucleotide probe
actctggatccagtgacagcaggtg

SEQ ID NO: 1332 - DNA, 25 bp, Artificial Sequence
  synthetic nucleotide probe
acagcaggtgtcatgggtcaagcat

SEQ ID NO: 1333 - DNA, 25 bp, Artificial Sequence
  synthetic nucleotide probe
aatcatatatagcattttcaggcat

SEQ ID NO: 1334 - DNA, 25 bp, Artificial Sequence
  synthetic nucleotide probe
ggcatgttcctggtagttcttttga

SEQ ID NO: 1335 - DNA, 25 bp, Artificial Sequence
  synthetic nucleotide probe
ctggtagttcttttgagtctgacat

SEQ ID NO: 1336 - DNA, 25 bp, Artificial Sequence
  synthetic nucleotide probe
gtatgatggtttgactgtatggcag

SEQ ID NO: 1337 - DNA, 25 bp, Artificial Sequence
  synthetic nucleotide probe
ggatcttgattgataactgccatga

SEQ ID NO: 1338 - DNA, 25 bp, Artificial Sequence
  synthetic nucleotide probe
gtgtgttcatcctagagttattttt

SEQ ID NO: 1339 - DNA, 25 bp, Artificial Sequence
  synthetic nucleotide probe
ccttcccctccaaattgtatacatt

SEQ ID NO: 1340 - DNA, 25 bp, Artificial Sequence
  synthetic nucleotide probe
tgttgtagcagcctcttgttttttt

SEQ ID NO: 1341 - DNA, 25 bp, Artificial Sequence
  synthetic nucleotide probe
gcgtggcagcggaagacgattccca

SEQ ID NO: 1342 - DNA, 25 bp, Artificial Sequence
  synthetic nucleotide probe
aattcctatgttcagtagcgtggtt

SEQ ID NO: 1343 - DNA, 25 bp, Artificial Sequence
  synthetic nucleotide probe
aactgccatgatattttgctttgat

SEQ ID NO: 1344 - DNA, 25 bp, Artificial Sequence
  synthetic nucleotide probe
acatgtagttgcacacggttcagta

SEQ ID NO: 1345 - DNA, 25 bp, Artificial Sequence
  synthetic nucleotide probe
tttcctcagtcttcaatgacgagag

SEQ ID NO: 1346 - DNA, 25 bp, Artificial Sequence
  synthetic nucleotide probe
atgttcacaacttgcgtgcgtggca

SEQ ID NO: 1347 - DNA, 25 bp, Artificial Sequence
  synthetic nucleotide probe
agtccttgaagctttacagttaatt

SEQ ID NO: 1348 - DNA, 25 bp, Artificial Sequence
  synthetic nucleotide probe
acctttatgctcgttccatatttgg

SEQ ID NO: 1349 - DNA, 25 bp, Artificial Sequence
  synthetic nucleotide probe
tatggcagccaacctttatgctcgt

SEQ ID NO: 1350 - DNA, 25 bp, Artificial Sequence
  synthetic nucleotide probe
gtttcatgtaccaagacccttttca

SEQ ID NO: 1351 - DNA, 25 bp, Artificial Sequence
  synthetic nucleotide probe
gtttgtcttttgtcttaacagttct

SEQ ID NO: 1352 - DNA, 25 bp, Artificial Sequence
  synthetic nucleotide probe
gaatgctgtcctcaaagtatataat

SEQ ID NO: 1353 - DNA, 25 bp, Artificial Sequence
  synthetic nucleotide probe
tgctgtcctcaaagtatataatgtt

SEQ ID NO: 1354 - DNA, 25 bp, Artificial Sequence
  synthetic nucleotide probe
gatgcacttgcaaatgtcagcattg

SEQ ID NO: 1355 - DNA, 25 bp, Artificial Sequence
  synthetic nucleotide probe
accaagacccttttcacagtacaat

SEQ ID NO: 1356 - DNA, 25 bp, Artificial Sequence
  synthetic nucleotide probe
gaatacttttcagccaataatttat

SEQ ID NO: 1357 - DNA, 25 bp, Artificial Sequence
  synthetic nucleotide probe
gacccttttcacagtacaataaaca

SEQ ID NO: 1358 - DNA, 25 bp, Artificial Sequence
  synthetic nucleotide probe
taatgtttcatgtaccaagaccctt

SEQ ID NO: 1359 - DNA, 25 bp, Artificial Sequence
  synthetic nucleotide probe
ctgctgttaccggccatataagaat

SEQ ID NO: 1360 - DNA, 25 bp, Artificial Sequence
  synthetic nucleotide probe
catgtaccaagacccttttcacagt

SEQ ID NO: 1361 - DNA, 25 bp, Artificial Sequence
  synthetic nucleotide probe
aaatgactacttacagcacatatta

SEQ ID NO: 1362 - DNA, 25 bp, Artificial Sequence
  synthetic nucleotide probe
ggtatgggcttactggactccaaca

SEQ ID NO: 1363 - DNA, 25 bp, Artificial Sequence
  synthetic nucleotide probe
taacagttctgaatgctgtcctcaa

SEQ ID NO: 1364 - DNA, 25 bp, Artificial Sequence
  synthetic nucleotide probe
gtcttttgtcttaacagttctgaat

SEQ ID NO: 1365 - DNA, 25 bp, Artificial Sequence
  synthetic nucleotide probe
gaagccaattcaccagggaccagat

SEQ ID NO: 1366 - DNA, 25 bp, Artificial Sequence
  synthetic nucleotide probe
actccaacatcttttgtactctttc

SEQ ID NO: 1367 - DNA, 25 bp, Artificial Sequence
  synthetic nucleotide probe
agttctgaatgctgtcctcaaagta

SEQ ID NO: 1368 - DNA, 25 bp, Artificial Sequence
  synthetic nucleotide probe
gccctttctggttactgtggcttta

SEQ ID NO: 1369 - DNA, 25 bp, Artificial Sequence
  synthetic nucleotide probe
gagatctgaggaggcttcgtgggct

SEQ ID NO: 1370 - DNA, 25 bp, Artificial Sequence
  synthetic nucleotide probe
gcactggcacctagacatgtacaac

SEQ ID NO: 1371 - DNA, 25 bp, Artificial Sequence
  synthetic nucleotide probe
cgggtggttcactatgagatctgag

SEQ ID NO: 1372 - DNA, 25 bp, Artificial Sequence
  synthetic nucleotide probe
tggtatagctgggaccagccgggcc

SEQ ID NO: 1373 - DNA, 25 bp, Artificial Sequence
  synthetic nucleotide probe
tgggtcctctaactaggactccctc

SEQ ID NO: 1374 - DNA, 25 bp, Artificial Sequence
  synthetic nucleotide probe
ccctgaccgctcacagcatgagaga

SEQ ID NO: 1375 - DNA, 25 bp, Artificial Sequence
  synthetic nucleotide probe
gccgggcctgaggttgaactggggt

SEQ ID NO: 1376 - DNA, 25 bp, Artificial Sequence
  synthetic nucleotide probe
cggtgatccctccaaagaaccagag

SEQ ID NO: 1377 - DNA, 25 bp, Artificial Sequence
  synthetic nucleotide probe
tacgaaccttacccggatgatggca

SEQ ID NO: 1378 - DNA, 25 bp, Artificial Sequence
  synthetic nucleotide probe
acacctgtttcttggcatgtcatgt

SEQ ID NO: 1379 - DNA, 25 bp, Artificial Sequence
  synthetic nucleotide probe
aaccgtgtggatacatcccccacac

SEQ ID NO: 1380 - DNA, 25 bp, Artificial Sequence
  synthetic nucleotide probe
tcatgtgctgggtgggggacgtgta

SEQ ID NO: 1381 - DNA, 25 bp, Artificial Sequence
  synthetic nucleotide probe
tctaactaggactccctcattccta

SEQ ID NO: 1382 - DNA, 25 bp, Artificial Sequence
  synthetic nucleotide probe
ggactccctcattcctagaaattta

SEQ ID NO: 1383 - DNA, 25 bp, Artificial Sequence
  synthetic nucleotide probe
catgaccaaggacatgttcccgggg

SEQ ID NO: 1384 - DNA, 25 bp, Artificial Sequence
  synthetic nucleotide probe
gatccctccaaagaaccagagcggg

SEQ ID NO: 1385 - DNA, 25 bp, Artificial Sequence
  synthetic nucleotide probe
ccagagcgggtggttcactatgaga

SEQ ID NO: 1386 - DNA, 25 bp, Artificial Sequence
  synthetic nucleotide probe
attggccttttacctggatataaat

SEQ ID NO: 1387 - DNA, 25 bp, Artificial Sequence
  synthetic nucleotide probe
accatccaacagacctggtgctcta

SEQ ID NO: 1388 - DNA, 25 bp, Artificial Sequence
  synthetic nucleotide probe
ccatccaacagacctggtgctctaa

SEQ ID NO: 1389 - DNA, 25 bp, Artificial Sequence
  synthetic nucleotide probe
tgctctaatgccaagttatacacgg

SEQ ID NO: 1390 - DNA, 25 bp, Artificial Sequence
  synthetic nucleotide probe
ataggctctcagtaagaagtctgat

SEQ ID NO: 1391 - DNA, 25 bp, Artificial Sequence
  synthetic nucleotide probe
ggtataaagctctcaaatgtgacca

SEQ ID NO: 1392 - DNA, 25 bp, Artificial Sequence
  synthetic nucleotide probe
aagctctcaaatgtgaccatgtgaa

SEQ ID NO: 1393 - DNA, 25 bp, Artificial Sequence
  synthetic nucleotide probe
taatggactcagctctgtctgctca

SEQ ID NO: 1394 - DNA, 25 bp, Artificial Sequence
  synthetic nucleotide probe
aatgccattgtgcagagaagcaccc

SEQ ID NO: 1395 - DNA, 25 bp, Artificial Sequence
  synthetic nucleotide probe
gaagcaccctaatgcataagctttt

SEQ ID NO: 1396 - DNA, 25 bp, Artificial Sequence
  synthetic nucleotide probe
ctaatgcataagctttttaatgctg

SEQ ID NO: 1397 - DNA, 25 bp, Artificial Sequence
  synthetic nucleotide probe
aattaaatgccactttttcagaggt

SEQ ID NO: 1398 - DNA, 25 bp, Artificial Sequence
  synthetic nucleotide probe
ccactttttcagaggtgaattaatg

SEQ ID NO: 1399 - DNA, 25 bp, Artificial Sequence
  synthetic nucleotide probe
taaatggaactattccatcaatagg

SEQ ID NO: 1400 - DNA, 25 bp, Artificial Sequence
  synthetic nucleotide probe
cactgtataccgatcaggaatcttg

SEQ ID NO: 1401 - DNA, 25 bp, Artificial Sequence
  synthetic nucleotide probe
ataccgatcaggaatcttgctccaa

SEQ ID NO: 1402 - DNA, 25 bp, Artificial Sequence
  synthetic nucleotide probe
ttgccactatgaccaaacgcacagt

SEQ ID NO: 1403 - DNA, 25 bp, Artificial Sequence
  synthetic nucleotide probe
aaacgcacagtctgttctgcagcaa

SEQ ID NO: 1404 - DNA, 25 bp, Artificial Sequence
  synthetic nucleotide probe
ctgcagcaacaacgggattcaatca

SEQ ID NO: 1405 - DNA, 25 bp, Artificial Sequence
  synthetic nucleotide probe
tcaactcagtcgtgattcagccgta

SEQ ID NO: 1406 - DNA, 25 bp, Artificial Sequence
  synthetic nucleotide probe
tcagccgtagaaatgcttttccttt

SEQ ID NO: 1407 - DNA, 25 bp, Artificial Sequence
  synthetic nucleotide probe
ttatcttgtttgagcttttcctttc

SEQ ID NO: 1408 - DNA, 25 bp, Artificial Sequence
  synthetic nucleotide probe
gaacttgtgttgtactctgtagaaa

SEQ ID NO: 1409 - DNA, 25 bp, Artificial Sequence
  synthetic nucleotide probe
gtcccaatggggaacctaaatctgt

SEQ ID NO: 1410 - DNA, 25 bp, Artificial Sequence
  synthetic nucleotide probe
gttttaattgcacagacacatggac

SEQ ID NO: 1411 - DNA, 25 bp, Artificial Sequence
  synthetic nucleotide probe
aaagtcattttgtatctgccaagtg

SEQ ID NO: 1412 - DNA, 25 bp, Artificial Sequence
  synthetic nucleotide probe
atctgccaagtgtggtaccttcctt

SEQ ID NO: 1413 - DNA, 25 bp, Artificial Sequence
  synthetic nucleotide probe
tagggagcattttccttctagtcta

SEQ ID NO: 1414 - DNA, 25 bp, Artificial Sequence
  synthetic nucleotide probe
gcattgtctgaagttagcacctctt

SEQ ID NO: 1415 - DNA, 25 bp, Artificial Sequence
  synthetic nucleotide probe
gcacctcttggactgaatcgtttgt

SEQ ID NO: 1416 - DNA, 25 bp, Artificial Sequence
  synthetic nucleotide probe
gaatcgtttgtctagactacatgta

SEQ ID NO: 1417 - DNA, 25 bp, Artificial Sequence
  synthetic nucleotide probe
gatcatgtgcatatcatcccattgt

SEQ ID NO: 1418 - DNA, 25 bp, Artificial Sequence
  synthetic nucleotide probe
atcatcccattgtaaagcgacttca

SEQ ID NO: 1419 - DNA, 25 bp, Artificial Sequence
  synthetic nucleotide probe
gtgtgtgctgtcgcttgtcgacaac

SEQ ID NO: 1420 - DNA, 25 bp, Artificial Sequence
  synthetic nucleotide probe
gtcgcttgtcgacaacagctttttg

SEQ ID NO: 1421 - DNA, 25 bp, Artificial Sequence
  synthetic nucleotide probe
atttgtgagccttcattaactcgaa

SEQ ID NO: 1422 - DNA, 25 bp, Artificial Sequence
  synthetic nucleotide probe
gttagaataggctgcatctttttaa

SEQ ID NO: 1423 - DNA, 25 bp, Artificial Sequence
  synthetic nucleotide probe
acaactctggcttttgagatgactt

SEQ ID NO: 1424 - DNA, 25 bp, Artificial Sequence
  synthetic nucleotide probe
ttcacctgcagtcgcctagaaaact

SEQ ID NO: 1425 - DNA, 25 bp, Artificial Sequence
  synthetic nucleotide probe
aaacttgctctcaaacttcagggtt

SEQ ID NO: 1426 - DNA, 25 bp, Artificial Sequence
  synthetic nucleotide probe
aagtctcatttctgtgttttgcctg

SEQ ID NO: 1427 - DNA, 25 bp, Artificial Sequence
  synthetic nucleotide probe
cctctgatgctgggacccggaaggc

SEQ ID NO: 1428 - DNA, 25 bp, Artificial Sequence
  synthetic nucleotide probe
atacctgttgtgagacccgaggggc

SEQ ID NO: 1429 - DNA, 25 bp, Artificial Sequence
  synthetic nucleotide probe
cggcgcggttttttatggtgacaca

SEQ ID NO: 1430 - DNA, 25 bp, Artificial Sequence
  synthetic nucleotide probe
tccaggctcagtattgtgaccgcgg

SEQ ID NO: 1431 - DNA, 25 bp, Artificial Sequence
  synthetic nucleotide probe
tgccttacccgatggcttgtgacgc

SEQ ID NO: 1432 - DNA, 25 bp, Artificial Sequence
  synthetic nucleotide probe
tgttcgctgtggacgctgtagaggc

SEQ ID NO: 1433 - DNA, 25 bp, Artificial Sequence
  synthetic nucleotide probe
gttggccagtctgtacctggacttc

SEQ ID NO: 1434 - DNA, 25 bp, Artificial Sequence
  synthetic nucleotide probe
gaataaatcttctgtatcctcaaaa

SEQ ID NO: 1435 - DNA, 25 bp, Artificial Sequence
  synthetic nucleotide probe
gtttacagggtctgtttcacccagc

SEQ ID NO: 1436 - DNA, 25 bp, Artificial Sequence
  synthetic nucleotide probe
ttcacccagcccggaaagtcagaga

SEQ ID NO: 1437 - DNA, 25 bp, Artificial Sequence
  synthetic nucleotide probe
caactccatctacattggttcccag

SEQ ID NO: 1438 - DNA, 25 bp, Artificial Sequence
  synthetic nucleotide probe
ctcatagcacattacccaagaatct

SEQ ID NO: 1439 - DNA, 25 bp, Artificial Sequence
  synthetic nucleotide probe
gaacacctatattcgttatgaactt

SEQ ID NO: 1440 - DNA, 25 bp, Artificial Sequence
  synthetic nucleotide probe
ttaatgcacagctacttcacacctt

SEQ ID NO: 1441 - DNA, 25 bp, Artificial Sequence
  synthetic nucleotide probe
cacaccttaaacttgctttgatttg

SEQ ID NO: 1442 - DNA, 25 bp, Artificial Sequence
  synthetic nucleotide probe
aataacctgtctttgtttttgatgt

SEQ ID NO: 1443 - DNA, 25 bp, Artificial Sequence
  synthetic nucleotide probe
gtaaatgccagtagtgaccaagaac

SEQ ID NO: 1444 - DNA, 25 bp, Artificial Sequence
  synthetic nucleotide probe
tacactatactggagggatttcatt

SEQ ID NO: 1445 - DNA, 25 bp, Artificial Sequence
  synthetic nucleotide probe
gatttagaactcattccttgtgttt

SEQ ID NO: 1446 - DNA, 25 bp, Artificial Sequence
  synthetic nucleotide probe
actggataatcgtagcttttaatgt

SEQ ID NO: 1447 - DNA, 25 bp, Artificial Sequence
  synthetic nucleotide probe
gtagcttttaatgttgcgcctcttc

SEQ ID NO: 1448 - DNA, 25 bp, Artificial Sequence
  synthetic nucleotide probe
gtgacaacattggctacattacctt

SEQ ID NO: 1449 - DNA, 25 bp, Artificial Sequence
  synthetic nucleotide probe
tgcgcctcttcaggttcttaaggga

SEQ ID NO: 1450 - DNA, 25 bp, Artificial Sequence
  synthetic nucleotide probe
gctgtgcttgcaaagacttcatagt

SEQ ID NO: 1451 - DNA, 25 bp, Artificial Sequence
  synthetic nucleotide probe
atcttccggcatccaaggattccat

SEQ ID NO: 1452 - DNA, 25 bp, Artificial Sequence
  synthetic nucleotide probe
gagctgaaagacacagacgccgctg

SEQ ID NO: 1453 - DNA, 25 bp, Artificial Sequence
  synthetic nucleotide probe
aaagaaggacgcagagccagccaca

SEQ ID NO: 1454 - DNA, 25 bp, Artificial Sequence
  synthetic nucleotide probe
atctgcagaaacgagctgtgcttgc

SEQ ID NO: 1455 - DNA, 25 bp, Artificial Sequence
  synthetic nucleotide probe
gtctctttgctatatgaccttgaaa

SEQ ID NO: 1456 - DNA, 25 bp, Artificial Sequence
  synthetic nucleotide probe
gaggaagcggctggcaactgaaggc

SEQ ID NO: 1457 - DNA, 25 bp, Artificial Sequence
  synthetic nucleotide probe
ctcttttccaagctgtttcgctttg

SEQ ID NO: 1458 - DNA, 25 bp, Artificial Sequence
  synthetic nucleotide probe
cgttttcatcccgctaatcttggga

SEQ ID NO: 1459 - DNA, 25 bp, Artificial Sequence
  synthetic nucleotide probe
gtttcgctttgcaatatattactgg

SEQ ID NO: 1460 - DNA, 25 bp, Artificial Sequence
  synthetic nucleotide probe
ggaacacttgctactggataatcgt

SEQ ID NO: 1461 - DNA, 25 bp, Artificial Sequence
  synthetic nucleotide probe
gaagcgaaattgttttgcctctgtc

SEQ ID NO: 1462 - DNA, 25 bp, Artificial Sequence
  synthetic nucleotide probe
gagtcacagtagtcttcagcacagt

SEQ ID NO: 1463 - DNA, 25 bp, Artificial Sequence
  synthetic nucleotide probe
ggaagcggctggcaactgaaggctg

SEQ ID NO: 1464 - DNA, 25 bp, Artificial Sequence
  synthetic nucleotide probe
tgcagtcataacttgttttctccta

SEQ ID NO: 1465 - DNA, 25 bp, Artificial Sequence
  synthetic nucleotide probe
tcctctttagccacagggaacctcc

SEQ ID NO: 1466 - DNA, 25 bp, Artificial Sequence
  synthetic nucleotide probe
gaccttgaaaatcttccggcatcca

SEQ ID NO: 1467 - DNA, 25 bp, Artificial Sequence
  synthetic nucleotide probe
ggattccattgtgcatcaagctggc

SEQ ID NO: 1468 - DNA, 25 bp, Artificial Sequence
  synthetic nucleotide probe
ttcatcccgctaatcttgggaataa

SEQ ID NO: 1469 - DNA, 25 bp, Artificial Sequence
  synthetic nucleotide probe
gagactcaacatcaggatccacagc

SEQ ID NO: 1470 - DNA, 25 bp, Artificial Sequence
  synthetic nucleotide probe
acagactttatcgctctgtggctca

SEQ ID NO: 1471 - DNA, 25 bp, Artificial Sequence
  synthetic nucleotide probe
aagcagcaacagctgaggcgcacca

SEQ ID NO: 1472 - DNA, 25 bp, Artificial Sequence
  synthetic nucleotide probe
gcttccatttctttaacgtctgttc

SEQ ID NO: 1473 - DNA, 25 bp, Artificial Sequence
  synthetic nucleotide probe
tctgttcccttaacatcgctgaaat

SEQ ID NO: 1474 - DNA, 25 bp, Artificial Sequence
  synthetic nucleotide probe
gaagagatgccttgcggtgtggcca

SEQ ID NO: 1475 - DNA, 25 bp, Artificial Sequence
  synthetic nucleotide probe
gactcagaaaccttggtactcgccc

SEQ ID NO: 1476 - DNA, 25 bp, Artificial Sequence
  synthetic nucleotide probe
actggctcctgcattaacccagaaa

SEQ ID NO: 1477 - DNA, 25 bp, Artificial Sequence
  synthetic nucleotide probe
taacccagaaatacctcgcttctat

SEQ ID NO: 1478 - DNA, 25 bp, Artificial Sequence
  synthetic nucleotide probe
ctcgcttctatctgtgcacttagct

SEQ ID NO: 1479 - DNA, 25 bp, Artificial Sequence
  synthetic nucleotide probe
gggaacttacccactgtaatcacct

SEQ ID NO: 1480 - DNA, 25 bp, Artificial Sequence
  synthetic nucleotide probe
ttgtgccaaggaagtagctgcccca

SEQ ID NO: 1481 - DNA, 25 bp, Artificial Sequence
  synthetic nucleotide probe
ccttctccgcgtcattgttggaaga

SEQ ID NO: 1482 - DNA, 25 bp, Artificial Sequence
  synthetic nucleotide probe
aggagagatgcatcgagcagtccca

SEQ ID NO: 1483 - DNA, 25 bp, Artificial Sequence
  synthetic nucleotide probe
gctgcttttcatttattacttcttc

SEQ ID NO: 1484 - DNA, 25 bp, Artificial Sequence
  synthetic nucleotide probe
cttcttctttccaggacctgacaga

SEQ ID NO: 1485 - DNA, 25 bp, Artificial Sequence
  synthetic nucleotide probe
ttatgtccaaacttagcacctgcaa

SEQ ID NO: 1486 - DNA, 25 bp, Artificial Sequence
  synthetic nucleotide probe
tgtgcgtctgcgagcgcacacacat

SEQ ID NO: 1487 - DNA, 25 bp, Artificial Sequence
  synthetic nucleotide probe
aggagttgcggttgctccatgttct

SEQ ID NO: 1488 - DNA, 25 bp, Artificial Sequence
  synthetic nucleotide probe
gctccatgttctgacttagggcaat

SEQ ID NO: 1489 - DNA, 25 bp, Artificial Sequence
  synthetic nucleotide probe
ctgcacttggggtctgtctgtacag

SEQ ID NO: 1490 - DNA, 25 bp, Artificial Sequence
  synthetic nucleotide probe
gtctgtacagttactcatgtcattg

SEQ ID NO: 1491 - DNA, 25 bp, Artificial Sequence
  synthetic nucleotide probe
ttcagtgagacctctgctttcatgc

SEQ ID NO: 1492 - DNA, 25 bp, Artificial Sequence
  synthetic nucleotide probe
agcatgtttgcatagcaaccagtca

SEQ ID NO: 1493 - DNA, 25 bp, Artificial Sequence
  synthetic nucleotide probe
gttgccagtgatgattttcctattc

SEQ ID NO: 1494 - DNA, 25 bp, Artificial Sequence
  synthetic nucleotide probe
atttctgctgatatactcaccttag

SEQ ID NO: 1495 - DNA, 25 bp, Artificial Sequence
  synthetic nucleotide probe
gctgcctttcttcagcaacagaccc

SEQ ID NO: 1496 - DNA, 25 bp, Artificial Sequence
  synthetic nucleotide probe
agcaaccagtcaagagcatttacac

SEQ ID NO: 1497 - DNA, 25 bp, Artificial Sequence
  synthetic nucleotide probe
tgaaggtgccccaggggctcctatt

SEQ ID NO: 1498 - DNA, 25 bp, Artificial Sequence
  synthetic nucleotide probe
agtatggtttcctgaagtattctga

SEQ ID NO: 1499 - DNA, 25 bp, Artificial Sequence
  synthetic nucleotide probe
taaaactaaatttctttcatgtcct

SEQ ID NO: 1500 - DNA, 25 bp, Artificial Sequence
  synthetic nucleotide probe
tgctcagaaccctggtgctttattt

SEQ ID NO: 1501 - DNA, 25 bp, Artificial Sequence
  synthetic nucleotide probe
gcgtacagcaacctcttggtcaaac

SEQ ID NO: 1502 - DNA, 25 bp, Artificial Sequence
  synthetic nucleotide probe
ttctcttccactcaatattgccatt

SEQ ID NO: 1503 - DNA, 25 bp, Artificial Sequence
  synthetic nucleotide probe
ggactagtcctcattagcatgtttg

SEQ ID NO: 1504 - DNA, 25 bp, Artificial Sequence
  synthetic nucleotide probe
catacccacatagttagcaccagca

SEQ ID NO: 1505 - DNA, 25 bp, Artificial Sequence
  synthetic nucleotide probe
gagcatttacactatttctgctgat

SEQ ID NO: 1506 - DNA, 25 bp, Artificial Sequence
  synthetic nucleotide probe
agaaggattgaccatgcatacccac

SEQ ID NO: 1507 - DNA, 25 bp, Artificial Sequence
  synthetic nucleotide probe
gcaccagcaacttcagtgagacctc

SEQ ID NO: 1508 - DNA, 25 bp, Artificial Sequence
  synthetic nucleotide probe
ttgaggatcttattcagcgctgcct

SEQ ID NO: 1509 - DNA, 25 bp, Artificial Sequence
  synthetic nucleotide probe
gccagttctcaaagttgttctcacc

SEQ ID NO: 1510 - DNA, 25 bp, Artificial Sequence
  synthetic nucleotide probe
tactcaccttagaactgctcagaac

SEQ ID NO: 1511 - DNA, 25 bp, Artificial Sequence
  synthetic nucleotide probe
gtatttcctggattacacatagtat

SEQ ID NO: 1512 - DNA, 25 bp, Artificial Sequence
  synthetic nucleotide probe
gtctatgagcgtctgacctggacca

SEQ ID NO: 1513 - DNA, 25 bp, Artificial Sequence
  synthetic nucleotide probe
gttactcctgtatcattgctcataa

SEQ ID NO: 1514 - DNA, 25 bp, Artificial Sequence
  synthetic nucleotide probe
agcctctgtgcactgtttggtggcc

SEQ ID NO: 1515 - DNA, 25 bp, Artificial Sequence
  synthetic nucleotide probe
tgtatttaaagccctcagtctgtcc

SEQ ID NO: 1516 - DNA, 25 bp, Artificial Sequence
  synthetic nucleotide probe
gcctgaatggacaggggccacttca

SEQ ID NO: 1517 - DNA, 25 bp, Artificial Sequence
  synthetic nucleotide probe
atcactgtcacacaattccaatgga

SEQ ID NO: 1518 - DNA, 25 bp, Artificial Sequence
  synthetic nucleotide probe
gacctttgctcatctgtgtcagcaa

SEQ ID NO: 1519 - DNA, 25 bp, Artificial Sequence
  synthetic nucleotide probe
cccggcgtgtgccgggcctgaatgg

SEQ ID NO: 1520 - DNA, 25 bp, Artificial Sequence
  synthetic nucleotide probe
gctggagcgcaagacgtgctgacac

SEQ ID NO: 1521 - DNA, 25 bp, Artificial Sequence
  synthetic nucleotide probe
aggtgatgtcctgttcacatacctg

SEQ ID NO: 1522 - DNA, 25 bp, Artificial Sequence
  synthetic nucleotide probe
gccttcaagcgccagtttgtggagc

SEQ ID NO: 1523 - DNA, 25 bp, Artificial Sequence
  synthetic nucleotide probe
cctccatgggcattcttgttgttgg

SEQ ID NO: 1524 - DNA, 25 bp, Artificial Sequence
  synthetic nucleotide probe
tcatggtttccatggttaccggcct

SEQ ID NO: 1525 - DNA, 25 bp, Artificial Sequence
  synthetic nucleotide probe
cccagccatcactcatctttgagga

SEQ ID NO: 1526 - DNA, 25 bp, Artificial Sequence
  synthetic nucleotide probe
gcgaagtgatggactctgccaggtg

SEQ ID NO: 1527 - DNA, 25 bp, Artificial Sequence
  synthetic nucleotide probe
gccacttcacagcatgtcagggaaa

SEQ ID NO: 1528 - DNA, 25 bp, Artificial Sequence
  synthetic nucleotide probe
gtcctgttgtgtggggcgaagtgat

SEQ ID NO: 1529 - DNA, 25 bp, Artificial Sequence
  synthetic nucleotide probe
gtgctgacacagtgagttttctctg

SEQ ID NO: 1530 - DNA, 25 bp, Artificial Sequence
  synthetic nucleotide probe
gcagcttgtcatcagctacactggc

SEQ ID NO: 1531 - DNA, 25 bp, Artificial Sequence
  synthetic nucleotide probe
gtgtcagcaagttgacgtcacccgg

SEQ ID NO: 1532 - DNA, 25 bp, Artificial Sequence
  synthetic nucleotide probe
ctgccaggtggacatgctgtgggtg

SEQ ID NO: 1533 - DNA, 25 bp, Artificial Sequence
  synthetic nucleotide probe
caatgagatcttggactctgcctct

SEQ ID NO: 1534 - DNA, 25 bp, Artificial Sequence
  synthetic nucleotide probe
agatcccgcggttgttggtgggtgc

SEQ ID NO: 1535 - DNA, 25 bp, Artificial Sequence
  synthetic nucleotide probe
tctacttcactcttcctgttgaaaa

SEQ ID NO: 1536 - DNA, 25 bp, Artificial Sequence
  synthetic nucleotide probe
cagactctgcattccaaaccaaggc

SEQ ID NO: 1537 - DNA, 25 bp, Artificial Sequence
  synthetic nucleotide probe
gactgactgaacttgacctgtgacc

SEQ ID NO: 1538 - DNA, 25 bp, Artificial Sequence
  synthetic nucleotide probe
agcaacagagagtaggcggctgggc

SEQ ID NO: 1539 - DNA, 25 bp, Artificial Sequence
  synthetic nucleotide probe
tgcctggactcgctggagcaaagga

SEQ ID NO: 1540 - DNA, 25 bp, Artificial Sequence
  synthetic nucleotide probe
ccgcccatgattcttcggactgact

SEQ ID NO: 1541 - DNA, 25 bp, Artificial Sequence
  synthetic nucleotide probe
gccccttagtcactcagacatacgg

SEQ ID NO: 1542 - DNA, 25 bp, Artificial Sequence
  synthetic nucleotide probe
cgccgacgggtacctgtacatgtac

SEQ ID NO: 1543 - DNA, 25 bp, Artificial Sequence
  synthetic nucleotide probe
gtcatgtgcctttctattttcatct

SEQ ID NO: 1544 - DNA, 25 bp, Artificial Sequence
  synthetic nucleotide probe
taggggagctagaagccactttcca

SEQ ID NO: 1545 - DNA, 25 bp, Artificial Sequence
  synthetic nucleotide probe
gggcttcctacctgtgtgagaggtc

SEQ ID NO: 1546 - DNA, 25 bp, Artificial Sequence
  synthetic nucleotide probe
tcgcttcccttttcatatttacaga

SEQ ID NO: 1547 - DNA, 25 bp, Artificial Sequence
  synthetic nucleotide probe
acttgaaaggttgcctggactcgct

SEQ ID NO: 1548 - DNA, 25 bp, Artificial Sequence
  synthetic nucleotide probe
gcatgaacgtgccaagccagcatag

SEQ ID NO: 1549 - DNA, 25 bp, Artificial Sequence
  synthetic nucleotide probe
ccctgcgcctggatgaggacagcga

SEQ ID NO: 1550 - DNA, 25 bp, Artificial Sequence
  synthetic nucleotide probe
cagaactcaagtgtggtggccgtct

SEQ ID NO: 1551 - DNA, 25 bp, Artificial Sequence
  synthetic nucleotide probe
aattggatcgctctgggatttcttc

SEQ ID NO: 1552 - DNA, 25 bp, Artificial Sequence
  synthetic nucleotide probe
ggacagcgaggttctttctgatact

SEQ ID NO: 1553 - DNA, 25 bp, Artificial Sequence
  synthetic nucleotide probe
cccaccaggtgtgctgggcagactt

SEQ ID NO: 1554 - DNA, 25 bp, Artificial Sequence
  synthetic nucleotide probe
aaatgatctgttcttctacttcact

SEQ ID NO: 1555 - DNA, 25 bp, Artificial Sequence
  synthetic nucleotide probe
aaacaacctcaagtacctcagactc

SEQ ID NO: 1556 - DNA, 25 bp, Artificial Sequence
  synthetic nucleotide probe
gggcagacttcagctgggacagaag

SEQ ID NO: 1557 - DNA, 25 bp, Artificial Sequence
  synthetic nucleotide probe
ttcgggaaagtgctcatggcctcca

SEQ ID NO: 1558 - DNA, 25 bp, Artificial Sequence
  synthetic nucleotide probe
caagcttcagtatttgcctcgcttc

SEQ ID NO: 1559 - DNA, 25 bp, Artificial Sequence
  synthetic nucleotide probe
gcgtaaggaaaccgtggcgtcgcgc

SEQ ID NO: 1560 - DNA, 25 bp, Artificial Sequence
  synthetic nucleotide probe
ccacaaaaacatctgctcgctagcc

SEQ ID NO: 1561 - DNA, 25 bp, Artificial Sequence
  synthetic nucleotide probe
tctgcttgtcaaggccagttctgca

SEQ ID NO: 1562 - DNA, 25 bp, Artificial Sequence
  synthetic nucleotide probe
gcgagtgtgccctgatgaagcagca

SEQ ID NO: 1563 - DNA, 25 bp, Artificial Sequence
  synthetic nucleotide probe
gaggtcgtagcgggagacagcaaca

SEQ ID NO: 1564 - DNA, 25 bp, Artificial Sequence
  synthetic nucleotide probe
tgggacagaagtccgatctccctag

SEQ ID NO: 1565 - DNA, 25 bp, Artificial Sequence
  synthetic nucleotide probe
ggcagtctgcctaaagattcctttc

SEQ ID NO: 1566 - DNA, 25 bp, Artificial Sequence
  synthetic nucleotide probe
gccttctcccatacattccaaaagg

SEQ ID NO: 1567 - DNA, 25 bp, Artificial Sequence
  synthetic nucleotide probe
gttcaacagtaagcagcacctccaa

SEQ ID NO: 1568 - DNA, 25 bp, Artificial Sequence
  synthetic nucleotide probe
tctcctttcggccagtatcataaga

SEQ ID NO: 1569 - DNA, 25 bp, Artificial Sequence
  synthetic nucleotide probe
tggacgccataatcctgaggctcct

SEQ ID NO: 1570 - DNA, 25 bp, Artificial Sequence
  synthetic nucleotide probe
ggctcctagaggctgagggggcaac

SEQ ID NO: 1571 - DNA, 25 bp, Artificial Sequence
  synthetic nucleotide probe
tgagggggcaacggtgtgatccagc

SEQ ID NO: 1572 - DNA, 25 bp, Artificial Sequence
  synthetic nucleotide probe
gcaagccagttgtcaaacacagcca

SEQ ID NO: 1573 - DNA, 25 bp, Artificial Sequence
  synthetic nucleotide probe
gtgagagaggcagtggccgtcctcc

SEQ ID NO: 1574 - DNA, 25 bp, Artificial Sequence
  synthetic nucleotide probe
ttcctgtacctttgactaacgctca

SEQ ID NO: 1575 - DNA, 25 bp, Artificial Sequence
  synthetic nucleotide probe
cttccgggcctgcatgcagtagaca

SEQ ID NO: 1576 - DNA, 25 bp, Artificial Sequence
  synthetic nucleotide probe
atcagccattttatcgaggcacgtg

SEQ ID NO: 1577 - DNA, 25 bp, Artificial Sequence
  synthetic nucleotide probe
tagctaatgtgctgagcttgtgcct

SEQ ID NO: 1578 - DNA, 25 bp, Artificial Sequence
  synthetic nucleotide probe
tagaccacgtaaccttcaagtatgt

SEQ ID NO: 1579 - DNA, 25 bp, Artificial Sequence
  synthetic nucleotide probe
gaactactctcccaaggaaaatatt

SEQ ID NO: 1580 - DNA, 25 bp, Artificial Sequence
  synthetic nucleotide probe
taaggaagcgcgggtcccgcatgag

SEQ ID NO: 1581 - DNA, 25 bp, Artificial Sequence
  synthetic nucleotide probe
gccatcatcttgttgaatcagccat

SEQ ID NO: 1582 - DNA, 25 bp, Artificial Sequence
  synthetic nucleotide probe
tacaacgggcacagacagagcacct

SEQ ID NO: 1583 - DNA, 25 bp, Artificial Sequence
  synthetic nucleotide probe
tttacttccggaatactcaagcaaa

SEQ ID NO: 1584 - DNA, 25 bp, Artificial Sequence
  synthetic nucleotide probe
tatggtgaccaatgaatctccctta

SEQ ID NO: 1585 - DNA, 25 bp, Artificial Sequence
  synthetic nucleotide probe
gattgttttaactgattactgtaga

SEQ ID NO: 1586 - DNA, 25 bp, Artificial Sequence
  synthetic nucleotide probe
ttcctagtcttctgtgtatgtgatg

SEQ ID NO: 1587 - DNA, 25 bp, Artificial Sequence
  synthetic nucleotide probe
aggatgtctttcaggattattttaa

SEQ ID NO: 1588 - DNA, 25 bp, Artificial Sequence
  synthetic nucleotide probe
taattacttacttattacctagatt

SEQ ID NO: 1589 - DNA, 25 bp, Artificial Sequence
  synthetic nucleotide probe
gactacaggagacgacggggccttt

SEQ ID NO: 1590 - DNA, 25 bp, Artificial Sequence
  synthetic nucleotide probe
gacagaactgtttgttatgtaccat

SEQ ID NO: 1591 - DNA, 25 bp, Artificial Sequence
  synthetic nucleotide probe
actgtgccttgtgttacttaatcat

SEQ ID NO: 1592 - DNA, 25 bp, Artificial Sequence
  synthetic nucleotide probe
gcgacgaacgccgggatttcggcgg

SEQ ID NO: 1593 - DNA, 25 bp, Artificial Sequence
  synthetic nucleotide probe
gtatagccccacagattaaatttaa

SEQ ID NO: 1594 - DNA, 25 bp, Artificial Sequence
  synthetic nucleotide probe
ttgccaccatttgtagaccacgtaa

SEQ ID NO: 1595 - DNA, 25 bp, Artificial Sequence
  synthetic nucleotide probe
gaagacaatgctttccatattgtga

SEQ ID NO: 1596 - DNA, 25 bp, Artificial Sequence
  synthetic nucleotide probe
gctttaatgtgcttttacttccgga

SEQ ID NO: 1597 - DNA, 25 bp, Artificial Sequence
  synthetic nucleotide probe
atttttttgcgtgaaagtgttacat

SEQ ID NO: 1598 - DNA, 25 bp, Artificial Sequence
  synthetic nucleotide probe
cggataaggaagcgcgggtcccgca

SEQ ID NO: 1599 - DNA, 25 bp, Artificial Sequence
  synthetic nucleotide probe
ctgggctcggggtgactacaggaga

SEQ ID NO: 1600 - DNA, 25 bp, Artificial Sequence
  synthetic nucleotide probe
aaagatggctactgtgccttgtgtt

SEQ ID NO: 1601 - DNA, 25 bp, Artificial Sequence
  synthetic nucleotide probe
aaggccatcacgtatgccaaaggat

SEQ ID NO: 1602 - DNA, 25 bp, Artificial Sequence
  synthetic nucleotide probe
gactcttcttgcagtttacaacggg

SEQ ID NO: 1603 - DNA, 25 bp, Artificial Sequence
  synthetic nucleotide probe
gagacattgatatatccttttgcta

SEQ ID NO: 1604 - DNA, 25 bp, Artificial Sequence
  synthetic nucleotide probe
gattactgtagatcaacctgatgat

SEQ ID NO: 1605 - DNA, 25 bp, Artificial Sequence
  synthetic nucleotide probe
ggctcggggtgactacaggagacga

SEQ ID NO: 1606 - DNA, 25 bp, Artificial Sequence
  synthetic nucleotide probe
gcctcgttttccggataaggaagcg

SEQ ID NO: 1607 - DNA, 25 bp, Artificial Sequence
  synthetic nucleotide probe
ccatttgtagaccacgtaaccttca

SEQ ID NO: 1608 - DNA, 25 bp, Artificial Sequence
  synthetic nucleotide probe
ttcgtgttgccatcatcttgttgaa

SEQ ID NO: 1609 - DNA, 25 bp, Artificial Sequence
  synthetic nucleotide probe
ttacctacatctcatacttgcttta

SEQ ID NO: 1610 - DNA, 25 bp, Artificial Sequence
  synthetic nucleotide probe
gagcttgtgccttggtgattgattg

SEQ ID NO: 1611 - DNA, 25 bp, Artificial Sequence
  synthetic nucleotide probe
caaggcttttcggagaggttttcat

SEQ ID NO: 1612 - DNA, 25 bp, Artificial Sequence
  synthetic nucleotide probe
gggtgactacaggagacgacggggc

SEQ ID NO: 1613 - DNA, 25 bp, Artificial Sequence
  synthetic nucleotide probe
tgttacatattctttcacttgtatg

SEQ ID NO: 1614 - DNA, 25 bp, Artificial Sequence
  synthetic nucleotide probe
gataaggtaacatggggtttttctg

SEQ ID NO: 1615 - DNA, 25 bp, Artificial Sequence
  synthetic nucleotide probe
gaattacattgtatagccccacaga

SEQ ID NO: 1616 - DNA, 25 bp, Artificial Sequence
  synthetic nucleotide probe
gatatatccttttgctacaagctat

SEQ ID NO: 1617 - DNA, 25 bp, Artificial Sequence
  synthetic nucleotide probe
tgacggtggctataccagggactct

SEQ ID NO: 1618 - DNA, 25 bp, Artificial Sequence
  synthetic nucleotide probe
gaaactattactcctaagaattaca

SEQ ID NO: 1619 - DNA, 25 bp, Artificial Sequence
  synthetic nucleotide probe
gctggcgacgaacgccgggatttcg

SEQ ID NO: 1620 - DNA, 25 bp, Artificial Sequence
  synthetic nucleotide probe
atgcagctttcaaatcattgggggg

SEQ ID NO: 1621 - DNA, 25 bp, Artificial Sequence
  synthetic nucleotide probe
gaggcacgtgatcagtgttgcaaca

SEQ ID NO: 1622 - DNA, 25 bp, Artificial Sequence
  synthetic nucleotide probe
gggcaggagcctcgcaatatgtggc

SEQ ID NO: 1623 - DNA, 25 bp, Artificial Sequence
  synthetic nucleotide probe
ggctcctcagcctaagactatggct

SEQ ID NO: 1624 - DNA, 25 bp, Artificial Sequence
  synthetic nucleotide probe
agaaacactcaggcctgacctaggc

SEQ ID NO: 1625 - DNA, 25 bp, Artificial Sequence
  synthetic nucleotide probe
taaccatcatgtatgcccacgaggg

SEQ ID NO: 1626 - DNA, 25 bp, Artificial Sequence
  synthetic nucleotide probe
taaccatcatgtatgcccacgaggg

SEQ ID NO: 1627 - DNA, 25 bp, Artificial Sequence
  synthetic nucleotide probe
gacccctctgagtgtggtcagtgcc

SEQ ID NO: 1628 - DNA, 25 bp, Artificial Sequence
  synthetic nucleotide probe
tccctgtgattgccctgttaagtat

SEQ ID NO: 1629 - DNA, 25 bp, Artificial Sequence
  synthetic nucleotide probe
tccctgtgattgccctgttaagtat

SEQ ID NO: 1630 - DNA, 25 bp, Artificial Sequence
  synthetic nucleotide probe
tgctcctgcttacgaagtattccca

SEQ ID NO: 1631 - DNA, 25 bp, Artificial Sequence
  synthetic nucleotide probe
tgctcctgcttacgaagtattccca

SEQ ID NO: 1632 - DNA, 25 bp, Artificial Sequence
  synthetic nucleotide probe
gattgcttgtgacaacggctgcatc

SEQ ID NO: 1633 - DNA, 25 bp, Artificial Sequence
  synthetic nucleotide probe
ggctcctgtctgactattccaggat

SEQ ID NO: 1634 - DNA, 25 bp, Artificial Sequence
  synthetic nucleotide probe
ccctaccgatagaacagtggctcag

SEQ ID NO: 1635 - DNA, 25 bp, Artificial Sequence
  synthetic nucleotide probe
gcatgaaggctcctgtctgactatt

SEQ ID NO: 1636 - DNA, 25 bp, Artificial Sequence
  synthetic nucleotide probe
tgcatctgggacctcaagttctgcc

SEQ ID NO: 1637 - DNA, 25 bp, Artificial Sequence
  synthetic nucleotide probe
ccaccaacacctagctgctggatat

SEQ ID NO: 1638 - DNA, 25 bp, Artificial Sequence
  synthetic nucleotide probe
cacagacaccctaccgatagaacag

SEQ ID NO: 1639 - DNA, 25 bp, Artificial Sequence
  synthetic nucleotide probe
tggcctgctcagacgggaaagtact

SEQ ID NO: 1640 - DNA, 25 bp, Artificial Sequence
  synthetic nucleotide probe
gtatctgcatgaaggctcctgtctg

SEQ ID NO: 1641 - DNA, 25 bp, Artificial Sequence
  synthetic nucleotide probe
caaggaataccacagacaccctacc

SEQ ID NO: 1642 - DNA, 25 bp, Artificial Sequence
  synthetic nucleotide probe
aaccatagctatcatgtgtttccca

SEQ ID NO: 1643 - DNA, 25 bp, Artificial Sequence
  synthetic nucleotide probe
atagctatcatgtgtttcccaaatc

SEQ ID NO: 1644 - DNA, 25 bp, Artificial Sequence
  synthetic nucleotide probe
acagggcccactttgtctatgggat

SEQ ID NO: 1645 - DNA, 25 bp, Artificial Sequence
  synthetic nucleotide probe
ttcccaatcactggtcatctgaccc

SEQ ID NO: 1646 - DNA, 25 bp, Artificial Sequence
  synthetic nucleotide probe
ttcccaatcactggtcatctgaccc

SEQ ID NO: 1647 - DNA, 25 bp, Artificial Sequence
  synthetic nucleotide probe
ggaaatctagtcatcttccctgtga

SEQ ID NO: 1648 - DNA, 25 bp, Artificial Sequence
  synthetic nucleotide probe
ggaaatctagtcatcttccctgtga

SEQ ID NO: 1649 - DNA, 25 bp, Artificial Sequence
  synthetic nucleotide probe
gacatgaatgagacacacccactga

SEQ ID NO: 1650 - DNA, 25 bp, Artificial Sequence
  synthetic nucleotide probe
gcaactctgcaggtggggtctatgc

SEQ ID NO: 1651 - DNA, 25 bp, Artificial Sequence
  synthetic nucleotide probe
aagtactgctattcagtctacccca

SEQ ID NO: 1652 - DNA, 25 bp, Artificial Sequence
  synthetic nucleotide probe
tattactgccttctgaaacttcctc

SEQ ID NO: 1653 - DNA, 25 bp, Artificial Sequence
  synthetic nucleotide probe
tattactgccttctgaaacttcctc

SEQ ID NO: 1654 - DNA, 25 bp, Artificial Sequence
  synthetic nucleotide probe
gttactgatttcttgtatctcccag

SEQ ID NO: 1655 - DNA, 25 bp, Artificial Sequence
  synthetic nucleotide probe
gctacatgtgtaagtctgcctaaat

SEQ ID NO: 1656 - DNA, 25 bp, Artificial Sequence
  synthetic nucleotide probe
aatctagccccagacatactgtgtt

SEQ ID NO: 1657 - DNA, 25 bp, Artificial Sequence
  synthetic nucleotide probe
cctcccattttgttctcggaagatt

SEQ ID NO: 1658 - DNA, 25 bp, Artificial Sequence
  synthetic nucleotide probe
gtccatttgagattctgcactccat

SEQ ID NO: 1659 - DNA, 25 bp, Artificial Sequence
  synthetic nucleotide probe
cccctcctgctaggccagtgaaggg

SEQ ID NO: 1660 - DNA, 25 bp, Artificial Sequence
  synthetic nucleotide probe
taattggatttgtaccgtcctccca

SEQ ID NO: 1661 - DNA, 25 bp, Artificial Sequence
  synthetic nucleotide probe
gtcaagtatgtctcaacactagcat

SEQ ID NO: 1662 - DNA, 25 bp, Artificial Sequence
  synthetic nucleotide probe
gaaaagttcacttggacgctggggc

SEQ ID NO: 1663 - DNA, 25 bp, Artificial Sequence
  synthetic nucleotide probe
ttgtcaatatatcgaactgttccca

SEQ ID NO: 1664 - DNA, 25 bp, Artificial Sequence
  synthetic nucleotide probe
taactctgcattctggcttctgtat

SEQ ID NO: 1665 - DNA, 25 bp, Artificial Sequence
  synthetic nucleotide probe
tgtctaagtgtttttcttcgtggtc

SEQ ID NO: 1666 - DNA, 25 bp, Artificial Sequence
  synthetic nucleotide probe
aggcctcctgaattgagtttgatgc

SEQ ID NO: 1667 - DNA, 25 bp, Artificial Sequence
  synthetic nucleotide probe
gactggaatgggaccaggccgtcgc

SEQ ID NO: 1668 - DNA, 25 bp, Artificial Sequence
  synthetic nucleotide probe
gatgcagagctttttagccatgaag

SEQ ID NO: 1669 - DNA, 25 bp, Artificial Sequence
  synthetic nucleotide probe
atacagagagcacccatatggacgt

SEQ ID NO: 1670 - DNA, 25 bp, Artificial Sequence
  synthetic nucleotide probe
gtagatgcttttttctttgttgttt

SEQ ID NO: 1671 - DNA, 25 bp, Artificial Sequence
  synthetic nucleotide probe
tgactttttcattacgtgggttttg

SEQ ID NO: 1672 - DNA, 25 bp, Artificial Sequence
  synthetic nucleotide probe
gtatctcccaggattcctgttgctt

SEQ ID NO: 1673 - DNA, 25 bp, Artificial Sequence
  synthetic nucleotide probe
ttgctttacccacaacagacaagta

SEQ ID NO: 1674 - DNA, 25 bp, Artificial Sequence
  synthetic nucleotide probe
ccttattccattcttaactctgcat

SEQ ID NO: 1675 - DNA, 25 bp, Artificial Sequence
  synthetic nucleotide probe
gttgaatgactacatcttctccttt

SEQ ID NO: 1676 - DNA, 25 bp, Artificial Sequence
  synthetic nucleotide probe
agctgcttacttgttgtatgccttc

SEQ ID NO: 1677 - DNA, 25 bp, Artificial Sequence
  synthetic nucleotide probe
gtatgccttcactagaatcaggtct

SEQ ID NO: 1678 - DNA, 25 bp, Artificial Sequence
  synthetic nucleotide probe
aatcaggtctattgcacgtctggcc

SEQ ID NO: 1679 - DNA, 25 bp, Artificial Sequence
  synthetic nucleotide probe
ggaaactaggccggtgcattttacg

SEQ ID NO: 1680 - DNA, 25 bp, Artificial Sequence
  synthetic nucleotide probe
gagctggcaactgctttcacagagt

SEQ ID NO: 1681 - DNA, 25 bp, Artificial Sequence
  synthetic nucleotide probe
gaacatgtggcgtatcttgtgtgaa

SEQ ID NO: 1682 - DNA, 25 bp, Artificial Sequence
  synthetic nucleotide probe
ctggcccaagggtgtaatccctcac

SEQ ID NO: 1683 - DNA, 25 bp, Artificial Sequence
  synthetic nucleotide probe
aatccctcacaggtttgaaccctgt

SEQ ID NO: 1684 - DNA, 25 bp, Artificial Sequence
  synthetic nucleotide probe
ttttcccaagtggccattggccctg

SEQ ID NO: 1685 - DNA, 25 bp, Artificial Sequence
  synthetic nucleotide probe
gctttttttcaatcttgtgggcaca

SEQ ID NO: 1686 - DNA, 25 bp, Artificial Sequence
  synthetic nucleotide probe
gcaactggcaccatacaggaagatt

SEQ ID NO: 1687 - DNA, 25 bp, Artificial Sequence
  synthetic nucleotide probe
caaattccagccaacgtccttgttg

SEQ ID NO: 1688 - DNA, 25 bp, Artificial Sequence
  synthetic nucleotide probe
aacgtccttgttgcactttgggtat

SEQ ID NO: 1689 - DNA, 25 bp, Artificial Sequence
  synthetic nucleotide probe
gcactttgggtattctgagattttc

SEQ ID NO: 1690 - DNA, 25 bp, Artificial Sequence
  synthetic nucleotide probe
tcttgccattcccttaggctttagc

SEQ ID NO: 1691 - DNA, 25 bp, Artificial Sequence
  synthetic nucleotide probe
ggctttagcagctttgcatttcctg

SEQ ID NO: 1692 - DNA, 25 bp, Artificial Sequence
  synthetic nucleotide probe
ttgcatttcctgttgtatttattct

SEQ ID NO: 1693 - DNA, 25 bp, Artificial Sequence
  synthetic nucleotide probe
tattctcagccattttgggcatatg

SEQ ID NO: 1694 - DNA, 25 bp, Artificial Sequence
  synthetic nucleotide probe
cagactggaaacgggactttctatt

SEQ ID NO: 1695 - DNA, 25 bp, Artificial Sequence
  synthetic nucleotide probe
cttctcccccaataactgtgggtct

SEQ ID NO: 1696 - DNA, 25 bp, Artificial Sequence
  synthetic nucleotide probe
tcagagaaagttagttcggctcgat

SEQ ID NO: 1697 - DNA, 25 bp, Artificial Sequence
  synthetic nucleotide probe
gatagttaatgttttatgcttccat

SEQ ID NO: 1698 - DNA, 25 bp, Artificial Sequence
  synthetic nucleotide probe
tattccatttgcaacttatccccaa

SEQ ID NO: 1699 - DNA, 25 bp, Artificial Sequence
  synthetic nucleotide probe
gcaaaagtacccctttgcacagata

SEQ ID NO: 1700 - DNA, 25 bp, Artificial Sequence
  synthetic nucleotide probe
gaaatgcggctagttcagagagatt

SEQ ID NO: 1701 - DNA, 25 bp, Artificial Sequence
  synthetic nucleotide probe
aattttttgtatcaagtccctgaat

SEQ ID NO: 1702 - DNA, 25 bp, Artificial Sequence
  synthetic nucleotide probe
gacaggcttgccgaaattgaggaca

SEQ ID NO: 1703 - DNA, 25 bp, Artificial Sequence
  synthetic nucleotide probe
aacagccaaggttacggtggttatg

SEQ ID NO: 1704 - DNA, 25 bp, Artificial Sequence
  synthetic nucleotide probe
gtgtcctccctgtccaaattgggaa

SEQ ID NO: 1705 - DNA, 25 bp, Artificial Sequence
  synthetic nucleotide probe
gattcatttgaaggtggctcctgcc

SEQ ID NO: 1706 - DNA, 25 bp, Artificial Sequence
  synthetic nucleotide probe
ataatacttccttatgtagccatta

SEQ ID NO: 1707 - DNA, 25 bp, Artificial Sequence
  synthetic nucleotide probe
aatgtcaatttgtttgttggttgtt

SEQ ID NO: 1708 - DNA, 25 bp, Artificial Sequence
  synthetic nucleotide probe
gagtggttatgggaaggtatccagg

SEQ ID NO: 1709 - DNA, 25 bp, Artificial Sequence
  synthetic nucleotide probe
gactacactggttacaacaactact

SEQ ID NO: 1710 - DNA, 25 bp, Artificial Sequence
  synthetic nucleotide probe
ggtggtcatcaaaatagctacaaac

SEQ ID NO: 1711 - DNA, 25 bp, Artificial Sequence
  synthetic nucleotide probe
aagtttggaagacaggcttgccgaa

SEQ ID NO: 1712 - DNA, 25 bp, Artificial Sequence
  synthetic nucleotide probe
ggaaccagggatatagtaactattg

SEQ ID NO: 1713 - DNA, 25 bp, Artificial Sequence
  synthetic nucleotide probe
gagctgtggtggacttcatagatga

SEQ ID NO: 1714 - DNA, 25 bp, Artificial Sequence
  synthetic nucleotide probe
agtccctgaatggaagtatgacgtt

SEQ ID NO: 1715 - DNA, 25 bp, Artificial Sequence
  synthetic nucleotide probe
aaaagcccagtgtgacagtgtcatg

SEQ ID NO: 1716 - DNA, 25 bp, Artificial Sequence
  synthetic nucleotide probe
gaagtttaattctgagttctcatta

SEQ ID NO: 1717 - DNA, 25 bp, Artificial Sequence
  synthetic nucleotide probe
ggaggatatgactacactggttaca

SEQ ID NO: 1718 - DNA, 25 bp, Artificial Sequence
  synthetic nucleotide probe
gagagatttttagagctgtggtgga

SEQ ID NO: 1719 - DNA, 25 bp, Artificial Sequence
  synthetic nucleotide probe
ttattccatttgcaacttatcccca

SEQ ID NO: 1720 - DNA, 25 bp, Artificial Sequence
  synthetic nucleotide probe
aggttacggtggttatggaggatat

SEQ ID NO: 1721 - DNA, 25 bp, Artificial Sequence
  synthetic nucleotide probe
atttgctttcattgttttatttctt

SEQ ID NO: 1722 - DNA, 25 bp, Artificial Sequence
  synthetic nucleotide probe
agaaatttgctttcattgttttatt

SEQ ID NO: 1723 - DNA, 25 bp, Artificial Sequence
  synthetic nucleotide probe
cctttcccccagtattgtagagcaa

SEQ ID NO: 1724 - DNA, 25 bp, Artificial Sequence
  synthetic nucleotide probe
ggtatccaggcgaggtggtcatcaa

SEQ ID NO: 1725 - DNA, 25 bp, Artificial Sequence
  synthetic nucleotide probe
gtatgacgttgggtccctctgaagt

SEQ ID NO: 1726 - DNA, 25 bp, Artificial Sequence
  synthetic nucleotide probe
gtatgacgttgggtccctctgaagt

SEQ ID NO: 1727 - DNA, 25 bp, Artificial Sequence
  synthetic nucleotide probe
aacaactactatggatatggtgatt

SEQ ID NO: 1728 - DNA, 25 bp, Artificial Sequence
  synthetic nucleotide probe
tgtgctttttagaacaaatctggat

SEQ ID NO: 1729 - DNA, 25 bp, Artificial Sequence
  synthetic nucleotide probe
gagagcgcgtgcagagacttggtgg

SEQ ID NO: 1730 - DNA, 25 bp, Artificial Sequence
  synthetic nucleotide probe
tggcgagatgtgtctctcaatcctg

SEQ ID NO: 1731 - DNA, 25 bp, Artificial Sequence
  synthetic nucleotide probe
tctctcaatcctgtggctttggtga

SEQ ID NO: 1732 - DNA, 25 bp, Artificial Sequence
  synthetic nucleotide probe
gtttttgttcttagataacccacat

SEQ ID NO: 1733 - DNA, 25 bp, Artificial Sequence
  synthetic nucleotide probe
tgaaatgatacttgtactcccccta

SEQ ID NO: 1734 - DNA, 25 bp, Artificial Sequence
  synthetic nucleotide probe
ctttgtcaactgctgtgaatgctgt

SEQ ID NO: 1735 - DNA, 25 bp, Artificial Sequence
  synthetic nucleotide probe
gaatgctgtatggtgtgtgttctct

SEQ ID NO: 1736 - DNA, 25 bp, Artificial Sequence
  synthetic nucleotide probe
ggactgagcttgtggtgtgctttgc

SEQ ID NO: 1737 - DNA, 25 bp, Artificial Sequence
  synthetic nucleotide probe
gcagagttcaccagtgagctcaggt

SEQ ID NO: 1738 - DNA, 25 bp, Artificial Sequence
  synthetic nucleotide probe
gttctaatgtctgttagctacccat

SEQ ID NO: 1739 - DNA, 25 bp, Artificial Sequence
  synthetic nucleotide probe
aagaatgctgtttgctgcagttctg

SEQ ID NO: 1740 - DNA, 25 bp, Artificial Sequence
  synthetic nucleotide probe
aagctagtgctttgtcttagtagtt

SEQ ID NO: 1741 - DNA, 25 bp, Artificial Sequence
  synthetic nucleotide probe
ggggcaatcgtgggggcaatgtagg

SEQ ID NO: 1742 - DNA, 25 bp, Artificial Sequence
  synthetic nucleotide probe
tcgtttcaggcttcattttagcttc

SEQ ID NO: 1743 - DNA, 25 bp, Artificial Sequence
  synthetic nucleotide probe
tcacacctttttgaaatctgcccta

SEQ ID NO: 1744 - DNA, 25 bp, Artificial Sequence
  synthetic nucleotide probe
accctccagattactacggctatga

SEQ ID NO: 1745 - DNA, 25 bp, Artificial Sequence
  synthetic nucleotide probe
attgttataacttcacacctttttg

SEQ ID NO: 1746 - DNA, 25 bp, Artificial Sequence
  synthetic nucleotide probe
tggatatggctaccctccagattac

SEQ ID NO: 1747 - DNA, 25 bp, Artificial Sequence
  synthetic nucleotide probe
aaacaagctgggcacactgttaaat

SEQ ID NO: 1748 - DNA, 25 bp, Artificial Sequence
  synthetic nucleotide probe
gctcttggacattattgggcttgca

SEQ ID NO: 1749 - DNA, 25 bp, Artificial Sequence
  synthetic nucleotide probe
catgattttgcagaacctttggttt

SEQ ID NO: 1750 - DNA, 25 bp, Artificial Sequence
  synthetic nucleotide probe
caatgcttttatcgtttcaggcttc

SEQ ID NO: 1751 - DNA, 25 bp, Artificial Sequence
  synthetic nucleotide probe
gttcccgtggatctcggggcaatcg

SEQ ID NO: 1752 - DNA, 25 bp, Artificial Sequence
  synthetic nucleotide probe
gattccaagcgtcgtcagaccaaca

SEQ ID NO: 1753 - DNA, 25 bp, Artificial Sequence
  synthetic nucleotide probe
tcaacagcagagaggccgtggttcc

SEQ ID NO: 1754 - DNA, 25 bp, Artificial Sequence
  synthetic nucleotide probe
ggctatgaagatccctactacggct

SEQ ID NO: 1755 - DNA, 25 bp, Artificial Sequence
  synthetic nucleotide probe
aaagccgtgacaatttgttctttga

SEQ ID NO: 1756 - DNA, 25 bp, Artificial Sequence
  synthetic nucleotide probe
tcacagaggggggcacctttgggac

SEQ ID NO: 1757 - DNA, 25 bp, Artificial Sequence
  synthetic nucleotide probe
acctttgggaccaccaagaggctct

SEQ ID NO: 1758 - DNA, 25 bp, Artificial Sequence
  synthetic nucleotide probe
gtatttccaatttcttgttcatgta

SEQ ID NO: 1759 - DNA, 25 bp, Artificial Sequence
  synthetic nucleotide probe
gtcgtcagaccaacaaccaacagaa

SEQ ID NO: 1760 - DNA, 25 bp, Artificial Sequence
  synthetic nucleotide probe
tgggcttgcagagttcccttattct

(19) United States
(12) Patent Application Publication
(10) Pub. No.: US 2014/0018253 A1
(43) Pub. Date: Jan. 16, 2014

(54) GENE EXPRESSION PANEL FOR BREAST CANCER PROGNOSIS

(71) Applicants: The Regents of the University of California, (US); Oregon Health and Science University, (US)

(72) Inventors: Obi L. Griffith, St. Louis, MO (US); Oana M. Enache, Palo Alto, CA (US); Francois Pepin, San Bruno, CA (US); Paul T. Spellman, Portland, OR (US); Joe W. Gray, Lake Oswego, OR (US)

(73) Assignees: Oregon Health and Science University, Portland, OR (US); The Regents of the University of California, Oakland, CA (US)

(21) Appl. No.: 13/857,536

(22) Filed: Apr. 5, 2013

Related U.S. Application Data
(60) Provisional application No. 61/620,907, filed on Apr. 5, 2012.
(60) Provisional application No. 61/789,071, filed on Mar. 15, 2013.

(51) Int. Cl. C12Q 1/68; G06F 19/00

(52) U.S. Cl. CPC C12Q 1/6886; G06F 19/345
USPC 506/9; 506/16; 435/6.12; 435/7.92; 702/20

(57) ABSTRACT

The invention described in the application relates to a panel of gene expression markers for node-negative, ER-positive, HER2-negative breast cancer patients. The invention thus provides methods and compositions, e.g., kits and/or microarrays, for evaluating gene expression levels of the markers and methods of using such gene expression levels to evaluate the likelihood of relapse of a node-negative, ER-positive, HER2-negative breast cancer patient. Such information can be used in determining treatment options for patients.

### CROSS-REFERENCE TO RELATED APPLICATIONS

This application claims priority benefit of U.S. provisional application No. 61/789,071, filed Mar. 15, 2013 and U.S. provisional application No. 61/620,907, filed Apr. 5, 2012, which applications are herein incorporated by reference.

### STATEMENT AS TO RIGHTS TO INVENTIONS MADE UNDER FEDERALLY SPONSORED RESEARCH AND DEVELOPMENT

This invention was made with government support under Contract No. DE-ACO2-05CH11231 awarded by the U.S. Department of Energy. The government has certain rights in this invention.

### REFERENCE TO A SEQUENCE LISTING SUBMITTED AS AN ASCII TEXT FILE

The Sequence Listing written in file SEQTXT 77429-871826-010220US.txt, created on Apr. 4, 2013, 332,697 bytes, machine format IBM-PC, MS-Windows operating system, is hereby incorporated by reference in its entirety for all purposes.

### BACKGROUND OF THE INVENTION

Large randomized trials have shown that chemotherapy administered in the perioperative setting (e.g., adjuvant chemotherapy) can cure patients otherwise destined to recur with systemic, incurable cancer (1). Once this recurrence has happened, the same chemotherapy is not curative. Therefore, the adjuvant window is a privileged period of time, when the decision to administer additional therapy or not, as well as the type, duration and intensity of such therapy takes center stage. Node-negative, estrogen receptor (ER)-positive, HER2-negative patients generally show a favorable prognosis when treated with adjuvant hormonal therapy only. However, because an unknown subset of these patients develops recurrences, most are currently treated not only with hormonal therapy but also cytotoxic chemotherapy, even though it is probably unnecessary for most. Our goal was to stratify these patients into those that are most or least likely to develop a recurrence within 10 years after surgery. Our approach was to develop a multi-gene transcription-level-based classifier of 10-year-relapse (disease recurrence within 10 years) using a large database of existing, publicly available microarray datasets. The probability of relapse and relapse risk score group reported by our method can be used to assign systemic chemotherapy to only those patients most likely to benefit from it.

### BRIEF SUMMARY OF THE INVENTION

The present invention is based, in part, on the identification of a panel of gene expression markers for node-negative, ER-positive, HER2-negative breast cancer patients. The probability of relapse and relapse risk score group using the panel of gene expression markers of the invention can be used to assign systemic chemotherapy to only those patients most likely to benefit from it.

The invention can be used on tissue from LN−, ER+, HER2−breast cancer patients by any assay where transcript levels (or their expression products) of primary genes (or their alternate genes) in the Random Forest Relapse Score (RFRS) signature are measured. These measurements can be used to assign an RFRS value and to determine the likelihood of breast cancer relapse. Those breast cancer patients with tumors at high risk of relapse can be treated more aggressively whereas those at low risk of relapse can more safely avoid the risks and side effects of systemic chemotherapy. Thus, this method can provide rapid and useful information for clinical decision making.

Thus, in one embodiment, the invention relates to a method of evaluating the likelihood of a relapse for a patient that has a lymph node-negative, estrogen receptor-positive, HER2-negative breast cancer, the method comprising: providing a sample comprising breast tumor tissue from the patient; determining the levels of expression of the 17 genes, or one or more corresponding alternates thereof, identified in Table 1; or of the 8 genes, or one or more corresponding alternates thereof, identified in Table 2; in the sample; and correlating the levels of expression with the likelihood of a relapse. In some embodiments, the method further comprises detecting the level of expression of one or more reference genes, e.g., one or more reference genes selected from the genes identified in Table 3, or one or more reference genes selected from the genes ARPC2, ATF4, ATP5B, B2M, CDH4, CELF1, CLTA, CLTC, COPB1, CTBP1, CYC1, CYFIP1, DAZAP2, DHX15, DIMT1, EEF1A1, FLOT2, GADPH, GUSB, HADHA, HDLBP, HMBS, HNRNPC, HPRT1, HSP90AB1, MTCH1, MYL12B, NACA, NDUFB8, PGK1, PPIA, PPIB, PTBP1, RPL13A, RPLPO, RPS13, RPS23, RPS3, S100A6, SDHA, SEC31A, SET, SF3B1, SFRS3, SNRNP200, STARD7, SUMO1, TBP, TFRC, TMBIM6, TPT1, TRA2B, TUBA1C, UBB, UBC, UBE2D2, UBE2D3, VAMP3, XPO1, YTHDC1, YWHAZ, and 18S rRNA. In some embodiments, the step of determining the levels of expression of the gene comprises detecting the level of expression of RNA. In some embodiments, the determining step comprises detecting the level of expression of protein. The RNA may be detected using any known methods, e.g., a method comprising a quantitative PCR reaction. In some embodiments, detecting the level of expression of the RNA comprises hybridizing a nucleic acid obtained from the sample to an array that comprises probes to the 17 genes set forth in Table 1, and/or one or more corresponding alternates thereof; or hybridizing a nucleic acid obtained from the sample to an array that comprises probes to the 8 genes set forth in Table 2, and/or one or more corresponding alternates thereof.

In a further aspect, the invention provides a kit for detecting RNA expression comprising primers and/or probes for detecting the level of expression of the 17 genes set forth in Table 1, and/or one or more corresponding alternates thereof; or for detecting the level of expression of the 8 genes set forth in Table 2, and/or one or more alternates thereof. In some embodiments, the kit further comprises primers and/or probes for detecting the level of RNA expression of one or more reference genes, e.g., one or more reference genes selected from the genes identified in Table 3, or one or more reference genes selected from the genes ARPC2, ATF4, ATP5B, B2M, CDH4, CELF1, CLTA, CLTC, COPB1, CTBP1, CYC1, CYFIP1, DAZAP2, DHX15, DIMT1, EEF1A1, FLOT2, GADPH, GUSB, HADHA, HDLBP, HMBS, HNRNPC, HPRT1, HSP90AB1, MTCH1, MYL12B, NACA, NDUFB8, PGK1, PPIA, PPIB, PTBP1, RPL13A, RPLPO, RPS13, RPS23, RPS3, S100A6, SDHA, SEC31A, SET, SF3B1, SFRS3, SNRNP200, STARD7, SUMO1, TBP, TFRC, TMBIM6, TPT1, TRA2B, TUBA1C, UBB, UBC, UBE2D2, UBE2D3, VAMP3, XPO1, YTHDC1, YWHAZ, and 18S rRNA.

In a further aspect, the invention relates to a microarray comprising probes for detecting the level of expression of the 17 genes set forth in Table 1, and/or one or more corresponding alternates thereof; or for detecting the level of expression of the 8 genes set forth in Table 2, and/or one or more alternates thereof. In some embodiments, the microarray further comprises probes for detecting the level of expression of one or more reference genes, e.g., one or more reference genes selected from the genes identified in Table 3, or one or more reference genes selected from the genes ARPC2, ATF4, ATP5B, B2M, CDH4, CELF1, CLTA, CLTC, COPB1, CTBP1, CYC1, CYFIP1, DAZAP2, DHX15, DIMT1, EEF1A1, FLOT2, GADPH, GUSB, HADHA, HDLBP, HMBS, HNRNPC, HPRT1, HSP90AB1, MTCH1, MYL12B, NACA, NDUFB8, PGK1, PPIA, PPIB, PTBP1, RPL13A, RPLPO, RPS13, RPS23, RPS3, S100A6, SDHA, SEC31A, SET, SF3B1, SFRS3, SNRNP200, STARD7, SUMO1, TBP, TFRC, TMBIM6, TPT1, TRA2B, TUBA1C, UBB, UBC, UBE2D2, UBE2D3, VAMP3, XPO1, YTHDC1, YWHAZ, and 18S rRNA.

In an additional aspect, the invention relates to a computer-implemented method for evaluating the likelihood of a relapse for a patient that has a lymph node-negative, estrogen receptor-positive, HER2-negative breast cancer, the method comprising: receiving, at one or more computer systems, information describing the level of expression of the 17 genes set forth in Table 1, or one or more corresponding alternates thereof; or information describing the level of expression of the 8 genes set forth in Table 2, or one or more corresponding alternates thereof; in a breast tumor tissue sample obtained from the patient; performing, with one or more processors associated with the computer system, a random forest analysis in which the level of expression of each gene in the analysis is assigned to a terminal leaf of each decision tree, representing a vote for either “relapse” or no “relapse”; generating, with the one or more processors associated with the one or more computer systems, a random forest relapse score (RFRS). In some embodiments in which the level of expression of the 17 genes, or at least one alternate, set forth in Table 1 is determined, if the RFRS is greater than or equal to 0.606 the patient is assigned to a high risk group, if greater than or equal to 0.333 and less than 0.606 the patient is assigned to an intermediate risk group and if less than 0.333 the patient is assigned to low risk group. In some embodiments in which the level of expression of the 8 genes, or at least one alternate, set forth in Table 2 is determined, if the RFRS is greater than or equal to 0.606 the patient is assigned to a high risk group, if greater than or equal to 0.333 and less than 0.606 the patient is assigned to an intermediate risk group and if less than 0.333 the patient is assigned to a low risk group.

In some embodiments, the computer-implemented method further comprises generating, with the one or more processors associated with the one or more computer systems, a likelihood of relapse by comparison of the RFRS score for the patient to a loess fit of RFRS versus likelihood of relapse for a training dataset.

In another aspect, the invention relates to a non-transitory computer-readable medium storing program code for evaluating the likelihood of a relapse for a patient that has a lymph node-negative, estrogen receptor-positive, HER2-negative breast cancer, the computer-readable medium comprising:

code for receiving information describing the level of expression of the 17 genes identified in Table 1, or one or more corresponding alternates thereof; or information describing the level of expression of the 8 genes identified in Table 2, or one or more corresponding alternates thereof; in a breast tumor tissue sample obtained from the patient;
code for performing a random forest analysis in which the level of expression of each gene in the analysis is assigned to a terminal leaf of each decision tree, representing a vote for either “relapse” or no “relapse”; and
code for generating a random forest relapse score (RFRS). In some embodiments in which the level of expression of the 17 genes, or one or more designated alternates, identified in Table 1 is determined, if the RFRS is greater than or equal to 0.606 the patient is assigned to a high risk group, if greater than or equal to 0.333 and less than 0.606 the patient is assigned to an intermediate risk group and if less than 0.333 the patient is assigned to low risk group. In some embodiments in which the level of expression of the 8 genes, or one or more designated alternates, identified in Table 2, is determined, if the RFRS is greater than or equal to 0.606 the patient is assigned to a high risk group, if greater than or equal to 0.333 and less than 0.606 the patient is assigned to an intermediate risk group and if less than 0.333 the patient is assigned to a low risk group. In some embodiments, the non-transitory computer-readable medium storing program code further comprises code for generating a likelihood of relapse by comparison of the RFRS score for the patient to a loess fit of RFRS versus likelihood of relapse for a training dataset.

### BRIEF DESCRIPTION OF THE DRAWINGS

FIG. 1 shows an analysis of the studies employed in Example 1 to identify duplicates. The diagram shows the approximate overlap between GEO datasets used. Three studies show zero overlap while the other six show significant overlap.

FIG. 2 shows estrogen receptor and HER2 status for 998 samples employed in Example 1. Expression status was determined using the “205225_at” probe set for ER and the rank sum of the 216835_s_at (ERBB2), 210761_s_at (GRB7), 202991_at (STARD3) and 55616_at (PGAP3) probe sets for HER2. Threshold values were chosen by mixed model clustering. A total of 68 samples were determined to be ER-negative and 89 samples were determined to be HER2-positive. In total, 140 samples were either HER2-positive or ER-negative (17 were both) and were filtered from further analysis.

FIG. 3 illustrates the breakdown of samples for analysis. A total of 858 samples passed all filtering steps including 487 samples with 10-year follow-up data (213 relapse; 274 no relapse). The remaining 371 samples had insufficient follow-up for 10-year classification analysis but were retained for use in survival analysis. The 858 samples were broken into two-thirds training and one-third testing sets resulting in: a training set of 572 samples for use in survival analysis and 325 samples with 10yr follow-up (143 relapse; 182 no relapse) for classification analysis; and a testing set of 286 samples for use in survival analysis and 162 samples with 10-year follow-up (70 relapse; 92 no relapse) for classification analysis

FIG. 4 illustrates risk group threshold determination. The distribution of RFRS scores was determined for patients in the training dataset (N=325) comparing those with a known relapse (right side) versus those with no known relapse (left side). As expected, patients without a known relapse tend to have a higher predicted likelihood of relapse (by RFRS) and vice versa. Mixed model clustering was used to identify thresholds (0.333 and 0.606) for defining low, intermediate, and high-risk groups as indicated.

FIGS. 5A-C provide data illustrating likelihood of relapse according to RFRS group. The survival plot shows relapse-free survival comparing (from top to bottom) low-risk, intermediate-risk, and high-risk groups as determined by RFRS for: (A) the full-gene-set model on training data; (B) the 8-gene-set model on independent test data; (C) the 8-gene-set model on the independent NKI data set. Significance between risk groups was determined by Kaplan-Meier logrank test (with test for linear trend).

FIG. 6 illustrates likelihood of relapse according to RFRS group with breakdown into additional risk groups. The survival plot shows relapse-free survival comparing (from top to bottom) very-low-risk, low-risk, intermediate-risk, high-risk, and very-high-risk groups as determined by RFRS. Significance between risk groups was determined by Kaplan-Meier logrank test (with test for linear trend).

FIG. 7 illustrates estimated likelihood of relapse at 10 years for any RFRS value. The likelihood of relapse was calculated in the training data set (N=505) for 50 RFRS intervals (from 0 to 1). A smooth curve was fitted using a loess function and 95% confidence intervals plotted to represent the error in the fit. Short vertical marks just above the x-axis, one for each patient, represent the distribution of RFRS values observed in the training data. Thresholds for risk groups are indicated. The plot shows a linear relationship between RFRS and likelihood of relapse at 10 years with the likelihood ranging from approximately 0 to 40%.

FIG. 8 shows a gene ontology analysis of the genes identified for the 17-gene signature panel. A Gene Ontology (GO) analysis was performed using DAVID to identify the associated GO biological processes for the 17-gene model. The diagram represents the approximate overlap between GO terms. To simplify, redundant terms were grouped together. Genes in the 17-gene list are involved in a wide range of biological processes known to be involved in breast cancer biology including cell cycle, hormone response, cell death, DNA repair, transcription regulation, wound healing and others. Since the 8-gene set is entirely contained in the 17-gene set it would be involved in many of the same processes.

FIG. 9 provides a sample patient report of risk of relapse generated in accordance with the invention. Using the RFRS algorithm, a patient would be assigned an RFRS value. If RFRS is greater than or equal to 0.606 the patient is assigned to the “high-risk” group, if greater than or equal to 0.333 and less than 0.606 the patient is assigned to “intermediate-risk” group and if less than 0.333 the patient is assigned to “low-risk” group. The patient's RFRS value is also used to determine a likelihood of relapse by comparison to a pre-calculated loess fit of RFRS versus likelihood of relapse for the training dataset. The patient's estimated likelihood of relapse is determined, added to the summary plot, and output as a new report.

FIG. 10 (FIG. 10) is a flowchart of a method for identifying LN⁻ ER⁺HER2⁻ breast cancer patients that are candidates for additional treatment in one embodiment.

FIG. 11 (FIG. 11) is a flowchart of a method for generating an RF model for identifying LN⁻ER⁺HER2⁻ breast cancer patients that are candidates for additional treatment in one embodiment.

FIG. 12 (FIG. 12) is a block diagram of computer system 1200 that may incorporate an embodiment, be incorporated into an embodiment, or be used to practice any of the innovations, embodiments, and/or examples found within this disclosure.

FIGS. 13A and B illustrate likelihood of relapse according to RFRS group stratified by treatment status. The survival plot shows relapse-free survival comparing (from top to bottom) low-risk, intermediate-risk, and high-risk groups as determined by RFRS for: (A) hormone-therapy-treated and (B) untreated. Significance between risk groups was determined by Kaplan-Meier logrank test (with test for linear trend).

### DETAILED DESCRIPTION OF THE INVENTION

An “estrogen receptor positive, lymph node-negative, HER2-negative” or “ER⁺N⁻ HER2⁻” patient as used herein refers to a patient that has no discernible breast cancer in the lymph nodes; and has breast tumor cells that express estrogen receptor and do not show evidence of HER2 genomic (DNA) amplification or HER2 over-expression. LN− status is typically determined when the sentinel node is surgically removed and examined by microscopy for cytological evidence of disease. Patients are considered LN− (N0) if zero positive nodes were observed. Patients are considered LN+ if one or more lymph nodes were considered positive for disease (1-2 positive=N1; 3-6 positive=N2, etc). ER⁺ status is typically assessed by immunohistochemistry (IHC) where a positive determination is made when greater than a small percentage (typically greater than 3%, 5% or 10%) of cells stain positive. ER status can also be tested by quantitative PCR or biochemical assays. HER2⁻ status is generally determined by either IHC, fluorescence in situ hybridization (FISH) or some combination of the two methods. Typically, a patient is first tested by IHC and scored on a scale from 0 to 3 where a “3+” score indicates strong complete membrane staining on >5-10% of tumor cells and is considered positive. No staining (score of “0”) or a “1+” score, indicating faint partial membrane staining in greater than 5-10% of cells, is considered negative. An intermediate score of “2+”, indicating weak to moderate complete membrane staining in more than 5-10% of cells, may prompt further testing by FISH. A typical HER2 FISH scheme would consider a patient HER2⁺ if the ratio of a HER2 probe to a centromeric (reference) probe is more than 4:1 in ˜5% or more of cells after examining 20 or more metaphase spreads. Otherwise the patient is considered HER2⁻. Quantitative PCR, array-based hybridization, and other methods may also be used to determine HER2 status. The specific methods and cutoff points for determining LN, ER and HER2 status may vary from hospital to hospital. For the purpose of this invention, a patient will be considered “ER⁺LN⁻HER2⁻” if reported as such by their health care provider or if determined by any accepted and approved methods, including but not limited to those detailed above.

In the current invention, a “gene set forth in” a table or a “gene identified in” a table are used interchangeably to refer to the gene that is listed in that table. For example, a gene “identified in” Table 4 refers to the gene that corresponds to the gene listed in Table 4. As understood in the art, there are naturally occurring polymorphisms for many gene sequences. Genes that are naturally occurring allelic variations for the purposes of this invention are those genes encoded by the same genetic locus. The proteins encoded by allelic variations of a gene set forth in Table 4 (or in any of Tables 1-3 or Table 4) typically have at least 95% amino acid sequence identity to one another, i.e., an allelic variant of a gene indicated in Table 4 typically encodes a protein product that has at least 95% identity, often at least 96%, at least 97%, at least 98%, or at least 99%, or greater, identity to the amino acid sequence encoded by the nucleotide sequence denoted by the Entrez Gene ID number (Apr. 1, 2012) shown in Table 4 for that gene. For example, an allelic variant of a gene encoding CCNB2 (gene: cyclin B2) typically has at least 95% identity, often at least 96%, at least 97%, at least 98%, or at least 99%, or greater, to the CCNB2 protein sequence encoded by the nucleic acid sequence available under Entrez Gene ID no. 9133). A “gene identified in” a table, such as Table 4, also refers to a gene that can be unambiguously mapped to the same genetic locus as that of a gene assigned to a genetic locus using the probes for the gene that are listed in Appendix 3. Similarly, a “gene identified in Table 1” or a “gene identified in Table 2” refers to a gene that can be unambiguously mapped to a genetic locus using the probes for that gene that are listed in Appendix 4 (panel of 17 genes from Table 1, which includes the genes for the 8 gene panel identified in Table 2); and a “gene identified in Table 3” refers to a gene that can be unambiguously mapped to a genetic locus using the probes for that gene that are listed in Appendix 5.

The terms “identical” or “100% identity,” in the context of two or more nucleic acids or proteins refer to two or more sequences or subsequences that are the same sequences. Two sequences are “substantially identical” or a certain percent identity if two sequences have a specified percentage of amino acid residues or nucleotides that are the same (i.e., 70% identity, optionally 75%, 80%, 85%, 90%, or 95% identity, over a specified region, or, when not specified, over the entire sequence), when compared and aligned for maximum correspondence over a comparison window, or designated region as measured using known sequence comparison algorithms, e.g., BLAST using the default parameters, or by manual alignment and visual inspection.

A “gene product” or “gene expression product” in the context of this invention refers to an RNA or protein encoded by the gene.

The term “evaluating a biomarker” in an LN⁻ER⁺HER2⁻ patient refers to determining the level of expression of a gene product encoded by a gene, or allelic variant of the gene, listed in Table 4. Preferably, the gene is listed in Table 1 or Table 2 as either a primary or alternate gene. Typically, the RNA expression level is determined.

### INTRODUCTION

The invention is based, in part, on the identification of a panel of at least eight genes whose gene expression level correlates with breast cancer prognosis. In some embodiments, the panel of at least eight genes comprises at least eight genes, or at least 9, 10, 11, 12, 13, 14, 15, 16, 17, 18, 19, 20, 25, 30, 35, 40, 45, or 50, or more genes, identified in Table 4 with the proviso that the gene is one of those also listed in Table 5. In some embodiments, the panel of genes comprises at least 8 primary genes, or at least 9, 10, 11, 12, 13, 14, 15, 16, or all 17 primary genes identified in Table 1; or the 8 primary genes set forth in Table 2. Table 1 also shows alternate genes for each of the seventeen that can replace the specific primary gene in the analysis. At least one alternate gene can be evaluated in place of the corresponding primary gene listed in Table 1, or can be evaluated in addition to the corresponding primary gene listed in Table 1. Similarly, Table 2 shows alternate genes for each of the eight that can replace, or be assayed in addition to, the specific primary gene in the analysis. The results of the expression analysis are then evaluated using an algorithm to determine breast cancer patients that are likely to have a recurrence, and accordingly, are candidates for treatment with more aggressive therapy, such as chemotherapy.

The invention therefore relates to measurement of expression levels of a biomarker panel, e.g., a 17-gene expression panel, or an 8-gene expression panel, in a breast cancer patient prior to the patient undergoing chemotherapy. In some embodiments, probes to detect such transcripts may be applied in the form of a diagnostic device to predict which LN⁻ER⁺HER2⁻ breast cancer patients have a greater risk for relapse.

Typically, the methods of the invention comprise determining the expression levels of all seventeen primary genes, and/or at least one corresponding alternate gene shown in Table 1. However, in some embodiments, the expression level of fewer genes, e.g., 5, 6, 7, 8, 9, 10, 11, 12, 13, 14, 15, or 16 genes, may be evaluated. In some embodiments, the methods of the invention comprise determining the expression level of all eight gene and/or at least one corresponding alternate gene shown in Table 2. Gene expression levels may be measured using any number of methods known in the art. In typical embodiments, the method involves measuring the level of RNA. RNA expression can be quantified using any method, e.g., employing a quantitative amplification method such as qPCR. In other embodiments, the methods employ array-based assays. In still other embodiments, protein products may be detected. The gene expression patterns are determined using a sample obtained from breast tumor.

In the context of this invention, an “alternate gene” refers to a gene that can be evaluated for expression levels instead of, or in addition to, the gene for which the “alternate gene” is the designated alternate in Table 1. For example, one of the genes in Table 1 is CCNB2. MELK and GINS1 are both alternatives that can be evaluated for expression instead of CCNB2 or in addition to CCNB2, when evaluating the gene expression levels of the 17 genes set forth in Table 1. With respect to Table 2, an “alternate gene” refers to a gene that can be evaluated for expression levels instead of, or in addition to, the gene for which the “alternate gene” is the designated alternate in Table 2. For example, one of the genes in Table 2 is CCNB2. MELK and TOP2A are both alternatives that can be evaluated for expression instead of CCNB2 or in addition to CCNB2 when evaluating the gene expression levels of the 8 genes set forth in Table 2.

### Methods for Quantifying RNA

The quantity of RNA encoded by a gene set forth in Table 1 or Table 2 and optionally, a gene set forth in Table 3 or an alternative reference gene, can be readily determined according to any method known in the art for quantifying RNA. Various methods involving amplification reactions and/or reactions in which probes are linked to a solid support and used to quantify RNA may be used. Alternatively, the RNA may be linked to a solid support and quantified using a probe to the sequence of interest.

An “RNA nucleic acid sample” analyzed in the invention is obtained from a breast tumor sample obtained from the patient. An “RNA nucleic acid sample” comprises RNA, but need not be purely RNA, e.g., DNA may also be present in the sample. Techniques for obtaining an RNA sample from tumors are well known in the art.

In some embodiments, the target RNA is first reverse transcribed and the resulting cDNA is quantified. In some embodiments, RT-PCR or other quantitative amplification techniques are used to quantify the target RNA. Amplification of cDNA using PCR is well known (see U.S. Pat. Nos. 4,683,195 and 4,683,202; PCR PROTOCOLS: A GUIDE TO METHODS AND APPLICATIONS (Innis et al., eds, 1990)). Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol Biotechnol. 20(2):163-79 (2002). Alternative methods for determining the level of a mRNA of interest in a sample may involve other nucleic acid amplification methods such as ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art.

In general, quantitative amplification is based on the monitoring of the signal (e.g., fluorescence of a probe) representing copies of the template in cycles of an amplification (e.g., PCR) reaction. One method for detection of amplification products is the 5′-3′ exonuclease “hydrolysis” PCR assay (also referred to as the TaqMan™ assay) (U.S. Pat. Nos. 5,210,015 and 5,487,972; Holland et al., PNAS USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the “TaqMan™” probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

Another method of detecting amplification products that relies on the use of energy transfer is the “beacon probe” method described by Tyagi and Kramer, Nature Biotech. 14:303-309 (1996), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched. When employed in PCR, the molecular beacon probe, which hybridizes to one of the strands of the PCR product, is in “open conformation,” and fluorescence is detected, while those that remain unhybridized will not fluoresce (Tyagi and Kramer, Nature Biotechnol. 14: 303-306 (1996)). As a result, the amount of fluorescence will increase as the amount of PCR product increases, and thus may be used as a measure of the progress of the PCR. Those of skill in the art will recognize that other methods of quantitative amplification are also available.

Various other techniques for performing quantitative amplification of nucleic acids are also known. For example, some methodologies employ one or more probe oligonucleotides that are structured such that a change in fluorescence is generated when the oligonucleotide(s) is hybridized to a target nucleic acid. For example, one such method involves a dual fluorophore approach that exploits fluorescence resonance energy transfer (FRET), e.g., LightCycler™ hybridization probes, where two oligo probes anneal to the amplicon. The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: Scorpions™ probes (e.g., Whitcombe et al., Nature Biotechnology 17:804-807, 1999, and U.S. Pat. No. 6,326,145), Sunrise™ (or Amplifluor™) probes (e.g., Nazarenko et al., Nuc. Acids Res. 25:2516-2521, 1997, and U.S. Pat. No. 6,117,635), and probes that form a secondary structure that results in reduced signal without a quencher and that emits increased signal when hybridized to a target (e.g., Lux Probes™).

In other embodiments, intercalating agents that produce a signal when intercalated in double stranded DNA may be used. Exemplary agents include SYBR GREEN™ and SYBR GOLD™. Since these agents are not template-specific, it is assumed that the signal is generated based on template-specific amplification. This can be confirmed by monitoring signal as a function of temperature because melting point of template sequences will generally be much higher than, for example, primer-dimers, etc.

In other embodiments, the mRNA is immobilized on a solid surface and contacted with a probe, e.g., in a dot blot or Northern format. In an alternative embodiment, the probe(s) are immobilized on a solid surface and the mRNA is contacted with the probe(s), for example, in a gene chip array. A skilled artisan can readily adapt known mRNA detection methods for use in detecting the level of mRNA encoding the biomarkers or other proteins of interest.

In some embodiments, microarrays, e.g., are employed. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, U.S. Pat. Nos. 6,040,138, 5,800,992 and 6,020,135, 6,033,860, and 6,344,316. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261. Although a planar array surface is often employed the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device.

Primer and probes for use in amplifying and detecting the target sequence of interest can be selected using well-known techniques.

In some embodiments, the methods of the invention further comprise detecting level of expression of one or more reference genes that can be used as controls to determine expression levels. Such genes are typically expressed constitutively at a high level and can act as a reference for determining accurate gene expression level estimates. Examples of control genes are provided in Table 3 and the following list: ARPC2, ATF4, ATP5B, B2M, CDH4, CELF1, CLTA, CLTC, COPB1, CTBP1, CYC1, CYFIP1, DAZAP2, DHX15, DIMT1, EEF1A1, FLOT2, GADPH, GUSB, HADHA, HDLBP, HMBS, HNRNPC, HPRT1, HSP90AB1, MTCH1, MYL12B, NACA, NDUFB8, PGK1, PPIA, PPIB, PTBP1, RPL13A, RPLPO, RPS13, RPS23, RPS3, S100A6, SDHA, SEC31A, SET, SF3B1, SFRS3, SNRNP200, STARD7, SUMO1, TBP, TFRC, TMBIM6, TPT1, TRA2B, TUBA1C, UBB, UBC, UBE2D2, UBE2D3, VAMP3, XPO1, YTHDC1, YWHAZ, and 18S rRNA genes. Accordingly, a determination of RNA expression levels of the genes of interest, e.g., the gene expression levels of the panel of genes identified in Table 1, and/or an alternate; or the gene expression levels of the panel of genes identified in Table 2, and/or an alternate; may also comprise determining expression levels of one or more reference genes set forth in Table 3 or one or more reference genes selected from the genes ARPC2, ATF4, ATP5B, B2M, CDH4, CELF1, CLTA, CLTC, COPB1, CTBP1, CYC1, CYFIP1, DAZAP2, DHX15, DIMT1, EEF1A1, FLOT2, GADPH, GUSB, HADHA, HDLBP, HMBS, HNRNPC, HPRT1, HSP90AB1, MTCH1, MYL12B, NACA, NDUFB8, PGK1, PPIA, PPIB, PTBP1, RPL13A, RPLPO, RPS13, RPS23, RPS3, S100A6, SDHA, SEC31A, SET, SF3B1, SFRS3, SNRNP200, STARD7, SUMO1, TBP, TFRC, TMBIM6, TPT1, TRA2B, TUBA1C, UBB, UBC, UBE2D2, UBE2D3, VAMP3, XPO1, YTHDC1, YWHAZ, and 18S rRNA.

In the context of this invention, “determining the levels of expression” of an RNA of interest encompasses any method known in the art for quantifying an RNA of interest.

### Detection of Protein Levels

In some embodiments, e.g., where the expression level of a protein encoded by a biomarker gene set forth in Table 1 or Table 2 is measured. Often, such measurements may be performed using immunoassays. Protein expression level is determined using a breast tumor sample obtained from the patient.

A general overview of the applicable technology can be found in Harlow & Lane, Antibodies: A Laboratory Manual (1988) and Harlow & Lane, Using Antibodies (1999). Methods of producing polyclonal and monoclonal antibodies that react specifically with an allelic variant are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975)). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

Polymorphic alleles can be detected by a variety of immunoassay methods. For a review of immunological and immunoassay procedures, see Basic and Clinical Immunology (Stites & Terr eds., 7th ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Ten, eds., 7th ed. 1991).

Commonly used assays include noncompetitive assays, e.g., sandwich assays, and competitive assays. Typically, an assay such as an ELISA assay can be used. The amount of the polypeptide variant can be determined by performing quantitative analyses.

Other detection techniques, e.g., MALDI, may be used to directly detect the presence of proteins correlated with treatment outcomes.

As indicated above, evaluation of protein expression levels may additionally include determining the levels of protein expression of control genes, e.g., of one or more genes identified in Table 3.

### Devices and Kits

In a further aspect, the invention provides diagnostic devices and kits for identifying gene expression products of a panel of genes that is associated with prognosis for a LN⁻ ER⁺HER2⁻ breast cancer patient.

In some embodiments, a diagnostic device comprises probes to detect at least 8, 9, 10, 11, 12, 13, 14, 15, 16, or all 17 gene expression products set forth in Table 1, and/or alternates. In some embodiments, a diagnostic device comprises probes to detect the expression products of the 8 genes set forth in Table 2, and/or alternates. In some embodiments, the present invention provides oligonucleotide probes attached to a solid support, such as an array slide or chip, e.g., as described in DNA Microarrays: A Molecular Cloning Manual, 2003, Eds. Bowtell and Sambrook, Cold Spring Harbor Laboratory Press. Construction of such devices are well known in the art, for example as described in US Patents and Patent Publications U.S. Pat. No. 5,837,832; PCT application WO95/11995; U.S. Pat. No. 5,807,522; U.S. Pat. Nos. 7,157,229, 7,083,975, 6,444,175, 6,375,903, 6,315,958, 6,295,153, and 5,143,854, 2007/0037274, 2007/0140906, 2004/0126757, 2004/0110212, 2004/0110211, 2003/0143550, 2003/0003032, and 2002/0041420. Nucleic acid arrays are also reviewed in the following references: Biotechnol Annu Rev 8:85-101 (2002); Sosnowski et al, Psychiatr Genet 12(4):181-92 (December 2002); Heller, Annu Rev Biomed Eng 4: 129-53 (2002); Kolchinsky et al, Hum. Mutat 19(4):343-60 (April 2002); and McGail et al, Adv Biochem Eng Biotechnol 77:21-42 (2002).

An array can be composed of a large number of unique, single-stranded polynucleotides, usually either synthetic antisense polynucleotides or fragments of cDNAs, fixed to a solid support. Typical polynucleotides are preferably about 6-60 nucleotides in length, more preferably about 15-30 nucleotides in length, and most preferably about 18-25 nucleotides in length. For certain types of arrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length.

A person skilled in the art will recognize that, based on the known sequence information, detection reagents can be developed and used to assay any gene expression product set forth in Table 1 or Table 2 (or in some embodiments Table 3 or another reference gene described herein) and that such detection reagents can be incorporated into a kit. The term “kit” as used herein in the context of detection reagents, are intended to refer to such things as combinations of multiple gene expression detection reagents, or one or more gene expression detection reagents in combination with one or more other types of elements or components (e.g., other types of biochemical reagents, containers, packages such as packaging intended for commercial sale, substrates to which gene expression detection reagents are attached, electronic hardware components, etc.). Accordingly, the present invention further provides gene expression detection kits and systems, including but not limited to, packaged probe and primer sets (e.g., TaqMan probe/primer sets), arrays/microarrays of nucleic acid molecules where the arrays/microarrays comprise probes to detect the level of RNA transcript, and beads that contain one or more probes, primers, or other detection reagents for detecting one or more RNA transcripts encoded by a gene in a gene expression panel of the present invention. The kits can optionally include various electronic hardware components; for example, arrays (“DNA chips”) and microfluidic systems (“lab-on-a-chip” systems) provided by various manufacturers typically comprise hardware components. Other kits (e.g., probe/primer sets) may not include electronic hardware components, but may be comprised of, for example, one or more biomarker detection reagents (along with, optionally, other biochemical reagents) packaged in one or more containers.

In some embodiments, a detection kit typically contains one or more detection reagents and other components (e.g. a buffer, enzymes such as DNA polymerases) necessary to carry out an assay or reaction, such as amplification for detecting the level of transcript. A kit may further contain means for determining the amount of a target nucleic acid, and means for comparing the amount with a standard, and can comprise instructions for using the kit to detect the nucleic acid molecule of interest. In one embodiment of the present invention, kits are provided which contain the necessary reagents to carry out one or more assays to detect one or more RNA transcripts of a gene disclosed herein. In one embodiment of the present invention, biomarker detection kits/systems are in the form of nucleic acid arrays, or compartmentalized kits, including microfluidic/lab-on-a-chip systems.

Detection kits/systems for detecting expression of a panel of genes in accordance with the invention may contain, for example, one or more probes, or pairs or sets of probes, that hybridize to a nucleic acid molecule encoded by a gene set forth in Table 1 or Table 2. In some embodiments, the presence of more than one biomarker can be simultaneously evaluated in an assay. For example, in some embodiments probes or probe sets to different biomarkers are immobilized as arrays or on beads. For example, the same substrate can comprise probes for detecting expression of at least 8, 9, 10, 11, 12, 13, 14, 15, 16, or 17 or more of the genes set forth in Table 1, and/or alternates to the genes. In some embodiments, the same substrate can comprise probes for detecting expression of 8 or more genes set forth in Table 2, and/or alternates to the genes.

Using such arrays or other kits/systems, the present invention provides methods of identifying the levels of expression of a gene described herein in a test sample. Such methods typically involve incubating a test sample of nucleic acids obtained from a breast tumor from a LN⁻ER⁺HER2⁻patient with an array comprising one or more probes that selectively hybridizes to a nucleic acid encoded by a gene identified in Table 1 or Table 2. Such an array may additionally comprise probes to one or more reference genes identified in Table 3, or one or more reference genes selected from the genes ARPC2, ATF4, ATP5B, B2M, CDH4, CELF1, CLTA, CLTC, COPB1, CTBP1, CYC1, CYFIP1, DAZAP2, DHX15, DIMT1, EEF1A1, FLOT2, GADPH, GUSB, HADHA, HDLBP, HMBS, HNRNPC, HPRT1, HSP90AB1, MTCH1, MYL12B, NACA, NDUFB8, PGK1, PPIA, PPIB, PTBP1, RPL13A, RPLPO, RPS13, RPS23, RPS3, S100A6, SDHA, SEC31A, SET, SF3B1, SFRS3, SNRNP200, STARD7, SUMO1, TBP, TFRC, TMBIM6, TPT1, TRA2B, TUBA1C, UBB, UBC, UBE2D2, UBE2D3, VAMP3, XPO1, YTHDC1, YWHAZ, and 18S rRNA. In some embodiments, the array comprises probes to all 17 genes identified in Table 1, and/or alternates; or all 8 genes identified in Table 2, and/or alternates. Conditions for incubating a gene detection reagent (or a kit/system that employs one or more such biomarker detection reagents) with a test sample vary. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect a gene set forth in Table 1 or Table 2.

A gene expression detection kit of the present invention may include components that are used to prepare nucleic acids from a test sample for the subsequent amplification and/or detection of a gene transcript.

In some embodiments, a gene expression kit comprises one or more reagents, e.g., antibodies, for detecting protein products of a gene identified in Table 1 or Table 2 and optionally Table 3.

Correlating Gene Expression Levels with Prognostic Outcomes

The present invention provides methods of determining the levels of a gene expression product to evaluate the likelihood that a LN-ER+HER2−breast cancer patient will have a relapse. Accordingly, the method provides a way of identifying LN⁻ER⁺HER2⁻ breast cancer patients that are candidates for additional treatment, e.g., chemotherapy.

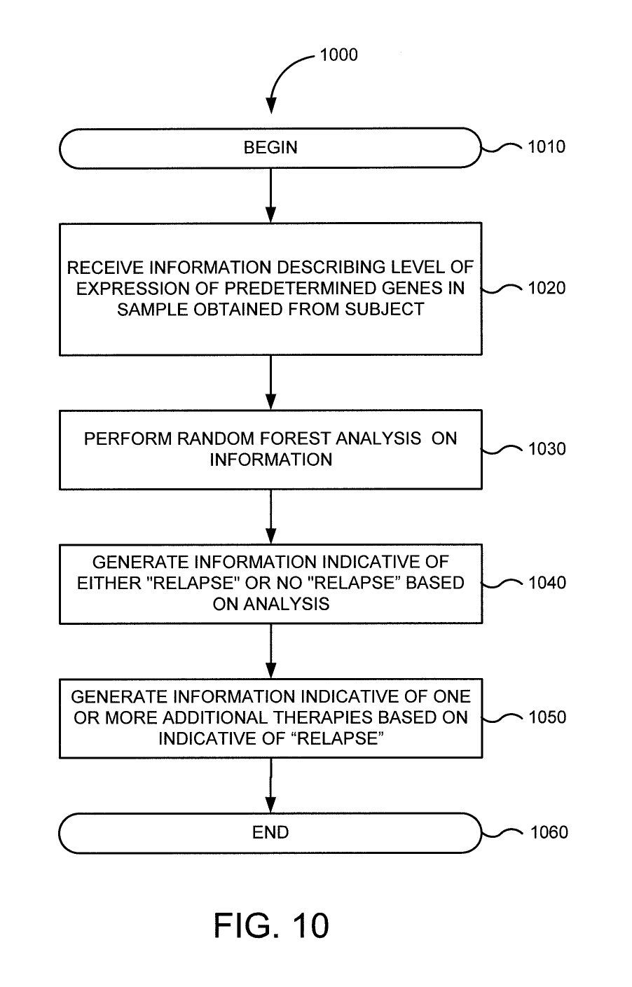

FIG. 10 is a flowchart of a method for identifying LN⁻ER⁺HER2⁻ breast cancer patients that are candidates for additional treatment in one embodiment. Implementations of or processing in method 1000 depicted in FIG. 10 may be performed by software (e.g., instructions or code modules) when executed by a central processing unit (CPU or processor) of a logic machine, such as a computer system or information processing device, by hardware components of an electronic device or application-specific integrated circuits, or by combinations of software and hardware elements. Method 1000 depicted in FIG. 10 begins in step 1010.

In step 1020, information is received describing one or more levels of expression of one or more predetermined genes in a sample obtained from a subject. For example, the level of a gene expression product associated with a prognostic outcome for a LN⁻ER⁺HER2⁻ breast cancer patient may be recorded. In one embodiment, input data includes a text file (e.g., a tab-delimited text file) of normalized expression values for 17 transcripts from primary genes (or an indicated alternative) from Table 1. In one embodiment, input data includes a text file (e.g., a tab-delimited text file) of normalized expression values for 8 transcripts from the primary genes (or an indicated alternative) from Table 2. For example, the text file may have the gene expression values for the 17 transcripts/genes as columns and patient(s) as rows. An illustrative patient data file (patient_data.txt) is presented in Appendix 1.

In step 1030, a random forest analysis is performed on the information describing the one or more levels of expression of the one or more predetermined genes in the sample obtained from the subject. A Random Forest (RF) algorithm is used to determine a Relapse Score (RS) when applied to independent patient data. A sample R program for running the RF algorithm is presented in Appendix 2. A Random Forest Relapse Score (RFRS) algorithm as used herein typically consists of a predetermined number of decision trees suitably adapted to ensure at least a fully deterministic model. Each node (branch) in each tree represents a binary decision based on transcript levels for transcripts described herein. Based on these decisions, the subject is assigned to a terminal leaf of each decision tree, representing a vote for either “relapse” or no “relapse”. The fraction of votes for “relapse” to votes for “no relapse” represents the RFRS—a measure of the probability of relapse. In some embodiments, if a subject's RFRS is greater than or equal to 0.606, the subject is assigned to one or more “high risk” groups. If an RFRS is greater than or equal to 0.333 and less than 0.606, the subject is assigned to one or more “intermediate risk” group. If an RFRS is less than 0.333, the subject is assigned to one or more “low risk” groups. In further embodiments, a subject's RFRS value is also used to determine a likelihood of relapse by comparison to a loess fit of RFRS versus likelihood of relapse for a training dataset. A subject's estimated likelihood of relapse is determined, added to a summary plot, and output as a new report.

In step 1040, information indicative of either “relapse” or no “relapse” is generated based on the random forest analysis. In some embodiments, information indicative of either “relapse” or no “relapse” is generated to include one or more summary statistics. For example, information indicative of either “relapse” or no “relapse” may be representative of how assignments to a terminal leaf of each decision tree, representing a vote for either “relapse” or no “relapse”, are made. In further embodiment, information indicative of either “relapse” or no “relapse” is generated for the fraction of votes for “relapse” to votes for “no relapse” as discussed above to represent the RFRS.

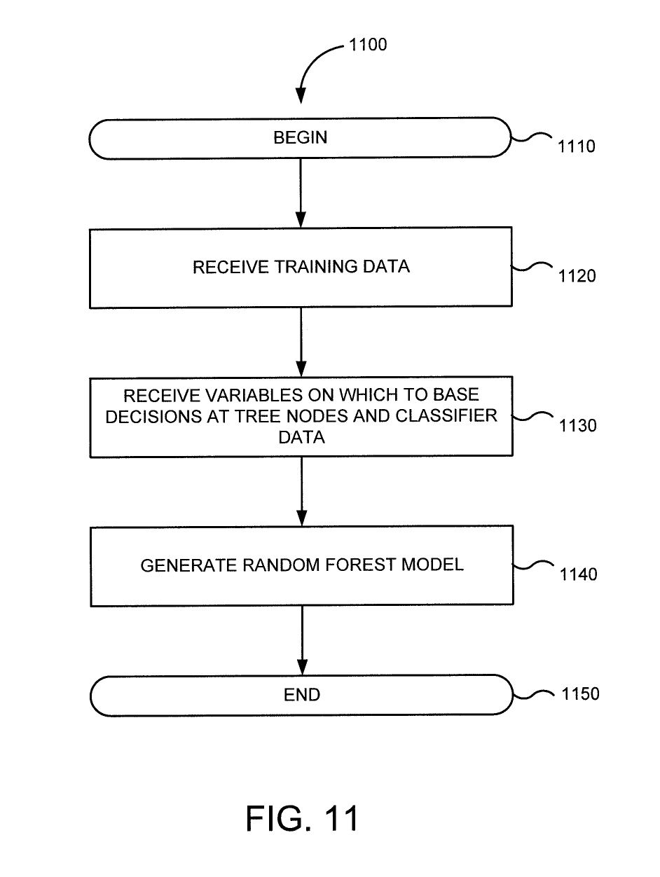

In step 1050, information indicative of one or more additional therapies is generated based on indicative of “relapse”. For example, if an RFRS is greater than or equal to 0.606, the subject is assigned to a “high risk” group from which the one or more additional therapies may be selected. If an RFRS score is greater than or equal to 0.333 and less than 0.606, the subject is assigned to an “intermediate risk” group from which all or none of the one or more additional therapies may be selected. If an RFRS is less than 0.333, the subject is assigned to a “low risk” group. In various embodiments, a subject's RFRS value is also used to determine a likelihood of relapse by comparison to a loess fit of RFRS versus likelihood of relapse for a training dataset described in FIG. 11 and in the Examples section. FIG. 10 ends in step 1060.

FIG. 11 is a flowchart of a method for generating an RF model for identifying LN⁻ ER⁺HER2⁻ breast cancer patients that are candidates for additional treatment in one embodiment. Implementations of or processing in method 1100 depicted in FIG. 11 may be performed by software (e.g., instructions or code modules) when executed by a central processing unit (CPU or processor) of a logic machine, such as a computer system or information processing device, by hardware components of an electronic device or application-specific integrated circuits, or by combinations of software and hardware elements. Method 1100 depicted in FIG. 11 begins in step 1110.

In step 1120, training data is received. For example, training data was generated as discussed below in the Examples section. In step 1130, variables on which to base decisions at tree nodes and classifier data are received. In one embodiment, classification was performed on training samples with either a relapse or no relapse after 10yr follow-up. In one example, a binary classification (e.g., relapse versus no relapse) is specified. However, additional classifier data may be included, such as a probability (proportion of “votes”) for relapse which is termed the Random Forests Relapse Score (RFRS). Risk group thresholds can be determined from the distribution of relapse probabilities using mixed model clustering to set cutoffs for low, intermediate and high risk groups.

In step 1140, a random forest model is generated. For example, a random forest model may be generated with at least 100,001 trees (i.e., using an odd number to ensure a substantially fully deterministic model). FIG. 11 ends in step 1150.

### Hardware Description

The invention thus includes a computer system to implement the algorithm. Such a computer system can comprise code for interpreting the results of an expression analysis evaluating the level of expression of the 17 genes, or a designated alternate gene) identified in Table 1; or code for interpreting the results of an expression analysis evaluating the level of expression of the 8 genes, or a designated alternate gene, identified in Table 2. Thus in an exemplary embodiment, the expression analysis results are provided to a computer where a central processor executes a computer program for determining the propensity for relapse for a LN⁻ER⁺HER2⁻ breast cancer patient.

The invention also provides the use of a computer system, such as that described above, which comprises: (1) a computer; (2) a stored bit pattern encoding the expression results obtained by the methods of the invention, which may be stored in the computer; and, optionally, (3) a program for determining the likelihood of relapse.

The invention further provides methods of generating a report based on the detection of gene expression products for a LN⁻ER⁺HER2⁻ breast cancer patient. Such a report is based on the detection of gene expression products encoded by the 17 genes, or one of the designated alternates, set forth in Table 1; or detection of gene expression products encoded by the 8 genes, or one of the designated alternates, set forth in Table 2.

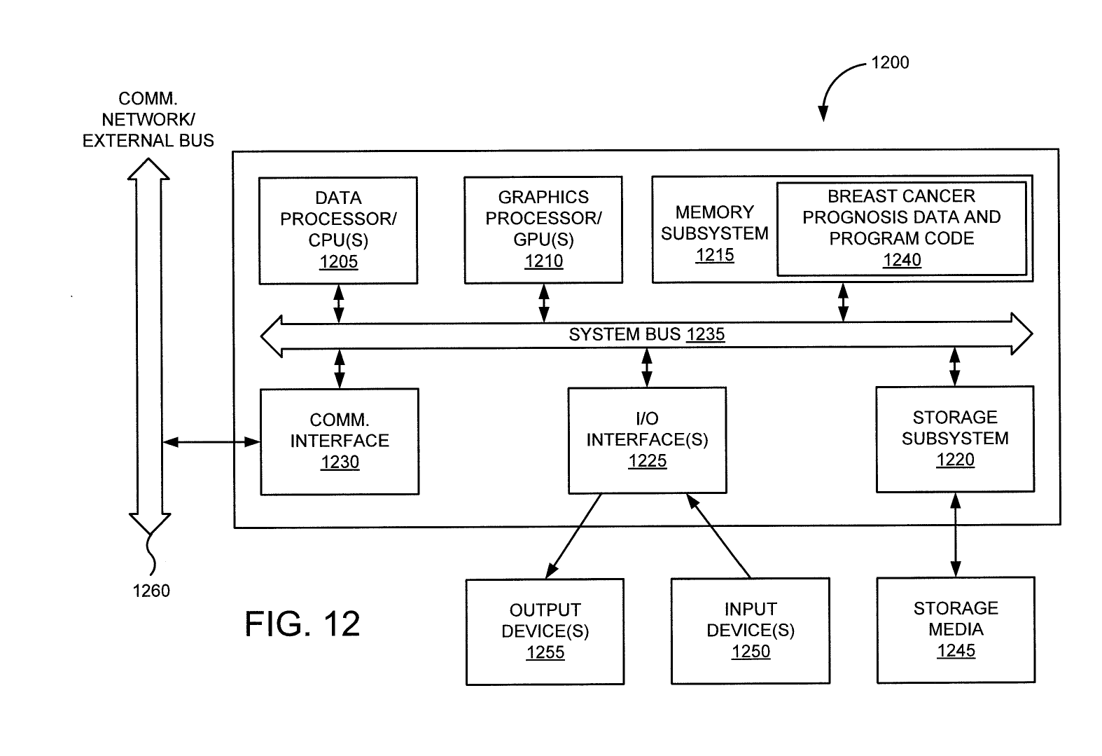

FIG. 12 is a block diagram of a computer system 1200 that may incorporate an embodiment, be incorporated into an embodiment, or be used to practice any of the innovations, embodiments, and/or examples found within this disclosure. FIG. 12 is merely illustrative of a computing device, general-purpose computer system programmed according to one or more disclosed techniques, or specific information processing device for an embodiment incorporating an invention whose teachings may be presented herein and does not limit the scope of the invention as recited in the claims. One of ordinary skill in the art would recognize other variations, modifications, and alternatives.

Computer system 1200 can include hardware and/or software elements configured for performing logic operations and calculations, input/output operations, machine communications, or the like. Computer system 1200 may include familiar computer components, such as one or more one or more data processors or central processing units (CPUs) 1205, one or more graphics processors or graphical processing units (GPUs) 1210, memory subsystem 1215, storage subsystem 1220, one or more input/output (I/O) interfaces 1225, communications interface 1230, or the like. Computer system 1200 can include system bus 1235 interconnecting the above components and providing functionality, such connectivity and inter-device communication. Computer system 1200 may be embodied as a computing device, such as a personal computer (PC), a workstation, a mini-computer, a mainframe, a cluster or farm of computing devices, a laptop, a notebook, a netbook, a PDA, a smartphone, a consumer electronic device, a gaming console, or the like.

The one or more data processors or central processing units (CPUs) 1205 can include hardware and/or software elements configured for executing logic or program code or for providing application-specific functionality. Some examples of CPU(s) 1205 can include one or more microprocessors (e.g., single core and multi-core) or micro-controllers. CPUs 1205 may include 4-bit, 8-bit, 12-bit, 16-bit, 32-bit, 64-bit, or the like architectures with similar or divergent internal and external instruction and data designs. CPUs 1205 may further include a single core or multiple cores. Commercially available processors may include those provided by Intel of Santa Clara, Calif. (e.g., x86, x86_{—}64, PENTIUM, CELERON, CORE, CORE 2, CORE ix, ITANIUM, XEON, etc.) or by Advanced Micro Devices of Sunnyvale, Calif. (e.g., x86, AMC_{—}64, ATHLON, DURON, TURION, ATHLON XP/64, OPTERON, PHENOM, etc). Commercially available processors may further include those conforming to the Advanced RISC Machine (ARM) architecture (e.g., ARMv7-9), POWER and POWERPC architecture, CELL architecture, and or the like. CPU(s) 1205 may also include one or more field-gate programmable arrays (FPGAs), application-specific integrated circuits (ASICs), or other microcontrollers. The one or more data processors or central processing units (CPUs) 1205 may include any number of registers, logic units, arithmetic units, caches, memory interfaces, or the like. The one or more data processors or central processing units (CPUs) 1205 may further be integrated, irremovably or moveably, into one or more motherboards or daughter boards.

The one or more graphics processor or graphical processing units (GPUs) 1210 can include hardware and/or software elements configured for executing logic or program code associated with graphics or for providing graphics-specific functionality. GPUs 1210 may include any conventional graphics processing unit, such as those provided by conventional video cards. Some examples of GPUs are commercially available from NVIDIA, ATI, and other vendors. In various embodiments, GPUs 1210 may include one or more vector or parallel processing units. These GPUs may be user programmable, and include hardware elements for encoding/decoding specific types of data (e.g., video data) or for accelerating 2D or 3D drawing operations, texturing operations, shading operations, or the like. The one or more graphics processors or graphical processing units (GPUs) 1210 may include any number of registers, logic units, arithmetic units, caches, memory interfaces, or the like. The one or more data processors or central processing units (CPUs) 1205 may further be integrated, irremovably or moveably, into one or more motherboards or daughter boards that include dedicated video memories, frame buffers, or the like.

Memory subsystem 1215 can include hardware and/or software elements configured for storing information. Memory subsystem 1215 may store information using machine-readable articles, information storage devices, or computer-readable storage media. Some examples of these articles used by memory subsystem 1270 can include random access memories (RAM), read-only-memories (ROMS), volatile memories, non-volatile memories, and other semiconductor memories. In various embodiments, memory subsystem 1215 can include data and program code 1240.

Storage subsystem 1220 can include hardware and/or software elements configured for storing information. Storage subsystem 1220 may store information using machine-readable articles, information storage devices, or computer-readable storage media. Storage subsystem 1220 may store information using storage media 1245. Some examples of storage media 1245 used by storage subsystem 1220 can include floppy disks, hard disks, optical storage media such as CD-ROMS, DVDs and bar codes, removable storage devices, networked storage devices, or the like. In some embodiments, all or part of breast cancer prognosis data and program code 1240 may be stored using storage subsystem 1220.

In various embodiments, computer system 1200 may include one or more hypervisors or operating systems, such as WINDOWS, WINDOWS NT, WINDOWS XP, VISTA, WINDOWS 7 or the like from Microsoft of Redmond, Wash., Mac OS or Mac OS X from Apple Inc. of Cupertino, Calif., SOLARIS from Sun Microsystems, LINUX, UNIX, and other UNIX-based or UNIX-like operating systems. Computer system 1200 may also include one or more applications configured to execute, perform, or otherwise implement techniques disclosed herein. These applications may be embodied as breast cancer prognosis data and program code 1240. Additionally, computer programs, executable computer code, human-readable source code, shader code, rendering engines, or the like, and data, such as image files, models including geometrical descriptions of objects, ordered geometric descriptions of objects, procedural descriptions of models, scene descriptor files, or the like, may be stored in memory subsystem 1215 and/or storage subsystem 1220.

The one or more input/output (I/O) interfaces 1225 can include hardware and/or software elements configured for performing I/O operations. One or more input devices 1250 and/or one or more output devices 1255 may be communicatively coupled to the one or more I/O interfaces 1225.

The one or more input devices 1250 can include hardware and/or software elements configured for receiving information from one or more sources for computer system 1200. Some examples of the one or more input devices 1250 may include a computer mouse, a trackball, a track pad, a joystick, a wireless remote, a drawing tablet, a voice command system, an eye tracking system, external storage systems, a monitor appropriately configured as a touch screen, a communications interface appropriately configured as a transceiver, or the like. In various embodiments, the one or more input devices 1250 may allow a user of computer system 1200 to interact with one or more non-graphical or graphical user interfaces to enter a comment, select objects, icons, text, user interface widgets, or other user interface elements that appear on a monitor/display device via a command, a click of a button, or the like.

The one or more output devices 1255 can include hardware and/or software elements configured for outputting information to one or more destinations for computer system 1200. Some examples of the one or more output devices 1255 can include a printer, a fax, a feedback device for a mouse or joystick, external storage systems, a monitor or other display device, a communications interface appropriately configured as a transceiver, or the like. The one or more output devices 1255 may allow a user of computer system 1200 to view objects, icons, text, user interface widgets, or other user interface elements.

A display device or monitor may be used with computer system 1200 and can include hardware and/or software elements configured for displaying information. Some examples include familiar display devices, such as a television monitor, a cathode ray tube (CRT), a liquid crystal display (LCD), or the like.

Communications interface 1230 can include hardware and/or software elements configured for performing communications operations, including sending and receiving data. Some examples of communications interface 1230 may include a network communications interface, an external bus interface, an Ethernet card, a modem (telephone, satellite, cable, ISDN), (asynchronous) digital subscriber line (DSL) unit, FireWire interface, USB interface, or the like. For example, communications interface 1230 may be coupled to communications network/external bus 1280, such as a computer network, to a FireWire bus, a USB hub, or the like. In other embodiments, communications interface 1230 may be physically integrated as hardware on a motherboard or daughter board of computer system 1200, may be implemented as a software program, or the like, or may be implemented as a combination thereof.

In various embodiments, computer system 1200 may include software that enables communications over a network, such as a local area network or the Internet, using one or more communications protocols, such as the HTTP, TCP/IP, RTP/RTSP protocols, or the like. In some embodiments, other communications software and/or transfer protocols may also be used, for example IPX, UDP or the like, for communicating with hosts over the network or with a device directly connected to computer system 1200.

As suggested, FIG. 12 is merely representative of a general-purpose computer system appropriately configured or specific data processing device capable of implementing or incorporating various embodiments of an invention presented within this disclosure. Many other hardware and/or software configurations may be apparent to the skilled artisan which are suitable for use in implementing an invention presented within this disclosure or with various embodiments of an invention presented within this disclosure. For example, a computer system or data processing device may include desktop, portable, rack-mounted, or tablet configurations. Additionally, a computer system or information processing device may include a series of networked computers or clusters/grids of parallel processing devices. In still other embodiments, a computer system or information processing device may perform techniques described above as implemented upon a chip or an auxiliary processing board.

Many hardware and/or software configurations of a computer system may be apparent to the skilled artisan, which are suitable for use in implementing a RFRS algorithm as described herein. For example, a computer system or data processing device may include desktop, portable, rack-mounted, or tablet configurations. Additionally, a computer system or information processing device may include a series of networked computers or clusters/grids of parallel processing devices. In still other embodiments, a computer system or information processing device may use techniques described above as implemented upon a chip or an auxiliary processing board.

Various embodiments of an algorithm as described herein can be implemented in the form of logic in software, firmware, hardware, or a combination thereof. The logic may be stored in or on a machine-accessible memory, a machine-readable article, a tangible computer-readable medium, a computer-readable storage medium, or other computer/machine-readable media as a set of instructions adapted to direct a central processing unit (CPU or processor) of a logic machine to perform a set of steps that may be disclosed in various embodiments of an invention presented within this disclosure. The logic may form part of a software program or computer program product as code modules become operational with a processor of a computer system or an information-processing device when executed to perform a method or process in various embodiments of an invention presented within this disclosure. Based on this disclosure and the teachings provided herein, a person of ordinary skill in the art will appreciate other ways, variations, modifications, alternatives, and/or methods for implementing in software, firmware, hardware, or combinations thereof any of the disclosed operations or functionalities of various embodiments of one or more of the presented inventions.

### EXAMPLES

The experiments outlined in the initial examples that identified markers for prognosis stratified node-negative, ER-positive, HER2-negative breast cancer patients into those that are most or least likely to develop a recurrence within 10 years after surgery. A multi-gene transcription-level-based classifier of 10-year-relapse (disease recurrence within 10 years) was developed using a large database of existing, publicly available microarray datasets. The probability of relapse and relapse risk score group using the panel of gene expression markers of the invention can be used to assign systemic chemotherapy to only those patients most likely to benefit from it.

Methods:

Literature Search and Curation:

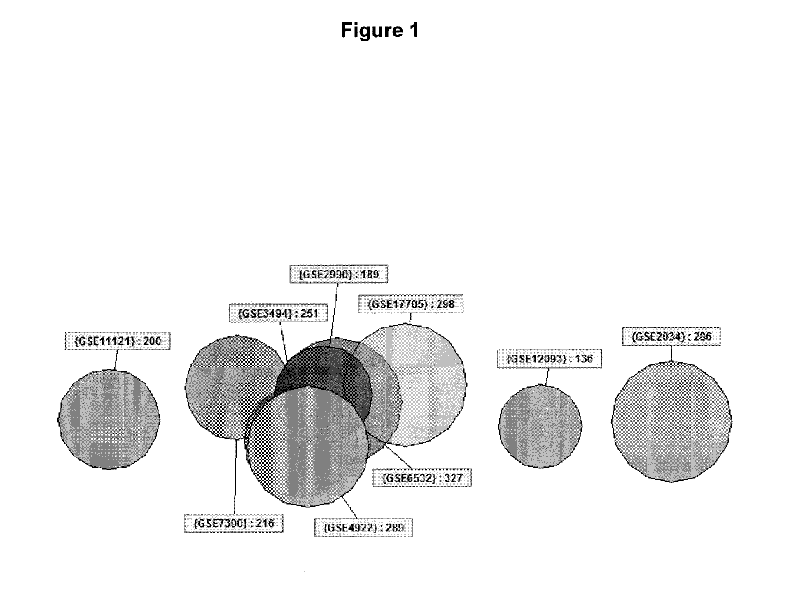

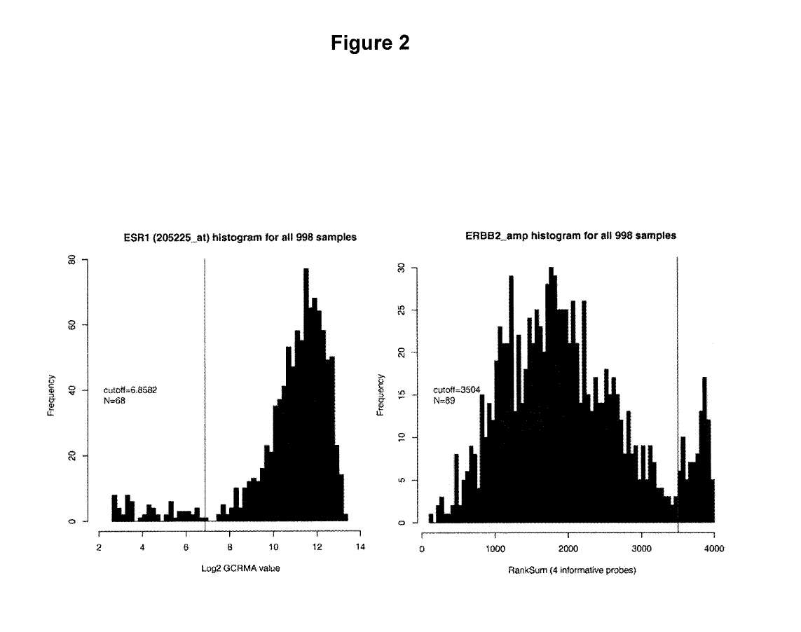

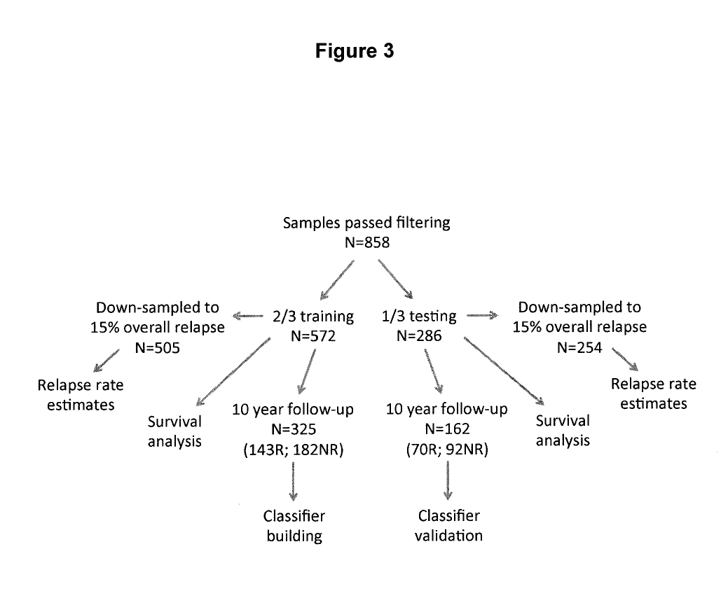

Studies were collected which provided gene expression data for ER+, LN−, HER2− patients with no systemic chemotherapy (hormonal-therapy allowed). Each study was required to have a sample size of at least 100, report LN status, and include time and events for either recurrence free survival (RFS) or distant metastasis free survival (DMFS). The latter were grouped together for survival analysis where all events represent either a local or distant relapse. If ER or HER2 status was not reported, it was determined by array, but preference was given to studies with clinical determination first. A minimum of 10 years follow up was required for training the classifier. However, patients with shorter follow-up were included in survival analyses. Patients with immediately postoperative events (time=0) were excluded. Nine studies¹⁻⁹ meeting the above criteria were identified by searching Pubmed and the Gene Expression Omnibus (GEO) database¹⁰. To allow combination of the largest number of samples, only the common Affymetrix U133A gene expression platform was used. 2175 breast cancer samples were identified. After filtering for only those samples which were ER+, node-negative, and had not received systemic chemotherapy, 1403 samples remained. Duplicate analysis removed a further 405 samples due to the significant amount of redundancy between studies (FIG. 1). Filtering for ER+ and HER2−status using array determinations eliminated another 140 samples (FIG. 2). Some ER−samples were from the Schmidt et al. Cancer Res 68, 5405-5413 (2008)⁵ dataset (31/201) which did not provide clinical ER status and thus for that study we relied solely on arrays for determination of ER status. However, there were also a small number (37/760) from the remaining studies, which represent discrepancies between array status and clinical determination. In such cases, both the clinical and array-based determinations were required to be positive for inclusion in further analysis. A total of 858 samples passed all filtering steps including 487 samples with 10 year follow-up data (213 relapse; 274 no relapse). The remaining 371 samples had insufficient follow-up for 10-year classification analysis, but were retained for use in the survival analysis. None of the 858 samples were treated with systemic chemotherapy but 302 (35.2%) were treated with adjuvant hormonal therapy of which 95.4% were listed as tamoxifen. The 858 samples were broken into two-thirds training and one-third testing sets resulting in: (A) a training set of 572 samples for use in survival analysis and 325 samples with 10yr follow-up (143 relapse; 182 no relapse) for classification analysis; and (B) a testing set of 286 samples for use in survival analysis and 162 samples with 10 year follow-up (70 relapse; 92 no relapse) for classification analysis. Table 6 outlines the datasets used in the analysis and FIG. 3 illustrates the breakdown of samples for analysis.

Pre-Processing:

All data processing and analyses were completed with open source R/Bioconductor packages. Raw data (Cel files) were downloaded from GEO. Duplicate samples were identified and removed if they had the same database identifier (e.g., GSM accession), same sample/patient id, or showed a high correlation (r>0.99) compared to any other sample in the dataset. Raw data were normalized and summarized using, the ‘affy’ and ‘gcrma’ libraries. Probes were mapped to Entrez gene symbols using both standard and custom annotation files¹¹. ER and HER2 expression status was determined using standard probes. For the Affymetrix U133A array we and others have found the probe “205225_at” to be most effective for determining ER status¹². Similarly a rank sum of the best probes for ERBB2 (216835_s_at), GRB7 (210761_s_at), STARD3 (202991_at) and PGAP3 (55616_at) was used to determine HER2 amplicon status. Cutoff values for ER and HER2 status were chosen by mixed model clustering (‘mclust’ library). Unsupervised clustering was performed to assess the extent of batch effects. Once all pre-filtering was complete, data were randomly split into training (⅔) and test (⅓) data sets while balancing for study of origin and number of relapses with 10 year follow-up. The test data set was put aside, left untouched, and only used for final validation, once each for the full-gene, 17-gene and 8-gene classifiers. Probes sets were then filtered for a minimum of 20% samples with expression above background threshold (raw value>100) and coefficient of variation between 0.7 and 10. A total of 3048 probesets/genes passed this filtering and formed the basis for the ‘full-gene set’ model described below.

Classification:

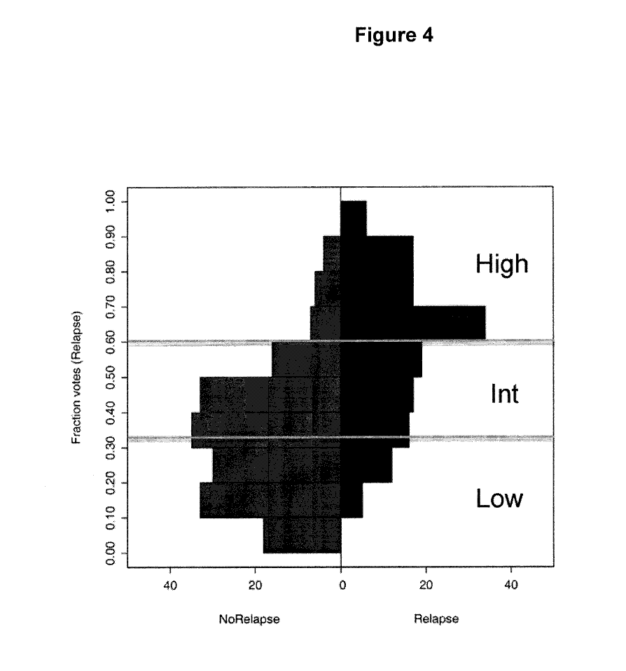

Classification was performed on only training samples with either a relapse or no relapse after 10yr follow-up using the ‘randomForest’ library. Forests were created with at least 100,001 trees (odd number ensures fully deterministic model) and otherwise default settings. Performance was assessed by area under the curve (AUC) of a receiver operating characteristic (ROC) curve, calculated with the ‘ROCR’ package, from Random Forests internal out-of-bag (00B) testing results. By default, RF performs a binary classification (e.g., relapse versus no relapse). However it also reports a probability (proportion of “votes”) for relapse which we term Random Forests Relapse Score (RFRS). Risk group thresholds were determined from the distribution of relapse probabilities using mixed model clustering to set cutoffs for low, intermediate and high risk groups (FIG. 4).

Determination of Optimal 17-Gene and 8-Gene Sets:

Initially an optimal set of 20 genes was selected by removing redundant probe sets and extracting the top 100 genes (by reported Gini variable importance), k-means clustering (k=20) these genes and selecting the best gene from each cluster (again by variable importance). Additional genes in each cluster serve as robust alternates in case of failure to migrate primary genes to an assay platform. A gene might fail to migrate due to problems with prober/primer design or differences in the sensitivity of a specific assay for that gene. The top 100 genes/probesets were also manually checked for sequence correctness by alignment to the reference genome. Seven genes/probesets with ambiguous or erroneous alignments were marked for exclusion. Three genes/probesets were also excluded because of their status as hypothetical proteins (KIAA0101, KIAA0776, KIAA1467). After these removals, a set of 17 primary genes and 73 alternate genes remained. All but two primary genes have two or more alternates (TXNIP is without alternate, and APOC 1 has a single alternate). Table 1 lists the final gene set, their top two alternate genes (where available) and their variable importance values (See Table 4 for complete list). The above procedure was repeated to produce an optimal set of 8 genes, this time starting from the top 90 non-redundant probe-sets (excluding the 10 genes with problems identified above), k-means clustering (k=8) these genes and selecting the best gene from each cluster. All 8 genes were also included in the 17-gene set and have at least two alternates (Table 2, Table 5). Using the final optimized 17-gene and 8-gene sets as input, new RF models were built on training data.

Validation (testing and survival analysis): Survival analysis on all training data, now also including those patients with less than 10 years of follow-up, was performed with risk group as a factor, for the full-gene, 17-gene, and 8-gene models, using the ‘survival’ package. Note, the risk scores and groups for samples used in training were assigned from internal 00B cross-validation. Only those patients not used in initial training (without 10 year follow-up) were assigned a risk score and group by de novo classification. Significance between risk groups was determined by Kaplan-Meier logrank test (with test for linear trend). However, to directly compare relapse rates per risk group to that reported by Paik et al., N Engl J Med 351: 2817-2826 (2004)¹³, the overall relapse rates in our patient cohort were randomly down-sampled to the same rate (15%) as in their cohort¹³ and results averaged over 1000 iterations. To illustrate, the training data set includes 572 samples with 143 relapse events (I.e., 25.0% relapse rate). Samples with relapse events were randomly eliminated from the cohort until only 15% of remaining samples had relapse events (76/505=15%). This “down-sampled” dataset was then classified using the RFRS model to assign each sample to a risk group and the rates of relapse determined for each group. The entire down-sampling procedure was then repeated 1000 times to obtain average estimated rates of relapse for each risk group given the overall rate of relapse of 15%. Setting the overall relapse rate to 15% is also useful because this more closely mirrors the general population rate of relapse. Without this down-sampling, expected relapse rates in each risk group would appear unrealistically high. See FIG. 2 for explanation of the break-down of samples into training and test sets used for classifier building and survival analysis.

Next, the full-gene, 17-gene and 8-gene RF models along with risk group cutoffs were applied to the independent test data. The same performance metrics, survival analysis and estimates of 10 year relapse rates were performed as above. The 17-gene model was also tested on the independent test data, stratified by treatment (untreated vs hormone therapy treated), to evaluate whether performance of the signature was biased towards one patient subpopulation or the other. These independent test data were not used in any way during the training phase. However, these samples represent a random subset of the same patient populations that were used in training Therefore, they are not as fully independent as recommended by the Institute of Medicine (IOM) ‘committee on the review of omics-based tests for predicting patient outcomes in clinical trials’¹⁸. Therefore, an additional independent validation was performed against the NKI dataset¹⁹ obtained from the http address bioinformatics.nki.nl/data.php. These data represent a set of 295 consecutive patients with primary stage I or II breast carcinomas. The dataset was filtered down to the 89 patients who were node-negative, ER-positive, HER2-negative and not treated by systemic chemotherapy¹⁹. Relapse times and events were defined by any of distant metastasis, regional recurrence or local recurrence. Expression values from the NKI Agilent array data were re-scaled to the same distribution as that used in training using the ‘preprocessCore’ package. Values for the 8-gene and 17-gene-set RFRS models were extracted for further analysis. If more than one Agilent probe set could be mapped to an RFRS gene then the probe set with greatest variance was used. The full-gene-set model was not applied to NKI data because only 2530/3048 Affymetrix-defined genes (probe sets) in the full-gene-set could be mapped to Agilent genes (probe sets) in the NKI dataset. However, the 17-gene and 8-gene RFRS models were applied to NKI data to calculate predicted probabilities of relapse. Patients were divided into low, intermediate, and high risk groups by ranking according to probability of relapse and then dividing so that the proportions in each risk group were identical to that observed in training ROC AUC, survival p-values and estimated rates of relapse were then calculated as above. It should be noted that while the NKI clinical data described here (N=89) had an average follow-up time of 9.55 years (excluding relapse events), 34 patients had a follow-up time less than 10 years (range 1.78-9.83 years). These patients would not have met our criteria for inclusion in the training dataset and likely represent some events which have not occurred yet. If anything, this is likely to reduce the AUC estimate and underestimate p-value significance in survival analysis.

Selection of Control Genes:

While not necessary for Affymetrix, migration to other assay technologies (e.g., RT-PCR approaches) may employ highly expressed and invariant genes to act as a reference for determining accurate gene expression level estimates. To this end, we developed two sets of reference genes. The first was chosen by the following criteria: (1) filtered if not expressed above background threshold (raw value>100) in 99% of samples; (2) filtered if not in top 5th percentile (overall) for mean expression; (3) Filtered if not in top 10th percentile (remaining genes) for standard deviation; (4) ranked by coefficient of variation. The top 30 control genes from set #1 are listed in Table 3. Control genes underwent the same manual checks for sequence correctness by alignment to the reference genome as above and five genes were marked for exclusion. The second set of control genes were chosen to represent three ranges of mean expression levels encompassed by genes in the 17-gene signature (low: 0-400; medium: 500-900; high: 1200-1600). For each mean expression range, genes were (1) filtered if not expressed above background threshold (raw value>100) in 99% of samples; (2) ranked by coefficient of variation. The top 5 genes from each range in set #2 are listed in Table 3 along with previously reported reference genes (Paik et al., supra)¹³

### Results:

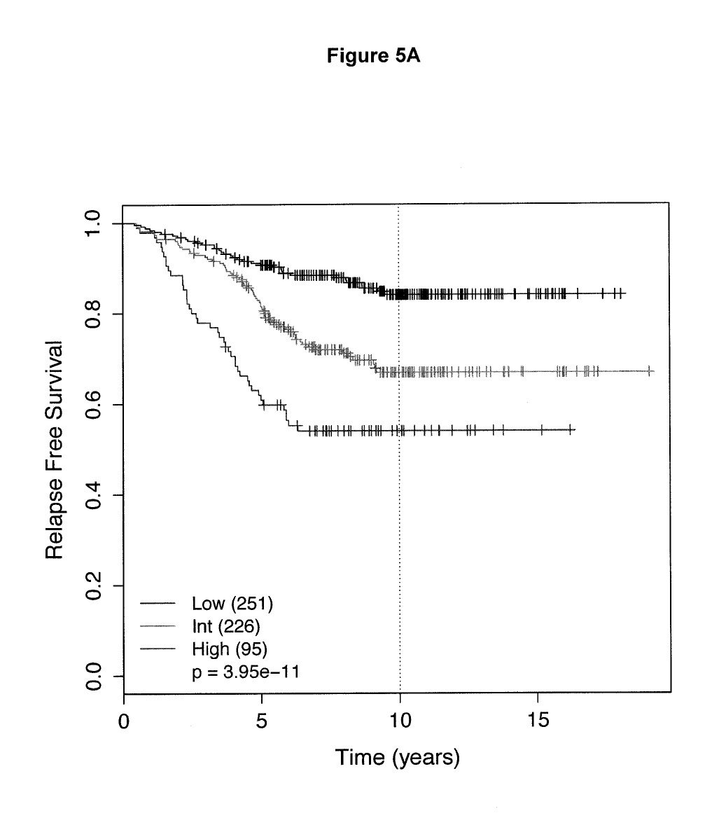

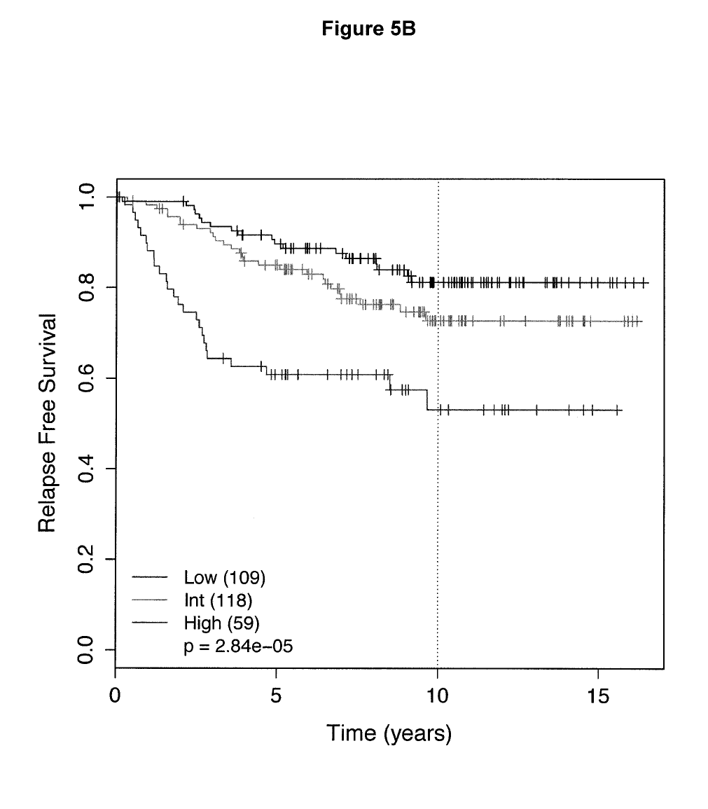

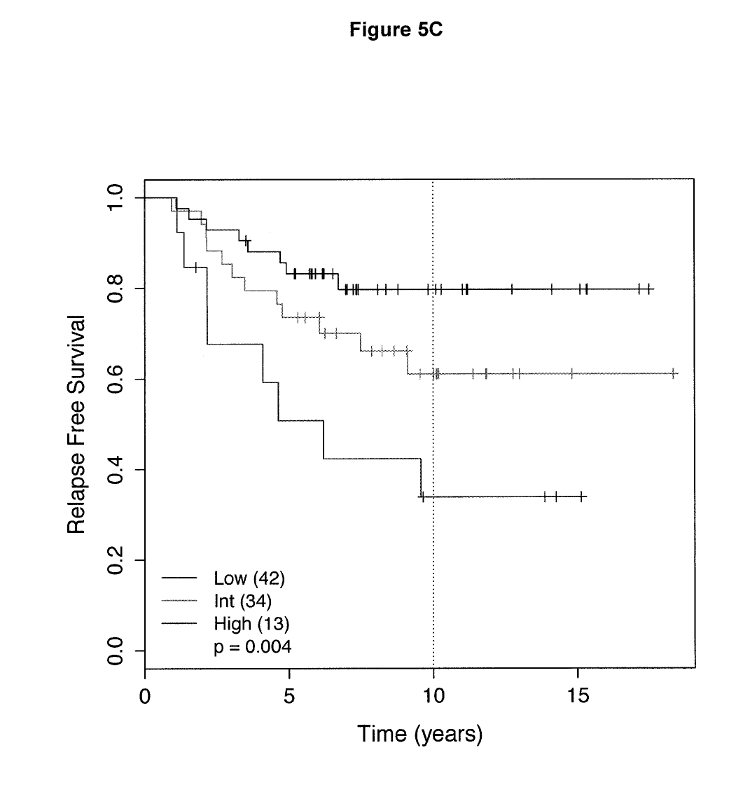

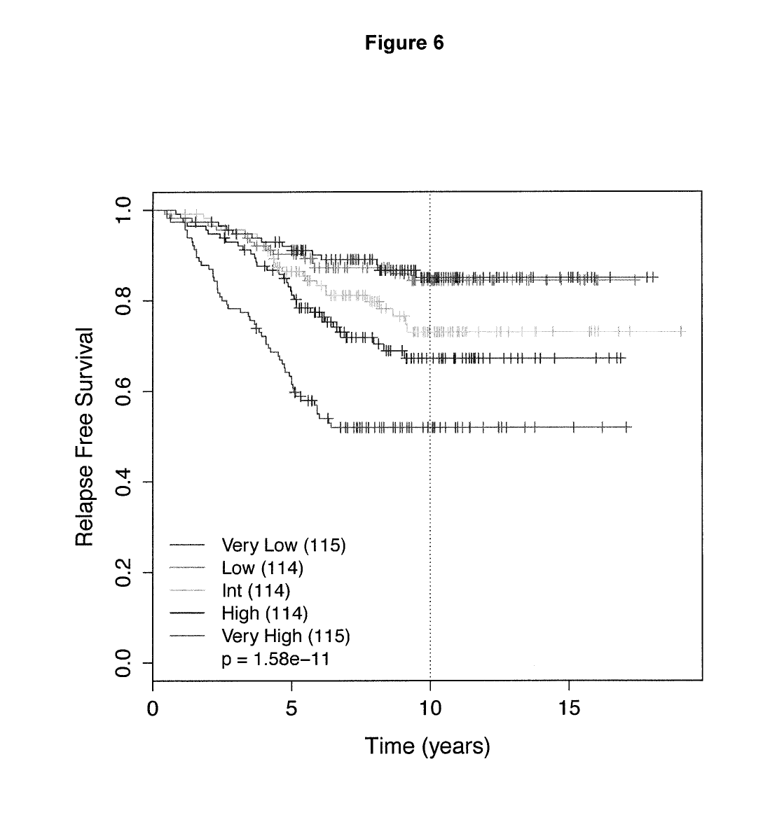

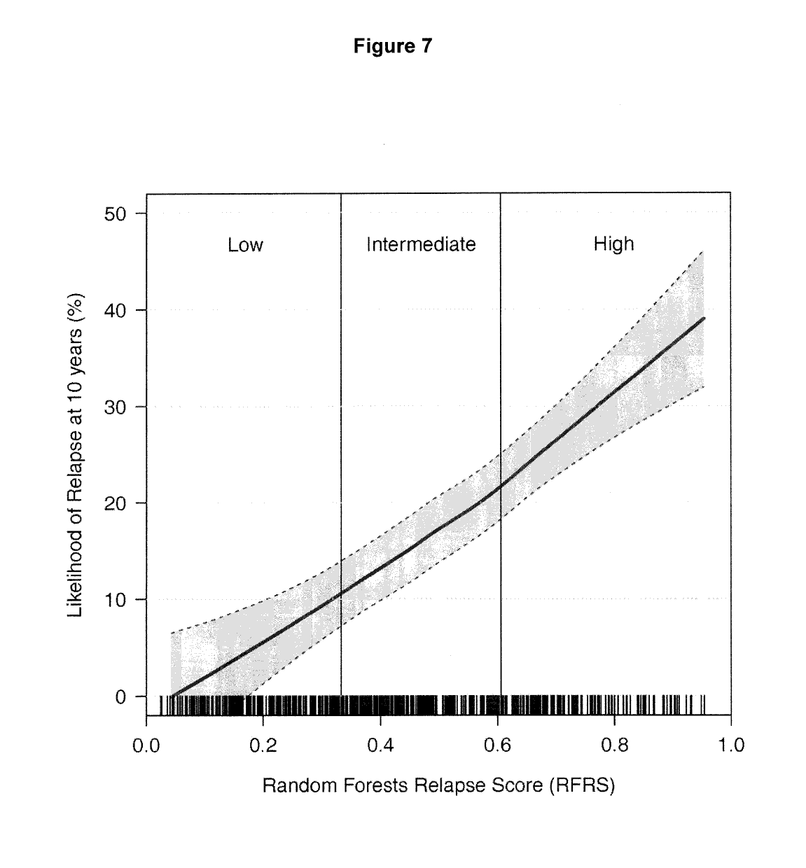

Internal OOB cross-validation for the initial (full-gene-set) model on training data reported an ROC AUC of 0.704. This was comparable or better than reported by Johannes et al (2010) who tested a number of different classifiers on a smaller subset of the same data and found AUCs of 0.559 to 0.671¹⁴. It also compares favorably to the AUC value of 0.688 when the OncotypeDX algorithm was applied to this same training dataset. Mixed model clustering analysis identified three risk groups with probabilities for low risk<0.333; 0.333≦intermediate risk<0.606; and high risk≧0.606 (FIG. 4). Survival analysis determined a highly significant difference in relapse rate between risk groups (p=3.95E-11) (FIG. 5A). After down-sampling to a 15% overall rate of relapse, approximately 46.7% (n=235) of patients were placed in the low-risk group and were found to have a 10yr risk of relapse of only 8.0%. Similarly, 38.6% (n=195) and 14.9% (n=75) of patients were placed in the intermediate and high risk groups with rates of relapse of 17.6% and 30.3% respectively. These results are very similar to those for which Paik et al., supra reported as 51% of patients in the low-risk category with a rate of distant recurrence at 10 years of 6.8% (95% CI: 4.0-9.6); 22% in intermediate-risk category with recurrence rate of 14.3% (95% CI: 8.3-20.3); and 27% in high-risk category with recurrence rate of 30.5% (95% CI: 23.6-37.4)¹³. The linear relationship between risk group and rate of relapse continues if groups are broken down further. For example, if “very low-risk” and “very high-risk” groups are defined these have even lower (7.1%) and higher (32.8%) rates of relapse (FIG. 6). This observation is consistent with the idea that the random forests relapse score (RFRS) is a quantitative, linear measure directly related to probability of relapse. FIG. 7 shows the likelihood of relapse at 10 years, calculated for 50 RFRS intervals (from 0 to 1), with a smooth curve fitted, using a loess function and 95% confidence intervals representing error in the fit. The distribution of RFRS values observed in the training data is represented by short vertical marks just above the x axis, one for each patient.

Validation of the models against the independent test dataset also showed very similar results to training estimates. The full-gene-set model had an AUC of 0.730 and the 17-gene and 8-gene optimized models had minimal reduction in performance with AUC of 0.715 and 0.690 respectively. Again, this compared favorably to the AUC value of 0.712 when the OncotypeDX algorithm was applied to the same test dataset. Survival analysis again found very significant differences between the risk groups for the full-gene (p=6.54E-06), 17-gene (p=9.57E-06) and 8-gene (p=2.84E-05; FIG. 5B) models. For the 17-gene model, approximately 38.2% (n=97) of patients were placed in the low-risk group and were found to have a 10-year risk of relapse of only 7.8%. Similarly, 40.5% (n=103) and 21.3% (n=54) of patients were placed in the intermediate and high-risk groups with rates of relapse of 15.3% and 26.8% respectively. Very similar results were observed for the full-gene and 8-gene models (Table 7). Validation against the additional, independent, NKI dataset also had very similar results. The 17-gene and 8-gene models had AUC values of 0.688 and 0.699 respectively, nearly identical to the results for the previous independent dataset. Differences between risk groups in survival analysis were also significant for both 17-gene (p=0.023) and 8-gene (p=0.004, FIG. 5C) models.

The linear relationship between risk group and rate of relapse continues if groups are broken down further (using training data) into five equal groups instead of the three groups defined above (FIG. 6). This observation is consistent with the idea that the random forests relapse score (RFRS) is a quantitative, linear measure directly related to probability of relapse.

FIG. 7 shows the likelihood of relapse at 10 years, calculated for 50 RFRS intervals (from 0 to 1), with a smooth curve fitted, using a loess function and 95% confidence intervals representing error in the fit. The distribution of RFRS values observed in the training data is represented by short vertical marks just above the x axis, one for each patient.

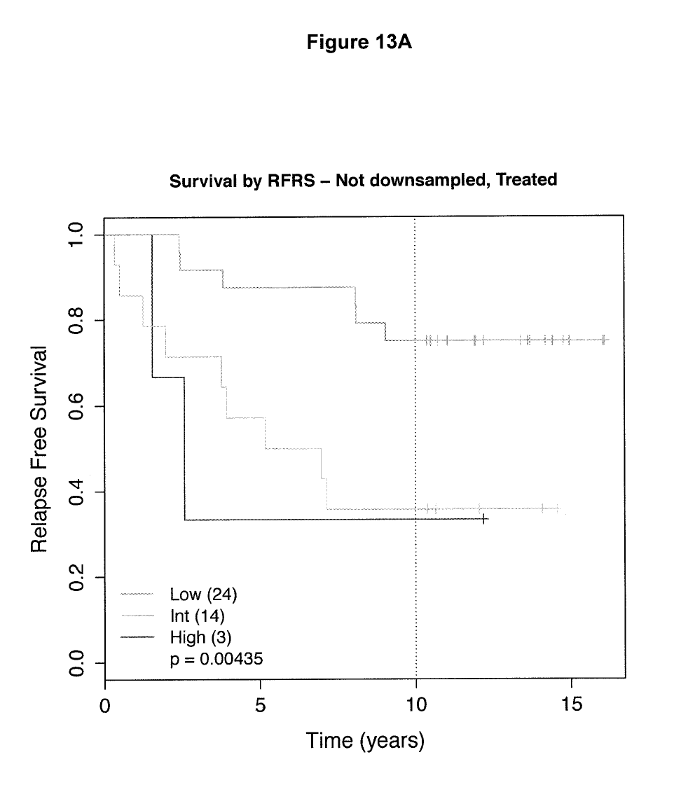

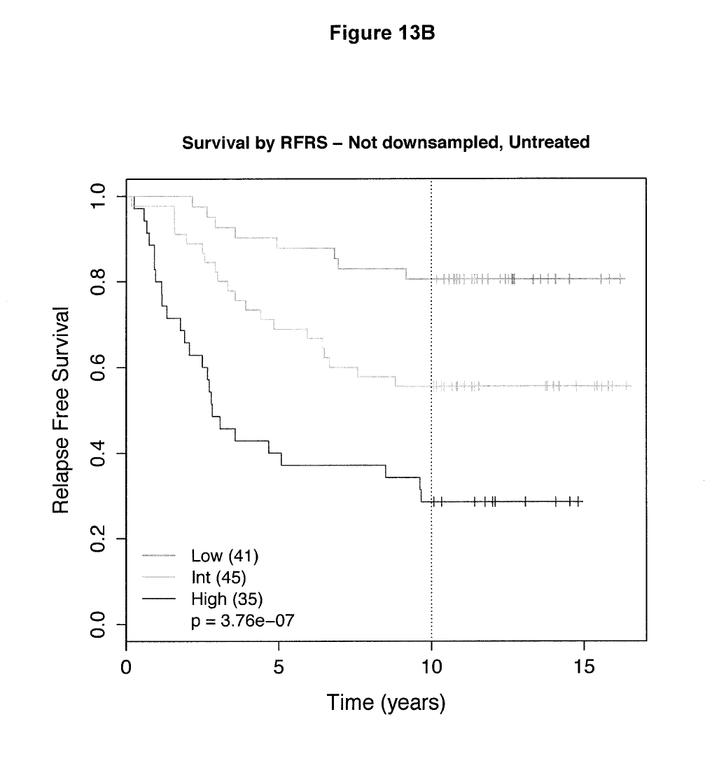

In order to maximize the total size of our training dataset we allowed samples to be included from both untreated patients and those who received adjuvant hormonal therapy such as tamoxifen. Since outcomes likely differ between these two groups, and they may represent fundamentally different subpopulations, it is possible that performance of our predictive signatures is biased towards one group or the other. To assess this issue we performed validation against the independent test dataset, stratified by treatment status, using the 17-gene model. Both groups were found to have comparable AUC values with the slightly better value of 0.740 for hormone-treated versus 0.709 for untreated. Survival curves were also highly similar and significant with p-value of 0.004 and 3.76E-07 for treated and untreated respectively (FIGS. 13A and 13B). The difference in p-value appears more likely due to differences in the respective sample sizes than actual difference in survival curves.

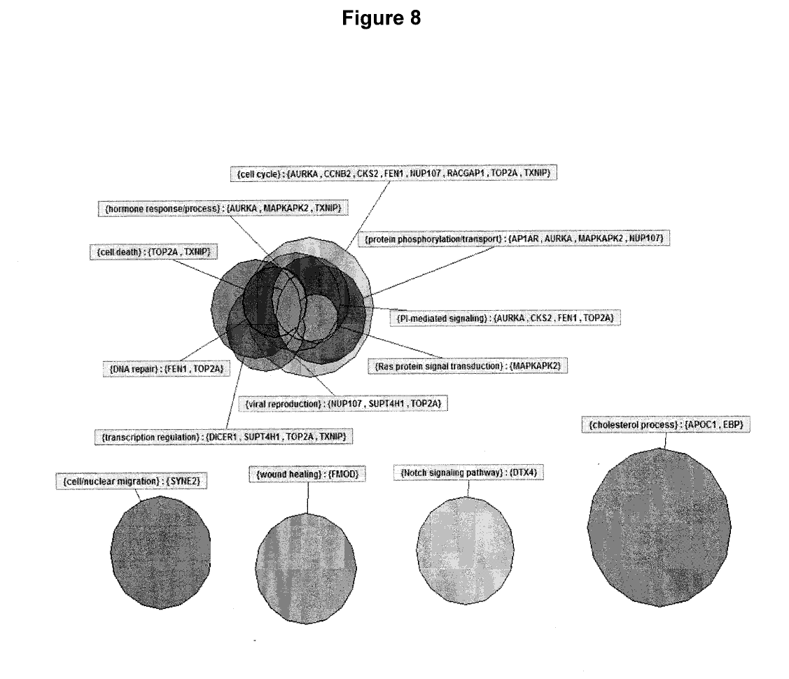

The genes utilized in the RFRS model have only minimal overlap with those identified in other breast cancer outcome signatures. Specifically, the entire set of 100 genes (full-gene set before filtering) has only 6/65 genes in common with the gene expression panel proposed by van de Vijver, et al. N Engl J Med 347, 1999-2009 (2002)¹⁵, 2/21 with that proposed by Paik et al., supra, and 4/77 with that proposed by Wang et al. Lancet 365:671-679 (2005)²⁰. The 17-gene and 8-gene optimized sets have only a single gene (AURKA) in common with the panel proposed by Paik et al., a single gene (FEN1) in common with Wang et al., and none with that of van de Vijver et al. A Gene Ontology analysis using DAVID^{16,17} revealed that genes in the 17-gene list are involved in a wide range of biological processes known to be involved in breast cancer biology including cell cycle, hormone response, cell death, DNA repair, transcription regulation, wound healing and others (FIG. 8). Since the 8-gene set is entirely contained in the 17-gene set it would be involved in many of the same processes.

While methods such as those proposed by Paik et al., and de Vijver, et al. (both supra)^{13,15} exist to predict outcome in breast cancer, the RFRS is advantageous in several respects: (1) The signature was built from the largest and purest training dataset available to date; (2) Patients with HER2+ tumors were excluded, thus focusing only on patients without an existing clear treatment course; (3) The gene signature predicts relapse with equal success for both patients that went on to receive adjuvant hormonal therapy and those who did not (4) The gene signature was designed for robustness with (in most cases) several alternate genes available for each primary gene; (5) probe set sequences have been manually validated by alignment and manual assessment. These features, particularly the latter two, make this signature an especially strong candidate for efficient migration to multiple low-cost platforms for use in a clinical setting. Development of a panel for use in the clinic could take advantage of not only primary genes but also some number of alternate genes to increase the chance of a successful migration. Given the small but significant number of discrepencies observed between clinical and array based determination of ER status we also recommend inclusion of standard biomarkers such as ER, PR and HER2 on any design. Finally, we provide a list of consistently expressed genes, specific to breast tumor tissue, for use as control genes for those platforms that require them.

### Implementation of Algorithm Using 17-Gene Model as Example:

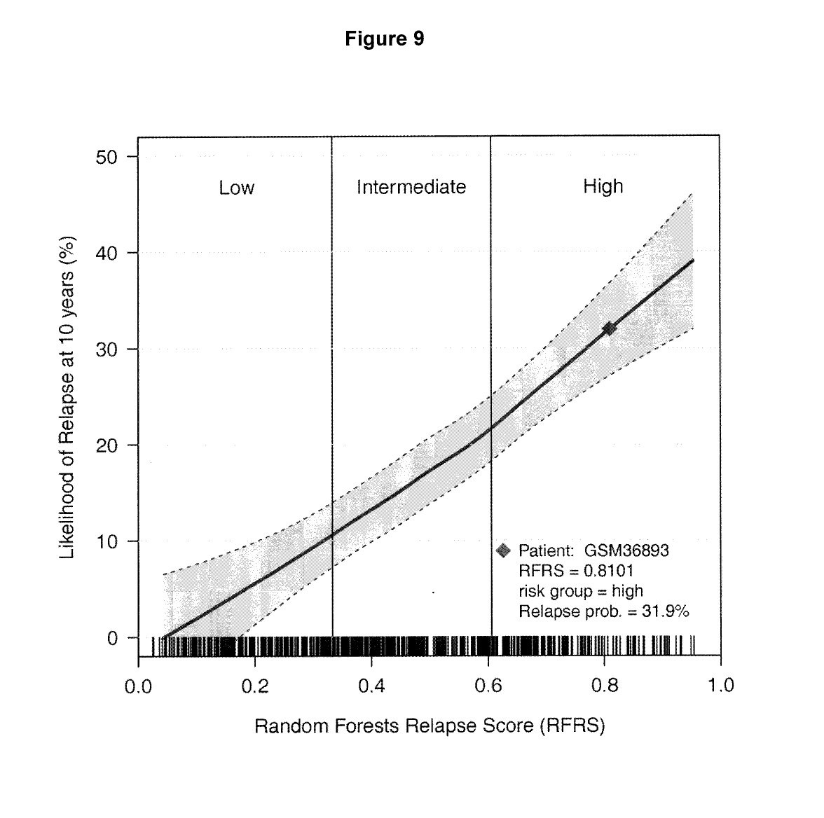

The RFRS algorithm is implemented in the R programming language and can be applied to independent patient data. Input data is a tab-delimited text file of normalized expression values with 17 transcripts/genes as columns and patient(s) as rows. A sample patient data file (patient_data.txt) is presented in Appendix 1. A sample R program (RFRS_sample_code.R) for running the algorithm is presented in Appendix 2. The RFRS algorithm consists of a Random Forest of 100,001 decision trees. This is pre-computed, provided as an R data object (RF_model_{—}17gene_optimized) based on the training set and is included in the working directory. Each node (branch) in each tree represents a binary decision based on transcript levels for transcripts described above. Based on these decisions, the patient is assigned to a terminal leaf of each decision tree, representing a vote for either “relapse” or no “relapse”. The fraction of votes for “relapse” to votes for “no relapse” represents the RFRS—a measure of the probability of relapse. If RFRS is greater than or equal to 0.606 the patient is assigned to the “high risk” group, if greater than or equal to 0.333 and less than 0.606 the patient is assigned to “intermediate risk” group and if less than 0.333 the patient is assigned to “low risk” group. The patient's RFRS value is also used to determine a likelihood of relapse by comparison to a loess fit of RFRS versus likelihood of relapse for the training dataset. Pre-computed R data objects for the loess fit (RelapseProbabilityFit.Rdata) and summary plot (RelapseProbabilityPlot.Rdata) are loaded from file. The patient's estimated likelihood of relapse is determined, added to the summary plot, and output as a new report (see, FIG. 9, for example).

### REFERENCES CITED IN EXAMPLES SECTION

1 Desmedt, C. et al. Strong time dependence of the 76-gene prognostic signature for node-negative breast cancer patients in the TRANSBIG multicenter independent validation series. Clin Cancer Res 13, 3207-3214 (2007).
2 Ivshina, A. V. et al. Genetic reclassification of histologic grade delineates new clinical subtypes of breast cancer. Cancer Res 66, 10292-10301 (2006).
3 Loi, S. et al. Definition of clinically distinct molecular subtypes in estrogen receptor-positive breast carcinomas through genomic grade. J Clin Oncol 25, 1239-1246 (2007).
4 Miller, L. D. et al. An expression signature for p53 status in human breast cancer predicts mutation status, transcriptional effects, and patient survival. Proc Natl Acad Sci USA 102, 13550-13555 (2005).
5 Schmidt, M. et al. The humoral immune system has a key prognostic impact in node-negative breast cancer. Cancer Res 68, 5405-5413 (2008).
6 Sotiriou, C. et al. Gene expression profiling in breast cancer: understanding the molecular basis of histologic grade to improve prognosis. J Natl Cancer Inst 98, 262-272 (2006).
7 Symmans, W. F. et al. Genomic index of sensitivity to endocrine therapy for breast cancer. J Clin Oncol 28, 4111-4119 (2010).
8 Wang, Y. et al. Gene-expression profiles to predict distant metastasis of lymph-node-negative primary breast cancer. Lancet 365, 671-679 (2005).
9 Zhang, Y. et al. The 76-gene signature defines high-risk patients that benefit from adjuvant tamoxifen therapy. Breast Cancer Res Treat 116, 303-309 (2009).
10 Barrett, T. et al. NCBI GEO: archive for functional genomics data sets—10 years on. Nucleic Acids Res 39 (2011).
11 Dai, M. et al. Evolving gene/transcript definitions significantly alter the interpretation of GeneChip data. Nucleic Acids Res 33, e175, (2005).
12 Gong, Y. et al. Determination of oestrogen-receptor status and ERBB2 status of breast carcinoma: a gene-expression profiling study. Lancet Oncol 8, 203-211 (2007).
13 Paik, S. et al. A multigene assay to predict recurrence of tamoxifen-treated, node-negative breast cancer. N Engl J Med 351, 2817-2826 (2004).
14 Johannes, M. et al. Integration of pathway knowledge into a reweighted recursive feature elimination approach for risk stratification of cancer patients. Bioinformatics 26, 2136-2144 (2010).
van de Vijver, M. J. et al. A gene-expression signature as a predictor of survival in breast cancer. N Engl J Med 347, 1999-2009 (2002).
16 Huang da, W., Sherman, B. T. & Lempicki, R. A. Systematic and integrative analysis of large gene lists using DAVID bioinformatics resources. Nature protocols 4, 44-57 (2009).
17 Huang da, W., Sherman, B. T. & Lempicki, R. A. Bioinformatics enrichment tools: paths toward the comprehensive functional analysis of large gene lists. Nucleic Acids Res 37, 1-13 (2009).
18. Committee on the Review of Omics-Based Tests for Predicting Patient Outcomes in Clinical Trials, Board on Health Care Services, Board on Health Sciences Policy, Institute of Medicine. Evolution of Translational Omics: Lessons Learned and the Path Forward. Christine M M, Sharly J N, Gilbert S O, editors: The National Academies Press; 2012.
19. van de Vijver M J, He Y D, van't Veer L J, Dai H, Hart A A, Voskuil D W, et al. A gene-expression signature as a predictor of survival in breast cancer. N Engl J Med 2002;347:1999-2009.
20. Wang, Y. et al. Gene-expression profiles to predict distant metastasis of lymph-node-negative primary breast cancer. Lancet 365, 671-679 (2005).

All publications, patents, accession numbers, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

TABLE 1
17-gene RFRS signature

| Primary Predictor | | Alternate 1 | | Alternate 2 | |
|---|---|---|---|---|---|
| CCNB2 | 0.785 | MELK | 0.739 | GINS1 | 0.476 |
| TOP2A | 0.590 | MCM2 | 0.428 | CDK1 | 0.379 |
| RACGAP1 | 0.588 | LSM1 | 0.139 | SCD | 0.125 |
| CKS2 | 0.515 | NUSAP1 | 0.491 | ZWINT | 0.272 |
| AURKA | 0.508 | PRC1 | 0.499 | CENPF | 0.306 |
| FEN1 | 0.403 | FADD | 0.313 | SMC4 | 0.170 |
| EBP | 0.341 | RFC4 | 0.264 | NCAPG | 0.234 |
| TXNIP | 0.292 | N/A | N/A | N/A | N/A |
| SYNE2 | 0.270 | SCARB2 | 0.225 | PDLIM5 | 0.167 |
| DICER1 | 0.209 | CALD1 | 0.129 | SOX9 | 0.125 |
| AP1AR | 0.201 | PBX2 | 0.134 | WASL | 0.126 |
| NUP107 | 0.197 | FAM38A | 0.165 | PLIN2 | 0.110 |
| APOC1 | 0.176 | APOE | 0.121 | N/A | N/A |
| DTX4 | 0.164 | AQP1 | 0.141 | LMO4 | 0.120 |
| FMOD | 0.154 | RGS5 | 0.120 | PIK3R1 | 0.103 |
| MAPKAPK2 | 0.151 | MTUS1 | 0.136 | DHX9 | 0.136 |
| SUPT4H1 | 0.111 | PHB | 0.106 | CD44 | 0.105 |

TABLE 2
8-gene RFRS signature

| Primary Predictor | | Alternate 1 | | Alternate 2 | |
|---|---|---|---|---|---|
| CCNB2 | 0.785 | MELK | 0.739 | TOP2A | 0.590 |
| RACGAP1 | 0.588 | TXNIP | 0.292 | APOC1 | 0.176 |
| CKS2 | 0.515 | NUSAP1 | 0.491 | FEN1 | 0.403 |
| AURKA | 0.508 | PRC1 | 0.499 | CENPF | 0.306 |
| EBP | 0.341 | FADD | 0.313 | RFC4 | 0.264 |
| SYNE2 | 0.270 | SCARB2 | 0.225 | PDLIM5 | 0.167 |
| DICER1 | 0.209 | FAM38A | 0.165 | FMOD | 0.154 |
| AP1AR | 0.201 | MAPKAPK2 | 0.151 | MTUS1 | 0.136 |

TABLE 3

| | | | | | | |
|---|---|---|---|---|---|---|
| Probe set | Gene Symbol | Mean (exp) | S.D. | Fraction (exp) | COV | CDF |

Top 25 RFRS Reference Genes

| | | | | | | |
|---|---|---|---|---|---|---|
| 103910_at | MYL12B | 1017.5 | 195.8 | 1.00 | 0.192 | custom |
| 208672_s_at | SFRS3 | 1713.0 | 380.0 | 1.00 | 0.222 | standard |
| 200960_x_at | CLTA | 1786.2 | 397.5 | 1.00 | 0.223 | standard |
| 200893_at | TRA2B | 1403.7 | 312.8 | 1.00 | 0.223 | standard |
| 23787_at | MTCH1 | 1120.0 | 269.8 | 1.00 | 0.241 | custom |
| 221767_x_at | HDLBP | 1174.4 | 284.9 | 1.00 | 0.243 | standard |
| 23191_at | CYFIP1 | 1345.1 | 329.4 | 1.00 | 0.245 | custom |
| 211069_s_at | SUMO1 | 1111.6 | 276.2 | 1.00 | 0.248 | standard |
| 201385_at | DHX15 | 1529.4 | 383.5 | 1.00 | 0.251 | standard |
| 200014_s_at | HNRNPC | 1517.7 | 385.3 | 1.00 | 0.254 | standard |
| 200667_at | UBE2D3 | 1090.1 | 279.3 | 1.00 | 0.256 | standard |
| 9802_at | DAZAP2 | 1181.2 | 303.6 | 1.00 | 0.257 | custom |
| 200058_s_at | SNRNP200 | 1104.4 | 285.9 | 1.00 | 0.259 | standard |
| 91746_at | YTHDC1 | 965.1 | 250.7 | 1.00 | 0.260 | custom |
| 1315_at | COPB1 | 1118.2 | 291.9 | 1.00 | 0.261 | custom |
| 4714_at | NDUFB8 | 1219.0 | 325.5 | 1.00 | 0.267 | custom |
| 40189_at | SET | 1347.9 | 360.7 | 1.00 | 0.268 | standard |
| 221743_at | CELF1 | 1094.0 | 294.2 | 1.00 | 0.269 | standard |
| 208775_at | XPO1 | 940.7 | 256.1 | 1.00 | 0.272 | standard |
| 211270_x_at | PTBP1 | 973.1 | 266.8 | 1.00 | 0.274 | standard |
| 211185_s_at | SF3B1 | 1077.9 | 297.9 | 1.00 | 0.276 | standard |
| 10109_at | ARPC2 | 1357.4 | 375.9 | 1.00 | 0.277 | custom |
| 201336_at | VAMP3 | 959.2 | 267.4 | 1.00 | 0.279 | standard |
| 200028_s_at | STARD7 | 1087.9 | 303.4 | 1.00 | 0.279 | standard |
| 22872_at | SEC31A | 1040.4 | 290.5 | 1.00 | 0.279 | custom |

Top 15 RFRS Reference Genes (Set #2)

| | | | | | | |
|---|---|---|---|---|---|---|
| 9927_at | MFN2 | 207.0 | 33.1 | 1.00 | 0.160 | custom |
| 26100_at | WIPI2 | 216.5 | 40.3 | 1.00 | 0.186 | custom |
| 201507_at | PFDN1 | 260.8 | 51.2 | 1.00 | 0.196 | standard |
| 7337_at | UBE3A | 225.3 | 46.5 | 0.99 | 0.207 | custom |
| 2976_at | GTF3C2 | 226.3 | 47.6 | 1.00 | 0.210 | custom |
| 10657_at | KHDRBS1 | 776.4 | 166.3 | 1.00 | 0.214 | custom |
| 201330_at | RARS | 502.6 | 117.1 | 1.00 | 0.233 | standard |
| 201319_at | MYL12A | 574.4 | 135.2 | 1.00 | 0.235 | standard |
| 3184_at | HNRNPD | 678.8 | 160.0 | 1.00 | 0.236 | custom |
| 10236_at | HNRNPR | 570.1 | 140.4 | 1.00 | 0.246 | custom |
| 200893_at | TRA2B | 1403.7 | 312.8 | 1.00 | 0.223 | standard |
| 221619_s_at | MTCH1* | 1401.9 | 342.6 | 1.00 | 0.244 | standard |
| 208923_at | CYFIP1* | 1339.2 | 333.6 | 1.00 | 0.249 | standard |
| 201385_at | DHX15 | 1529.4 | 383.5 | 1.00 | 0.251 | standard |
| 4714_at | NDUFB8 | 1219.0 | 325.5 | 1.00 | 0.267 | custom |

Oncotype DX ® (Genomic Health, Inc, Redwood City, CA) Reference Genes

| | | | | | | |
|---|---|---|---|---|---|---|
| 213867_x_at | ACTB | 19566.3 | 4360.8 | 1.00 | 0.223 | standard |
| 200801_x_at | ACTB | 17901.0 | 3995.4 | 1.00 | 0.223 | standard |
| 2597_at | GAPDH | 11873.9 | 3810.3 | 1.00 | 0.321 | standard |
| 212581_x_at | GAPDH | 11930.9 | 4172.5 | 1.00 | 0.350 | standard |
| 217398_x_at | GAPDH | 6595.6 | 2460.2 | 1.00 | 0.373 | standard |
| 213453_x_at | GAPDH | 6695.2 | 2726.8 | 1.00 | 0.407 | standard |
| 60_at | ACTB | 3786.2 | 1622.3 | 1.00 | 0.428 | standard |
| 7037_at | TFRC | 781.8 | 466.6 | 1.00 | 0.597 | standard |
| 208691_at | TFRC | 1035.1 | 630.8 | 1.00 | 0.609 | standard |
| 207332_s_at | TFRC | 506.9 | 341.6 | 0.97 | 0.674 | standard |

RPLPO and GUS are also listed as reference genes for the Oncotype DX ® breast cancer assay.

TABLE 4
100 probe sets including all primary, alternate, and excluded genes (k = 20 clusters)

| Gene (probe set) | EntrezID | CDF | Varlmp | Predictor group | Predictor status |
|---|---|---|---|---|---|
| CCNB2 (9133_at) | 9133 | custom | 0.785 | primary | predictor1 |
| MELK (9833_at) | 9833 | custom | 0.739 | alternate1 | predictor1 alternate1 |
| GINS1 (9837_at) | 9837 | custom | 0.476 | alternate2 | predictor1 alternate2 |
| RRM2 (6241_at) | 6241 | custom | 0.399 | alternate3 | predictor1 alternate3 |
| GINS2 (51659_at) | 51659 | custom | 0.354 | alternate4 | predictor1 alternate4 |
| CCNB1 (214710_s_at) | 891 | standard | 0.140 | alternate5 | predictor1 alternate5 |
| TOP2A (201291_s_at) | 7153 | standard | 0.590 | primary | predictor2 |
| MCM2 (4171_at) | 4171 | custom | 0.428 | alternate1 | predictor2 alternate1 |
| KIAA0101 (9768_at) | 9768 | custom | 0.409 | alternate2 | predictor2 alternate2 (excluded) |
| CDK1 (203213_at) | 983 | standard | 0.379 | alternate3 | predictor2 alternate3 |
| UBE2C (202954_at) | 11065 | standard | 0.365 | alternate4 | predictor2 alternate4 |
| TMEM97 (212281_s_at) | 27346 | standard | 0.147 | alternate5 | predictor2 alternate5 |
| DTL (218585_s_at) | 51514 | standard | 0.130 | alternate6 | predictor2 alternate6 |
| RACGAP1 (29127_at) | 29127 | custom | 0.588 | primary | predictor3 |
| LSM1 (27257_at) | 27257 | custom | 0.139 | alternate1 | predictor3 alternate1 |
| SCD (200832_s_at) | 6319 | standard | 0.125 | alternate2 | predictor3 alternate2 |
| HN1 (51155_at) | 51155 | custom | 0.104 | alternate3 | predictor3 alternate3 |
| CKS2 (1164_at) | 1164 | custom | 0.515 | primary | predictor4 |
| NUSAP1 (218039_at) | 51203 | standard | 0.491 | alternate1 | predictor4 alternate1 |
| PTTG1 (203554_x_at) | 9232 | standard | 0.408 | alternate2 | predictor4 alternate2 (excluded) |
| ZWINT (204026_s_at) | 11130 | standard | 0.272 | alternate3 | predictor4 alternate3 |
| TYMS (7298_at) | 7298 | custom | 0.269 | alternate4 | predictor4 alternate4 |
| MLF1IP (218883_s_at) | 79682 | standard | 0.204 | alternate5 | predictor4 alternate5 |
| SQLE (209218_at) | 6713 | standard | 0.174 | alternate6 | predictor4 alternate6 |
| AURKA (208079_s_at) | 6790 | standard | 0.508 | primary | predictor5 |
| PRC1 (9055_at) | 9055 | custom | 0.499 | alternate1 | predictor5 alternate1 |
| CENPF (207828_s_at) | 1063 | standard | 0.306 | alternate2 | predictor5 alternate2 |
| ASPM (219918_s_at) | 259266 | standard | 0.293 | alternate3 | predictor5 alternate3 |
| NEK2 (204641_at) | 4751 | standard | 0.134 | alternate4 | predictor5 alternate4 |
| ECT2 (1894_at) | 1894 | custom | 0.105 | alternate5 | predictor5 alternate5 |
| FEN1 (204767_s_at) | 2237 | standard | 0.403 | primary | predictor6 |
| FADD (8772_at) | 8772 | custom | 0.313 | alternate1 | predictor6 alternate1 |
| SMC4 (10051_at) | 10051 | custom | 0.170 | alternate2 | predictor6 alternate2 |
| SLC35E3 (55508_at) | 55508 | custom | 0.151 | alternate3 | predictor6 alternate3 |
| TXNRD1 (7296_at) | 7296 | custom | 0.136 | alternate4 | predictor6 alternate4 |
| RAE1 (211318_s_at) | 8480 | standard | 0.132 | alternate5 | predictor6 alternate5 |
| ACBD3 (202323_s_at) | 64746 | standard | 0.129 | alternate6 | predictor6 alternate6 |
| ZNF274 (204937_s_at) | 10782 | standard | 0.122 | alternate7 | predictor6 alternate7 |
| FRG1 (2483_at) | 2483 | custom | 0.108 | alternate8 | predictor6 alternate8 (excluded) |
| LPCAT1 (201818_at) | 79888 | standard | 0.106 | alternate9 | predictor6 alternate9 |
| EBP (10682_at) | 10682 | custom | 0.341 | primary | predictor7 |
| RFC4 (204023_at) | 5984 | standard | 0.264 | alternate1 | predictor7 alternate1 |
| NCAPG (218662_s_at) | 64151 | standard | 0.234 | alternate2 | predictor7 alternate2 |
| RNASEH2A (10535_at) | 10535 | custom | 0.205 | alternate3 | predictor7 alternate3 |
| MED24 (9862_at) | 9862 | custom | 0.191 | alternate4 | predictor7 alternate4 |
| DONSON (29980_at) | 29980 | custom | 0.186 | alternate5 | predictor7 alternate5 |
| RMI1 (80010_at) | 80010 | custom | 0.184 | alternate6 | predictor7 alternate6 |
| PTGES (9536_at) | 9536 | custom | 0.164 | alternate7 | predictor7 alternate7 |
| C19orf60 (51200_at) | 55049 | standard | 0.151 | alternate8 | predictor7 alternate8 |
| ISYNA1 (222240_s_at) | 51477 | standard | 0.135 | alternate9 | predictor7 alternate9 |
| SKP2 (203625_x_at) | 6502 | standard | 0.130 | alternate10 | predictor7 alternate10 |
| DPP3 (218567_x_at) | 10072 | standard | 0.126 | alternate11 | predictor7 alternate11 (excluded) |
| TYMP (204858_s_at) | 1890 | standard | 0.122 | alternate12 | predictor7 alternate12 |
| SNRPA1 (216977_x_at) | 6627 | standard | 0.116 | alternate13 | predictor7 alternate13 |
| DHCR7 (201791_s_at) | 1717 | standard | 0.113 | alternate14 | predictor7 alternate14 |
| TFPT (218996_at) | 29844 | standard | 0.105 | alternate15 | predictor7 alternate15 |
| CTTN (2017_at) | 2017 | custom | 0.102 | alternate16 | predictor7 alternate16 |
| MCM5 (216237_s_at) | 4174 | standard | 0.102 | alternate17 | predictor7 alternate17 |
| TXNIP (10628_at) | 10628 | custom | 0.292 | primary | predictor8 |
| SYNE2 (23224_at) | 23224 | custom | 0.270 | primary | predictor9 |
| SCARB2 (201646_at) | 950 | standard | 0.225 | alternate1 | predictor9 alternate1 |
| PDLIM5 (216804_s_at) | 10611 | standard | 0.167 | alternate2 | predictor9 alternate2 |
| TSC2 (7249_at) | 7249 | custom | 0.145 | alternate3 | predictor9 alternate3 |
| ELF1 (212420_at) | 1997 | standard | 0.119 | alternate4 | predictor9 alternate4 |
| DICER1 (23405_at) | 23405 | custom | 0.209 | primary | predictor10 |
| CALD1 (201616_s_at) | 800 | standard | 0.129 | alternate1 | predictor10 alternate1 |
| SOX9 (6662_at) | 6662 | custom | 0.125 | alternate2 | predictor10 alternate2 |
| FAM20B (202915_s_at) | 9917 | standard | 0.108 | alternate3 | predictor10 alternate3 |
| APH1A (218389_s_at) | 51107 | standard | 0.099 | alternate4 | predictor10 alternate4 |
| AP1AR (55435_at) | 55435 | custom | 0.201 | primary | predictor11 |
| PDCD6 (222380_s_at) | 10016 | standard | 0.154 | alternate1 | predictor11 alternate1 (excluded) |
| PBX2 (202876_s_at) | 5089 | standard | 0.134 | alternate2 | predictor11 alternate2 |
| WASL (205809_s_at) | 8976 | standard | 0.126 | alternate3 | predictor11 alternate3 |
| SLC11A2 (203123_s_at) | 4891 | standard | 0.119 | alternate4 | predictor11 alternate4 |
| KIAA0776 (212634_at) | 23376 | standard | 0.107 | alternate5 | predictor11 alternate5 (excluded) |
| C14orf101 (54916_at) | 54916 | custom | 0.101 | alternate6 | predictor11 alternate6 |
| NUP107 (57122_at) | 57122 | custom | 0.197 | primary | predictor12 |
| FAM38A (202771_at) | 9780 | standard | 0.165 | alternate1 | predictor11 alternate1 |
| PLIN2 (209122_at) | 123 | standard | 0.110 | alternate2 | predictor12 alternate2 |
| AIM1 (212543_at) | 202 | standard | 0.102 | alternate3 | predictor12 alternate3 |
| APOC1 (204416_x_at) | 341 | standard | 0.176 | primary | predictor13 |
| APOE (203382_s_at) | 348 | standard | 0.121 | alternate1 | predictor13 alternate1 |
| DTX4 (23220_at) | 23220 | custom | 0.164 | primary | predictor14 |
| AQP1 (358_at) | 358 | custom | 0.141 | alternate1 | predictor14 alternate1 |
| LMO4 (209205_s_at) | 8543 | standard | 0.120 | alternate2 | predictor14 alternate2 |
| TAF1D (218750_at) | 79101 | standard | 0.159 | primary | predictor15 (excluded) |
| SNORA25 (684959_at) | 684959 | custom | 0.127 | alternate1 | predictor15 alternate1 (excluded) |
| FMOD (202709_at) | 2331 | standard | 0.154 | primary | predictor16 |
| RGS5 (8490_at) | 8490 | custom | 0.120 | alternate1 | predictor16 alternate1 |
| PIK3R1 (212239_at) | 5295 | standard | 0.103 | alternate2 | predictor16 alternate2 |
| MBNL2 (203640_at) | 10150 | standard | 0.100 | alternate3 | predictor16 alternate3 |
| MAPKAPK2 (201461_s_at) | 9261 | standard | 0.151 | primary | predictor17 |
| MTUS1 (212093_s_at) | 57509 | standard | 0.136 | alternate1 | predictor17 alternate1 |
| DHX9 (212107_s_at) | 1660 | standard | 0.136 | alternate2 | predictor17 alternate2 |
| PPIF (201490_s_at) | 10105 | standard | 0.115 | alternate3 | predictor17 alternate3 |
| FOLR1 (211074_at) | 2348 | standard | 0.126 | primary | predictor18 (excluded) |
| KIAA1467 (57613_at) | 57613 | custom | 0.116 | primary | predictor19 (excluded) |
| SUPT4H1 (201483_s_at) | 6827 | standard | 0.111 | primary | predictor20 |
| PHB (200658_s_at) | 5245 | standard | 0.106 | alternate1 | predictor20 alternate1 |
| CD44 (204489_s_at) | 960 | standard | 0.105 | alternate2 | predictor20 alternate2 |

Excluded genes are indicated by the notation “(excluded)” in the last column

TABLE 5
90 probe sets (failed probes excluded) including
all primary and alternate genes (k = 8 clusters)

| Gene (probe set) | CDF | VarImp | predictor group | predictor status |
|---|---|---|---|---|
| CCNB2 (9133_at) | custom | 0.785 | primary | predictor1 |
| MELK (9833_at) | custom | 0.739 | alternate1 | predictor1 alternate1 |
| TOP2A (201291_s_at) | standard | 0.590 | alternate2 | predictor1 alternate2 |
| GINS1 (9837_at) | custom | 0.476 | alternate3 | predictor1 alternate3 |
| MCM2 (4171_at) | custom | 0.428 | alternate4 | predictor1 alternate4 |
| RRM2 (6241_at) | custom | 0.399 | alternate5 | predictor1 alternate5 |
| CDK1 (203213_at) | standard | 0.379 | alternate6 | predictor1 alternate6 |
| UBE2C (202954_at) | standard | 0.365 | alternate7 | predictor1 alternate7 |
| GINS2 (51659_at) | custom | 0.354 | alternate8 | predictor1 alternate8 |
| NCAPG (218662_s_at) | standard | 0.234 | alternate9 | predictor1 alternate9 |
| TMEM97 (212281_s_at) | standard | 0.147 | alternate10 | predictor1 alternate10 |
| CCNB1 (214710_s_at) | standard | 0.140 | alternate11 | predictor1 alternate11 |
| DTL (218585_s_at) | standard | 0.130 | alternate12 | predictor1 alternate12 |
| RACGAP1 (29127_at) | custom | 0.588 | primary | predictor2 |
| TXNIP (10628_at) | custom | 0.292 | alternate1 | predictor2 alternate1 |
| APOC1 (204416_x_at) | standard | 0.176 | alternate2 | predictor2 alternate2 |
| LSM1 (27257_at) | custom | 0.139 | alternate3 | predictor2 alternate3 |
| SCD (200832_s_at) | standard | 0.125 | alternate4 | predictor2 alternate4 |
| HN1 (51155_at) | custom | 0.104 | alternate5 | predictor2 alternate5 |
| CKS2 (1164_at) | custom | 0.515 | primary | predictor3 |
| NUSAP1 (218039_at) | standard | 0.491 | alternate1 | predictor3 alternate1 |
| FEN1 (204767_s_at) | standard | 0.403 | alternate2 | predictor3 alternate2 |
| ZWINT (204026_s_at) | standard | 0.272 | alternate3 | predictor3 alternate3 |
| TYMS (7298_at) | custom | 0.269 | alternate4 | predictor3 alternate4 |
| MLF1IP (218883_s_at) | standard | 0.204 | alternate5 | predictor3 alternate5 |
| NUP107 (57122_at) | custom | 0.197 | alternate6 | predictor3 alternate6 |
| SQLE (209218_at) | standard | 0.174 | alternate7 | predictor3 alternate7 |
| SMC4 (10051_at) | custom | 0.170 | alternate8 | predictor3 alternate8 |
| SLC35E3 (55508_at) | custom | 0.151 | alternate9 | predictor3 alternate9 |
| APOE (203382_s_at) | standard | 0.121 | alternate10 | predictor3 alternate10 |
| SUPT4H1 (201483_s_at) | standard | 0.111 | alternate11 | predictor3 alternate11 |
| PLIN2 (209122_at) | standard | 0.110 | alternate12 | predictor3 alternate12 |
| PHB (200658_s_at) | standard | 0.106 | alternate13 | predictor3 alternate13 |
| AURKA (208079_s_at) | standard | 0.508 | primary | predictor4 |
| PRC1 (9055_at) | custom | 0.499 | alternate1 | predictor4 alternate1 |
| CENPF (207828_s_at) | standard | 0.306 | alternate2 | predictor4 alternate2 |
| ASPM (219918_s_at) | standard | 0.293 | alternate3 | predictor4 alternate3 |
| NEK2 (204641_at) | standard | 0.134 | alternate4 | predictor4 alternate4 |
| DHCR7 (201791_s_at) | standard | 0.113 | alternate5 | predictor4 alternate5 |
| ECT2 (1894_at) | custom | 0.105 | alternate6 | predictor4 alternate6 |
| EBP (10682_at) | custom | 0.341 | primary | predictor5 |
| FADD (8772_at) | custom | 0.313 | alternate1 | predictor5 alternate1 |
| RFC4 (204023_at) | standard | 0.264 | alternate2 | predictor5 alternate2 |
| RNASEH2A (10535_at) | custom | 0.205 | alternate3 | predictor5 alternate3 |
| MED24 (9862_at) | custom | 0.191 | alternate4 | predictor5 alternate4 |
| DONSON (29980_at) | custom | 0.186 | alternate5 | predictor5alternate 5 |
| RMI1 (80010_at) | custom | 0.184 | alternate6 | predictor5 alternate6 |
| PTGES (9536_at) | custom | 0.164 | alternate7 | predictor5 alternate7 |
| DTX4 (23220_at) | custom | 0.164 | alternate8 | predictor5 alternate8 |
| C19orf60 (51200_at) | standard | 0.151 | alternate9 | predictor5 alternate9 |
| TXNRD1 (7296_at) | custom | 0.136 | alternate10 | predictor5 alternate10 |
| ISYNA1 (222240_s_at) | standard | 0.135 | alternate11 | predictor5 alternate11 |
| RAE1 (211318_s_at) | standard | 0.132 | alternate12 | predictor5 alternate12 |
| SKP2 (203625_x_at) | standard | 0.130 | alternate13 | predictor5 alternate13 |
| ACBD3 (202323_s_at) | standard | 0.129 | alternate14 | predictor5 alternate14 |
| ZNF274 (204937_s_at) | standard | 0.122 | alternate15 | predictor5 alternate15 |
| TYMP (204858_s_at) | standard | 0.122 | alternate16 | predictor5 alternate16 |
| SNRPA1 (216977_x_at) | standard | 0.116 | alternate17 | predictor5 alternate17 |
| LPCAT1 (201818_at) | standard | 0.106 | alternate18 | predictor5 alternate18 |
| TFPT (218996_at) | standard | 0.105 | alternate19 | predictor5 alternate19 |
| CTTN (2017_at) | custom | 0.102 | alternate20 | predictor5 alternate20 |
| MCM5 (216237_s_at) | standard | 0.102 | alternate21 | predictor5 alternate21 |
| SYNE2 (23224_at) | custom | 0.270 | primary | predictor6 |
| SCARB2 (201646_at) | standard | 0.225 | alternate1 | predictor6 alternate1 |
| PDLIM5 (216804_s_at) | standard | 0.167 | alternate2 | predictor6 alternate2 |
| TSC2 (7249_at) | custom | 0.145 | alternate3 | predictor6 alternate3 |
| AQP1 (358_at) | custom | 0.141 | alternate4 | predictor6 alternate4 |
| ELF1 (212420_at) | standard | 0.119 | alternate5 | predictor6 alternate5 |
| DICER1 (23405_at) | custom | 0.209 | primary | predictor7 |
| FAM38A (202771_at) | standard | 0.165 | alternate1 | predictor7 alternate1 |
| FMOD (202709_at) | standard | 0.154 | alternate2 | predictor7 alternate2 |
| CALD1 (201616_s_at) | standard | 0.129 | alternate3 | predictor7 alternate3 |
| SOX9 (6662_at) | custom | 0.125 | alternate4 | predictor7 alternate4 |
| RGS5 (8490_at) | custom | 0.120 | alternate5 | predictor7 alternate5 |
| FAM20B (202915_s_at) | standard | 0.108 | alternate6 | predictor7 alternate6 |
| CD44 (204489_s_at) | standard | 0.105 | alternate7 | predictor7 alternate7 |
| PIK3R1 (212239_at) | standard | 0.103 | alternate8 | predictor7 alternate8 |
| AIM1 (212543_at) | standard | 0.102 | alternate9 | predictor7 alternate9 |
| MBNL2 (203640_at) | standard | 0.100 | alternate10 | predictor7 alternate10 |
| APH1A (218389_s_at) | standard | 0.099 | alternate11 | predictor7 alternate11 |
| AP1AR (55435_at) | custom | 0.201 | primary | predictor8 |
| MAPKAPK2 (201461_s_at) | standard | 0.151 | alternate1 | predictor8 alternate1 |
| MTUS1 (212093_s_at) | standard | 0.136 | alternate2 | predictor8 alternate2 |
| DHX9 (212107_s_at) | standard | 0.136 | alternate3 | predictor8 alternate3 |
| PBX2 (202876_s_at) | standard | 0.134 | alternate4 | predictor8 alternate4 |
| WASL (205809_s_at) | standard | 0.126 | alternate5 | predictor8 alternate5 |
| LMO4 (209205_s_at) | standard | 0.120 | alternate6 | predictor8 alternate6 |
| SLC11A2 (203123_s_at) | standard | 0.119 | alternate7 | predictor8 alternate7 |
| PPIF (201490_s_at) | standard | 0.115 | alternate8 | predictor8 alternate8 |
| C14orf101 (54916_at) | custom | 0.101 | alternate9 | predictor8 alternate9 |

TABLE 6

| | | | ER+/LN−/ | | | | |
|---|---|---|---|---|---|---|---|
| | | Total | untreated*/ | Duplicates | ER+/HER− | 10 yr | 10 yr no |
| Study | GSE | samples | outcome | removed | array | relapse | relapse |
| Desmedt_2007¹ | GSE7390 | 198 | 135 | 135 | 116 | 42 | 60 |
| Ivshina_2006² | GSE4922 | 290 | 133 | 2 | 2 | 0 | 2 |
| Loi_2007³ | GSE6532 | 327 | 170 | 43 | 40 | 10 | 5 |
| Miller_2005⁴ | GSE3494 | 251 | 132 | 115 | 100 | 30 | 52 |
| Schmidt_2008⁵ | GSE11121 | 200 | 200** | 200 | 155 | 25 | 46 |
| Sotiriou_2006⁶ | GSE2990 | 189 | 113 | 48 | 45 | 12 | 15 |
| Symmans_2010⁷ | GSE17705 | 298 | 175 | 110 | 102 | 12 | 41 |
| Wang_2005⁸ | GSE2034 | 286 | 209 | 209 | 173 | 67 | 29 |
| Zhang_2009⁹ | GSE12093 | 136 | 136 | 136 | 125 | 15 | 24 |
| 9 studies | | 2175 | 1403 | 998 | 858 | 213 | 274 |

TABLE 7
Comparison of validation results in independent test data for full-gene-set,
17-gene and 8-gene RFRS models

| | | Relapse-Free Survival | | | | | | |
|---|---|---|---|---|---|---|---|---|
| RFRS Performance | | Low risk | | Int risk | | High risk | | |
| Model | AUC | RR | N (%) | RR | N (%) | RR | N (%) | KM (p) |
| Full-gene-set | 0.730 | 6.9 | 78 (30.7) | 15.8 | 133 (52.4) | 26.8 | 43 (16.9) | 6.54E−06 |
| 17-gene | 0.715 | 7.8 | 97 (38.2) | 15.3 | 103 (40.5) | 26.8 | 54 (21.3) | 9.57E−06 |
| 8-gene | 0.690 | 9.7 | 101 (39.8) | 13.9 | 105 (41.3) | 28.3 | 48 (18.9) | 2.84E−05 |

RR, relapse rate

APPENDIX 1
Sample patient data (tab-delimited text file: e.g., patient_data.txt)

| | TOP2A | MAPKAPK2 | SUPT4H1 | FMOD | APOC1 | FEN1 | AURKA | TXNIP | EBP |
|---|---|---|---|---|---|---|---|---|---|
| GSM36893 | 7.0874 | 3.9958 | 7.6561 | 6.7689 | 10.268 | 8.8817 | 6.6811 | 8.3538 | 7.033 |

| | CKS2 | DTX4 | SYNE2 | DICER1 | RACGAP1 | AP1AR | NUP107 | CCNB2 |
|---|---|---|---|---|---|---|---|---|
| GSM36893 | 8.0512 | 6.0171 | 3.2419 | 6.272 | 10.0237 | 6.3404 | 8.9953 | 7.3143 |

APPENDIX 2
RFRS algorithm code

library(randomForest)
#Set working directory and filenames for Input/output
setwd(“C:/path/to/RFRS/”)
#The following files should be in the working dir (except the reportfile which will be created by this program)
datafile=“patient_data.txt”
RelapseProbabilityPlotfile=“RelapseProbabilityPlot.Rdata”
RelapseProbabilityFitfile=“RelapseProbabilityFit.Rdata”
reportfile=“patient_results.pdf”
#Load model file, choose (1) OR (2) and comment out the other (contains “rf_model” object)
RF_model_file=“RF_model_17gene_optimized.Rdata” #1
#RF_model_file=“RF_model_8gene_optimized.Rdata” #2
load(file=RF_model_file)
#Read in data (expecting a tab-delimited file with Gene Symbols as colnames and patient_id as rowname)
patient_data=read.table(datafile, header = TRUE, row.names=1, na.strings = “NA”, sep=“\t”)
#Run test data through forest
RF_predictions_response=predict(rf_model, patient_data, type=“response”)
RF_predictions_prob=predict(rf_model, patient_data, type=“prob”)
RFRS=RF_predictions_prob[,“Relapse”]
#Determine RFRS group according to previously determined thresholds
RF_risk_group=RF_predictions_prob[,“Relapse”]
RF_risk_group[RF_predictions_prob[,“Relapse”]<0.333]=“low”
RF_risk_group[RF_predictions_prob[,“Relapse”]>=0.333 & RF_predictions_prob[,“Relapse”]<0.606]=“int”
RF_risk_group[RF_predictions_prob[,“Relapse”]>=0.606]=“high”
#Load existing relapse probability plot, and loess fit to allow current patient to be plotted
load(file=RelapseProbabilityPlotfile)
load(file=RelapseProbabilityFitfile)
RelapseProb=predict(fit, RFRS)
#Create report
pdf(file=reportfile)
replayPlot(RelapseProbabilityPlot)
points(x=RFRS, y=RelapseProb, pch=18, col=“red”,cex=2)
legend_text=c(paste(“Patient: ”, rownames(patient_data)), paste(“RFRS =”, round(RFRS, digits=4)), paste(“risk
group =”, RF_risk_group),
paste(“Relapse prob. = ”, round(RelapseProb, digits=1), “%”,sep=“”))
legend(x=0.6,y=11,legend=legend_text, bty=“n”,pch=c(18,NA,NA,NA),col=c(“red”,NA,NA,NA),pt.cex=2)
dev.off( )

APPENDIX 3
Probe sequences for top 100 probesets

CCNB2 probes (SEQ ID NO: 1-9)
ATGGAGCTGACTCTCATCGACTATG
ATATGGTGCATTATCATCCTTCTAA
AGTCCTCTGGTCTATCTCATGAAAC
CTTGCCTCCCCACTGATAGGAAGGT
CAAAAGCCGTCAAAGACCTTGCCTC
GATTTTGTACATAGTCCTCTGGTCT
GCCACTACACTTCTTAAGGCGAGCA
GATAGGAAGGTCCTAGGCTGCCGTG
ATCCTTCTAAGGTAGCAGCAGCTGC
TOP2A probes (SEQ ID NO: 10-20)
ACTCCGTAACAGATTCTGGACCAAC
GACCAACCTTCAACTATCTTCTTGA
GAAAGATGAACTCTGCAGGCTAAGA
ACAAGATGAACAAGTCGGACTTCCT
TGGCTCCTAGGAATGCTTGGTGCTG
GATATGATTCGGATCCTGTGAAGGC
AAAGAAAGAGTCCATCAGATTTGTG
GAATAATCAGGCTCGCTTTATCTTA
CTTGGTGCTGAATCTGCTAAACTGA
AAGAACAAGAGCTGGACACATTAAA
GAGACTTTTTTGAACTCAGACTTAA
RACGAP1 probes (SEQ ID NO: 21-25)
GTACAACTCGTATTTATCTCTGATG
GAATGTTTGACTTCGTATTGACCCT
GGATGCTGAAATTTTTCCCATGGAA
ACTTCGTATTGACCCTTATCTGTAA
CAATATATCATCCTTTGGCATCCCA
CKS2 probes (SEQ ID NO: 26-28)
CGCTCTCGTTTCATTTTCTGCAGCG
TATTCTTCTCTTTAGACGACCTCTT
TCTCTTTAGACGACCTCTTCCAAAA
AURKA probes (SEQ ID NO: 29-39)
CTACCTCCATTTAGGGATTTGCTTG
GTGTCTCAGAGCTGTTAAGGGCTTA
CCCTCAATCTAGAACGCTACACAAG
GAGGCCATGTGTCTCAGAGCTGTTA
TTAGGGATTTGCTTGGGATACAGAA
GTGCTCTACCTCCATTTAGGGATTT
AAATAGGAACACGTGCTCTACCTCC
GGGATACAGAAGAGGCCATGTGTCT
GAAGAGGCCATGTGTCTCAGAGCTG
CAGAGCTGTTAAGGGCTTATTTTTT
CATTGGAGTCATAGCATGTGTGTAA
FEN1 probes (SEQ ID NO: 40-50)
GAACTTGCTATGTAATTTGTGTCTA
GATGGTGATGTTCACCTGGCAATCA
GAGCCACCAGGAAGGCGCATCTTAG
TTGACCCACCTTGAGAGAGAGCCAC
GGACACTAAGTCCATTGTTACATGA
GAAATGATTTCCTGGCTGGCCAACT
ACACTGGTTTTCATGCGCTGTTTTT
ACTGATTACTGGCTGTGTCTTGGGT
TGGACCTAGACTGTGCTTTTCTGTC
TTGGGTGGGCAGAAACTCGAACTTG
ACCTGGCAATCAGCTGAGTTGAGAC
EBP probes (SEQ ID NO: 51-71)
GAAGGCACTGCTGGGAGCCATTAGA
CAGGCTCATGGGCAGGCACAAGAAG
GTCTTAGTCGTGACCACATGGCTGT
CACAGATACAAGAGAAGCCAGGAGG
AAGGGGCTGTGTGAAGGCACTGCTG
AGAAGAACTGAGGAGTGGTGGACCA
GCCAGGAGGTCTATGATGGTGACGA
CCCACCTGGCATATACTGGCTGGCC
ACATGGCTGTTGTCAGGTCGTGCTG
TCTATGGGGATGTGCTCTACTTCCT
GCATGGAAACCATCACAGCTTGCCT
GAGTGGTGGACCAGGCTCGAACACT
TTGGAGGGACAAAGCTAATTGATCT
GATGCCAAGGCCACAAAAGCCAAGA
CCAGGCTCGAACACTGGCCGAGGAG
TGACAGAGCACCGCGACGGATTCCA
GGGAGCCATTAGAACACAGATACAA
TTTGTCTTCATGAATGCCCTGTGGC
GGAGACCAAGCCTTCTTATCTCAAC
TGCAGTGTGTGGGTTCATTCACCTG
CTCCGCTTCATTCTACAGCTTGTGG
TXNIP probes (SEQ ID NO: 72-102)
TGTGTCAGAGCACTGAGCTCCACCC
TACAAGTTCGGCTTTGAGCTTCCTC
AAAGGATGCGGACTCATCCTCAGCC
ACTTTGTTCACTGTCCTGTGTCAGA
GAAAGGGTTGCTGCTGTCAGCCTTG
AGATAGGGATATTGGCCCCTCACTG
GGCAATCTCCTGGGCCTTAAAGGAT
CTTAGCCTCTGACTTCCTAATGTAG
GCAAAGGGGTTTCCTCGATTTGGAG
AAATGGCCTCCTGGCGTAAGCTTTT
AAACCAACTCAGTTCCATCATGGTG
TTCCACCGTCATTTCTAACTCTTAA
GGTTTTCTCTTCATGTAAGTCCTTG
CGGAGTACCTGCGCTATGAAGACAC
CCCTGCATCCTCAACAACAATGTGC
GTGTTCTCCTACTGCAAATATTTTC
AATTGAGGCCTTTTCGATAGTTTCG
GGAGGTGGTCAGCAGGCAATCTCCT
CCAGCGCCCATGTTGTGATACAGGG
GAAAAACTCAGGCCCATCCATTTTC
TGAGGTGGTCTTTAACGACCCTGAA
TGTTCTTAGCACTTTAATTCCTGTC
AGCTCCACCCTTTTCTGAGAGTTAT
CACTCTCAGCCATAGCACTTTGTTC
GAAGCAGCTTTACCTACTTGTTTCT
GAAGTTACTCGTGTCAAAGCCGTTA
GGTGGATGTCAATACCCCTGATTTA
CCGAGCCAGCCAACTCAAGAGACAA
TGGATGCAGGGATCCCAGCAGTGCA
GATCCTGGCTTGCGGAGTGGCTAAA
GCTGAAACTGGTCTACTGTGTCTCT
SYNE2 probes (SEQ ID NO: 103-113)
TTTCTAAGACTTTTTCACATCCAAA
GTTTTACTCCAATCAGCTGGCAATT
GGCACCCTTAGCTGATGGAAACAAT
ATTTTGAGCTGCCGGTTATACACCA
TGTTCTGTTCAGTACCTAGCTCTGC
GTAAATGCCAAACTACCGACTTGAT
TACGCTTAGAATCAGTTTTACTCCA
GTTCAGAAACTCATAGGCACCCTTA
TGAGCAGTGGTGTCCATCACATATA
ATGTACAACTCAGATGTTTCTCATT
GCTCTGCTCTTTTATATTGCTTTAA
DICER1 probes (SEQ ID NO: 114-142)
AATTTCTTACTATACTTTTCATAAT
ATTTCACCTACCAAAGCTGTGCTGT
ACTAGCTCATTATTTCCATCTTTGG
AAATGATTTTTCACAACTAACTTGT
TTGCAGTCTGCACCTTATGGATCAC
TGATACATCTGTGATTTAGGTCATT
GGAGACGCCAATAGCAATATCTAGG
CTGATGCCACATAGTCTTGCATAAA
AGCTGTGCTGTTAATGCCGTGAAAG
GAAGTGCGCCAATGTTGTCTTTTCT
GTGAAACCTTCATGGATAGTCTTTA
TTTACTAAAGTCCTCCTGCCAGGTA
GGACATCAACCACAGACAATTTAAA
TGTTGCATGCATATTTCACCTACCA
ATAAACCTTAGACATATCACACCTA
TAGTCTTTAATCTCTGATCTTTTTG
GAGACAGCGTGATACTTACAACTCA
GACCATTGTATTTTCCACTAGCAGT
CTGCAGCAGCAGGTTACATAGCAAA
GCCGTGAAAGTTTAACGTTTGCGAT
AACTGCCGTAATTTTGATACATCTG
TATTTACCATCACATGCTGCAGCTG
AACGTTTGCGATAAACTGCCGTAAT
GGAAATTTGCATTGAGACCATTGTA
GCACCTTATGGATCACAATTACCTT
AGAAGCAAAACACAGCACCTTTACC
CCCTTAGTCTCCTCACATAAATTTC
TGTGTAAGGTGATGTTCCCGGTCGC
CTGCCAGGTAGTTCCCACTGATGGA
AP1AR probes (SEQ ID NO: 143-153)
GCCTTCCTTTACCTTGTAGTACAAG
TTTTTCCTCTTGCAACAATGACGGT
GTCAATTTACAAGGCCAGGGATAGA
TTCCACTTCATTTTACATGCCACTA
GTGCTAGACAATTACTGTTCTTTTC
AATATCTATAACTGCATTTTGTGCT
GATAGAAAACACTCCATAATTGCTT
CATTGATTTTATTAAGCCTTCCTTT
TACATGCCACTATATTGACTTTAAT
TCTGGTATGAAAGGCTCCATTGATT
GCTTTCCTTGATTTTGCTGAGGATT
NUP107 probes (SEQ ID NO: 154-163)
GGATATCAGCGTTTCTCTGTGTGCT
GAAAGCTTTGTCTGCCAATGTTGTG
CAGAGAGTCCTCTCTAATGCTCCTA
GATATTGCACAGTACTGGTCAGTAT
GACCAGGGACTTGACCCATTAGGGT
AGATATGGTATCCTCTGAGCGCCAC
AATGCTCCTAGACCAGGGACTTGAC
ATCGTGACACTTTCAACATGTAGGG
TTGGATGCCCTAACTGCTGATGTGA
GTGTTTTCTGCTTCATACGATATTG
APOC1 probes (SEQ ID NO: 164-174)
AAGGGTGACATCCAGGAGGGGCCTC
CAGGAGGGGCCTCTGAAATTTCCCA
GATGCGGGAGTGGTTTTCAGAGACA
CAGCAAGGATTCAGGAGTGCCCCTC
GTGAACTTTCTGCCAAGATGCGGGA
CAAGGCTCGGGAACTCATCAGCCGC
AACACACTGGAGGACAAGGCTCGGG
GACGTCTCCAGTGCCTTGGATAAGC
CCAAGCCCTCCAGCAAGGATTCAGG
TCATCAGCCGCATCAAACAGAGTGA
GTTCTGTCGATCGTCTTGGAAGGCC
DTX4 probes (SEQ ID NO: 175-180)
ATCGCCACCTGGTGCTCATGAGGTG
ACTCGTCTTGGTATTGCACTGTTGT
ATTCTCTTCCCATTTTTGTACATTT
TGCTCCGTGAAAGGACATCGCCACC
GGAGACAAACCTCGTCAGATGCTCA
TGAAGTCTTTGGTGTTGCTCCGTGA
TAF1D probes (SEQ ID NO: 181-191)
TGATTGTTGCCATGTGAGAGTTTTA
ACTCCTAATGTTTGGTGCTATGTTT
GTATGGGTCATTTCAAAGAGGGCTT
TGGTGCTATGTTTTCCTGAGGAGAT
AAGTTTCTCTAGTGTTTTCTGTGGA
GTATTTTTGGCTCGAAGTTTCTCTA
GAAGCCATAGCACTCCTAATGTTTG
AAGAGGGCTTATGAGGCTGTGAAAC
CCCAGAGCTCTTAACGCTGTGACCA
GAGGCTGTGAAACCCAGAGCTCTTA
ATTTCTCTTCTTCAGGGCAAACTTG
FMOD probes (SEQ ID NO: 192-202)
GCTGGGGAGCACTTAATTCTTCCCA
GGAGCTCCGATGTGAGGGGCAAGGC
TCTGGCTGGGGTCCGTGAAGCCCAG
GCCAAACCAGCTCATTTCAACAAAG
ATGTGAACACCATCATGCCTTTATA
TGCCATCACATCCCTGATACTGTGT
TTTGGACTACGTTCTTGGCTCCAGA
GCAGCCAAATCTTGCCTGTGCTGGG
GCTTTGAAGCACCTTCCCTGAGAAG
TCTGCTTTCACATCTCTGAGCTATA
TAATGTTGCCTGGGGCTTAACCCAC
MAPKAPK2 probes (SEQ ID NO: 203-213)
GCTGAAGAGGCGGAAGAAAGCTCGG
CTCCTGCCCACGGGAGGACAAGCAA
CCTGCCCACGGGAGGACAAGCAATA
GGACAAGCAATAACTCTCTACAGGA
AACTCTCTACAGGAATATATTTTTT
GTTGACTACGAGCAGATCAAGATAA
AATGCGCGTTGACTACGAGCAGATC
CACAATGCGCGTTGACTACGAGCAG
GCGCGTTGACTACGAGCAGATCAAG
AAGCAATAACTCTCTACAGGAATAT
AGACAGAACTGTCCACATCTGCCTC
FOLR1 probes (SEQ ID NO: 214-224)
AATCTTTGAGACAAGCATATGCTAC
CGGCCGTGCGTACTTAGACATGCAT
CCATTCGCAGTTTCACTGTACCGGC
GTGCGTACTTAGACATGCATGGCTT
GGAGCGAGCGACCAAAGGAACCATA
GCATATGCTACTGGCAGGATCAACC
AACCATAACTGATTTAATGAGCCAT
GACATGCATGGCTTAATCTTTGAGA
GAGCGACCAAAGGAACCATAACTGA
CAAGTAGGAGAGGAGCGAGCGACCA
AATGAGCCATTCGCAGTTTCACTGT
KIAA1467 probes (SEQ ID NO: 225-235)
TCTCTAATCCCATCCTGAGGTTGCC
GGAAGCTTCATCTGACCAATGTGGG
AAATGCAAGGGTCTTACCCTCCTCT
CCACCCACCCAGGTGTCTAAGATAG
GCAAAGCCAATATGACCACTACTGA
ATCCCCTGAATGTGAATTGCTATCC
AGATAGGACATGCTCCTTTCTTTCT
TTGCTATCCTTATTGCCCTATTAAA
TGGTATGGTGAAACTAATCCCCTGA
TTGCCATCCCCCAAATGTGTGGTAT
CTTGTGAAATGTGTCCCTAAGCCTC
SUPT4H1 probes (SEQ ID NO: 236-246)
TACCCTCCAATTCAGACTCAGCTGA
CAGAACTTCAAATACTTCCTACCCT
CCTGCCCCAAGGAATCGTGCGGGAG
GACAGCTGGGTCTCCAAGTGGCAGC
ATCTTCTTTGGACTACAGGTGGGGT
TAGGATGCTGATTTTCCTACCCGTG
GTATATGACTGCACTAGCTCTTCCT
GAGAGCAGCACATCATTTTATCATT
GTCGAGGAGTGGCCTACAAATCCAG
TGCAAGGCTGCCAGCATCTTTGCTC
ATATGCGGTGTCAGTCACTGGTCGC
MELK probes (SEQ ID NO: 247-257)
AAGACTGTTATGATCGCTTTGATTT
GCCCATCTGTCATTATGTTACTGTC
AGGGCGATGCCTGGGTTTACAAAAG
AGCTCTTAACTATGTCTCTTTGTAA
GATTCTTCCATCCTGCCGGATGAGT
GAATCTAAATCAAGCCCATCTGTCA
GAGCTATCTTAAGACCAATATCTCT
GGAAGACATCCTATCTAGCTGCAAG
GTGTGGGTGTGATACAGCCTACATA
ATGTGGTGGGTATCAGGAGGCAGCG
GGAGGCAGCGGCTTAAGGGCGATGC
MCM2 probes (SEQ ID NO: 258-268)
TTGTGCTTCTCACCTTTGGGTGGGA
GGATGCCTGCGTGTGGTTTAGGTGT
TAGCAGGATGTCTGGCTGCACCTGG
TCTCCACTCAGTACCTTGGATCAGA
GAGTCATGCGGATTATCCACTCGCC
CTGGCATGACTGTTTGTTTCTCCAA
CCCCACTCTCTTATTTGTGCATTCG
AGCACTTGATGAACTCGGGGTACTA
GCCAGTGTGTCTTACTTGGTTGCTG
CCCTCTTGGCGTGAGTTGCGTATTC
TTGGTTGCTGAACATCTTGCCACCT
LSM1 probes (SEQ ID NO: 269-278)
GAAGGACCGAGGTCTTTCCATTCCT
GAGTACTAATCTTTTGCCCAGAGGC
AGTGAAAGTGACATCCTGGCCACCT
ACAGTGGCATAGACTCCTTCACACA
ACAGGGACAGTCTTCATTTACTTGT
TCCATTCCTCGAGCAGATACTCTTG
GCACCAGCAACTACTTCTTTATATT
AAAAGGAGAGTGACACACCCCTCCA
CACCTCACGCATTTGATCACAGACT
CCTTCACACATCACTGTGGCACCAG
NUSAP1 probes (SEQ ID NO: 279-289)
CCTTCACCTCAGTGGAGCTTCTGAG
GGCTTTGCTTAGTATCATGTCCATG
TGTACCTTCGTTCAAATATCCTCAT
CATCTGTCACTCACTATATTCACAA
GTTTTATACTGCTCAAGATCGTCAT
GGGATAGAAAGGCCACCTCTTCACT
AACTGCAGTCTTCTGCTAGCCAATA
ACTCATTCTAACATTGCTTACTTAA
CACCTCTTCACTCTCTATAGAATAT
GCTACATAGCCCTATCGAAATGCGA
TCCTCATGTAATTGCCATCTGTCAC
PRC1 probes (SEQ ID NO: 290-300)
TTGCACATGTCACTACTGGGGAGGT
CCTCTCAATCACTACTCTTCTTGAA
GTTCTCAAAAGCTTACCAGTGTGGA
GTGTTCAGTTCTGTTACACAGTGCA
GAGCTGTCTTTGTCGTGGAGATCTG
ACACAGTGCATTGCCCTTTGTTGGG
ACACATGCTTGTCGGAACGCTTTCT
ACTTGGTGTTAGCCACGCTGTTTAC
GTGTCCGAAGTTGAGATGGCCTGCC
GGGAGTCTGTTTGTTCCAATGGGTT
GGAGATCTGGAACTTTGCACATGTC
FADD probes (SEQ ID NO: 301-311)
GATGAGCAGTCACACTGTTACTCCA
GCACTCTCTAAATCTTCCTTGTGAG
GGATTATGGGTCCTGCAATTCTACA
GAAAGGATGTTTTGTCCCATTTCCT
AATTGCCAAGGCAGCGGGATCTCGT
TCCTCTCTGAGACTGCTAAGTAGGG
TGCTCAACCACTGTGGCGTTCTGCT
TGATTGACACACAGCACTCTCTAAA
CTGGACACTAGGGTCAGGCGGGGTG
AGAGGCCCAGGAATCGGAGCGAAGC
GGGGCAGTGATGGTTGCCAGGACGA
RFC4 probes (SEQ ID NO: 312-322)
TCATGCAGCAACTCAGCTCGTCAAT
ATGTTCAAAATTCCGCTTCAAGCCT
AAAGCGCTACTCGATTAACAGGTGG
ACTCATCAGCCTTTGTGCAACTGTG
GAACATTTGCAACTCATCAGCCTTT
TCAACAGCAGCGATTACTAGACATT
ACCCCTGACCTCTAGATGTTCAAAA
GTGATGCAGCAGTTATCTCAGAATT
GATGGAGTATTTGCTGCCTGTCAGA
AAGCCATTACATTTCTTCAAAGCGC
TCAGCTCGTCAATCAACTCCATGAT
SCARB2 probes (SEQ ID NO: 323-333)
GTGACAATCATTTTGCTGACAGAAT
AAGGGCATTTTCTTTGATTCTCAAA
GGAGCCATCATATGTCACAGTGTTC
AGAGAAACGTGTGCCCTATACTTCC
GAAATCCATCTATCTACAGCCTAAG
TAGCTCACTGTCACTCACTGAATAG
GAGACACCACTTTTCAAAGGACTTC
AGTTCTTTCCAGTGTTTTGTAGCTC
GGACTTCTTGGTTTCAGCATAACCT
GAGAAGCCTATACATTTAGCTGACA
TGCCCTATACTTCCTGTGACAATCA
CALD1 probes (SEQ ID NO: 334-344)
CTTCCCCCACTAAGGTTTGAGACAG
GACGCAGGACGAGCTCAGTTGTAGA
GACGTATCCAGCAAGCGGAACCTCT
TTCAATATCCCAGTAAACCCATGTA
AGCAGTGATACCAACCACATCTGAA
CTTGAGACCAGGAGACGTATCCAGC
ACTGATCATCATAACTCTGTATCTG
GAACCCAAGCTCAAGACGCAGGACG
GCAAGCGGAACCTCTGGGAAAAGCA
GCGGAATGTGTGCAGTATCTAGAAA
TCTGTGGATAAGGTCACTTCCCCCA
PDCD6 probes (SEQ ID NO: 345-355)
GGTTGGTGCAGCAGTCATTAAAAGT
GAGTCAAGGCCAGACTAGATCAGCC
TTCTCATGGAGCTTCCTTTCTAGAG
CAAAGGGGCGTGTCATGTGCCTCAT
CAAGGCCAGACTAGATCAGCCTAAG
CATGGAGCTTCCTTTCTAGAGGGGA
CTCTATTCTCATGGAGCTTCCTTTC
ATTTGAGTAGATTTGGCCTCTATTC
GACTTTCAAAGGGGCGTGTCATGTG
TTGGCCTCTATTCTCATGGAGCTTC
GATTCTAATAGGTTGGTGCAGCAGT
FAM38A probes (SEQ ID NO: 356-366)
GCTACGGCATCATGGGGCTGTACGT
ATCATGGGGCTGTACGTGTCCATCG
CATTATGTTCGAGGAGCTGCCGTGC
GCTGGCGCCCGAGAGGGAAGGAGCC
GCTGGTCATCGGCAAGTTCGTGCGC
GAGGAGTTGTACGCCAAGCTCATCT
CGCTCACCGGAGACCATGATCAAGT
GCGGATTCTTCAGCGAGATCTCGCA
TTCGTGCGCGGATTCTTCAGCGAGA
TCCCCCACGTGTACTGTAGAGTTTT
AGATCTCGCACTCCATTATGTTCGA
APOE probes (SEQ ID NO: 367-377)
GGCCCCTGGTGGAACAGGGCCGCGT
TGGTGGAAGACATGCAGCGCCAGTG
GAAGCGCCTGGCAGTGTACCAGGCC
AGCAGGCCCAGCAGATACGCCTGCA
GTGCCCAGCGACAATCACTGAACGC
TGGGGCCCCTGGTGGAACAGGGCCG
AAGCGCCTGGCAGTGTACCAGGCCG
GCCCAGCGACAATCACTGAACGCCG
GCGCGCGCGGATGGAGGAGATGGGC
GCGACAATCACTGAACGCCGAAGCC
CCCTGGTGGAACAGGGCCGCGTGCG
AQP1 probes (SEQ ID NO: 378-399)
CATAAGTCCTTTCAATTCCACCAGG
GCTAGACAATGATTTGGCCAGGCCT
CAGTGCATCACATCTGCACACTCTC
CTGACCTTGGAATCGTCCCTATATC
TGGAATCGTCCCTATATCAGGGCCT
GCAGCCCCTAAGTGCAAACACAGCA
TCTGCATATATGTCTCTTTGGAGTT
GAAGGCTGGATTCTATCTACATAAG
GCCCTTAACTATCACCAGTGCATCA
CACCACTGTGCACTTAGCCATGATG
ACCACGAGGCTGATTCCTCTCATTT
TGCAAAGTGGCAGGGACCGGCAGAG
GCAAACACAGCATGGGTCCAGAAGA
GCATATATGTCTCTTTGGAGTTGGA
AGACGTGGTCTAGACCAGGGCTGCT
ACTTACTGCCTGACCTTGGAATCGT
GGCCTAGTAACCAAGGCCCTGTCTC
GCATGGGTCCAGAAGACGTGGTCTA
GCATCTGTCTGCTCTGCATATATGT
TCTCAGTTTCTGCCTGGGCAATGGC
TTACTGCCTGACCTTGGAATCGTCC
GCAGGAACTTCTAGCTCATTTAACA
SNORA25 probes (SEQ ID NO: 400-405)
ACTCCTAATGTTTGGTGCTATGTTT
TGGTGCTATGTTTTCCTGAGGAGAT
GAAGCCATAGCACTCCTAATGTTTG
AAGAGGGCTTATGAGGCTGTGAAAC
CCCAGAGCTCTTAACGCTGTGACCA
GAGGCTGTGAAACCCAGAGCTCTTA
RGS5 probes (SEQ ID NO: 406-438)
TGCTCCATTGGAGTAGTCTCCCACC
GGTAGAGGCCTTCTAGGTGAGACAC
TACTTATCTACTGTCCGAAGGCCTT
CCTGCATTTCCCATTAATCTACATA
AATGCTGAGAAATTTGCCACTGGAG
TATACAGTTTAATAAGCCTCTTGCA
ATTTAAAATATTGATCCTTCCCTTG
ATCTCACTTGTTTTAGTTCTGATCC
ATTTGGGTCCAACTTCAATAATGTA
GACTGTGGGTCAAATGTTTCCATTT
AAATGAAACTGTTGCTCCATTGGAG
GTATCTGTAACCACAATCACACATA
GGACCACCTTCATGTTAGTTGGGTA
TTGCAAGTTACTTGTTCTCTCACCT
CTTTTTGCCCACACTGCTTTGGATA
AGATCACCCCTCTAATTATTTCTGA
TATTTCCTCCATAATAACCCTGCAT
GGGATGTTGCTTACTCTTTTTGCCC
GTACTATGTGACTCATGCTTCTGGA
GTTCTCTCACCTGAGGTATTTTTTT
GCCACTGGAGACAAGCAATCTGAAT
TCATCCTGTGAGTTATTTCCTCCAT
TGCAACTAGCAACTCATCTTCGGAA
CTGCCCATAGTCACCAAATTCTGTT
TGGAAAAGGATTCTCTGCCTCGCTT
GCTAATTGTCCTATGATGCTATTAT
TTCCTCTTCTCCCTTTGCAAGAGGA
ATGACATTTATCTTCAAAACACCAA
GAGTAGTCTCCCACCTAAATATCAA
TTCCCACAGCAGCTTTGCTCAGTGA
CTCGCTTTGTGCGCTCTGAGTTTTA
ATCCATTTGTAAGCATTTATCCCAT
ATGTATTTATGCTGCTAGACTGTGG
MTUS1 probes (SEQ ID NO: 439-449)
TCTTCACCACAGACACCTTCTTGTG
GAGCCTAACACTATCCTGTAATTCA
GTCCCTGTCTATACATTCTCTGTAT
TAACCTTTGTAATGTTCTTCACCAC
ACTCTGCTCAGCCCTGTAACAGGGT
TTTTACTTACCCATGTGAGCCTAAC
TTCATTGCCTTTTTCACCTAAGCAT
TTCTCTGTATCTTTTGGGGGTAACT
AGGAAGAGCTTTGACTTGTCCCTGT
GTTTTTCAGTGTTCAGCCATGTCAG
ATTATGATCATCTACCACCAACTCT
PHB probes (SEQ ID NO: 450-460)
GCAGGGGATGGCCTGATCGAGCTGC
TGAGCGACGACCTTACAGAGCGAGC
GACCTTCGGGAAGGAGTTCACAGAA
GAGTTCACAGAAGCGGTGGAAGCCA
CAGCCCCGATGATTCTTAACACAGC
GCAGGTGAGCGACGACCTTACAGAG
CAGGGGATGGCCTGATCGAGCTGCG
GAGCAACAGAAAAAGGCGGCCATCA
TCCTGGATGACGTGTCCTTGACACA
TCGGGAAGGAGTTCACAGAAGCGGT
TGGATGACGTGTCCTTGACACATCT
GINS1 probes (SEQ ID NO: 461-470)
TGTTGAACTTGTATCCTTCAGCCTT
TAATATTGAGTCTTCTGGCCTATAA
GGTCTGTCTTCCTAGGTATTAATGT
AGTTTTCAGTGTACAGGTCTACCAT
GCCTTGCTAAACTGTGAGTTCTCAT
GGCCTATAAACAAGGTCTGTCTTCC
GTAGTCACAGTTACACGGCAGGCTG
GTTGGGCACCTTGATTGAGATTGCA
AATTCTAACCACTTGTTGCTAGTAA
AGGTCTACCATGTCAGCATTTCATA
KIAA0101 probes (SEQ ID NO: 471-490)
AATGGTGCCATATTGTCACTCCTTC
ACCAGCCCAGGCAACATAGCGTAAA
GTGTTTGTTCCAATTAGCTTTGTTG
TAGGTTGTCCCCTAAAGATTCTGAA
TGCTTAGATTGTTGTACTGCTGCCA
TTAAACGGTTGATAATGCCTCTACA
TATTCTACCCTCTTTTTTGGCAAGG
CAAGTCATTGCATTGTGTTCTAATT
CATAGCGTAAACCCTATCTCTAAAA
AACCTTGGATGGATATCTTCTCTTT
ATTGTTGTACTGCTGCCATTTTTAT
CACAGTGGCTTCTCAGGAGGCTGAG
GGATAGAATCATGGTGGGCACAGTG
TCTCCTTGTTTACCCTGGTATTCTA
AAGTGTCTAGTTCTTGCTAAAATCA
TGGAGAATTCTTTAGGTTGTCCCCT
GGAGGGAGGTTTGCTTGAGTCCAGG
TGGCAAGGAGGACAAATACGCAATG
TCATCTTTGAATAACGTCTCCTTGT
GATAATGCCTCTACAACAACAAGAA
SCD probes (SEQ ID NO: 491-501)
TGAACTTGATACGTCCGTGTGTCCC
GGGCAGTTTTGAGGCATGACTAATG
AAAAGCGAGGTGGCCATGTTATGCT
TAACTATAAGGTGCCTCAGTTTTCC
AGATGCTGTCATTAGTCTATATGGT
GGAATTCTCAAGACCTGAGTATTTT
CTGACCTACCTCAAAGGGCAGTTTT
ACAACGCATTGCCACGGAAACATAC
AGCATTTTGGGATCCTTCAGCACAG
GAAGCTAATTGTACTAATCTGAGAT
ATGTCCACCATGAACTTGATACGTC
PTTG1 probes (SEQ ID NO: 502-512)
CATTCTGTCGACCCTGGATGTTGAA
TTGAGAGTTTTGACCTGCCTGAAGA
AATTGCCACCTGTTTGCTGTGACAT
GTGCCTCTCATGATCCTTGACGAGG
TGCAGTCTCCTTCAAGCATTCTGTC
CCTGCCTCAGATGATGCCTATCCAG
AAAACAGCCAAGCTTTTCTGCCAAA
GGGAATCCAATCTGTTGCAGTCTCC
TGAAGAGCACCAGATTGCGCACCTC
AAGCAAAAAGCTCTGTTCCTGCCTC
TTCCCTTCAATCCTCTAGACTTTGA
CENPF probes (SEQ ID NO: 513-523)
GGTCAAAGTTGCTCAGCGGAGCCCA
TGCACAGAAGTTAGCGCTATCCCCA
TACCCCTGGGAGGTGCCAGTCATTG
GTTTGGAAGCACTGATCACCTGTTA
GAAGGCACTTTGTGTGTCAGTACCC
GATCACCTGTTAGCATTGCCATTCC
GAGCCCAGTAGATTCAGGCACCATC
GTACTCTTTAGATCTCCCATGTGTA
TGAGGGTCAAGCGAGGCCGACTTGT
TTGCCATTCCTCTACTGCAATGTAA
CGAAATCCGTCCCAGTCAATAATCT
SMC4 probes (SEQ ID NO: 524-544)
GGACAGTGTTTCAACAAGCCTAGGC
GCATCTAAGGGACTTTGTTGAACTT
GATGGCCTCTGATTTACACTGGTTC
AGAAGTCTGCCCTAGCTGTTAAATT
GAGTTAATTGTTCCTTTCTTCAGTG
TAGACAGCTTGGATCCTTTCTCTGA
GGTTTACCAGGATGTAGTCCCACTG
GAAAACACTTAGTTCATTGGCTTTA
GCGTATTTTTACACTATTGGCTCAA
ATTTACACAGCTAGATTTGGAAGAT
GGATGAGATTGATGCAGCCCTTGAT
ATTGATGCAGCCCTTGATTTTAAAA
AGTTCATAATAATTTCTCTTCGAAA
GGAAGGACTTTCGGTATTGTATTAG
CCTTTCTTCAGTGGGCCATTGTTTT
TTAGTATTTGCTCTTCACCACTACA
GATACCTTGAGTAATGTTTGCCTAT
CACTCCCCTTTACTTCATGGATGAG
AAGCCTAGGCTATCTCGTAAGTTGA
GGACGCCGAACTCGAGCTTGTAGAC
AATATCCCACTATAGTTGCTTCATG
NCAPG probes (SEQ ID NO: 545-555)
CCCAATTTCTCAATGAAGATCTAAG
GATTATGTCCAGTTATTTGCTTTAA
GGTGGAATCCTTTAAGATTATGTCC
AAGACGATGGAGGTGGAATCCTTTA
GAGCCAAAACCGCAGCACTAGAAAA
GAGACTACCAAGACGAGCCAAAACC
TTCCAGAACCAGAATCAGAAATGAA
CCAAGACGAGCCAAAACCGCAGCAC
GGACGAACAGGAGGTGTCAGACTGC
GAAGATGAGACTACCAAGACGAGCC
GTGTCAGACTGCTGAAGCCGACTCT
PDLIM5 probes (SEQ ID NO: 556-566)
CTTGCTTTGTATGCTCAGTGTGTTG
GCCCTCTTTGGTACTATATGCCATG
ACACCTGGCATGACACTTGCTTTGT
GAATTTCCCATAGAAGCTGGTGACA
GAAGCTGGTGACATGTTCCTGGAAG
CAAGAAGGACAAGCCCCTGTGTAAG
TGACATGTTCCTGGAAGCTCTGGGC
ATATGCCATGGATGTGAATTTCCCA
GCCCCTGTGTAAGAAACATGCTCAT
GGAAGGTCAGACCTTTTTCTCCAAG
TTATTATGCCCTCTTTGGTACTATA
SOX9 probes (SEQ ID NO: 567-588)
GAGAGGACCAACCAGAATTCCCTTT
AAGCATGTGTCATCCATATTTCTCT
CTACCTGGAGGGGATCAGCCCACTG
AGTTGAACAGTGTGCCCTAGCTTTT
GGAGAATCGTGTGATCAGTGTGCTA
GTAGTGTATCACTGAGTCATTTGCA
TGGGCTGCCTTATATTGTGTGTGTG
TGTTTTCTGCCACAGACCTTTGGGC
TGTTCTCTCCGTGAAACTTACCTTT
AAATGCTCTTATTTTTCCAACAGCT
CCTAGCTTTTCTTGCAACCAGAGTA
GAATTCCCTTTGGACATTTGTGTTT
GCCAACCTTGGCTAAATGGAGCAGC
ATTACTGCTGTGGCTAGAGAGTTTG
TTGGAGTGAGGGAGGCTACCTGGAG
ATATGGCATCCTTCAATTTCTGTAT
CAGCCCACTGACAGACCTTAATCTT
ATCAGTGGCCAGGCCAACCTTGGCT
TTTTCCAACAGCTAAACTACTCTTA
GCAACTCGTACCCAAATTTCCAAGA
ACATGACCTATCCAAGCGCATTACC
GTAAAAGCTTTGGTTTGTGTTCGTG
PBX2 probes (SEQ ID NO: 589-599)
TAGTTCTCTCCTCACTTGTAAACTT
GTATATGTATCTTCCTCAATTTCCC
GGAGGCAGTGAAGGGCTTGCCCTGC
CATCTTCCCCTGTGAGTGACATGTC
AGGTTGGAAGTGTGATGGGTGGGGG
GGTATCTTTTTGTCACACCAAAATC
CCCCTCCCATTAAAGATCCGGGCAG
AAAGTAACATCAACACTGTCCCATC
GATCCCCTCAGACATTCTCAGGATT
GACTGTCAGAGTGGGGAACCCCTCC
GGGTTGGGGTGCTTGTATATGTATC
PLIN2 probes (SEQ ID NO: 600-610)
TATGTTCTCATTCTATGGCCATTGT
GAGTCTCAGAATGCTCAGGACCAAG
TGTGGCCAGACAGATGACACCTTTT
GTCTGCTCTGGTGTGATCTGAAAAG
GCTTTATCTCATGATGCTTGCTTGT
GGGGTAGAAACTGGTGTCTGCTCTG
CAGGAGACCCAGCGATCTGAGCATA
GAAAAGGCGTCTTCACTGCTTTATC
TATGGCCATTGTGTTGCCTCTGTTA
ATCACTAGTGCATGCTGTGGCCAGA
AACATCTTCATGTGGGCTGGGGTAG
LMO4 probes (SEQ ID NO: 611-621)
CCCTTCCCGCATTTATTGGTGTATT
ACCTTTGTAGCTAGCACCAGTGCCA
TTCATCTCAGATTTGTTCATCACAG
GTCTTCAGTAGACAAGTCACCTTTG
TTAAGGACTCCATGAACCTGGGCTA
TAATGTTGCTACTCCCATGGCAAAG
GTTTTTGTCCTAATGTTGCTACTCC
CAGAGGACATCTTGGGGAGGGGGAG
CACCTTCTTTAGTCTTGATTGCCCT
CCATTGCACCTTCTTTAGTCTTGAT
GATGTGGCTTTTGTGATATTCTATC
PIK3R1 probes (SEQ ID NO: 622-632)
CACGGTCAGTTGTAACTTTGCCTTC
GACTATCCAACTTAACATGAAACTT
GAGATAGCATTAGCTGCCCAGGATG
AATGGAGCTATGTCTTGTTTTAAGT
GAGAGGGAGGATGTCACGGTCAGTT
AGTTGGTCTTTTGACGAGAGGGAGG
GTGCCTCCTTGACATTTCGTTCAAG
GAAACTTGTCACCATGAGATAGCAT
AAAGCTACAATCTGTTCAATGTTTT
CTGCCCAGGATGCTGCTATATATAT
AAAAACTCATTTATACCTGTGTATT
DHX9 probes (SEQ ID NO: 633-643)
TGACCGAGCAGCAGAGTGTAACATC
GTAACATCGTAGTAACTCAGCCCAG
ATCAGTGCGGTTTCTGTGGCAGAGC
GACTTTATCCAGAATGACCGAGCAG
TAGAGGGGCTACTGGATGTGGGAAA
CTCAGCCCAGAAGAATCAGTGCGGT
GGCTTATCCTGAAGTTCGCATTGTT
TGGGAAAACCACACAGGTTCCCCAG
TGTACTGTAGGTGTGCTCCTGAGAA
GCGTGATGTTGTTCAGGCTTATCCT
GGAGGACTTACCCAGTTCAAGAATA
CD44 probes (SEQ ID NO: 644-654)
GGATGGCTTCTAACAAAAACTACAC
GTGTGCTATGGATGGCTTCTAACAA
TAGTTACACATCTTCAACAGACCCC
AGGGTGAAGCTATTTATCTGTAGTA
TTAGGGCCCAATTAATAATCAGCAA
CTTCCATAGCCTAATCCCTGGGCAT
CACATATGTATTCCTGATCGCCAAC
CAGACCCCCTCTAGAAATTTTTCAG
TTGAATGGGTCCATTTTGCCCTTCC
CAGGGTTAATAGGGCCTGGTCCCTG
TTAAACCCTGGATCAGTCCTTTGAT
RRM2 probes (SEQ ID NO: 655-671)
GTATTCAGTATTTGAACGTCGTCCT
GTCTTGCATTGTGAGGTACAGGCGG
TTTTACCTTGGATGCTGACTTCTAA
GTACAGGCGGAAGTTGGAATCAGGT
GACCCTTTAGTGAGCTTAGCACAGC
CCTGGCTGGCTGTGACTTACCATAG
GAACGTCGTCCTGTTTATTGTTAGT
CTCACAACCAGTCCTGTCTGTTTAT
GAAGTGTTACCAACTAGCCACACCA
ATGTGAGGATTAACTTCTGCCAGCT
CTAGCCACACCATGAATTGTCCGTA
CAGCCTCACTGCTTCAACGCAGATT
TTAGGATTCTGTCTCTCATTAGCTG
GTGCTGGTAGTATCACCTTTTGCCA
TATGGTCCTTATATGTGTACAACAT
GAAGATGTGCCCTTACTTGGCTGAT
TAAACAGTCCTTTAACCAGCACAGC
CDK1 probes (SEQ ID NO: 672-682)
TGAAGTATTTTTATGCTCTGAATGT
CAAAGATCAAGGGCTGTCCGCAACA
GATGAATATTTTTCTACTGGTATTT
GACATAGTGTTTATTAGCAGCCATC
GAAAGCTTTTTGTCTAAGTGAATTC
GTGAATTCTTATGCCTTGGTCAGAG
TGTTAACTATACAACCTGGCTAAAG
AAATGTTCTCATCAGTTTCTTGCCA
TGCTAAGTTCAAGTTTCGTAATGCT
AAGGGCTGTCCGCAACAGGGAAGAA
CTTATCTTGGCTTTCGAGTCTGAGT
HN1 probes (SEQ ID NO: 683-691)
GGCCTCTAATATCTTTGGGACACCT
GAAGGAACTCCTCTGAAGCAAGCTC
GACTTGGAGTCATCTGGACTGCAGA
AGAGTGAAGAGAAGCCCGTGCCTGC
GACCCCAACAGCAGGAATAGCTCCC
GTGGTGGATCCAATTTTTCATTAGG
CATCCAGAAGAAATCCCCCTGGCGG
ACAACCACCACCTTCAAGGGAGTCG
GCAAGCTCCGGAGACTTCTTAGATC
ZWINT probes (SEQ ID NO: 692-702)
GATGTACCTTTTTTGTCAACTCTTA
TAGTGATACCTTGATCTTTCCCACT
GTTTCATTGACCTCTAGTGATACCT
GTACAGCCTAGTGTTAACATTCTTG
GATTGGCTTTTGTCATCCACTATTG
AACATTTCTCGATCACTGGTTTCAG
TTTCCCACTTTCTGTTTTCGGATTG
GGCCTCCTATGATGCAGACATGGTG
TCTTGGTATCTTTTTGTGCCTTATC
AGGAGCTGGGACTGGTTTGAACACA
CAGATGGGGAGGGGGTACTGGCCTT
ASPM probes (SEQ ID NO: 703-713)
ATAGAGCCTCTGATGTACGAAGTAG
CAGTCTCTACAAACTTACAGCTCAT
AATCCCCTGCAAGCTATTCAAATGG
GAAGAAATCACAAATCCCCTGCAAG
GTGATGGATACGCTTGGCATTCCTT
GCATTCCTTTTATCCCAGAAACACC
GTTGTTTGTTGGCTATTTTACTGAA
GGAGCTTTTGCAGATATACCGAGAA
TCAGATATGCTGTGCAAGTCTTGCT
GTTGTTGACCGTATTTACAGTCTCT
GTTGTAATCGCAGTATTCCTTGTAT
SLC35E3 probes (SEQ ID NO: 714-723)
AGTAGCTCTCTGCTTGCTGATAGAT
ATTTTAGTTTAGCTTCCTGATTTAT
GATGGTTTCCCAGTGTGAGATTTGT
ATGGTTTGGTTGGTCCCAGCAAAGT
GTTCTCGGTGTTCAAACTCTTCTAA
TGCAAATATGCTGTGGGTTCTCGGT
AAGGAATTGCTGTTACTGTACTGCA
TAAATCTAGTGTTTCTATTTTAGTT
ATATACCATCCCATATATATGTGGG
CTGCTTGCTGATAGATGGTTTCCCA
RNASEH2A probe (SEQ ID NO: 724-731)
ATGCCAGAGACATACCAGGCGCAGC
GGAAGATCACATCCTACTTCCTCAA
ACTGATTATGGCTCAGGCTACCCCA
TGCAGCAAAGTTTTCCCGGGATTGA
CTCAATGAAGGGTCCCAAGCCCGTC
TCGTGGACACCGTAGGGATGCCAGA
GAGGACTCAGCATCCGAGAATCAGG
TCTTCCCACCGATATTTCCTGGAAC
TSC2 probes (SEQ ID NO: 732-738)
TGGCCTCACAGGTGCATCATAGCCG
GGGCAACGACTTTGTGTCCATTGTC
CCCTGATGCCCACCAAGGACGTGGA
AGCACCGCTGCGACAAGAAGCGCCA
TCAACTTTGTCCACGTGATCGTCAC
GTGAGGACTTCAAGCTTGGCACCAT
TGGACTACGAGTGCAACCTGGTGTC
FAM20B probes (SEQ ID NO: 739-749)
GTATTAATAAGGCATTGCCCCCTGT
TGTAAGGCTGCATTGTGGGTTTGGG
GTTTTGTAACACTGTCCTACTTTAT
AGTCGTTGCAGGGTTTGGATCAGCT
GGATCAGCTGTAAGTTAGGTATGCC
AAGGCTGTTACAATCAAGTCGTTGC
AGATGAGTCCTATACGTGGCAATTT
TCTGAAGCCAGCATTATCTTCCAAA
GCCCCCTGTTTGCACTCAGGGTTAA
CGTGGCAATTTTTCAATGTCATCTG
TCAGAACTCATGGCCATTTCCTGCC
WASL probes (SEQ ID NO: 750-760)
GAGATACTTGTCAAGTTGCTCTTAA
TGGGCCGTCGACAAAGGAAATCTGA
AATTTCGAAAAGCAGTTACAGACCT
AATCTACCCATGGCTACAGTTGATA
GTGGGAACAAGAGCTATACAATAAC
TAGTCCTAGAGGATATTTTCATACC
GATCCCCCAAATGGTCCTAATCTAC
AGAAAAGACGAGATCCCCCAAATGG
TACCCATGGCTACAGTTGATATAAA
TTCATACCTTTGCTGGAGATACTTG
ATACCTTTGCTGGAGATACTTGTCA
AIM1 probes (SEQ ID NO: 761-771)
GCCTGTGCTGAACTGATCTCTTAAA
AATACTGGTGCTCTTGTCACAGGTA
AAAATGCTGATCTTCTCTGGAGTCT
TGCTCTTCCAACAGTGGGTTCTAGC
ACAACTGACAAGACACCAGCCCATA
TTAGGCCTTTTGTGCATACCATTAC
GGTGCATGTACAACAGCATCCAACA
TCTTTTTGTCCTCATCACTCAATAC
GCAATCTTGGAATCCTCAACTGCAG
GGTAGAACAGCTTGTTTCTTTTCCA
GCATCCAACATATCTGTCTTGTTCC
MBNL2 probes (SEQ ID NO: 772-782)
TTCAGCCCTTTAATAATGGAGCATC
TTTACTATGATATCCATTTTCCAGA
GAGACTAACTCTCCACTTGTATGGG
GGGAACTACATTTCACTCTTGGTTT
ACCTGTAACCCCAAGCAAATATAGA
AACTCTCCACTTGTATGGGAACTAC
TCAGGATATAACAGCACTTCACCGA
ATTTCACTCTTGGTTTTCAGGATAT
TAACAGCACTTCACCGAAATATTCT
TATTAGCACACAACTATTTTCAGCC
AAGTTTGTTTATATTCAGAAGTCTG
PPIF probes (SEQ ID NO: 783-793))
GCTGAAGGCAGATGTCGTCCCAAAG
TGGCAAGCATGTTGTGTTCGGTCAC
GCCTGAAACGATACGTGTGCCCACT
TAATGCTGGTCCTAACACCAACGGC
CCGCTTTCCTGACGAGAACTTTACA
GTGGCCAGGGTGCTGGCATGGTGGC
AGAAGGGCTTCGGCTACAAAGGCTC
AAGTCCATCTACGGAAGCCGCTTTC
TGTCCTGTCCATGGCTAATGCTGGT
GTTCGGTCACGTCAAAGAGGGCATG
GACTGTGGCCAGTTGAGCTAATCTG
GINS2 probes (SEQ ID NO: 794-803)
TACAGCAGGAGTGGCCATGTGGTCC
GATGAGGTACTCGTGGTTCTGGAGC
GGCAGATGGTGCAGCCAACAATGCT
AACAATGCTGACCGGTGCTTATCCT
GGACATTCTTCAATTCCACATCTGT
GGGCTGAATTTAGACTCTCTCACAG
CAGCCTCTGGAGAGTACTCAGTCTC
AAATAAGTCATTCTCCCTAGCAGAG
CTCTAAGCCCTGATCCACAATAAAA
GGAGCTCTAGAAACACTTCTGATGC
UBE2C probes (SEQ ID NO: 804-814)
TATAAGCTCTCGCTAGAGTTCCCCA
GAGCTCTGGAAAAACCCCACAGCTT
GGAAAAGTGGTCTGCCCTGTATGAT
GGGTAACATATGCCTGGACATCCTG
CAATGCGCCCACAGTGAAGTTCCTC
GGGTAGGGACCATCCATGGAGCAGC
GCCTTCCCTGAATCAGACAACCTTT
TCCATCCAGAGCCTTCTAGGAGAAC
ATGATGTCAGGACCATTCTGCTCTC
AGATGGTCTGTCCTTTTTGTGATTT
TGATAGTCCCTTGAACACACATGCT
TYMS probes (SEQ ID NO: 815-825)
GAGGGTATCTGACAATGCTGAGGTT
GCATTTCAATCCCACGTACTTATAA
ATCTGTCCGTGACCTATCAGTTATT
GTACAATCCGCATCCAACTATTAAA
AAATGGCTGTTTAGGGTGCTTTCAA
TCACAAGCTATTCCCTCAAATCTGA
AACTGTGCCAGTTCTTTCCATAATA
GAGGGAGCTGAGTAACACCATCGAT
GGGGTTGGGCTGGATGCCGAGGTAA
AAAGCTCAGGATTCTTCGAAAAGTT
GAACTAGGTCAAAAATCTGTCCGTG
NEK2 probes (SEQ ID NO: 826-837)
ATTAATACCATGACATCTTGCTTAT
GCTGTAGTGTTGAATACTTGGCCCC
GCCATGCCTTTCTGTATAGTACACA
TGAGCTGTCTGTCATTTACCTACTT
GTAGCACTCACTGAATAGTTTTAAA
TTGGTTGGGCTTTTAATCCTGTGTG
CTCTGTAGTTCAAATCTGTTAGCTT
GATATTTCGGAATTGGTTTTACTGT
AAATATTCCATTGCTCTGTAGTTCA
TGAATACTTGGCCCCATGAGCCATG
GGTATGCTTACAATTGTCATGTCTA
TXNRD1 probes (SEQ ID NO: 839-844)
ACCTGTATTTCTCAGTTGCAGCACT
CCCATGCATCTGCCTGGCATTTAGG
GCAATTGAGGCAGTTGACCATATTC
TCCTCATCTCATTTGGCTGTGTAAA
CCTGCCAGCAGTTCTTGAAGCTTCT
TCCAAGTCCACCAGTCTCTGAAATT
TGGCATTTAGGCAGCAGAGCCCCTG
GGAGTGGAATGTTCTATCCCCACAA
MED24 probes (SEQ ID NO: 845-854)
CAGCCCAGGAGTAGTCTTACCTCTG
CTTACCTCTGAGGAACTTTCTAGAT
GGCTGCTAAAGCCATTGCTGCACTC
GATGAGGCATCGTGCCTCACATCCG
TAAAGCCATTGCTGCACTCTGAGGG
GTGAGGGAAATCTACCTTCGTTCAT
TGGTGCAAGAGCCTCTAGCGGCTTC
TTCTTCTTTCAAAATTTCCTCTCCA
AACTGGTGAAGGTGTCAGCCATGTC
CATCCGCTCCACATGGTGCAAGAGC
ELF1 probes (SEQ ID NO: 855-865)
AGATGACATGGTTGTTGCCCCAGTC
AAATATGCAGACTCACCGGGAGCCT
CATGTTCCTGGTGCTGATATTCTCA
TTATGCCGGTCTAGCCTGTGTGGAA
TCACCCATGTGTCCGTCACATTAGA
GATATTCTCAATAGTTATGCCGGTC
GATGAACGACAGCTTGGTGATCCAG
TTGGTGATCCAGCTATTTTTCCTGC
GGCACTCCTCAATATGGATTCCCCT
GCTGCCTGATACGTGAATCTTCTTG
GACATCACCCTTACAGTTGAAGCTT
APH1A probes (SEQ ID NO: 866-876)
GTGCATGTTTGGGAACTGGCATTAC
TTCTCAGTACTCCCTCAAGACTGGA
ACTCCAGAGCTGCAGTGCCACTGGA
GTGCCACTGGAGGAGTCAGACTACC
ATAGATGAGCTCTGAGTTTCTCAGT
GTCAGACTACCATGACATCGTAGGG
TCTTCTAACCTCCTTGGGCTATATT
CAGGCCTGAGGGGGAACCATTTTTG
GGACATCTTGGTCTTTTTCTCAGGC
TGCTGAGGGTGGAGTGTCCCATCCT
GAGGTATATTGGAACTCTTCTAACC
SLC11A2 probes (SEQ ID NO: 877-887)
ACTGACCATACATTTTTCTTAGCCC
GACATTTTTACATACCGAGCCTGAG
TTCATCTGAGCCCCCAAAAGCATTA
TTCTTAGCCCCTCAAGTAATATAGC
AAACTGGTCATAAAGGCACTCTGTG
CCTTCCAGAGTCCTGGCTGATTGGT
GGGACTGACATCTTAAGCTCTCACC
TACACTACTGTGTTTCACTGACCAT
TGATTGGTGTTCGCTGTTCATCTGA
GGACTTCTCATTTTTGGAGCTTTCC
GAATGACAATTCCCCTAACCATTCC
CCNB1 probes (SEQ ID NO: 888-898)
TTTGCACTTCCTTCGGAGAGCATCT
CTTCCAGTTATGCAGCACCTGGCTA
CAACATTACCTGTCATATACTGAAG
TGGACACCAACTCTACAACATTACC
TGGCACCATGTGCCATCTGTACATA
ACTATGACATGGTGCACTTTCCTCC
GGAACTAACTATGTTGGACTATGAC
GAATCTCTTCTTCCAGTTATGCAGC
GCACCTGGCTAAGAATGTAGTCATG
TCCTCCTTCTCAAATTGCAGCAGGA
AGATTGGAGAGGTTGATGTCGAGCA
TMEM97 probes (SEQ ID NO: 899-909)
ATTCCATCAGCTTTCTCTAAGTCTT
ATAGAGGGTCTCTTCACGTTGATGC
ATCCACTGTGTGCATAGAGGGTCTC
TCAGCTTTCTCTAAGTCTTTGCTCA
AAGTTCAACCTTAAAATGATGTTAG
TCTCTTCACGTTGATGCTTGGCATT
GCCAGGCATAACATATCCACTGTGT
TCACGTTGATGCTTGGCATTCCATC
GTGTGCATAGAGGGTCTCTTCACGT
TACAGCCAGGCATAACATATCCACT
CCATCAGCTTTCTCTAAGTCTTTGC
MLF1IP probes (SEQ ID NO: 910-920)
AGGCCATAATCATCTTTTCTGGTTA
AAATAGCATCAGTTTGTCCAATAGT
ATGTTGACACCTTAATCGGTCCCAG
GAAGCTCCTTGACCAGGGATGAGAA
CAACCATCAGTTAGAGAAGCTCCTT
AAGAACACTTCTGGGAGCCGAAAGC
GTGCCTATAGGAAGACTAGTCTCAT
GCCATCTGCGAAATATCAACCATCA
AAACGTATGATTCATCCAGCCTTCC
ATCGGTCCCAGGTATGAGCTATAAT
GGAGCCGAAAGCCATCTGCGAAATA
ECT2 probes (SEQ ID NO: 921-931)
AGACTGTTTGTACCCTTCATGAAAT
TCACAATAGCCTTTTTATAGTCAGT
GTAAATGACTCTTTGCTACATTTTA
GCATGTTCAACTTTTTATTGTGGTC
GGTAATTTTATCCACTAGCAAATCT
GCATAGATATGCGCATGTTCAACTT
GAAGTTGCCATCAGTTTTACTAATC
CAGTTTTACTAATCTTCTGTGAAAT
TAGCTGTTTCAGAGAGAGTACGGTA
TTCCTATTTCTTTAGGGAGTGCTAC
GTATGTGCCACTTCTGAGAGTAGTA
RAE1 probes (SEQ ID NO: 932-942)
AGCCTTGTTGGGTTGTCAGCCATGG
AACAGTTAGATCAGCCCATCTCAGC
ATGGCACCCTTGCAACTGTGGGATC
AGAAGTAGTGGCTGGAGACTCTGGC
CTGCCTCATCTCTGTACGAATTTGG
GATGGTAGATTCAGCTTCTGGGACA
CTCAGCTTGCTGTTTCAATCACAAT
AATGGAATCGCGTTCCATCCTGTTC
TGGATTTCAACCCCTGGAGAAAACG
ACGAATTTGGGTCCCAGCCTTGTTG
CATATTTGCATACGCTTCCAGCTAC
DONSON probes (SEQ ID NO: 943-949)
GGAAATCACATAGCAGTTACCCCAT
GTGGTGCTGAGAGACTACATTTATA
ATACCGTTACTTGGGAAATCATCTT
CAACTGCTGTATTTAACATCTGCCT
GATATCTATATCCCTACAACCTAAT
TAACTGTGGTTTGCACCCTAACACT
GTTACCCCATGGCATTGTGACTAAT
KIAA0776 probes (SEQ ID NO: 950-960)
CCACCTCATACACACACAATTCAGT
GTCATATAGTAAGCATTTTCCCCCA
CATATTTCAGGTTTGTTCTCTTTCC
AAAGTAGTCACTATACAACTCCCCT
AAGACCTTGTTCTCAAATCTAGGAA
GAAGGATTCATTTGTTGCGAGTGCC
GTTGCGAGTGCCAGTATACTTAAAT
TTTTCCAATCCATTTATCCCTGGGG
CCTGATTTTCACACAAATACTATAT
GTAAATTGGGTTCTTCATGGAAGTT
GGAAATCATCTGTGACGGAAGAGTA
>DTL_probe1 (SEQ ID NO: 961)
ATGATTTTGTTTGTATCCCTACCCA
DTL probes (SEQ ID NO: 962-971)
GGAAGCCATAGAATTGCTCTGGTCA
AGCACACCATAGCCTTAACTGAATA
TGGGTGCCAAAGGTCAACTGTAATG
GACCAATATCTGCCAGTAACGCTGT
AATTGGGATACATTTGGCTGTCAGA
TAACGCTGTTTATCTCACTTGCTTT
GACAACTTTTTAATTCCTTTGATCT
GTCTACTGGGTATAACATGTCTCAC
GGAAATCTGCCTAATCTGCTTATAT
GCTCTGGTCAAAACCAAGCACACCA
SQLE probes (SEQ ID NO: 972-982)
GATTCCCTGCATCAACTAAGAAAAG
TTGGTGGCGAATGTGTTGCGGGTCC
GCGTGTTCTGTAATATTTCCTCTAA
TCTCCTAACCCTCTAGTTTTAATTG
TCTCAGTAGTGGTGCTGTATTGTAC
AATTGGACACTTCTTTGCTGTTGCA
CTTGGATTACAAAACCTCGAGCCCT
CTGTTGGGCTGCTTTCTGTATTGTC
ATCTATGCCGTGTATTTTTGCTTTA
TTGCTGTTGCAATCTATGCCGTGTA
ATAGCATAGTACCATACCACTTATA
ACBD3 probes (SEQ ID NO: 983-993)
GAACGCAGAGCGACTCGAGGTGTCC
GGCAAAGCATTTCATCCAACTTATG
GCCTGGAGGAGTTGTACGGCCTGGC
GCAGCAGCCGGAGATGGCGGCGGTG
TTAAATAGGTGTTGCCATCTCTTTT
GACACTTGTCCTGAGGTTGGATTCT
GGCAGCCCTGGGAAACATGTCTAAA
TCAACATATGTTGCGTCCCACAAAA
TGGCACTGCGCTTCTTCAAAGAAAA
TAAGCAAGTTCTTATGGGCCCATAT
GGCCCATATAATCCAGACACTTGTC
RMI1 probes (SEQ ID NO: 994-1004)
CCCTGAACATGCCTGAGCTTGTCAT
TCCCTTTCTGAATTAGCTGTACATA
GCATTTATCTATGTCTTTAGGTGTC
GGTAATTTCCTTCTAATATGTTGGT
TACCATTCTTCCACTGTGCTGTTAT
GTAGATCAGAACATCAGGCTTTCAG
ATGCCTGAGCTTGTCATAATATGTT
ATGTTGGTACTGTCTATGGCCATAC
TTAGGTGTCATTGTTCCCTTTCTGA
AGAGGGACTGTTTACCATTCTTCCA
GCTGTACATATAAGCCTTCCTTTGG
C14orf101 probes (SEQ ID NO: 1005-1015)
CAGAAAGCACCGAATGACCCACAGC
TTCCGTCTGTACTCTCAGAAAGCAC
GAAGTGCTGTTATCGGAAACCATCA
GACTTCCAGTCTTTCACCAGATGAC
GTATCATGGTCCAGCAGTACTGTTT
TATCCCGTGTTACCAAATTACCATT
GAAACCATCAGACATTTCCGTCTGT
ATGAAAAACCTGCTCATCGTTCAGC
TTAAAACTAAGTCATCTCCCAGATA
GCAAAATTCTGTTTATCCCGTGTTA
CTCATCGTTCAGCTTCCAAAATTCT
ZNF274 probes (SEQ ID NO: 1016-1026)
CCTTTTCAGCTTGACCCTGCAATAT
AATCTGCACTGATATTACATCCACA
ACCTCATAGCTCTCAAGCCAGTTGA
AGGAGACTGCCCAGCACATAATGAA
GATATTGTTTGTTCACTCATTTAGT
ACATGCACAGGCCTGCTTGTGAATC
GCCAGTTGAAGAAACCTTGCCTTTT
AAGATTTCCCATTCACTTGATATTG
TACATCCACAGTACCACAGTATTTA
GAAGAAACAGCCTACCTCATAGCTC
GAGCGCCCATATGCATGCAACAAAT
PTGES probes (SEQ ID NO: 1027-1048)
TGGATGTCTTTGCTGCAGTCTTCTC
GGTCTTGGGTTCCTGTATGGTGGAA
CAAAGGAACTTTCTGGTCCCTTCAG
TTGGCCACCAGACCATGGGCCAAGA
CAAAGGGCAGTGGGTGGAGGACCGG
TCTCCTAGACCCGTGACCTGAGATG
CGTGGCTATACCTGGGGACTTGATG
CAGCCACTCAAAGGAACTTTCTGGT
GGTTTGGAAACTGCAAATGTCCCCT
AGGTTTGAGTCCCTCCAAAGGGCAG
GGCCCACCGGAACGACATGGAGACC
TCTCTGGGCACAGTGGGCCTGTGTG
CTCTGGGCACAGTGGGCCTGTGTGT
TTTGGATGTCTTTGCTGCAGTCTTC
ACCTGGGGACTTGATGTTCCTTCCA
GGCTATACCTGGGGACTTGATGTTC
CTCCTAGACCCGTGACCTGAGATGT
TGGAGGACCGGGAGCTTTGGGTGAC
CACCAGACCATGGGCCAAGAGCCGC
GCAGTGGGTGGAGGACCGGGAGCTT
TTTCTGGTCCCTTCAGTATCTTCAA
CACCGGAACGACATGGAGACCATCT
FRG1 probes (SEQ ID NO: 1049-1053)
GGCTCGGAAAGATGGATTTTTGCAT
GCAGTTTTCGGCTGTCAAATTATCT
GGGCGTTCAGATGCAATTGGACCAA
TAGTCCTCCAGAGCAGTTTTCGGCT
ATTGCCCTGAAGTCTGGCTATGGAA
C19orf60 probes (SEQ ID NO: 1054-1069)
GCACGGTGGCCCTGCTGCAGTTGAT
GGCGATCAGCGAGGTTCTCCAGGAC
GGACCTTAGGTTTGATGCGGAATCT
GGTTCTCCAGGACCTTAGGTTTGAT
CAGCGAGGTTCTCCAGGACCTTAGG
AATTAAAACCATGGAGGCGATCAGC
CCTTAGGTTTGATGCGGAATCTGCC
CGCTGTACGGATGCAGCAGCTGAAA
TCTGCCGAGTGATGGCGGCTCCCCA
CATGGAGGCGATCAGCGAGGTTCTC
GTTTGATGCGGAATCTGCCGAGTGA
ACTGCGCTGCTGACCTTCCTGCAGT
ATTAAAACCATGGAGGCGATCAGCG
CCAGGACCTTAGGTTTGATGCGGAA
AGTGCACGGGGTGACCCAGGCCTTC
ATCTGCCGAGTGATGGCGGCTCCCC
LPCAT1 probes (SEQ ID NO: 1070-1080)
TGTGTGTGAGACAGGACGCAGCGGG
CAGACCCGTGGGCAGGTGGGGCATG
GTTGAGTTAAACCCCTTGTGTGTGA
TCCCTTCCGCAGGTCTGCAGATGAA
AATTTCAGGGCTCTTGGCGTGTTGG
TGAAATGCCACTGCGCATTTTCAGA
TCTTTTCTCTTCGTGGCGACTTAGA
GCCTTTGGTAGCTAACAGTCACTGA
AGAAATCCTAGTGCAGCCTTTGGTA
TGAATGGATGTTTGTTCCTCCTGAT
GAGTTGGCGGATATTCGGAACTGTG
ISYNA1 probes (SEQ ID NO: 1081-1091)
TACCTCGGAGCTGATGCTGGGCGGA
ACCAATGGCTGCACCGGTGATGCCA
CAACACGTGTGAGGACTCGCTGCTG
GCCTCAAGCGAGTTGGACCCGTGGC
GCCACCTACCCTATGTTGAACAAGA
GGAACCAACACACTGGTGCTGCACA
GTGAGCTTCTGCACTGACATGGACC
AACCACATGCTCCTGGAACACAAAA
GGGCATCTGCAAGAGGAGCCCCCAA
CAAAATGGAGCGCCCAGGGCCCAGC
CCAGCGCAGCTGCATCGAGAACATC
SKP2 probes (SEQ ID NO: 1092-1102)
AAATTGATGACTTGTTCGTATGTTC
GAAGTGCCTTTATCTGCTTAGACCT
TGCCCTCAAACATACAGAACTTCCA
CTCTGACATCGGATGCCCTCAAACA
AGCTATTTTGCCAACATGTCAGAGT
AGAACTTCCAAACTCAAGTCCAGCC
AAGTCCAGCCATAAGCTATTTTGCC
AGAGCTGGGGTTAGGATCCGGTTGG
TAGGATCCGGTTGGACTCTGACATC
AAAGCTAACACCAGTCATTTATATT
GATGATGCTTCAATTTCTTAATAGT
DPP3 probes (SEQ ID NO: 1103-1113)
AAACGTTCTCACCAAATCCAATGCT
ATACGAGGCGTCAGCTGCTGGCCTC
AGGAGCTTGGACCTTGGTACTACCT
GATGCCCGATTCTGGAAGGGCCCCA
CAGACCAAGGCTGCAAGTGGCCCTC
GCTTACCATCCTGTCTACCAGATGA
CTCTGTGATCTCATTTCATCTGCAC
GTGGCACGTGACAGCTAGGGTTCAA
TGAGCGTTTCCCAGAGGATGGACCC
TGAGGGTGGTGACACAACCCCTTCC
TCATCTGCACTGCCATACGTGGAGT
TYMP probes (SEQ ID NO: 1114-1124)
CCTGTGCTCGGGAAGTCCCGCAGAA
CCTTGGCCGCTTCGAGCGGATGCTG
CCGCTTCGAGCGGATGCTGGCGGCG
TGGCCCGAGCCCTGTGCTCGGGAAG
CCGAGCCCTGTGCTCGGGAAGTCCC
TGCTCGGGAAGTCCCGCAGAACGCC
CTGCTGGTCGACGTGGGTCAGAGGC
CTGGTCGACGTGGGTCAGAGGCTGC
GCCCGCCAGACTTAAGGGACCTGGT
CAGGCCCGCCAGACTTAAGGGACCT
ACTTAAGGGACCTGGTCACCACGCT
SNRPA1 probes (SEQ ID NO: 1125-1135)
GGTTGCTGCAGTCTGGTCAGATCCC
AGCTGACGGCGGAGCTGATCGAGCA
GTGGGCCATCTCCAGGGGATGTAGA
GTCAGATCCCTGGCAGAGAACGCAG
TAGCAAATGCTTCAACTCTGGCTGA
TGATCGAGCAGGCGGCGCAGTACAC
AAGGTTCCGCAAGTCAGAGTACTGG
TGGCATCTCTCAAATCGCTGACTTA
TCCAGGTGCTGGTTTGCCAACTGAC
TCCGGGGGTGATCTGAACCCTCTGG
AACGCAGATCAGGGCCCACTGATGA
DHCR7 probes (SEQ ID NO: 1136-1146)
TCTCCAGCGAGGAGGTCTCAGTCCC
GCGTGCACGGTGTTGAACTGGGACA
CTATGCTCCGAGTAGAGTTCATCTT
CTCCTTGGTAGCGTGCACGGTGTTG
TGACTGTGCAGACTCTGGCTCGAGC
AGGTGTAGGCAGGTGGGCTCTGCTT
GAAAGGGGCTTTCATGTCGTTTCCT
TCTTCCTCATCCCTAGGGTGTTGTG
GAACTCTTTTTAAACTCTATGCTCC
GTCTGCAGACCTCAGAGAGGTCCCA
GAACTGGGACACTGGGGAGAAAGGG
TFPT probes (SEQ ID NO: 1147-1157)
AAAGTACCAGGCACTAGGTCGGCGC
GCGCTGCCGGGAGATCGAGCAGGTG
TGGCCCCGGTGCAGATTAAGGTTGA
CAGCCAGTTCACCATTGTGCTGGAG
GCCGAGCAGGAAATGCGCTGACTCC
CCTGGATTCCAGTTGGGTTTCTCGG
GCTGGACTCCTACGGGGATGACTAC
AACGAGCGGGTCCTGAACAGGCTCC
TCGGGGTCCAGACAAACTGCTGCCC
GGTTCCTCATGAGAGTGCTGGACTC
GGCGGCGCCAGCGGGAATTAAATCG
CTTN probes (SEQ ID NO: 1158-1174)
TGTGTCTTTCCAGAAGGTCACGTGG
CAAAGATGGGGTGCCAAGACGGTGC
TCGCCCAGGATGACGCGGGGGCCGA
GTGGAAATGTCTCGGGACTTGGGTC
CGTGAACAGCCTTTTATCTCCAAGC
GAAACTCATCTCCTTCCTGAGGAGC
GAATTTCGTGAACAGCCTTTTATCT
CCAGGACACCGCTGTCCTGGCATTT
CAGCCTTTTATCTCCAAGCGGAAAG
TTCCTCATTGGATTACTGTGTTTTA
GAAGGTCACGTGGAAATGTCTCGGG
CTGGGAGACCGACCCTGATTTTGTG
AATCAGTCCCCAATGCCTGGAAATT
GCCTGGAAATTCCTCATTGGATTAC
CCTGAGGTGCATTTTCTCATCATCC
CATCCTTGCTTTACCACAATGAGCA
ATTTGTGGCCACTCACTTTGTAGGA
MCM5 probes (SEQ ID NO: 1175-1185)
GCATCGCATGCAGCGCAAGGTTCTC
GAGGAAGGAGCTGTAGTGTCCTGCT
CTGGGAAGTGTGCTTTTGGCATCCG
CGGCGAGATCCAGCATCGCATGCAG
CCAGCATCGCATGCAGCGCAAGGTT
CTGCCTGCCATTGACAATGTTGCTG
GCGAGATCCAGCATCGCATGCAGCG
GTTCTGGGAAGTGTGCTTTTGGCAT
GAAGGAGCTGTAGTGTCCTGCTGCC
TCGCATGCAGCGCAAGGTTCTCTAC
TTGACAATGTTGCTGGGACCTCTGC

APPENDIX 4
Probe sequences for 17-gene and 8-gene
panel of Tables 1 and 2.

CCNB2 probes (SEQ ID NO: 1-9)
ATGGAGCTGACTCTCATCGACTATG
ATATGGTGCATTATCATCCTTCTAA
AGTCCTCTGGTCTATCTCATGAAAC
CTTGCCTCCCCACTGATAGGAAGGT
CAAAAGCCGTCAAAGACCTTGCCTC
GATTTTGTACATAGTCCTCTGGTCT
GCCACTACACTTCTTAAGGCGAGCA
GATAGGAAGGTCCTAGGCTGCCGTG
ATCCTTCTAAGGTAGCAGCAGCTGC
TOP2A probes
(SEQ ID NO: 10-17 and SEQ ID NO: 19-20)
ACTCCGTAACAGATTCTGGACCAAC
GACCAACCTTCAACTATCTTCTTGA
GAAAGATGAACTCTGCAGGCTAAGA
ACAAGATGAACAAGTCGGACTTCCT
TGGCTCCTAGGAATGCTTGGTGCTG
GATATGATTCGGATCCTGTGAAGGC
AAAGAAAGAGTCCATCAGATTTGTG
GAATAATCAGGCTCGCTTTATCTTA
AAGAACAAGAGCTGGACACATTAAA
GAGACTTTTTTGAACTCAGACTTAA
RACGAP1 probes (SEQ ID NO: 21-25)
GTACAACTCGTATTTATCTCTGATG
GAATGTTTGACTTCGTATTGACCCT
GGATGCTGAAATTTTTCCCATGGAA
ACTTCGTATTGACCCTTATCTGTAA
CAATATATCATCCTTTGGCATCCCA
CKS2 probes (SEQ ID NO: 26-28)
CGCTCTCGTTTCATTTTCTGCAGCG
TATTCTTCTCTTTAGACGACCTCTT
TCTCTTTAGACGACCTCTTCCAAAA
AURKA probes (SEQ ID NO: 29-39)
CTACCTCCATTTAGGGATTTGCTTG
GTGTCTCAGAGCTGTTAAGGGCTTA
CCCTCAATCTAGAACGCTACACAAG
GAGGCCATGTGTCTCAGAGCTGTTA
TTAGGGATTTGCTTGGGATACAGAA
GTGCTCTACCTCCATTTAGGGATTT
AAATAGGAACACGTGCTCTACCTCC
GGGATACAGAAGAGGCCATGTGTCT
GAAGAGGCCATGTGTCTCAGAGCTG
CAGAGCTGTTAAGGGCTTATTTTTT
CATTGGAGTCATAGCATGTGTGTAA
FEN1 probes (SEQ ID NO: 40-50)
GAACTTGCTATGTAATTTGTGTCTA
GATGGTGATGTTCACCTGGCAATCA
GAGCCACCAGGAAGGCGCATCTTAG
TTGACCCACCTTGAGAGAGAGCCAC
GGACACTAAGTCCATTGTTACATGA
GAAATGATTTCCTGGCTGGCCAACT
ACACTGGTTTTCATGCGCTGTTTTT
ACTGATTACTGGCTGTGTCTTGGGT
TGGACCTAGACTGTGCTTTTCTGTC
TTGGGTGGGCAGAAACTCGAACTTG
ACCTGGCAATCAGCTGAGTTGAGAC
EBP probes (SEQ ID NO: 51-71)
GAAGGCACTGCTGGGAGCCATTAGA
CAGGCTCATGGGCAGGCACAAGAAG
GTCTTAGTCGTGACCACATGGCTGT
CACAGATACAAGAGAAGCCAGGAGG
AAGGGGCTGTGTGAAGGCACTGCTG
AGAAGAACTGAGGAGTGGTGGACCA
GCCAGGAGGTCTATGATGGTGACGA
CCCACCTGGCATATACTGGCTGGCC
ACATGGCTGTTGTCAGGTCGTGCTG
TCTATGGGGATGTGCTCTACTTCCT
GCATGGAAACCATCACAGCTTGCCT
GAGTGGTGGACCAGGCTCGAACACT
TTGGAGGGACAAAGCTAATTGATCT
GATGCCAAGGCCACAAAAGCCAAGA
CCAGGCTCGAACACTGGCCGAGGAG
TGACAGAGCACCGCGACGGATTCCA
GGGAGCCATTAGAACACAGATACAA
TTTGTCTTCATGAATGCCCTGTGGC
GGAGACCAAGCCTTCTTATCTCAAC
TGCAGTGTGTGGGTTCATTCACCTG
CTCCGCTTCATTCTACAGCTTGTGG
TXNIP probes (SEQ ID NO: 72-102)
TGTGTCAGAGCACTGAGCTCCACCC
TACAAGTTCGGCTTTGAGCTTCCTC
AAAGGATGCGGACTCATCCTCAGCC
ACTTTGTTCACTGTCCTGTGTCAGA
GAAAGGGTTGCTGCTGTCAGCCTTG
AGATAGGGATATTGGCCCCTCACTG
GGCAATCTCCTGGGCCTTAAAGGAT
CTTAGCCTCTGACTTCCTAATGTAG
GCAAAGGGGTTTCCTCGATTTGGAG
AAATGGCCTCCTGGCGTAAGCTTTT
AAACCAACTCAGTTCCATCATGGTG
TTCCACCGTCATTTCTAACTCTTAA
GGTTTTCTCTTCATGTAAGTCCTTG
CGGAGTACCTGCGCTATGAAGACAC
CCCTGCATCCTCAACAACAATGTGC
GTGTTCTCCTACTGCAAATATTTTC
AATTGAGGCCTTTTCGATAGTTTCG
GGAGGTGGTCAGCAGGCAATCTCCT
CCAGCGCCCATGTTGTGATACAGGG
GAAAAACTCAGGCCCATCCATTTTC
TGAGGTGGTCTTTAACGACCCTGAA
TGTTCTTAGCACTTTAATTCCTGTC
AGCTCCACCCTTTTCTGAGAGTTAT
CACTCTCAGCCATAGCACTTTGTTC
GAAGCAGCTTTACCTACTTGTTTCT
GAAGTTACTCGTGTCAAAGCCGTTA
GGTGGATGTCAATACCCCTGATTTA
CCGAGCCAGCCAACTCAAGAGACAA
TGGATGCAGGGATCCCAGCAGTGCA
GATCCTGGCTTGCGGAGTGGCTAAA
GCTGAAACTGGTCTACTGTGTCTCT
SYNE2 probes (SEQ ID NO: 103-113)
TTTCTAAGACTTTTTCACATCCAAA
GTTTTACTCCAATCAGCTGGCAATT
GGCACCCTTAGCTGATGGAAACAAT
ATTTTGAGCTGCCGGTTATACACCA
TGTTCTGTTCAGTACCTAGCTCTGC
GTAAATGCCAAACTACCGACTTGAT
TACGCTTAGAATCAGTTTTACTCCA
GTTCAGAAACTCATAGGCACCCTTA
TGAGCAGTGGTGTCCATCACATATA
ATGTACAACTCAGATGTTTCTCATT
GCTCTGCTCTTTTATATTGCTTTAA
DICER1 probes (SEQ ID NO: 114-142)
AATTTCTTACTATACTTTTCATAAT
ATTTCACCTACCAAAGCTGTGCTGT
ACTAGCTCATTATTTCCATCTTTGG
AAATGATTTTTCACAACTAACTTGT
TTGCAGTCTGCACCTTATGGATCAC
TGATACATCTGTGATTTAGGTCATT
GGAGACGCCAATAGCAATATCTAGG
CTGATGCCACATAGTCTTGCATAAA
AGCTGTGCTGTTAATGCCGTGAAAG
GAAGTGCGCCAATGTTGTCTTTTCT
GTGAAACCTTCATGGATAGTCTTTA
TTTACTAAAGTCCTCCTGCCAGGTA
GGACATCAACCACAGACAATTTAAA
TGTTGCATGCATATTTCACCTACCA
ATAAACCTTAGACATATCACACCTA
TAGTCTTTAATCTCTGATCTTTTTG
GAGACAGCGTGATACTTACAACTCA
GACCATTGTATTTTCCACTAGCAGT
CTGCAGCAGCAGGTTACATAGCAAA
GCCGTGAAAGTTTAACGTTTGCGAT
AACTGCCGTAATTTTGATACATCTG
TATTTACCATCACATGCTGCAGCTG
AACGTTTGCGATAAACTGCCGTAAT
GGAAATTTGCATTGAGACCATTGTA
GCACCTTATGGATCACAATTACCTT
AGAAGCAAAACACAGCACCTTTACC
CCCTTAGTCTCCTCACATAAATTTC
TGTGTAAGGTGATGTTCCCGGTCGC
CTGCCAGGTAGTTCCCACTGATGGA
AP1AR probes (SEQ ID NO: 143-153)
GCCTTCCTTTACCTTGTAGTACAAG
TTTTTCCTCTTGCAACAATGACGGT
GTCAATTTACAAGGCCAGGGATAGA
TTCCACTTCATTTTACATGCCACTA
GTGCTAGACAATTACTGTTCTTTTC
AATATCTATAACTGCATTTTGTGCT
GATAGAAAACACTCCATAATTGCTT
CATTGATTTTATTAAGCCTTCCTTT
TACATGCCACTATATTGACTTTAAT
TCTGGTATGAAAGGCTCCATTGATT
GCTTTCCTTGATTTTGCTGAGGATT
NUP107 probes (SEQ ID NO: 154-163)
GGATATCAGCGTTTCTCTGTGTGCT
GAAAGCTTTGTCTGCCAATGTTGTG
CAGAGAGTCCTCTCTAATGCTCCTA
GATATTGCACAGTACTGGTCAGTAT
GACCAGGGACTTGACCCATTAGGGT
AGATATGGTATCCTCTGAGCGCCAC
AATGCTCCTAGACCAGGGACTTGAC
ATCGTGACACTTTCAACATGTAGGG
TTGGATGCCCTAACTGCTGATGTGA
GTGTTTTCTGCTTCATACGATATTG
APOC1 probes (SEQ ID NO: 164-174)
AAGGGTGACATCCAGGAGGGGCCTC
CAGGAGGGGCCTCTGAAATTTCCCA
GATGCGGGAGTGGTTTTCAGAGACA
CAGCAAGGATTCAGGAGTGCCCCTC
GTGAACTTTCTGCCAAGATGCGGGA
CAAGGCTCGGGAACTCATCAGCCGC
AACACACTGGAGGACAAGGCTCGGG
GACGTCTCCAGTGCCTTGGATAAGC
CCAAGCCCTCCAGCAAGGATTCAGG
TCATCAGCCGCATCAAACAGAGTGA
GTTCTGTCGATCGTCTTGGAAGGCC
DTX4 probes (SEQ ID NO: 175-180)
ATCGCCACCTGGTGCTCATGAGGTG
ACTCGTCTTGGTATTGCACTGTTGT
ATTCTCTTCCCATTTTTGTACATTT
TGCTCCGTGAAAGGACATCGCCACC
GGAGACAAACCTCGTCAGATGCTCA
TGAAGTCTTTGGTGTTGCTCCGTGA
FMOD probes (SEQ ID NO: 192-202)
GCTGGGGAGCACTTAATTCTTCCCA
GGAGCTCCGATGTGAGGGGCAAGGC
TCTGGCTGGGGTCCGTGAAGCCCAG
GCCAAACCAGCTCATTTCAACAAAG
ATGTGAACACCATCATGCCTTTATA
TGCCATCACATCCCTGATACTGTGT
TTTGGACTACGTTCTTGGCTCCAGA
GCAGCCAAATCTTGCCTGTGCTGGG
GCTTTGAAGCACCTTCCCTGAGAAG
TCTGCTTTCACATCTCTGAGCTATA
TAATGTTGCCTGGGGCTTAACCCAC
MAPKAPK2 probes (SEQ ID NO: 203-213)
GCTGAAGAGGCGGAAGAAAGCTCGG
CTCCTGCCCACGGGAGGACAAGCAA
CCTGCCCACGGGAGGACAAGCAATA
GGACAAGCAATAACTCTCTACAGGA
AACTCTCTACAGGAATATATTTTTT
GTTGACTACGAGCAGATCAAGATAA
AATGCGCGTTGACTACGAGCAGATC
CACAATGCGCGTTGACTACGAGCAG
GCGCGTTGACTACGAGCAGATCAAG
AAGCAATAACTCTCTACAGGAATAT
AGACAGAACTGTCCACATCTGCCTC
SUPT4H1 probes (SEQ ID NO: 236-246)
TACCCTCCAATTCAGACTCAGCTGA
CAGAACTTCAAATACTTCCTACCCT
CCTGCCCCAAGGAATCGTGCGGGAG
GACAGCTGGGTCTCCAAGTGGCAGC
ATCTTCTTTGGACTACAGGTGGGGT
TAGGATGCTGATTTTCCTACCCGTG
GTATATGACTGCACTAGCTCTTCCT
GAGAGCAGCACATCATTTTATCATT
GTCGAGGAGTGGCCTACAAATCCAG
TGCAAGGCTGCCAGCATCTTTGCTC
ATATGCGGTGTCAGTCACTGGTCGC

APPENDIX 5
Probe sequences for top 25 reference probesets
(set #1) and top 15 reference probesets (set #2).
Overlapping probesets listed only once.

MYL12B probes (SEQ ID NO: 1186-1189)
GTTACATTGTCTTACTCTCTTTTAC
GTTACATTGTCTTACTCTCTTTTAC
GAGGCCCCAGGGCCAATCAATTTCA
GTACCATTCAGGAAGATTACCTAAG
SFRS3 probes (SEQ ID NO: 1190-1200)
GAAACACAGGCCATCAGGGAAAACG
GAAAAATCCAACTCTCATCCTGGGC
CATCCTGGGCAGAGGTTGCCTAGTT
GATACATGGCTGTTCGTGACATTCT
AATGTCCTGCCAGTTTAAGGGTACA
GGGTACATTGTAGAGCCGAACTTTG
GAGCCGAACTTTGAGTTACTGTGCA
TACTTTACAATGTTCCCTTAAGCAA
GATAATAAACCTCTAAACCTGCCCA
AACCTGCCCAGCGGAAGTGTGTTTT
TACTTTTTTTTCCATAGCTGGGATA
CLTA probes: (SEQ ID NO: 1201-1211)
CAAGAGTAGCCTCAACCTGTGCTTC
CAGGGTGGCAGATGAAGCTTTCTAC
ACAACCCTTCGCTGACGTGATTGGT
ACCATCCTTGCTACAGCCTAGAACA
TGACATTGACGAGTCGTCCCCAGGC
TAACCCCAAGTCTAGCAAGCAGGCC
GCAAGCAGGCCAAAGATGTCTCCCG
GCCACCCTGTGGAAACACTACATCT
ATCTGCAATATCTTAATCCTACTCA
GAAGCTCTTCACAGTCATTGGATTA
TGTTTGTGATTGCATGTTTCCTTCC
TRA2B probes: (SEQ ID NO: 1212-1222)
TACTTTTCTTTCTAACATATCAATG
ATACCATACTTATATACCTGCAACT
ATGCTCTGTAACTCTGTACTGCTAG
AATACAGCCAGTGCTTAATGCTTAT
AATGTGGATTTGTCGGCTTTTATGT
GCAAGTGACAATACATTCCACCACA
AATACACTCTTGTTCTTCTAGCTTT
AAACCGGGTGCTTCAAAGTACATGA
GGAACACTATACCTGTCATGGATGA
GGATGAACTGAAGACTTTGCCTGTT
GGAGGCCCAATTTCACTCAAATGTT
MTCH1 probes: (SEQ ID NO: 1223-1232)
GTTTTTCTCAACACTACTTTTCTGA
GCTCAGCTGGGAGCATCATTCTCCT
GCTCAGCTGGGAGCATCATTCTCCT
GAGAATGGCTTATGGGGGCCCAGGT
GTTTAATGGTGATGCCTCGCGTACA
TCTCTAGTCCTACCCAGTTTTAAAG
GCCTCGCGTACAGGATCTGGTTACC
GTTGGGCAGATCAGTGTCTCTAGTC
CACCATCATGTCTAGGCCTATGCTA
GACCTCATCTCCCGCAAATAAATGT
HDLBP probes (SEQ ID NO: 1233-1243)
AACGCCCGCAGCACAACGAAGAGGC
CCGCAGCACAACGAAGAGGCCAATG
AAGAGGCCAATGGGCACTCTTCCAG
CACTCTTCCAGAGGCTTTGTGGTGC
TCCAGAGGCTTTGTGGTGCGGGACC
ACCTGCTCCACTGTTTAACACTAAA
AACCAAGGTCATGAGCATTCGTGCT
TAAGATAACAGACTCCAGCTCCTGG
TAGGATTCCACTTCCTGTGTCATGA
CCACTTCCTGTGTCATGACCTCAGG
GACCTCAGGAAATAAACGTCCTTGA
CYFIP1 probes: (SEQ ID NO: 1244-1254)
TGGGATGTTCTGGCAGCTGTGTCAT
TTGTTGCCATCACGTTCCTACAAAA
GCCTTTCTCTCCGTAAACTATTTAG
AATAGTGAACTTGATTCCCCTGCTT
ATGCTGCTGGGTTCATTCATTCATT
CTGCTTCCACTAAATCCAGTTGTGA
GCACTCCGTAACTCAACATGGCATG
GAGAATATTGGCTGCTGATTGTTGC
GTTTAGGGATCTTTCTGATGGTCTT
TTTTCAGTATCTCTGTACCTGTTAA
CTTAGTTCTAAGTCATTGTTCCCAT
SUMO1 probes: (SEQ ID NO: 1255-1265)
AAATCTTGTCAGAAGATCCCAGAAA
AAAGTTCTAATTTTCATTAGCAATT
ATTTGTACTTTTTGGCCTGGGATAT
GCCTGGGATATGGGTTTTAAATGGA
AATGGACATTGTCTGTACCAGCTTC
CATTGTCTGTACCAGCTTCATTAAA
AATGACCTTTCCTTAACTTGAAGCT
GACCTTTCCTTAACTTGAAGCTACT
GAGGGTCTGGACCAAAAGAAGAGGA
AGGTGAGAGTAATGACTAACTCCAA
CTAACTCCAAAGATGGCTTCACTGA
DHX15 probes: (SEQ ID NO: 1266-1276)
AAGTTCGAGTTGTGCTCTTCACGTT
TGTGCTCTTCACGTTGGTTCGATAA
CACGTTGGTTCGATAATGGCCTTTA
GTAAATATTCCATTCTGATTTCATA
ATTAAACATTTATGCCTCCCTTTTG
CCTCCCTTTTGTGTTGACACTGTAG
GTTGACACTGTAGCTCATACTGGAA
GTGATTATCGACCATGGTATGCATG
GGTATGCATGATCGTTGTAATTGTT
TTTTTTGTTTCAGTACCAGAGGCAC
GTACCAGAGGCACTGACTTCAATAA
HNRNPC probes: (SEQ ID NO: 1277-1287)
AATAATCTCTTGTTATGCAGGGAGT
TATGCAGGGAGTACAGTTCTTTTCA
TCTTTTCATTCATACATAAGTTCAG
TAAGTTCAGTAGTTGCTTCCCTAAC
GTTGCTTCCCTAACTGCAAAGGCAA
ACTGCAAAGGCAATCTCATTTAGTT
GAGTAGCTCTTGAAAGCAGCTTTGA
AGAAGTATGTGTGTTACACCCTCAC
TGCTGTGTGGGGCAGTTCAACACAA
GTTGGCATGTCAAATGCATCCTCTA
ACAGCCTGATGTTTGGGACCTTTTT
UBE2D3 probes: (SEQ ID NO: 1288-1298)
GTTGGGATTTGCTTCATTGTTTGAC
TGCACAGTCTGTTACAGGTTGACAC
TTGACACATTGCTTGACCTGATTTA
TAGTGTAGCTTTAATGTGCTGCACA
GTGCTGCACATGATACTGGCAGCCC
ACTGGCAGCCCTAGAGTTCATAGAT
GTTCATAGATGGACTTTTGGGACCC
GGGACCCAGCAGTTTTGAAATGTGT
GCAGCCCCTGTCTAACTGAAATTTC
CTAACTGAAATTTCTCTTCACCTTG
CTTCACCTTGTACACTTGACAGCTG
DAZAP2 probes: (SEQ ID NO: 1299-1324)
AGAGTGTCTGATGCGGCCACTCATT
TGAAGCCGCCCTAAGGATTTTCCTT
GGGGAACTTCTTCATGGGTGGTTCA
TTGTGTGTTCTGTACATGTGATGTT
CTCCCAATGCTGCTCAGCTTGCAGT
GAGGAGGATGCATTTCAAAAGCTTG
GATGTCGTGCAAACTGTACTGTGAA
ATAGGTTGTCTCTGCATACACGAAC
GATTCTTTACTTAGCTTGTTTTTAG
ATTTATATCCCATCTAGAATTCAGC
TGCAGTCATGCAGGGAGCCAACGTC
GTGGTGCACTTAACTTGTGGAATTT
GTTTGACTGTACCATTGACTGTTAT
GATGAAGTTGCATTACACCTCACTG
AAGTTCAGCGTTGTATGTCTCTCTC
AATACTGTACCATACTGGTCTTTGC
TCTTTCTGGTGCCCAAACTTTCAGG
TACACGAACCTAACCCAAATTTGCT
GAATTCAGCTAGGTGCTGCTGCTGC
ACGTCCTCGTAACTCAGCGGAAGGG
CTCTCTCTACACTGTGGTGCACTTA
AATGACTTGAGTCCAGTGAAATCTC
TAGCAGTACCTCCCTAAAGCATTTT
ACACCTCACTGCAAGGATTCTTTAC
GGCTCCCCAGAATTCCTAGACTGGG
CTCTGTTTCCTTTGATGACGCTTTG
SNRNP200 probes: (SEQ ID NO: 1325-1335)
GAAGTCACAGGCCCTGTCATTGCGC
GATGCCAAGTCCAATAGCCTCATCT
CCCACAACTACACTCTGTACTTCAT
GGAGTACAAATTCAGCGTGGATGTG
GATTCAGATTGAGTCCTGAGGCATT
GTAGGAATCCTGGTTGTGGGGACCA
ACTCTGGATCCAGTGACAGCAGGTG
ACAGCAGGTGTCATGGGTCAAGCAT
AATCATATATAGCATTTTCAGGCAT
GGCATGTTCCTGGTAGTTCTTTTGA
CTGGTAGTTCTTTTGAGTCTGACAT
YTHDC1 probes: (SEQ ID NO: 1336-1346)
GTATGATGGTTTGACTGTATGGCAG
GGATCTTGATTGATAACTGCCATGA
GTGTGTTCATCCTAGAGTTATTTTT
CCTTCCCCTCCAAATTGTATACATT
TGTTGTAGCAGCCTCTTGTTTTTTT
GCGTGGCAGCGGAAGACGATTCCCA
AATTCCTATGTTCAGTAGCGTGGTT
AACTGCCATGATATTTTGCTTTGAT
ACATGTAGTTGCACACGGTTCAGTA
TTTCCTCAGTCTTCAATGACGAGAG
ATGTTCACAACTTGCGTGCGTGGCA
COPB1 probes: (SEQ ID NO: 1347-1368)
AGTCCTTGAAGCTTTACAGTTAATT
ACCTTTATGCTCGTTCCATATTTGG
TATGGCAGCCAACCTTTATGCTCGT
GTTTCATGTACCAAGACCCTTTTCA
GTTTGTCTTTTGTCTTAACAGTTCT
GAATGCTGTCCTCAAAGTATATAAT
TGCTGTCCTCAAAGTATATAATGTT
GATGCACTTGCAAATGTCAGCATTG
ACCAAGACCCTTTTCACAGTACAAT
GAATACTTTTCAGCCAATAATTTAT
GACCCTTTTCACAGTACAATAAACA
TAATGTTTCATGTACCAAGACCCTT
CTGCTGTTACCGGCCATATAAGAAT
CATGTACCAAGACCCTTTTCACAGT
AAATGACTACTTACAGCACATATTA
GGTATGGGCTTACTGGACTCCAACA
TAACAGTTCTGAATGCTGTCCTCAA
GTCTTTTGTCTTAACAGTTCTGAAT
GAAGCCAATTCACCAGGGACCAGAT
ACTCCAACATCTTTTGTACTCTTTC
AGTTCTGAATGCTGTCCTCAAAGTA
GCCCTTTCTGGTTACTGTGGCTTTA
NDUFB8 probes: (SEQ ID NO: 1369-1385)
GAGATCTGAGGAGGCTTCGTGGGCT
GCACTGGCACCTAGACATGTACAAC
CGGGTGGTTCACTATGAGATCTGAG
TGGTATAGCTGGGACCAGCCGGGCC
TGGGTCCTCTAACTAGGACTCCCTC
CCCTGACCGCTCACAGCATGAGAGA
GCCGGGCCTGAGGTTGAACTGGGGT
CGGTGATCCCTCCAAAGAACCAGAG
TACGAACCTTACCCGGATGATGGCA
ACACCTGTTTCTTGGCATGTCATGT
AACCGTGTGGATACATCCCCCACAC
TCATGTGCTGGGTGGGGGACGTGTA
TCTAACTAGGACTCCCTCATTCCTA
GGACTCCCTCATTCCTAGAAATTTA
CATGACCAAGGACATGTTCCCGGGG
GATCCCTCCAAAGAACCAGAGCGGG
CCAGAGCGGGTGGTTCACTATGAGA
SET probes: (SEQ ID NO: 1386-1401)
ATTGGCCTTTTACCTGGATATAAAT
ACCATCCAACAGACCTGGTGCTCTA
CCATCCAACAGACCTGGTGCTCTAA
TGCTCTAATGCCAAGTTATACACGG
ATAGGCTCTCAGTAAGAAGTCTGAT
GGTATAAAGCTCTCAAATGTGACCA
AAGCTCTCAAATGTGACCATGTGAA
TAATGGACTCAGCTCTGTCTGCTCA
AATGCCATTGTGCAGAGAAGCACCC
GAAGCACCCTAATGCATAAGCTTTT
CTAATGCATAAGCTTTTTAATGCTG
AATTAAATGCCACTTTTTCAGAGGT
CCACTTTTTCAGAGGTGAATTAATG
TAAATGGAACTATTCCATCAATAGG
CACTGTATACCGATCAGGAATCTTG
ATACCGATCAGGAATCTTGCTCCAA
CELF1 probes: (SEQ ID NO: 1402-1412)
TTGCCACTATGACCAAACGCACAGT
AAACGCACAGTCTGTTCTGCAGCAA
CTGCAGCAACAACGGGATTCAATCA
TCAACTCAGTCGTGATTCAGCCGTA
TCAGCCGTAGAAATGCTTTTCCTTT
TTATCTTGTTTGAGCTTTTCCTTTC
GAACTTGTGTTGTACTCTGTAGAAA
GTCCCAATGGGGAACCTAAATCTGT
GTTTTAATTGCACAGACACATGGAC
AAAGTCATTTTGTATCTGCCAAGTG
ATCTGCCAAGTGTGGTACCTTCCTT
XPO1 probes: (SEQ ID NO: 1413-1423)
TAGGGAGCATTTTCCTTCTAGTCTA
GCATTGTCTGAAGTTAGCACCTCTT
GCACCTCTTGGACTGAATCGTTTGT
GAATCGTTTGTCTAGACTACATGTA
GATCATGTGCATATCATCCCATTGT
ATCATCCCATTGTAAAGCGACTTCA
GTGTGTGCTGTCGCTTGTCGACAAC
GTCGCTTGTCGACAACAGCTTTTTG
ATTTGTGAGCCTTCATTAACTCGAA
GTTAGAATAGGCTGCATCTTTTTAA
ACAACTCTGGCTTTTGAGATGACTT
PTBP1 probes: (SEQ ID NO: 1424-1434)
TTCACCTGCAGTCGCCTAGAAAACT
AAACTTGCTCTCAAACTTCAGGGTT
AAGTCTCATTTCTGTGTTTTGCCTG
CCTCTGATGCTGGGACCCGGAAGGC
ATACCTGTTGTGAGACCCGAGGGGC
CGGCGCGGTTTTTTATGGTGACACA
TCCAGGCTCAGTATTGTGACCGCGG
TGCCTTACCCGATGGCTTGTGACGC
TGTTCGCTGTGGACGCTGTAGAGGC
GTTGGCCAGTCTGTACCTGGACTTC
GAATAAATCTTCTGTATCCTCAAAA
SF3B1 probes: (SEQ ID NO: 1435-1445)
GTTTACAGGGTCTGTTTCACCCAGC
TTCACCCAGCCCGGAAAGTCAGAGA
CAACTCCATCTACATTGGTTCCCAG
CTCATAGCACATTACCCAAGAATCT
GAACACCTATATTCGTTATGAACTT
TTAATGCACAGCTACTTCACACCTT
CACACCTTAAACTTGCTTTGATTTG
AATAACCTGTCTTTGTTTTTGATGT
GTAAATGCCAGTAGTGACCAAGAAC
TACACTATACTGGAGGGATTTCATT
GATTTAGAACTCATTCCTTGTGTTT
ARPC2 probes: (SEQ ID NO: 1446-1468)
ACTGGATAATCGTAGCTTTTAATGT
GTAGCTTTTAATGTTGCGCCTCTTC
GTGACAACATTGGCTACATTACCTT
TGCGCCTCTTCAGGTTCTTAAGGGA
GCTGTGCTTGCAAAGACTTCATAGT
ATCTTCCGGCATCCAAGGATTCCAT
GAGCTGAAAGACACAGACGCCGCTG
AAAGAAGGACGCAGAGCCAGCCACA
ATCTGCAGAAACGAGCTGTGCTTGC
GTCTCTTTGCTATATGACCTTGAAA
GAGGAAGCGGCTGGCAACTGAAGGC
CTCTTTTCCAAGCTGTTTCGCTTTG
CGTTTTCATCCCGCTAATCTTGGGA
GTTTCGCTTTGCAATATATTACTGG
GGAACACTTGCTACTGGATAATCGT
GAAGCGAAATTGTTTTGCCTCTGTC
GAGTCACAGTAGTCTTCAGCACAGT
GGAAGCGGCTGGCAACTGAAGGCTG
TGCAGTCATAACTTGTTTTCTCCTA
TCCTCTTTAGCCACAGGGAACCTCC
GACCTTGAAAATCTTCCGGCATCCA
GGATTCCATTGTGCATCAAGCTGGC
TTCATCCCGCTAATCTTGGGAATAA
VAMP3 probes: (SEQ ID NO: 1469-1479)
GAGACTCAACATCAGGATCCACAGC
ACAGACTTTATCGCTCTGTGGCTCA
AAGCAGCAACAGCTGAGGCGCACCA
GCTTCCATTTCTTTAACGTCTGTTC
TCTGTTCCCTTAACATCGCTGAAAT
GAAGAGATGCCTTGCGGTGTGGCCA
GACTCAGAAACCTTGGTACTCGCCC
ACTGGCTCCTGCATTAACCCAGAAA
TAACCCAGAAATACCTCGCTTCTAT
CTCGCTTCTATCTGTGCACTTAGCT
GGGAACTTACCCACTGTAATCACCT
STARD7 probes: (SEQ ID NO: 1480-1490)
TTGTGCCAAGGAAGTAGCTGCCCCA
CCTTCTCCGCGTCATTGTTGGAAGA
AGGAGAGATGCATCGAGCAGTCCCA
GCTGCTTTTCATTTATTACTTCTTC
CTTCTTCTTTCCAGGACCTGACAGA
TTATGTCCAAACTTAGCACCTGCAA
TGTGCGTCTGCGAGCGCACACACAT
AGGAGTTGCGGTTGCTCCATGTTCT
GCTCCATGTTCTGACTTAGGGCAAT
CTGCACTTGGGGTCTGTCTGTACAG
GTCTGTACAGTTACTCATGTCATTG
SEC31A probes: (SEQ ID NO: 1491-1511)
TTCAGTGAGACCTCTGCTTTCATGC
AGCATGTTTGCATAGCAACCAGTCA
GTTGCCAGTGATGATTTTCCTATTC
ATTTCTGCTGATATACTCACCTTAG
GCTGCCTTTCTTCAGCAACAGACCC
AGCAACCAGTCAAGAGCATTTACAC
TGAAGGTGCCCCAGGGGCTCCTATT
AGTATGGTTTCCTGAAGTATTCTGA
TAAAACTAAATTTCTTTCATGTCCT
TGCTCAGAACCCTGGTGCTTTATTT
GCGTACAGCAACCTCTTGGTCAAAC
TTCTCTTCCACTCAATATTGCCATT
GGACTAGTCCTCATTAGCATGTTTG
CATACCCACATAGTTAGCACCAGCA
GAGCATTTACACTATTTCTGCTGAT
AGAAGGATTGACCATGCATACCCAC
GCACCAGCAACTTCAGTGAGACCTC
TTGAGGATCTTATTCAGCGCTGCCT
GCCAGTTCTCAAAGTTGTTCTCACC
TACTCACCTTAGAACTGCTCAGAAC
GTATTTCCTGGATTACACATAGTAT
MFN2 probes: (SEQ ID NO: 1512-1532)
GTCTATGAGCGTCTGACCTGGACCA
GTTACTCCTGTATCATTGCTCATAA
AGCCTCTGTGCACTGTTTGGTGGCC
TGTATTTAAAGCCCTCAGTCTGTCC
GCCTGAATGGACAGGGGCCACTTCA
ATCACTGTCACACAATTCCAATGGA
GACCTTTGCTCATCTGTGTCAGCAA
CCCGGCGTGTGCCGGGCCTGAATGG
GCTGGAGCGCAAGACGTGCTGACAC
AGGTGATGTCCTGTTCACATACCTG
GCCTTCAAGCGCCAGTTTGTGGAGC
CCTCCATGGGCATTCTTGTTGTTGG
TCATGGTTTCCATGGTTACCGGCCT
CCCAGCCATCACTCATCTTTGAGGA
GCGAAGTGATGGACTCTGCCAGGTG
GCCACTTCACAGCATGTCAGGGAAA
GTCCTGTTGTGTGGGGCGAAGTGAT
GTGCTGACACAGTGAGTTTTCTCTG
GCAGCTTGTCATCAGCTACACTGGC
GTGTCAGCAAGTTGACGTCACCCGG
CTGCCAGGTGGACATGCTGTGGGTG
WIPI2 probes: (SEQ ID NO: 1533-1564)
CAATGAGATCTTGGACTCTGCCTCT
AGATCCCGCGGTTGTTGGTGGGTGC
TCTACTTCACTCTTCCTGTTGAAAA
CAGACTCTGCATTCCAAACCAAGGC
GACTGACTGAACTTGACCTGTGACC
AGCAACAGAGAGTAGGCGGCTGGGC
TGCCTGGACTCGCTGGAGCAAAGGA
CCGCCCATGATTCTTCGGACTGACT
GCCCCTTAGTCACTCAGACATACGG
CGCCGACGGGTACCTGTACATGTAC
GTCATGTGCCTTTCTATTTTCATCT
TAGGGGAGCTAGAAGCCACTTTCCA
GGGCTTCCTACCTGTGTGAGAGGTC
TCGCTTCCCTTTTCATATTTACAGA
ACTTGAAAGGTTGCCTGGACTCGCT
GCATGAACGTGCCAAGCCAGCATAG
CCCTGCGCCTGGATGAGGACAGCGA
CAGAACTCAAGTGTGGTGGCCGTCT
AATTGGATCGCTCTGGGATTTCTTC
GGACAGCGAGGTTCTTTCTGATACT
CCCACCAGGTGTGCTGGGCAGACTT
AAATGATCTGTTCTTCTACTTCACT
AAACAACCTCAAGTACCTCAGACTC
GGGCAGACTTCAGCTGGGACAGAAG
TTCGGGAAAGTGCTCATGGCCTCCA
CAAGCTTCAGTATTTGCCTCGCTTC
GCGTAAGGAAACCGTGGCGTCGCGC
CCACAAAAACATCTGCTCGCTAGCC
TCTGCTTGTCAAGGCCAGTTCTGCA
GCGAGTGTGCCCTGATGAAGCAGCA
GAGGTCGTAGCGGGAGACAGCAACA
TGGGACAGAAGTCCGATCTCCCTAG
PFDN1 probes: (SEQ ID NO: 1565-1575)
GGCAGTCTGCCTAAAGATTCCTTTC
GCCTTCTCCCATACATTCCAAAAGG
GTTCAACAGTAAGCAGCACCTCCAA
TCTCCTTTCGGCCAGTATCATAAGA
TGGACGCCATAATCCTGAGGCTCCT
GGCTCCTAGAGGCTGAGGGGGCAAC
TGAGGGGGCAACGGTGTGATCCAGC
GCAAGCCAGTTGTCAAACACAGCCA
GTGAGAGAGGCAGTGGCCGTCCTCC
TTCCTGTACCTTTGACTAACGCTCA
CTTCCGGGCCTGCATGCAGTAGACA
UBE3A probes: (SEQ ID NO: 1576-1621)
ATCAGCCATTTTATCGAGGCACGTG
TAGCTAATGTGCTGAGCTTGTGCCT
TAGACCACGTAACCTTCAAGTATGT
GAACTACTCTCCCAAGGAAAATATT
TAAGGAAGCGCGGGTCCCGCATGAG
GCCATCATCTTGTTGAATCAGCCAT
TACAACGGGCACAGACAGAGCACCT
TTTACTTCCGGAATACTCAAGCAAA
TATGGTGACCAATGAATCTCCCTTA
GATTGTTTTAACTGATTACTGTAGA
TTCCTAGTCTTCTGTGTATGTGATG
AGGATGTCTTTCAGGATTATTTTAA
TAATTACTTACTTATTACCTAGATT
GACTACAGGAGACGACGGGGCCTTT
GACAGAACTGTTTGTTATGTACCAT
ACTGTGCCTTGTGTTACTTAATCAT
GCGACGAACGCCGGGATTTCGGCGG
GTATAGCCCCACAGATTAAATTTAA
TTGCCACCATTTGTAGACCACGTAA
GAAGACAATGCTTTCCATATTGTGA
GCTTTAATGTGCTTTTACTTCCGGA
ATTTTTTTGCGTGAAAGTGTTACAT
CGGATAAGGAAGCGCGGGTCCCGCA
CTGGGCTCGGGGTGACTACAGGAGA
AAAGATGGCTACTGTGCCTTGTGTT
AAGGCCATCACGTATGCCAAAGGAT
GACTCTTCTTGCAGTTTACAACGGG
GAGACATTGATATATCCTTTTGCTA
GATTACTGTAGATCAACCTGATGAT
GGCTCGGGGTGACTACAGGAGACGA
GCCTCGTTTTCCGGATAAGGAAGCG
CCATTTGTAGACCACGTAACCTTCA
TTCGTGTTGCCATCATCTTGTTGAA
TTACCTACATCTCATACTTGCTTTA
GAGCTTGTGCCTTGGTGATTGATTG
CAAGGCTTTTCGGAGAGGTTTTCAT
GGGTGACTACAGGAGACGACGGGGC
TGTTACATATTCTTTCACTTGTATG
GATAAGGTAACATGGGGTTTTTCTG
GAATTACATTGTATAGCCCCACAGA
GATATATCCTTTTGCTACAAGCTAT
TGACGGTGGCTATACCAGGGACTCT
GAAACTATTACTCCTAAGAATTACA
GCTGGCGACGAACGCCGGGATTTCG
ATGCAGCTTTCAAATCATTGGGGGG
GAGGCACGTGATCAGTGTTGCAACA
GTF3C2 probes: (SEQ ID NO: 1622-1653)
GGGCAGGAGCCTCGCAATATGTGGC
GGCTCCTCAGCCTAAGACTATGGCT
AGAAACACTCAGGCCTGACCTAGGC
TAACCATCATGTATGCCCACGAGGG
TAACCATCATGTATGCCCACGAGGG
GACCCCTCTGAGTGTGGTCAGTGCC
TCCCTGTGATTGCCCTGTTAAGTAT
TCCCTGTGATTGCCCTGTTAAGTAT
TGCTCCTGCTTACGAAGTATTCCCA
TGCTCCTGCTTACGAAGTATTCCCA
GATTGCTTGTGACAACGGCTGCATC
GGCTCCTGTCTGACTATTCCAGGAT
CCCTACCGATAGAACAGTGGCTCAG
GCATGAAGGCTCCTGTCTGACTATT
TGCATCTGGGACCTCAAGTTCTGCC
CCACCAACACCTAGCTGCTGGATAT
CACAGACACCCTACCGATAGAACAG
TGGCCTGCTCAGACGGGAAAGTACT
GTATCTGCATGAAGGCTCCTGTCTG
CAAGGAATACCACAGACACCCTACC
AACCATAGCTATCATGTGTTTCCCA
ATAGCTATCATGTGTTTCCCAAATC
ACAGGGCCCACTTTGTCTATGGGAT
TTCCCAATCACTGGTCATCTGACCC
TTCCCAATCACTGGTCATCTGACCC
GGAAATCTAGTCATCTTCCCTGTGA
GGAAATCTAGTCATCTTCCCTGTGA
GACATGAATGAGACACACCCACTGA
GCAACTCTGCAGGTGGGGTCTATGC
AAGTACTGCTATTCAGTCTACCCCA
TATTACTGCCTTCTGAAACTTCCTC
TATTACTGCCTTCTGAAACTTCCTC
KHDRBS1 probes: (SEQ ID NO: 1654-1674)
GTTACTGATTTCTTGTATCTCCCAG
GCTACATGTGTAAGTCTGCCTAAAT
AATCTAGCCCCAGACATACTGTGTT
CCTCCCATTTTGTTCTCGGAAGATT
GTCCATTTGAGATTCTGCACTCCAT
CCCCTCCTGCTAGGCCAGTGAAGGG
TAATTGGATTTGTACCGTCCTCCCA
GTCAAGTATGTCTCAACACTAGCAT
GAAAAGTTCACTTGGACGCTGGGGC
TTGTCAATATATCGAACTGTTCCCA
TAACTCTGCATTCTGGCTTCTGTAT
TGTCTAAGTGTTTTTCTTCGTGGTC
AGGCCTCCTGAATTGAGTTTGATGC
GACTGGAATGGGACCAGGCCGTCGC
GATGCAGAGCTTTTTAGCCATGAAG
ATACAGAGAGCACCCATATGGACGT
GTAGATGCTTTTTTCTTTGTTGTTT
TGACTTTTTCATTACGTGGGTTTTG
GTATCTCCCAGGATTCCTGTTGCTT
TTGCTTTACCCACAACAGACAAGTA
CCTTATTCCATTCTTAACTCTGCAT
RARS probes: (SEQ ID NO: 1675-1685)
GTTGAATGACTACATCTTCTCCTTT
AGCTGCTTACTTGTTGTATGCCTTC
GTATGCCTTCACTAGAATCAGGTCT
AATCAGGTCTATTGCACGTCTGGCC
GGAAACTAGGCCGGTGCATTTTACG
GAGCTGGCAACTGCTTTCACAGAGT
GAACATGTGGCGTATCTTGTGTGAA
CTGGCCCAAGGGTGTAATCCCTCAC
AATCCCTCACAGGTTTGAACCCTGT
TTTTCCCAAGTGGCCATTGGCCCTG
GCTTTTTTTCAATCTTGTGGGCACA
MYL12A probes: (SEQ ID NO: 1686-1696)
GCAACTGGCACCATACAGGAAGATT
CAAATTCCAGCCAACGTCCTTGTTG
AACGTCCTTGTTGCACTTTGGGTAT
GCACTTTGGGTATTCTGAGATTTTC
TCTTGCCATTCCCTTAGGCTTTAGC
GGCTTTAGCAGCTTTGCATTTCCTG
TTGCATTTCCTGTTGTATTTATTCT
TATTCTCAGCCATTTTGGGCATATG
CAGACTGGAAACGGGACTTTCTATT
CTTCTCCCCCAATAACTGTGGGTCT
TCAGAGAAAGTTAGTTCGGCTCGAT
HNRNPD probes: (SEQ ID NO: 1697-1728)
GATAGTTAATGTTTTATGCTTCCAT
TATTCCATTTGCAACTTATCCCCAA
GCAAAAGTACCCCTTTGCACAGATA
GAAATGCGGCTAGTTCAGAGAGATT
AATTTTTTGTATCAAGTCCCTGAAT
GACAGGCTTGCCGAAATTGAGGACA
AACAGCCAAGGTTACGGTGGTTATG
GTGTCCTCCCTGTCCAAATTGGGAA
GATTCATTTGAAGGTGGCTCCTGCC
ATAATACTTCCTTATGTAGCCATTA
AATGTCAATTTGTTTGTTGGTTGTT
GAGTGGTTATGGGAAGGTATCCAGG
GACTACACTGGTTACAACAACTACT
GGTGGTCATCAAAATAGCTACAAAC
AAGTTTGGAAGACAGGCTTGCCGAA
GGAACCAGGGATATAGTAACTATTG
GAGCTGTGGTGGACTTCATAGATGA
AGTCCCTGAATGGAAGTATGACGTT
AAAAGCCCAGTGTGACAGTGTCATG
GAAGTTTAATTCTGAGTTCTCATTA
GGAGGATATGACTACACTGGTTACA
GAGAGATTTTTAGAGCTGTGGTGGA
TTATTCCATTTGCAACTTATCCCCA
AGGTTACGGTGGTTATGGAGGATAT
ATTTGCTTTCATTGTTTTATTTCTT
AGAAATTTGCTTTCATTGTTTTATT
CCTTTCCCCCAGTATTGTAGAGCAA
GGTATCCAGGCGAGGTGGTCATCAA
GTATGACGTTGGGTCCCTCTGAAGT
GTATGACGTTGGGTCCCTCTGAAGT
AACAACTACTATGGATATGGTGATT
TGTGCTTTTTAGAACAAATCTGGAT
TARDBP probes: (SEQ ID NO: 1729-1739)
GAGAGCGCGTGCAGAGACTTGGTGG
TGGCGAGATGTGTCTCTCAATCCTG
TCTCTCAATCCTGTGGCTTTGGTGA
GTTTTTGTTCTTAGATAACCCACAT
TGAAATGATACTTGTACTCCCCCTA
CTTTGTCAACTGCTGTGAATGCTGT
GAATGCTGTATGGTGTGTGTTCTCT
GGACTGAGCTTGTGGTGTGCTTTGC
GCAGAGTTCACCAGTGAGCTCAGGT
GTTCTAATGTCTGTTAGCTACCCAT
AAGAATGCTGTTTGCTGCAGTTCTG
HNRNPR probes: (SEQ ID NO: 1740-1760)
AAGCTAGTGCTTTGTCTTAGTAGTT
GGGGCAATCGTGGGGGCAATGTAGG
TCGTTTCAGGCTTCATTTTAGCTTC
TCACACCTTTTTGAAATCTGCCCTA
ACCCTCCAGATTACTACGGCTATGA
ATTGTTATAACTTCACACCTTTTTG
TGGATATGGCTACCCTCCAGATTAC
AAACAAGCTGGGCACACTGTTAAAT
GCTCTTGGACATTATTGGGCTTGCA
CATGATTTTGCAGAACCTTTGGTTT
CAATGCTTTTATCGTTTCAGGCTTC
GTTCCCGTGGATCTCGGGGCAATCG
GATTCCAAGCGTCGTCAGACCAACA
TCAACAGCAGAGAGGCCGTGGTTCC
GGCTATGAAGATCCCTACTACGGCT
AAAGCCGTGACAATTTGTTCTTTGA
TCACAGAGGGGGGCACCTTTGGGAC
ACCTTTGGGACCACCAAGAGGCTCT
GTATTTCCAATTTCTTGTTCATGTA
GTCGTCAGACCAACAACCAACAGAA
TGGGCTTGCAGAGTTCCCTTATTCT

1. A method of evaluating the likelihood of a relapse for a patient that has a lymph node-negative, estrogen receptor-positive, HER2-negative breast cancer, the method comprising:
providing a sample comprising breast tumor tissue from the patient;
detecting the levels of expression of the 17 genes, or one or more corresponding alternates thereof, identified in Table 1; or of the 8 genes, or one or more corresponding alternates thereof, identified in Table 2; in the sample; and
correlating the levels of expression with the likelihood of a relapse.

2. The method of claim 1, wherein the detecting step comprises detecting the levels of expression of the 17 genes, or one or more corresponding alternates thereof, identified in Table 1.

3. The method of claim 1, wherein the detecting step comprises detecting the levels of expression of the 8 genes, or one or more corresponding alternates thereof, identified in Table 2.

4. The method of claim 1, further comprising detecting the level of expression of at least one reference gene identified in Table 3.

5. The method of claim 1, wherein the detecting step comprises detecting the level of expression of RNA.

6. The method of claim 5, wherein detecting the level of expression of RNA comprises a quantitative PCR reaction.

7. The method of claim 5, wherein detecting the level of expression of RNA comprises hybridizing a nucleic acid obtained from the sample to an array that comprises probes to the 17 genes set forth in Table 1, and/or one or more corresponding alternates thereof; or hybridizing a nucleic acid obtained from the sample to an array that comprises probes to the 8 genes set forth in Table 2, and/or one or more corresponding alternates thereof.

8. The method of claim 1, wherein the detecting step comprises detecting the level of protein expression.

9. A kit comprising a microarray comprising probes to the 17 genes, or one or more corresponding alternates thereof, identified in Table 1; or probes to the 8 genes, or one or more corresponding alternates thereof, identified in Table 2; or comprising primers and probes for detecting expression of the 17 genes or one or more corresponding alternates thereof, identified in Table 1; or primers and probes for detecting expression of the 8 genes, or one or more corresponding alternates thereof, identified in Table 2.

10. The kit of claim 9, wherein the microarray further comprises a probe to at least one reference gene identified in Table 3.

11. The kit of claim 9, wherein the kit comprises primers and probes for detecting expression of the 17 genes, or one or more corresponding alternates thereof, identified in Table 1; or primers and probes for detecting expression of the 8 genes, or one or more corresponding alternates thereof, identified in Table 2.

12. The kit of claim 11, further comprising primers and probes for detecting expression of at least one reference gene identified in Table 3.

13. A computer-implemented method for evaluating the likelihood of a relapse for a patient that has a lymph node-negative, estrogen receptor-positive, HER2-negative breast cancer, the method comprising:
receiving, at one or more computer systems, information describing the level of expression of the 17 genes, or one or more corresponding alternates thereof, identified in Table 1 in a breast tumor tissue sample obtained from the patient;
performing, with one or more processors associated with the computer system, a random forest analysis in which the level of expression of each gene in the analysis is assigned to a terminal leaf of each decision tree, representing a vote for either “relapse” or no “relapse”;
generating, with the one or more processors associated with the one or more computer systems, a random forest relapse score (RFRS), wherein if the RFRS is greater than or equal to 0.606 the patient is assigned to a high risk group, if greater than or equal to 0.333 and less than 0.606 the patient is assigned to an intermediate risk group and if less than 0.333 the patient is assigned to low risk group.

14. The computer-implemented method of claim 13, further comprising generating, with the one or more processors associated with the one or more computer systems, a likelihood of relapse by comparison of the RFRS score for the patient to a loess fit of RFRS versus likelihood of relapse for a training dataset.

15. A non-transitory computer-readable medium storing program code for evaluating the likelihood of a relapse for a patient that has a lymph node-negative, estrogen receptor-positive, HER2-negative breast cancer in accordance with the method of claim 13, the computer-readable medium comprising:
code for receiving information describing the level of expression of the 17 genes, or one or more corresponding alternates, identified in Table 1 in a breast tumor tissue sample obtained from the patient;
code for performing a random forest analysis in which the level of expression of each gene in the analysis is assigned to a terminal leaf of each decision tree, representing a vote for either “relapse” or no “relapse”; and
code for generating a random forest relapse score (RFRS), wherein if the RFRS is greater than or equal to 0.606 the patient is assigned to a high risk group, if greater than or equal to 0.333 and less than 0.606 the patient is assigned to an intermediate risk group and if less than 0.333 the patient is assigned to low risk group.

16. The computer-readable medium of claim 15, further comprising code for generating a likelihood of relapse by comparison of the RFRS score for the patient to a loess fit of RFRS versus likelihood of relapse for a training dataset.

17. A computer-implemented method for evaluating the likelihood of a relapse for a patient that has a lymph node-negative, estrogen receptor-positive, HER2-negative breast cancer, the method comprising:
receiving, at one or more computer systems, information describing the level of expression of the 8 genes, or one or more corresponding alternates thereof, identified in Table 2 in a breast tumor tissue sample obtained from the patient;
performing, with one or more processors associated with the computer system, a random forest analysis in which the level of expression of each gene in the analysis is assigned to a terminal leaf of each decision tree, representing a vote for either “relapse” or no “relapse”;
generating, with the one or more processors associated with the one or more computer systems, a random forest relapse score (RFRS), wherein if the RFRS is greater than or equal to 0.606 the patient is assigned to a high risk group, if greater than or equal to 0.333 and less than 0.606 the patient is assigned to an intermediate risk group and if less than 0.333 the patient is assigned to low risk group.

18. The computer-implemented method of claim 17, further comprising generating, with the one or more processors associated with the one or more computer systems, a likelihood of relapse by comparison of the RFRS score for the patient to a loess fit of RFRS versus likelihood of relapse for a training dataset.

19. A non-transitory computer-readable medium storing program code for evaluating the likelihood of a relapse for a patient that has a lymph node-negative, estrogen receptor-positive, HER2-negative breast cancer in accordance with the method of claim 17, the computer-readable medium comprising:
code for receiving information describing the level of expression of the 8 genes, or one or more corresponding alternates, identified in Table 2 in a breast tumor tissue sample obtained from the patient;
code for performing a random forest analysis in which the level of expression of each gene in the analysis is assigned to a terminal leaf of each decision tree, representing a vote for either “relapse” or no “relapse”; and
code for generating a random forest relapse score (RFRS), wherein if the RFRS is greater than or equal to 0.606 the patient is assigned to a high risk group, if greater than or equal to 0.333 and less than 0.606 the patient is assigned to an intermediate risk group and if less than 0.333 the patient is assigned to low risk group.

20. The non-transitory computer-readable medium storing program of claim 19, further comprising code for generating a likelihood of relapse by comparison of the RFRS score for the patient to a loess fit of RFRS versus likelihood of relapse for a training dataset.
